(19) United States
(12) Patent Application Publication
(10) Pub. No.: US 2023/0192717 A1
(43) Pub. Date: Jun. 22, 2023

(54) COMPOSITIONS AND METHODS OF MODULATING SHORT-CHAIN DEHYDROGENASE ACTIVITY

(71) Applicants: RODEO THERAPEUTICS CORPORATION, Thousand Oaks, CA (US); Case Western Reserve University, Cleveland, OH (US); Board of Regents of the University of Texas System, Austin, TX (US)

(72) Inventors: Stephen L Gwaltney, Chapel Hill, NC (US); Joseph Ready, Carrollton, TX (US); Monika Antczak, Fort Worth, TX (US); Sanford Markowitz, Pepper Pike, OH (US); Amar Desai, Olmstead Falls, OH (US); Stanton Gerson, Hunting Valley, OH (US)

(21) Appl. No.: 17/926,214

(22) PCT Filed: May 19, 2021
(86) PCT No.: PCT/US2021/033170

§ 371 Date: Nov. 18, 2022

Related U.S. Application Data
(60) Provisional application No. 63/027,557, filed on May 20, 2020.

(51) Int. Cl. C07D 495/04; A61K 45/06; C07D 519/00

(52) U.S. Cl. CPC C07D 495/04; A61K 45/06; C07D 519/00

(57) ABSTRACT

Compounds and methods of modulating 15-PGDH activity, modulating tissue prostaglandin levels, treating disease, diseases disorders, or conditions in which it is desired to modulate 15-PGDH activity and/or prostaglandin levels include 15-PGDH inhibitors described herein.

### CROSS-REFERENCE TO RELATED APPLICATIONS

This application claims the benefit of U.S. Provisional Application No. 63/027,557, filed on May 20, 2020, which is incorporated by reference in its entirety.

### BACKGROUND

Short-chain dehydrogenases (SCDs) are a family of dehydrogenases that share only 15% to 30% sequence identity, with similarity predominantly in the coenzyme binding domain and the substrate binding domain. In addition to their role in detoxification of ethanol, SCDs are involved in synthesis and degradation of fatty acids, steroids, and some prostaglandins, and are therefore implicated in a variety of disorders such as lipid storage disease, myopathy, SCD deficiency, and certain genetic disorders.

The SCD, 15-hydroxy-prostaglandin dehydrogenase (15-PGDH), (hydroxyprostaglandin dehydrogenase 15-(nicotinamide adeninedinucleotide); 15-PGDH; Enzyme Commission number 1.1.1.141; encoded by the HPGD gene), represents the key enzyme in the inactivation of a number of active prostaglandins, leukotrienes and hydroxyeicosatetraenoic acids (HETEs) (e.g., by catalyzing oxidation of PGE₂ to 15-keto-prostaglandin E2, 15k-PGE). The human enzyme is encoded by the HPGD gene and consists of a homodimer with subunits of a size of 29 kDa. The enzyme belongs to the evolutionarily conserved superfamily of short-chain dehydrogenase/reductase enzymes (SDRs), and according to the recently approved nomenclature for human enzymes, it is named SDR36C1. Thus far, two forms of 15-PGDH enzyme activity have been identified, NAD+-dependent type I 15-PGDH that is encoded by the HPGD gene, and the type II NADP-dependent 15-PGDH, also known as carbonyl reductase 1 (CBR1, SDR21C1). However, the preference of CBR1 for NADP and the high Km values of CBR1 for most prostaglandin suggest that the majority of the in vivo activity can be attributed to type I 15-PGDH encoded by the HPGD gene, that hereafter, and throughout all following text, simply denoted as 15-PGDH.

Recent studies suggest that inhibitors of 15-PGDH and activators of 15-PGDH could be therapeutically valuable. It has been shown that there is an increase in the incidence of colon tumors in 15-PGDH knockout mouse models. A more recent study implicates increased 15-PGDH expression in the protection of thrombin-mediated cell death. It is well known that 15-PGDH is responsible for the inactivation of prostaglandin E2 (PGE₂), which is a downstream product of COX-2 metabolism. PGE₂ has been shown to be beneficial in a variety of biological processes, such as hair density, dermal wound healing, and bone formation.

### SUMMARY

Embodiments described herein relate to compounds and methods of modulating short chain dehydrogenase (SCD) (e.g., 15-PGDH) activities, modulating tissue prostaglandin levels, and/or treating diseases, disorders, or conditions in which it is desired to modulate SCD (e.g., 15-PGDH) activity and/or prostaglandin levels.

In embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having a structure of formula (I):

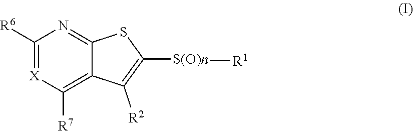

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);

R⁶ is

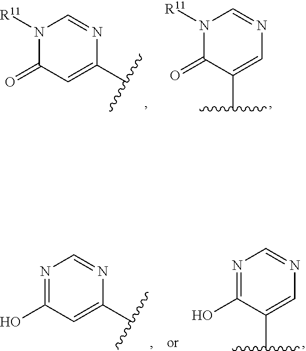

each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —OH, —O-alkeylene-OH, —O-alkeylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹⁰ is —OH, halogen, C₁-C₆ alkyl, or C₁-C₆ alkoxy;
R¹¹ is H or C₁-C₆ alkyl;
X is N or CH;
m is 0, 1, or 2; and
n is 0, 1, or 2.

In embodiments, the compound of formula (I) is not:

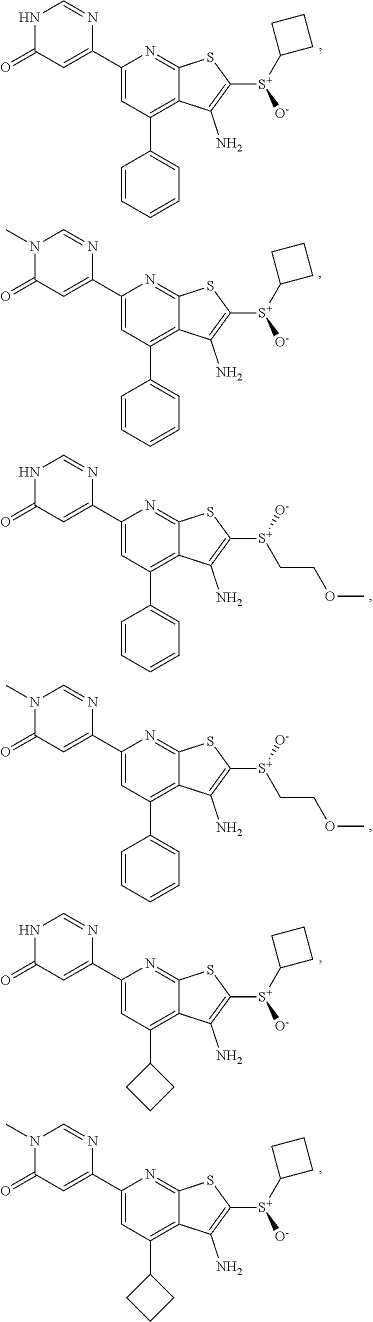 [SMILES: Nc1c([S@+]([O-])C2CCC2)sc2nc(-c3cc(=O)[nH]cn3)cc(-c3ccccc3)c12 ; Cn1cnc(-c2cc(-c3ccccc3)c3c(N)c([S@+]([O-])C4CCC4)sc3n2)cc1=O ; COCC[S@@+]([O-])c1sc2nc(-c3cc(=O)[nH]cn3)cc(-c3ccccc3)c2c1N ; COCC[S@@+]([O-])c1sc2nc(-c3cc(=O)n(C)cn3)cc(-c3ccccc3)c2c1N ; Nc1c([S@+]([O-])C2CCC2)sc2nc(-c3cc(=O)[nH]cn3)cc(C3CCC3)c12 ; Cn1cnc(-c2cc(C3CCC3)c3c(N)c([S@+]([O-])C4CCC4)sc3n2)cc1=O]

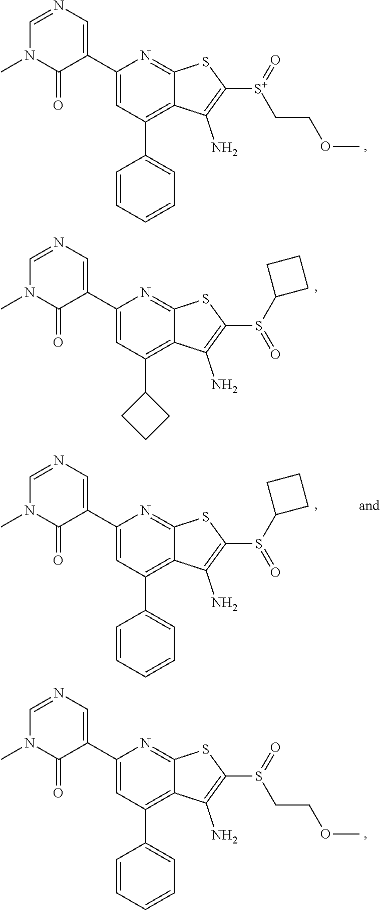

In embodiments of compounds of formula (I), R¹ is C₁-C₆ alkyl, C₃-C₆ cycloalkyl, or —(C₁-C₃ alkylene)-(C₁-C₃ alkoxy). In embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

In embodiments of compounds of formula (I), R² is NH₂.

In embodiments of compounds of formula (I), R⁶ is

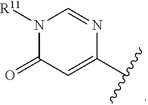

In embodiments of compounds of formula (I), R¹¹ is H or methyl.

In embodiments of compounds of formula (I), R⁷ is phenyl, alkyl, or cycloalkyl, each of which is optionally substituted with one or more R⁴.

In embodiments of compounds of formula (I), R⁷ is a linear or branched, non-cyclic C₁-C₆ alkyl. In embodiments, R⁷ is methyl, ethyl, n-propyl, i-propyl, n-butyl, s-butyl, or t-butyl. In embodiments, R⁷ is i-propyl.

In embodiments of compounds of formula (I), X is CH.

In embodiments of compounds of formula (I), n is 1.

The present disclosure also relates to compounds of formula (II):

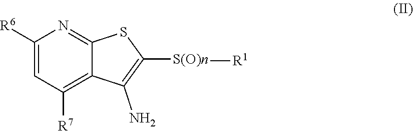

or a pharmaceutically acceptable salt, tautomer, or solvate thereof, wherein:
R¹ is C₁-C₆ alkyl, C₃-C₆ cycloalkyl, or —(C₁-C₃ alkylene)-(C₁-C₃ alkoxy);
R⁶ is

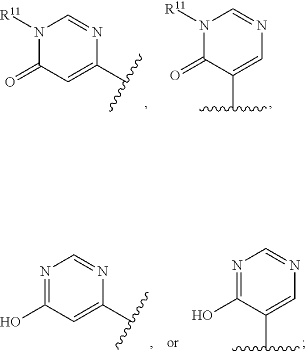

R⁷ is a linear or branched, non-cyclic C₁-C₆ alkyl;
R¹¹ is H or C₁-C₆ alkyl; and
n is 0, 1, or 2.

In embodiments of compounds of formula (I) or (II), the compound is selected from:

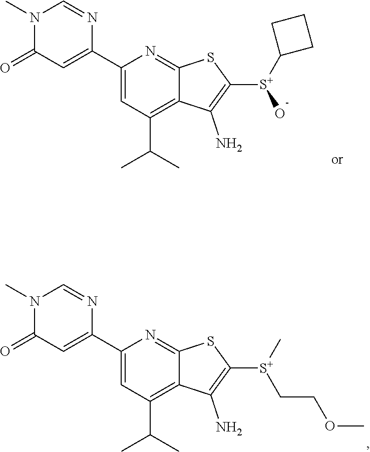

or a pharmaceutically acceptable salt, tautomer, or solvate thereof.

The present disclosure also relates to compounds of formula (III):

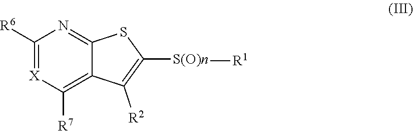

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

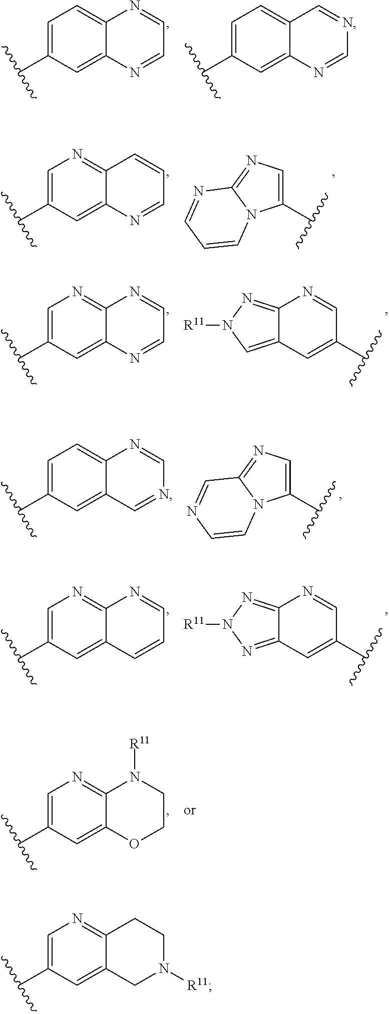

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R¹ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹⁰ is H or C₁-C₆ alkyl;
X is N or CH;
m is 0, 1, or 2; and
n is 0, 1, or 2.

In embodiments of the compounds of formula (III), the compound is not:

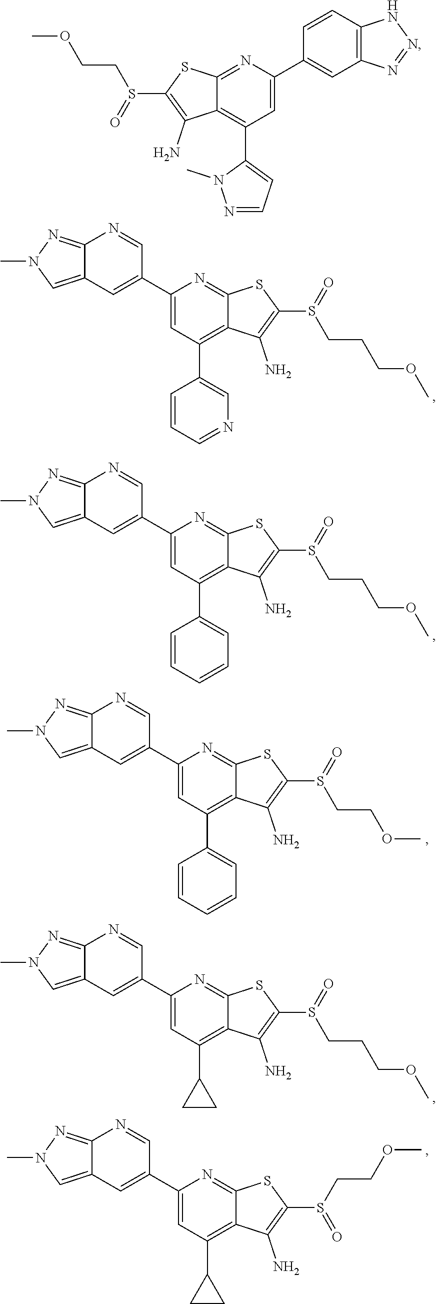

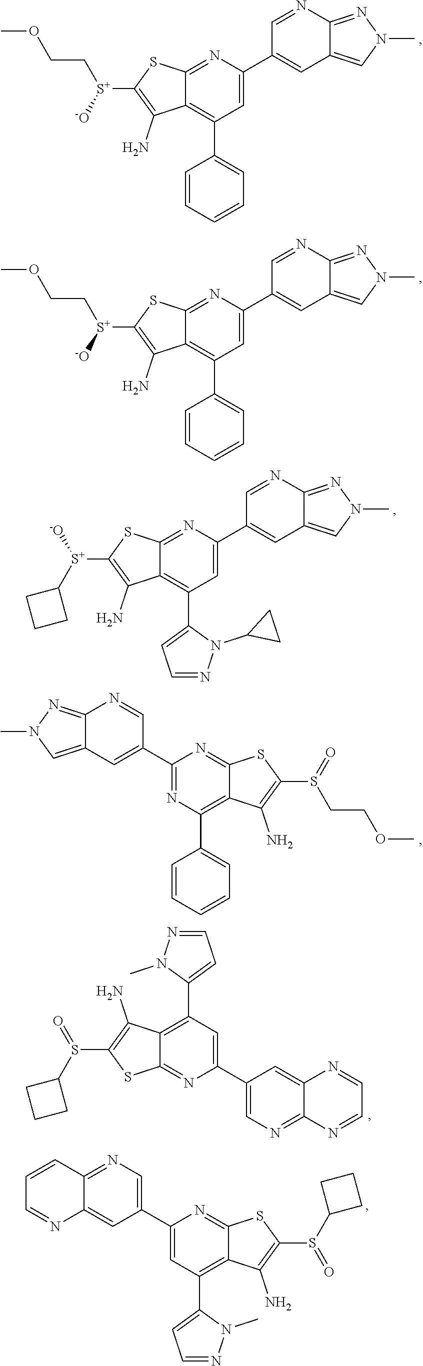

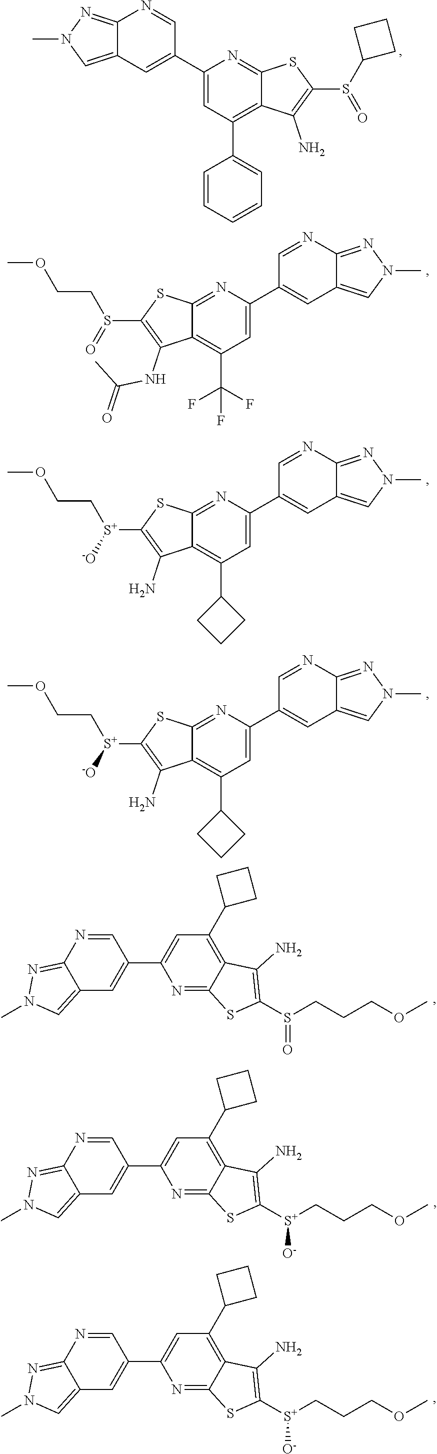

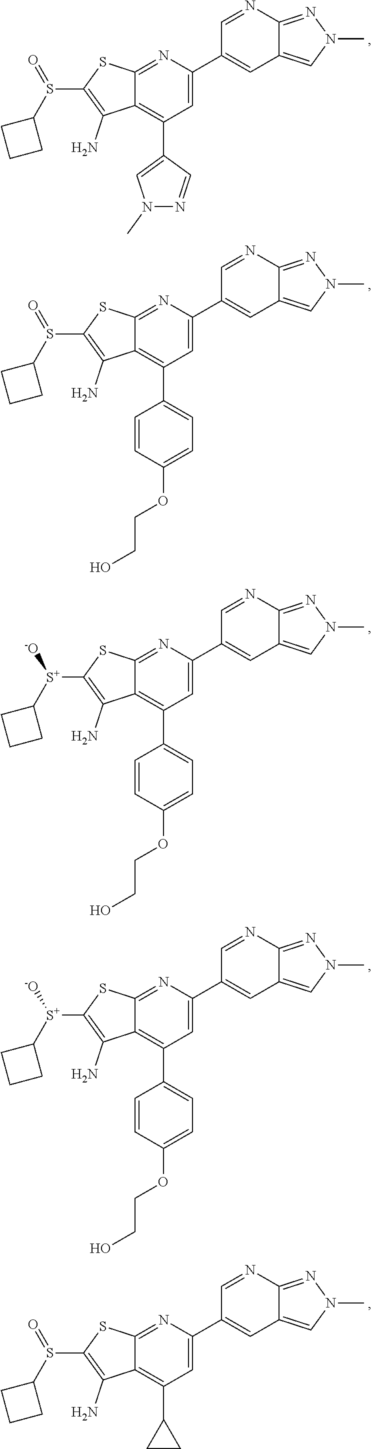

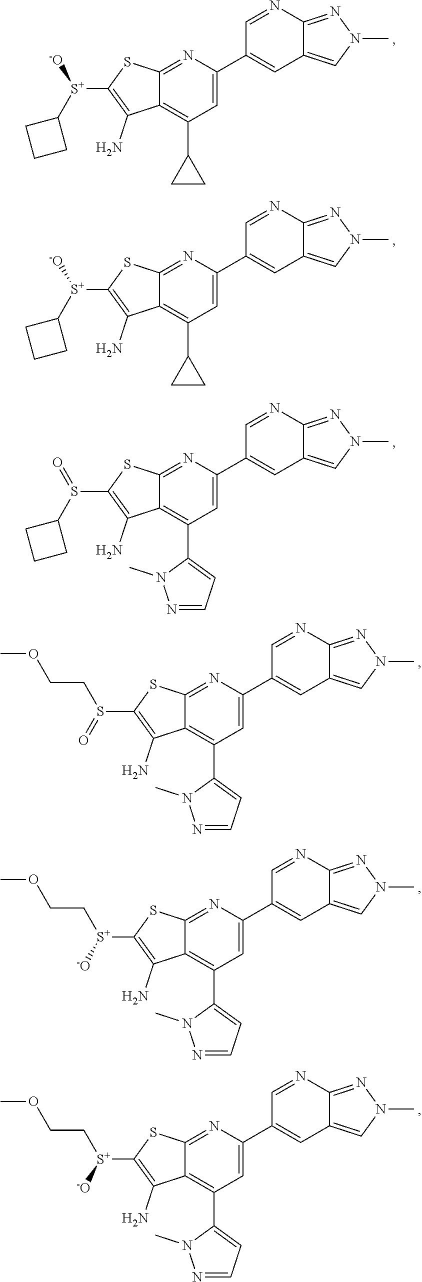

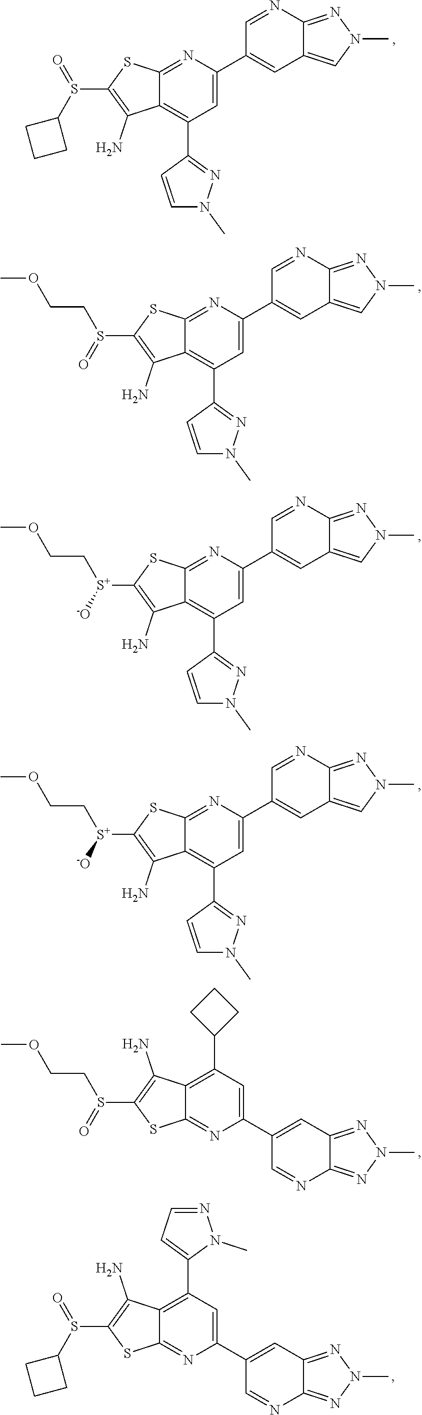

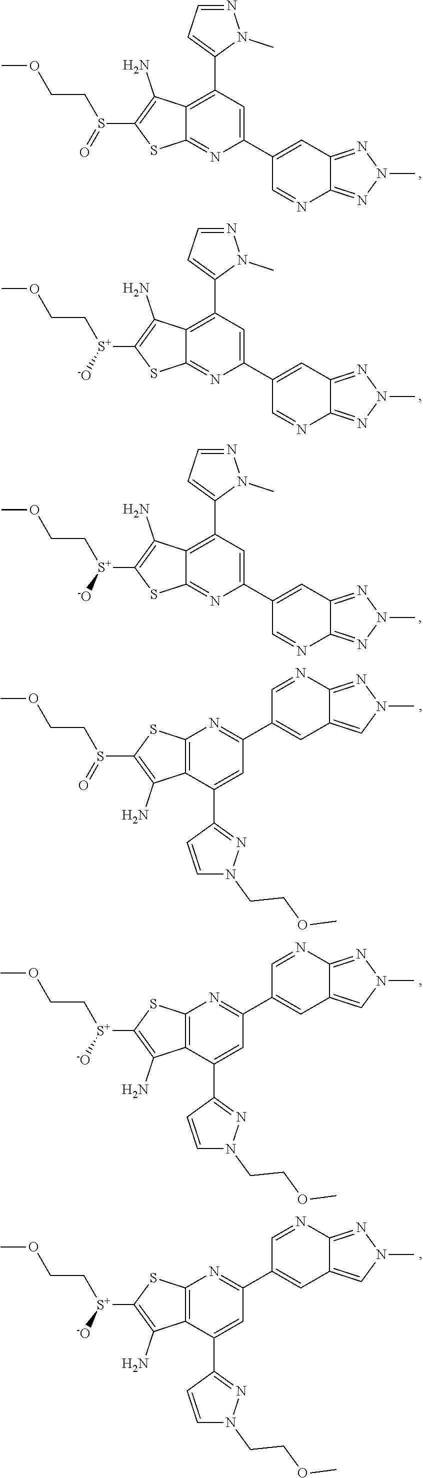

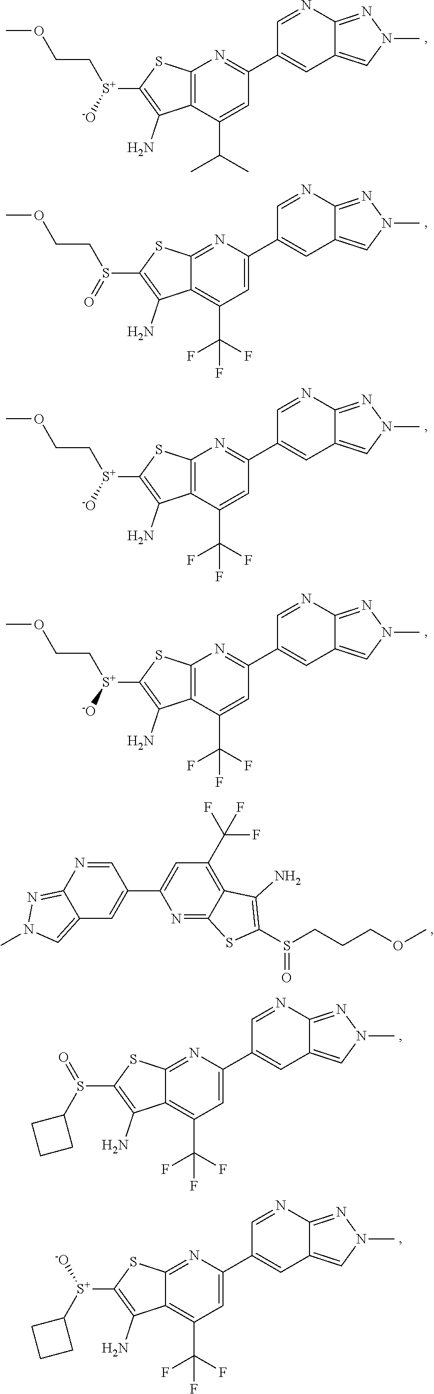

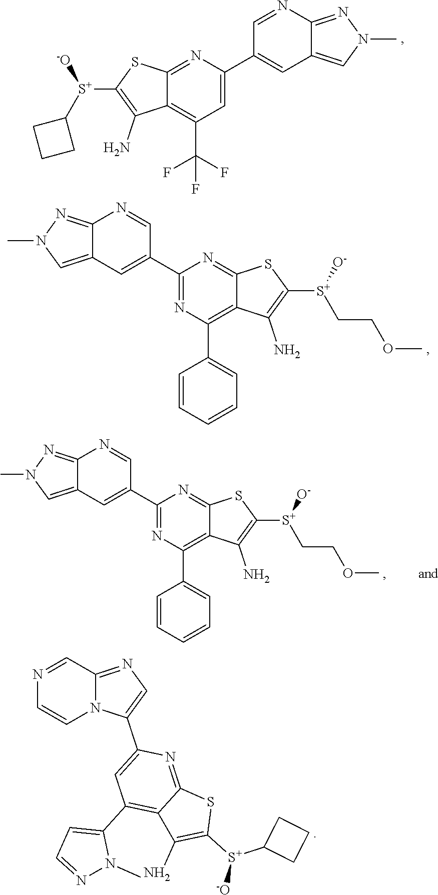

In embodiments of compounds of formula (III), R¹ is C₁-C₆ alkyl, C₃-C₆ cycloalkyl, or —(C₁-C₃ alkylene)-(C₁-C₃ alkoxy). In embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

In embodiments of compounds of formula (III), R² is NH₂ or —CN.

In embodiments of compounds of formula (III), R⁶ is

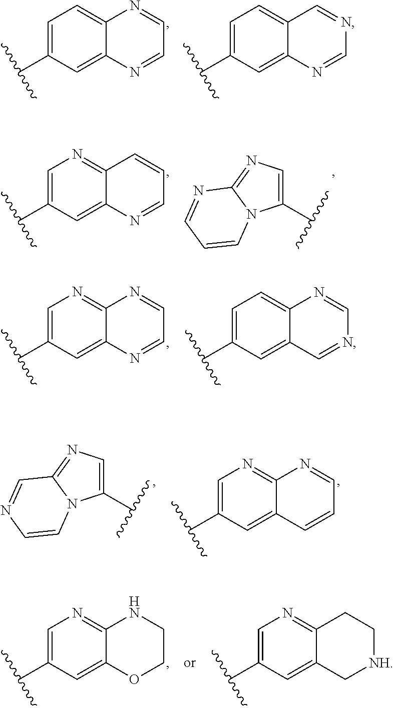

In embodiments of compounds of formula (III), R⁷ is alkyl, cycloalkyl, aryl, heterocyclyl, or heteroaryl, each of which is optionally substituted with one or more R⁴.

In embodiments of compounds of formula (III), n is 1.

In embodiments of compounds of formula (III), the compound is selected from:

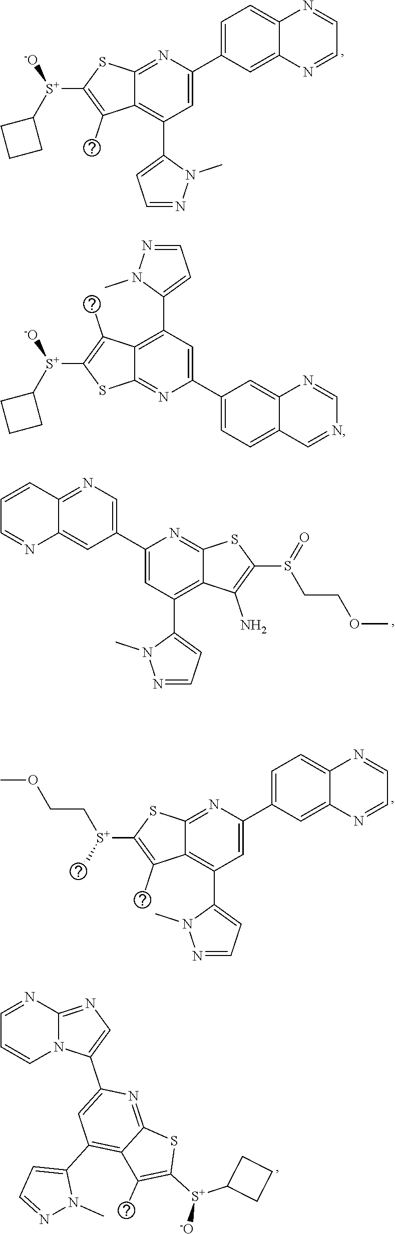

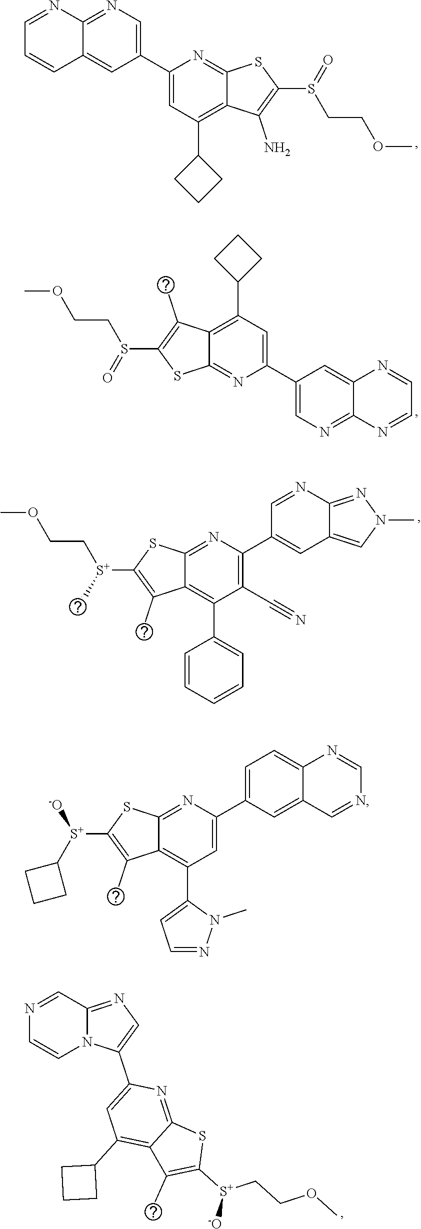

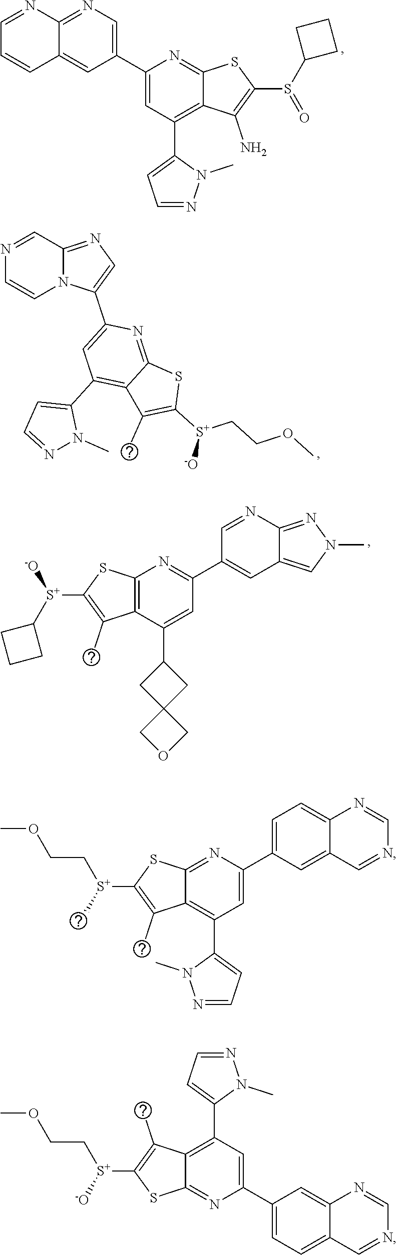

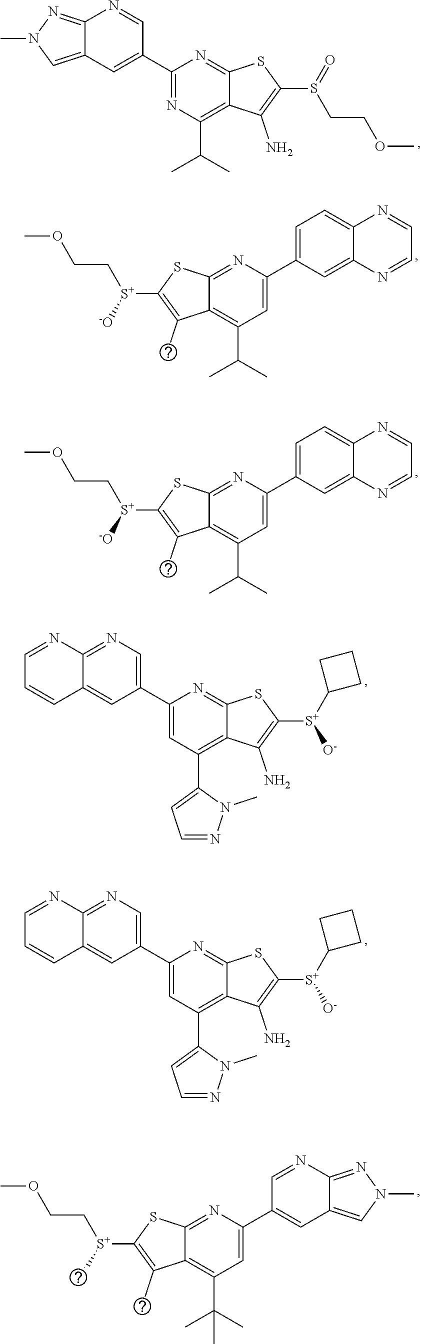

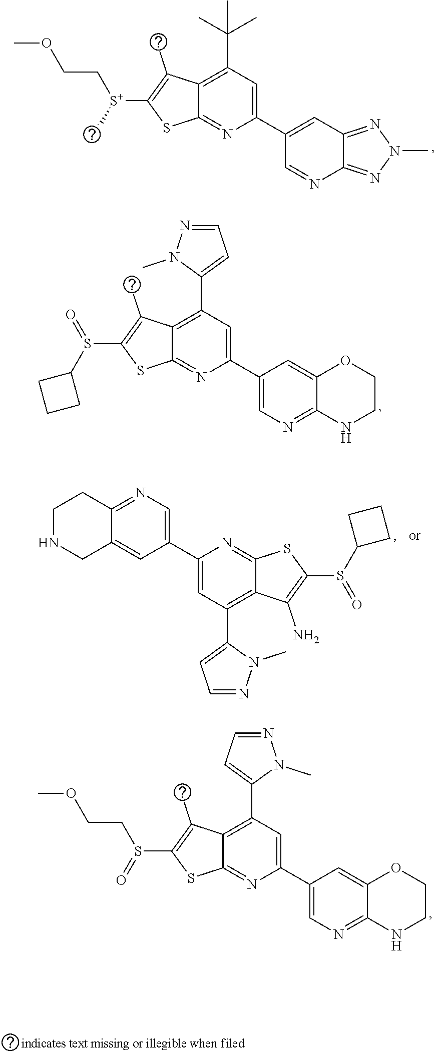

or a pharmaceutically acceptable salt, tautomer, or solvate thereof.

The present disclosure also relates to compounds of formula (IV):

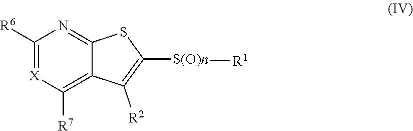

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

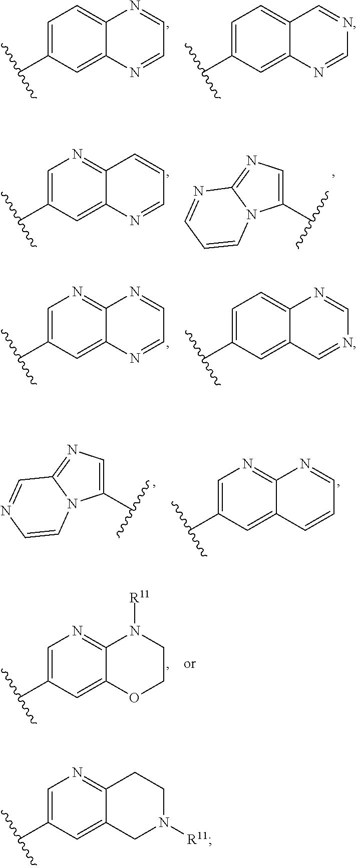

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2;
wherein the compound is not:

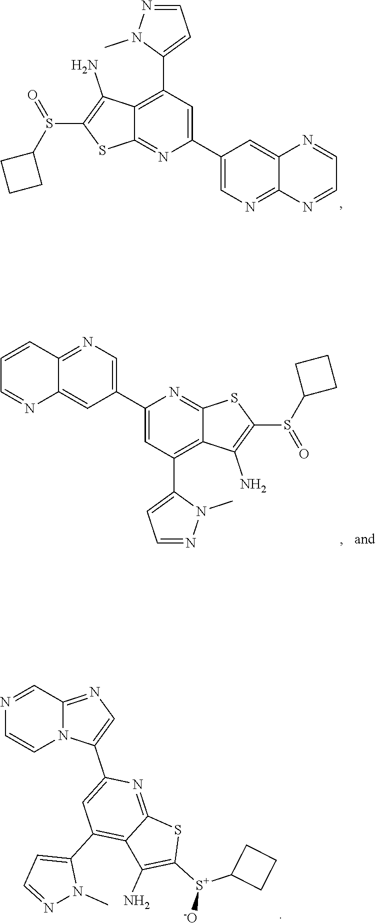

The present disclosure also relates to compounds of:

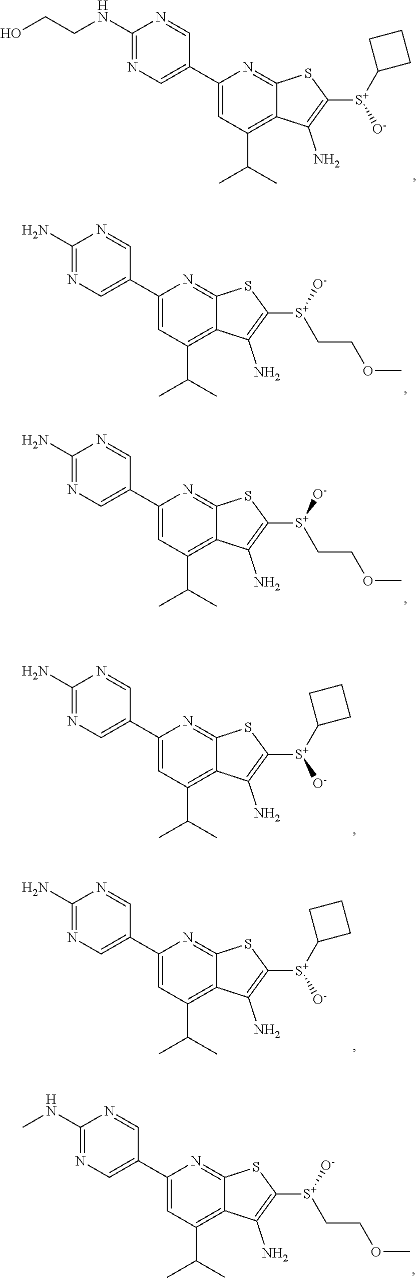

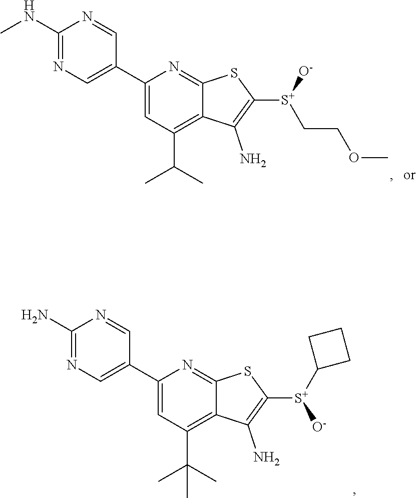

or a pharmaceutically acceptable salt, tautomer, or solvate thereof.

The present disclosure also relates to a pharmaceutical composition comprising any one of compounds of formula (I)-(IV) or compounds of Table 1, or a pharmaceutically acceptable salt, tautomer, or solvate thereof, and a pharmaceutically acceptable carrier or excipient.

In embodiments, the compound or 15-PGDH inhibitor of the present disclosure can inhibit the enzymatic activity of recombinant 15-PGDH at an IC₅₀ of less than or equal to 1 μM, an IC₅₀ of less than or equal to 250 nM, an IC₅₀ of less than or equal to 50 nM, an IC₅₀ of less than or equal to 10 nM, an IC₅₀ of less than or equal to 5 nM, an IC₅₀ of about 2.5 nM to about 10 nM, or an IC₅₀ of less than or equal to about 2.5 nM, at a 15-PGDH concentration of about 1 nM to about 10 nM.

In embodiments, the compound or 15-PGDH inhibitor of the present disclosure can inhibit the enzymatic activity of recombinant 15-PGDH at an IC₅₀ of less than or equal to 1 μM, an IC₅₀ of less than or equal to 250 nM, an IC₅₀ of less than or equal to 50 nM, an IC₅₀ of less than or equal to 10 nM, an IC₅₀ of less than or equal to 5 nM, at an IC₅₀ of about 2.5 nM to about 10 nM, or an IC₅₀ of less than or equal to about 2.5 nM, at a 15-PGDH concentration of about 0.5 nM to about 5 nM.

In embodiments, the compound or 15-PGDH inhibitor of the present disclosure can inhibit the enzymatic activity of recombinant 15-PGDH at an IC₅₀ of less than or equal to 1 μM, an IC₅₀ of less than or equal to 250 nM, an IC₅₀ of less than or equal to 50 nM, an IC₅₀ of less than or equal to 10 nM, an IC₅₀ of less than or equal to 5 nM, at an IC₅₀ of about 2.5 nM to about 10 nM, or an IC₅₀ of less than about or equal to 2.5 nM, at a 15-PGDH concentration of about 1 nM to about 2 nM. In embodiments, the compound or 15-PGDH inhibitor of the present disclosure can inhibit the enzymatic activity of recombinant 15-PGDH at an IC₅₀ of less than about 2.5 nM, at a 15-PGDH concentration of about 1 nM to about 2 nM.

The 15-PGDH inhibitor of the present disclosure can be provided in a topical composition that can be applied to skin of a subject to promote and/or stimulate pigmentation of the skin and/or hair growth and/or inhibiting hair loss, and/or treat skin damage or inflammation.

The 15-PGDH inhibitor of the present disclosure can also be administered to a subject to promote wound healing, tissue repair, and/or tissue regeneration and/or engraftment or regeneration of a tissue graft.

In embodiments, the 15-PGDH inhibitor of the present disclosure can be administered to a subject to treat at least one of oral ulcers, gum disease, colitis, ulcerative colitis, gastrointestinal ulcers, inflammatory bowel disease, vascular insufficiency, Raynaud's disease, Buerger's disease, diabetic neuropathy, pulmonary artery hypertension, cardiovascular disease, and renal disease.

In another embodiment, the 15-PGDH inhibitor of the present disclosure can be administered to a subject in combination with a prostanoid agonist for the purpose of enhancing the therapeutic effect of the agonist in prostaglandin responsive conditions.

In embodiments, the 15-PGDH inhibitor of the present disclosure can be administered to a subject and/or tissue of the subject to increase tissue stem cells. For example, the 15-PGDH inhibitor can be administered to bone marrow of a subject to increase stem cells in the subject.

In still other embodiments, the 15-PGDH inhibitor of the present disclosure can be administered to a tissue graft donor, bone marrow graft donor, and/or a hematopoietic stem cell donor, and/or a tissue graft, and/or a bone marrow graft, and/or a hematopoietic stem cell graft, to increase the fitness of a donor tissue graft, a donor bone marrow graft, and/or a donor hematopoietic stem cell graft. In embodiments, the 15-PGDH inhibitor is administered ex vivo to a tissue graft, and/or a bone marrow graft, and/or a hematopoietic stem cell graft. For example, the 15-PGDH inhibitor can be administered to a subject, and/or bone marrow of a subject to increase the fitness of the marrow as a donor graft, and/or to a preparation of hematopoietic stem cells of a subject to increase the fitness of the stem cell preparation as a donor graft, and/or to a preparation of peripheral blood hematopoietic stem cells of a subject to increase the fitness of the stem cell preparation as a donor graft, and/or to a preparation of umbilical cord blood stem cells to increase the fitness of the stem cell preparation as a donor graft, and/or to a preparation of umbilical cord blood stem cells to decrease the number of units of umbilical cord blood required for transplantation.

In embodiments, the 15-PGDH inhibitor of the present disclosure can be administered to a subject to mitigate tissue graft rejection, to enhance tissue and/or bone marrow graft engraftment, to enhance bone marrow graft engraftment, following treatment of the subject or the marrow of the subject with radiation therapy, chemotherapy, or immunosuppressive therapy, to enhance engraftment of a progenitor stem cell graft, hematopoietic stem cell graft, or an umbilical cord blood stem cell graft, to enhance engraftment of a hematopoietic stem cell graft, or an umbilical cord stem cell graft, following treatment of the subject or the marrow of the subject with radiation therapy, chemotherapy, or immunosuppressive therapy, and/or in order to decrease the number of units of umbilical cord blood required for transplantation into the subject.

In embodiments, the 15-PGDH inhibitor of the present disclosure can be administered to a recipient of a tissue graft transplant, bone marrow transplant, and/or hematopoietic stem cell transplant, or of an umbilical cord stem cell transplant, in order to decrease the administration of other treatments or growth factors.

In embodiments, the 15-PGDH inhibitor of the present disclosure can be administered to a subject or to a tissue graft of a subject to mitigate graft rejection, to enhance graft engraftment, and/or to enhance graft engraftment following treatment of the subject or the marrow of the subject with radiation therapy, chemotherapy, or immunosuppressive therapy.

In embodiments, the 15-PGDH inhibitor of the present disclosure can be administered to a subject or to the bone marrow of a subject to confer resistance to toxic or lethal effects of exposure to radiation, to confer resistance to the toxic effect of Cytoxan, the toxic effect of fludarabine, the toxic effect of chemotherapy, or the toxic effect of immunosuppressive therapy, to decrease pulmonary toxicity from radiation, and/or to decrease infection.

In still other embodiments, the 15-PGDH inhibitor of the present disclosure can be administered to a subject to increase neutrophil counts following a hematopoietic cell transplant with bone marrow, hematopoietic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoietic cell transplant with bone marrow, hematopoietic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoietic cell transplant with bone marrow, hematopoietic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoietic cell transplant with bone marrow, hematopoietic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In embodiments, the administration of a 15-PGDH inhibitor of the present disclosure can be used to modulate hematopoietic stem cells and hematopoiesis. For a 15-PGDH inhibitor can be administered alone or in combination with a cytokine to a subject in need thereof to increase and/or mobilize hematopoietic stem cells and/or neutrophils in the blood, marrow, and/or tissue of the subject.

In embodiments, the administration of a 15-PGDH inhibitor of the present disclosure can be in combination with G-CSF for the purpose of increasing neutrophils.

In embodiments, the administration of a 15-PGDH inhibitor of the present disclosure can be in combination with a hematopoietic cytokine for the purpose of increasing neutrophils.

In still other embodiments, the administration of a 15-PGDH inhibitor of the present disclosure can be in combination with G-CSF for the purpose of increasing numbers of and/or of mobilizing peripheral blood hematopoietic stem cells.

In embodiments, the administration of a 15-PGDH inhibitor of the present disclosure can be in combination with a hemopoietic cytokine for the purpose of increasing numbers of and/or of mobilizing peripheral blood hematopoietic stem cells.

In embodiments, the administration of a 15-PGDH inhibitor of the present disclosure can be in combination with a second agent, including Plerixafor, for the purpose of increasing numbers of and/or of mobilizing peripheral blood hematopoietic stem cells.

In embodiments, the administration of a 15-PGDH inhibitor of the present disclosure can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor of the present disclosure can be in combination with a hemopoietic cytokine for the purpose of increasing numbers of and/or of mobilizing peripheral blood hematopoietic stem cells for use in hematopoietic stem cell transplantation.

In embodiments, the administration of a 15-PGDH inhibitor of the present disclosure can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor of the present disclosure can be in combination with G-CSF for the purpose of increasing numbers of hematopoietic stem cells in blood or bone marrow.

In embodiments, the administration of a 15-PGDH inhibitor of the present disclosure can be in combination with a hemopoietic cytokine for the purpose of increasing numbers of hematopoietic stem cells in blood or bone marrow.

In embodiments, the 15-PGDH inhibitor of the present disclosure can be administered to a subject and/or tissue of the subject to increase tissue stem cells. For example, the 15-PGDH inhibitor can be administered to bone marrow of a subject to increase stem cells in the subject.

In embodiments, the 15-PGDH inhibitor of the present disclosure can be administered to a recipient of a tissue graft transplant, bone marrow transplant, and/or hematopoietic stem cell transplant, or of an umbilical cord stem cell transplant, in order to decrease the administration of other treatments or growth factors.

In still other embodiments, the 15-PGDH inhibitor of the present disclosure can be administered to a subject to increase neutrophil counts following a hematopoietic cell transplant with bone marrow, hematopoietic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoietic cell transplant with bone marrow, hematopoietic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoietic cell transplant with bone marrow, hematopoietic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoietic cell transplant with bone marrow, hematopoietic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In embodiments, the 15-PGDH inhibitor of the present disclosure can be administered to a subject to increase responsiveness to cytokines in the presence of cytopenias, with cytopenias including any of: neutropenia, thrombocytopenia, lymphocytopenia and anemia; and with cytokines having increased responsiveness potentiated by the 15-PGDH inhibitor including any of: G-CSF, GM-CSF, EPO, IL-3, IL-6, TPO, TPO-RA (thrombopoietin receptor agonist), and SCF.

In embodiments, the 15-PGDH inhibitor of the present disclosure can be administered to a subject to increase bone density, treat osteoporosis, promote healing of fractures, or promote healing after bone surgery or joint replacement and/or to promote healing of bone to bone implants, bone to artificial implants, dental implants, and bone grafts.

In embodiments, the 15-PGDH inhibitor of the present disclosure can be administered to a subject or to the intestine of a subject to increase stem cells or cell proliferation in the intestine and/or and confer resistance to toxic or lethal effects of exposure to radiation or the toxic, lethal, or mucositis effects resultant from treatment with chemotherapy.

In embodiments, the 15-PGDH inhibitor of the present disclosure can be administered to a subject or to intestine of a subject as a treatment for colitis, ulcerative colitis, or inflammatory bowel disease.

In embodiments, the 15-PGDH inhibitor of the present disclosure can be administered to a subject to increase liver regeneration following liver surgery, following live liver donation, following liver transplantation, or following liver injury by toxins and/or to promote recovery from or resistance to liver toxins, including acetaminophen and related compounds.

In still other embodiments, the 15-PGDH inhibitor of the present disclosure can be administered to a subject to treat erectile dysfunction.

In yet other embodiments, the 15-PGDH inhibitor of the present disclosure can be administered to inhibit at least one of the growth, proliferation, or metastasis of 15-PGDH expressing cancers.

Still other embodiments described herein relate to a method of treating a subject in need of cell therapy. The method includes administering to the subject a therapeutically effective amount of a preparation comprising human hematopoietic stem cell administered a 15-PGDH inhibitor described herein and/or a therapeutic composition comprising human hematopoietic stem cells and a 15-PGDH inhibitor described herein.

In embodiments, the subject has received human hematopoietic stem cells and/or has received the preparation and/or the therapeutic composition.

In embodiments, the subject has acute myelogenous leukemia (AML), acute lymphoblastic leukemia (ALL), chronic myelogenous leukemia (CML), chronic lymphocytic leukemia (CLL), juvenile myelomonocytic leukemia, Hodgkin's lymphoma, non-Hodgkin's lymphoma, multiple myeloma, severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, mucopolysaccharidoses, or Diamond Blackfan anemia.

Other embodiments relate to a method of treating a subject having at least one symptom associated with an ischemic tissue or a tissue damaged by ischemia. The method includes administering to the subject a therapeutically effective amount of a preparation comprising human hematopoietic stem cell administered a 15-PGDH inhibitor described herein and/or a therapeutic composition comprising human hematopoietic stem cells and a 15-PGDH inhibitor described herein.

In embodiments, the ischemia can be associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other embodiments relate to methods for treating and/or preventing fibrosis and various fibrotic diseases, disorders or conditions by administration of 15-PGDH inhibitors. In embodiments, a 15-PGDH inhibitor described herein can be administered to a subject in need thereof to decrease fibrotic symptoms, such as collagen deposition, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components.

Fibrotic diseases, disorders and conditions characterized, in whole or in part, by excess production of fibrotic material can include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myeloid leukemia, acute myelogenous leukemia, myelodysplastic syndrome, myeloproliferative syndrome, gynecological cancer, Kaposi's sarcoma, Hansen's disease, collagenous colitis, acute fibrosis, organ specific fibrosis, and the like.

In embodiments, a method of treating or preventing a fibrotic disease, disorder or condition includes administering to a subject in need thereof a therapeutically effect amount of a 15-PGDH inhibitor of the present disclosure.

In embodiments, the 15-PGDH inhibitors of the present disclosure can be used to treat or prevent lung fibrosis. Lung fibrosis, which can be treated, can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In embodiments, the 15-PGDH inhibitors of the present disclosure can be used to treat or prevent kidney fibrosis. The kidney fibrosis can result from dialysis following kidney failure, catheter placement, a nephropathy, glomerulosclerosis, glomerulonephritis, chronic renal insufficiency, acute kidney injury, end stage renal disease or renal failure, or combinations thereof.

In embodiments, the 15-PGDH inhibitors of the present disclosure can be used to treat or prevent liver fibrosis. The liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

In embodiments, the 15-PGDH inhibitors of the present disclosure can be used to treat or prevent heart fibrosis, for example, cardiac fibrosis and endomyocardial fibrosis.

In embodiments, the 15-PGDH inhibitors of the present disclosure can be used to treat or prevent systemic sclerosis.

In embodiments, the 15-PGDH inhibitors of the present disclosure can be used to treat or prevent fibrotic diseases, disorders or conditions caused by post-surgical adhesion formation.

In embodiments, the 15-PGDH inhibitors of the present disclosure can be used for reducing or preventing scar formation in a subject.

In embodiments, the 15-PGDH inhibitors of the present disclosure can be used to reduce or prevent scar formation on skin or scleroderma.

In various embodiments, the 15-PGDH inhibitors of the present disclosure can be administered at a therapeutically effective amount such that at least one symptom or feature of a fibrotic disease, disorder or condition, or other related diseases, disorders or conditions, is reduced in intensity, severity, or frequency, or has delayed onset.

In embodiments, the 15-PGDH inhibitors of the present disclosure can be used in a method for decreasing or reducing collagen secretion or collagen deposition in a tissue or organ, such as the lung, the liver, the intestines, the colon, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult can be organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

Other embodiments described herein relate to the use of 15-PGDH inhibitors of the present disclosure in combination with corticosteroids or TNF inhibitors to treat inflammation, reduce aberrant activity of the immune system, and/or promote wound healing in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory, disorders, immune disorders, and/or wounds by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects.

In embodiments, the inflammatory and/or immune disease or disorder treated with the combination of 15-PGDH inhibitor of the present disclosure and a corticosteroid or TNF inhibitor can include intestinal, gastrointestinal, or bowel disorders. As described below, it was found that inhibitors of short-chain dehydrogenase activity, such as 15-PGDH inhibitors, can be administered to a subject in need thereof alone or in combination with corticosteroids and tumor necrosis factor (TNF)-alpha antagonists to treat intestinal, gastrointestinal, or bowel disorders, such as oral ulcers, gum disease, gastritis, colitis, ulcerative colitis, gastric ulcers, inflammatory bowel disease, and Crohn's disease.

In embodiments, the 15-PGDH inhibitor of the present disclosure can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. For example, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids.

The 15-PGDH inhibitor of the present disclosure can also be administered in combination with a corticosteroid or TNF inhibitor to a subject to promote wound healing, tissue repair, and/or tissue regeneration and/or engraftment or regeneration of a tissue graft.

In embodiments, the 15-PGDH inhibitor of the present disclosure can be administered to a subject at an amount effective to increase prostaglandin levels in the subject and attenuate corticosteroid induced adverse and/or cytotoxic effects.

### DETAILED DESCRIPTION

### Definitions

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethyl amine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

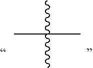

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

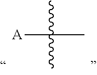

indicates that the chemical entity “A” is bonded to another chemical entity via the point of attachment bond. Furthermore, the specific point of attachment to the non-depicted chemical entity can be specified by inference. For example, the compound

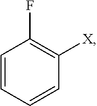

wherein X is

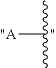

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

The term “neoplasm” refers to any abnormal mass of cells or tissue as a result of neoplasia. The neoplasm may be benign, potentially malignant (precancerous), or malignant (cancerous). An adenoma is an example of a neoplasm.

The terms “adenoma”, “colon adenoma” and “polyp” are used herein to describe any precancerous neoplasm of the colon.

The term “colon” as used herein is intended to encompass the right colon (including the cecum), the transverse colon, the left colon and the rectum.

The terms “colorectal cancer” and “colon cancer” are used interchangeably herein to refer to any cancerous neoplasia of the colon (including the rectum, as defined above).

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.) Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

Embodiments described herein relate to compounds and methods of modulating SCD activity (e.g., 15-PGDH activity), modulating tissue prostaglandin levels, and/or treating diseases, disorders, or conditions in which it is desired to modulate 15-PGDH activity and/or prostaglandin levels.

“Inhibitors,” “activators,” and “modulators” of 15-PGDH expression or of 15-PGDH activity are used to refer to inhibitory, activating, or modulating molecules, respectively, identified using in vitro and in vivo assays for 15-PGDH expression or 15-PGDH activity, e.g., ligands, agonists, antagonists, and their homologs and mimetics. The term “modulator” includes inhibitors and activators. Inhibitors are agents that, e.g., inhibit expression of 15-PGDH or bind to, partially or totally block stimulation, decrease, prevent, delay activation, inactivate, desensitize, or down regulate the activity of 15-PGDH, e.g., antagonists. Activators are agents that, e.g., induce or activate the expression of a 15-PGDH or bind to, stimulate, stabilize, increase, open, activate, facilitate, or enhance activation, sensitize or up regulate the activity of 15-PGDH, e.g., agonists. Modulators include naturally occurring and synthetic ligands, small chemical molecules, and the like.

### Compounds of the Disclosure

15-PGDH inhibitors described herein can provide a pharmacologic method for elevating prostaglandin levels in tissue. Known activities of prostaglandins include promoting hair growth, promoting skin pigmentation, and promoting skin darkening or the appearance of skin tanning. Known activities of prostaglandins also include ameliorating pulmonary artery hypertension. 15-PGDH inhibitors described herein may also be utilized to increase tissue stem cell numbers for purposes that would include increasing resistance to tissue damage by radiation, increasing resistance to environmental exposures to radiation, increasing stem cell numbers to increase fitness of bone marrow or other types of transplantation (through either in vivo exposure to 15-PGDH inhibitors described herein to increase stem cell numbers prior to harvest of a transplanted tissue, or through ex vivo exposure of a harvested tissue prior to transplant into a recipient host, or through treatment of the graft recipient). 15-PGDH inhibitors described herein may also be utilized for purposes that would include promoting liver regeneration, including liver regeneration after liver resection, and liver regeneration after toxic insults, which for example may be the toxic insult of acetaminophen overdose. Prostaglandin signaling is also known to promote wound healing, protect the stomach from ulceration, and promote healing of ulcers of stomach and intestines. Additionally, 15-PGDH inhibitors described herein can promote activity of human keratinocytes in “healing” scratches across cultures of keratinocyte cells. Hence, 15-PGDH inhibitors described herein may be utilized to also heal ulcers of other tissues, including, but not limited to skin, and including but not limited to diabetic ulcers. Further, 15-PGDH inhibitors described herein may be utilized for the treatment of erectile dysfunction.

15-PGDH inhibitors described herein can be identified using assays in which putative modulator compounds are applied to cells expressing 15-PGDH and then the functional effects on 15-PGDH activity are determined. Samples or assays comprising 15-PGDH that are treated with a potential activator, inhibitor, or modulator are compared to control samples without the inhibitor, activator, or modulator to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as modulators of SCD (e.g., 15-PGDH) can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays). Modulators also include agents designed to increase the level of 15-PGDH mRNA or the level of translation from an mRNA.

In embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having a structure of formula (I):

The 15-PGDH inhibitor can include a compound having a structure of formula (I):

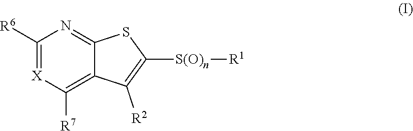

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

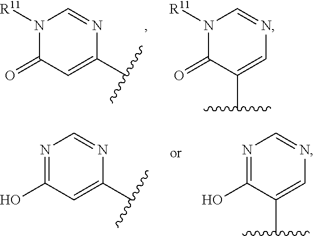

each of which is optionally substituted with one or more R³;
R⁷ is alkyl, haloalkyl, cycloalkyl, aryl, heterocyclyl, heteroaryl, —C(O)-alkyl,
—C(O)O-alkyl, or —C(O)NR⁵-alkyl, each of which is optionally substituted with one or more R⁴;
R³ is —OH, —O-alkeylene-OH, —O-alkeylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R^{m};
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹⁰ is —OH, halogen, C₁-C₆ alkyl, or C₁-C₆ alkoxy;
R¹¹ is H or C₁-C₆ alkyl;
X is N or CH;
m is 0, 1, or 2; and
n is 0, 1, or 2.

In embodiments, the compound of formula (I) is not:

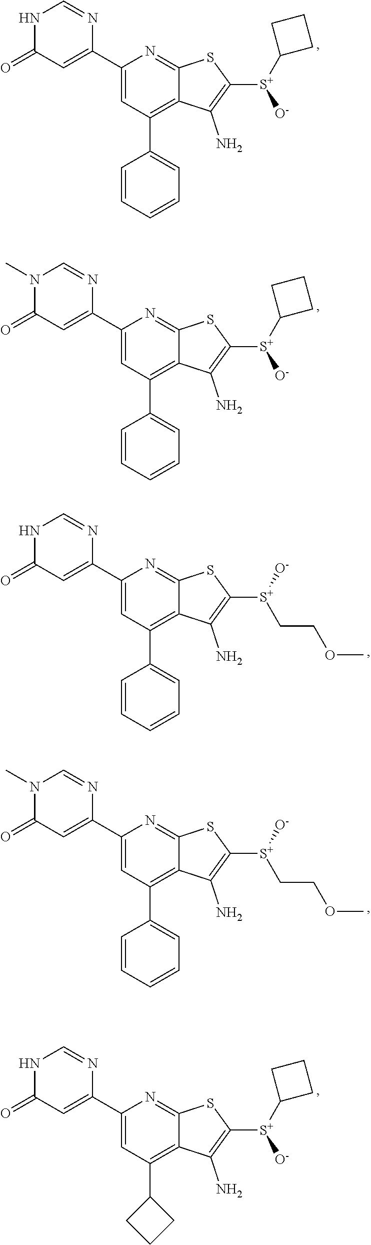 [SMILES: Nc1c([S@+]([O-])C2CCC2)sc2nc(-c3cc(=O)[nH]cn3)cc(-c3ccccc3)c12 ; Cn1cnc(-c2cc(-c3ccccc3)c3c(N)c([S@+]([O-])C4CCC4)sc3n2)cc1=O ; COCC[S@@+]([O-])c1sc2nc(-c3cc(=O)[nH]cn3)cc(-c3ccccc3)c2c1N ; COCC[S@@+]([O-])c1sc2nc(-c3cc(=O)n(C)cn3)cc(-c3ccccc3)c2c1N ; Nc1c([S@+]([O-])C2CCC2)sc2nc(-c3cc(=O)[nH]cn3)cc(C3CCC3)c12]

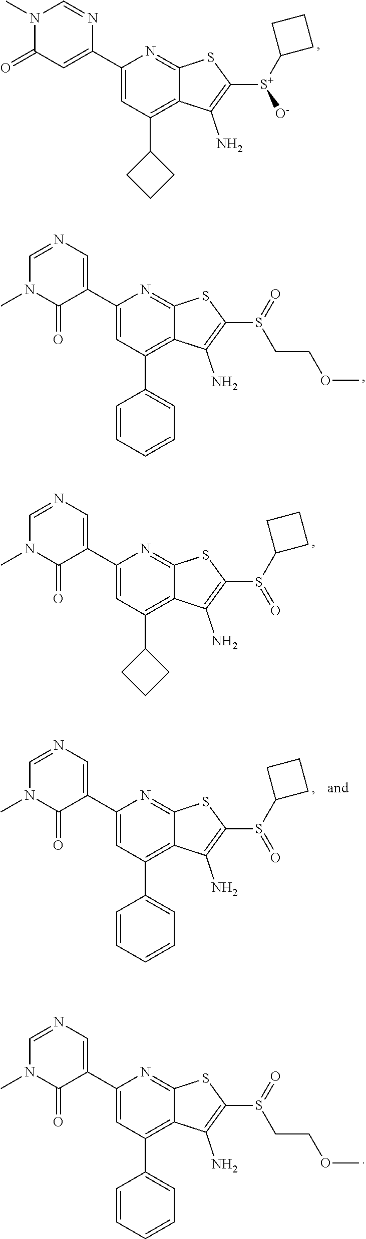

In embodiments of compounds of formula (I), R¹ is C₁-C₆ alkyl, C₃-C₆ cycloalkyl, or —(C₁-C₃ alkylene)-(C₁-C₃ alkoxy). In embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

In embodiments of compounds of formula (I), R² is NH₂.

In embodiments of compounds of formula (I), R³ is halogen, —OH, —NH₂, C₁-C₆ alkyl, C₂-C₆ alkenyl, C₂-C₆ alkynyl, or C₁-C₆ alkoxy.

In embodiments of compounds of formula (I), when R⁴ is oxo and R⁷ is aryl or heteroaryl, oxo does not violate the valency of the aryl or the heteroaryl. In embodiments, when R⁷ is aryl or heteroaryl, R⁴ is not oxo. In embodiments, R⁴ is halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸.

In embodiments of compounds of formula (I), R⁶ is

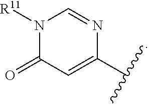

In embodiments of compounds of formula (I), R¹¹ is H or methyl.

In embodiments of compounds of formula (I), R⁷ is phenyl, alkyl, or cycloalkyl, each of which is optionally substituted with one or more R⁴.

In embodiments of compounds of formula (I), R⁷ is a linear or branched, non-cyclic C₁-C₆ alkyl. In embodiments, R⁷ is methyl, ethyl, n-propyl, i-propyl, n-butyl, s-butyl, or t-butyl. In embodiments, R⁷ is i-propyl.

In embodiments of compounds of formula (I), X is CH.

In embodiments of compounds of formula (I), n is 1.

The present disclosure also relates to compounds of formula (II)

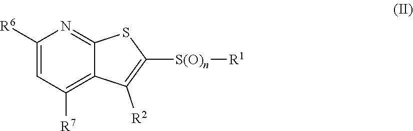

or a pharmaceutically acceptable salt, tautomer, or solvate thereof, wherein:
R¹ is C₁-C₆ alkyl, C₃-C₆ cycloalkyl, or —(C₁-C₃ alkylene)-(C₁-C₃ alkoxy);
R⁶ is

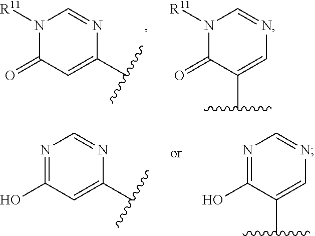

R⁷ is a linear or branched, non-cyclic C₁-C₆ alkyl (e.g., i-propyl).
R¹¹ is H or C₁-C₆ alkyl; and
n is 0, 1, or 2.

In embodiments of compounds of formula (I) or (II), the compound is selected from:

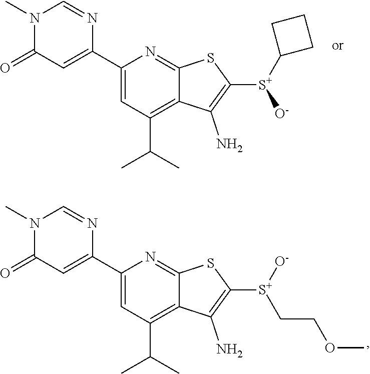

or a pharmaceutically acceptable salt, tautomer, or solvate thereof.

In embodiments and without being limited by theory, the Applicants unexpectedly and surprisingly discovered that linear or branched, non-cyclic alkyl groups at R⁷ position of the compounds of formula (I) and (II) improved solubility and metabolic stability of the compounds.

In embodiments, R⁷ is isopropyl.

The present disclosure also relates to compounds of formula (III):

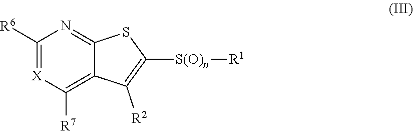

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

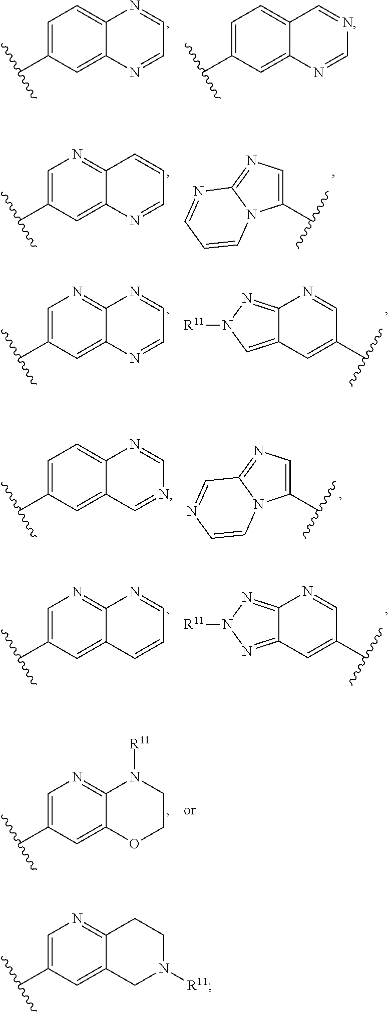

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2.

In embodiments of the compounds of formula (III), the compound is not:

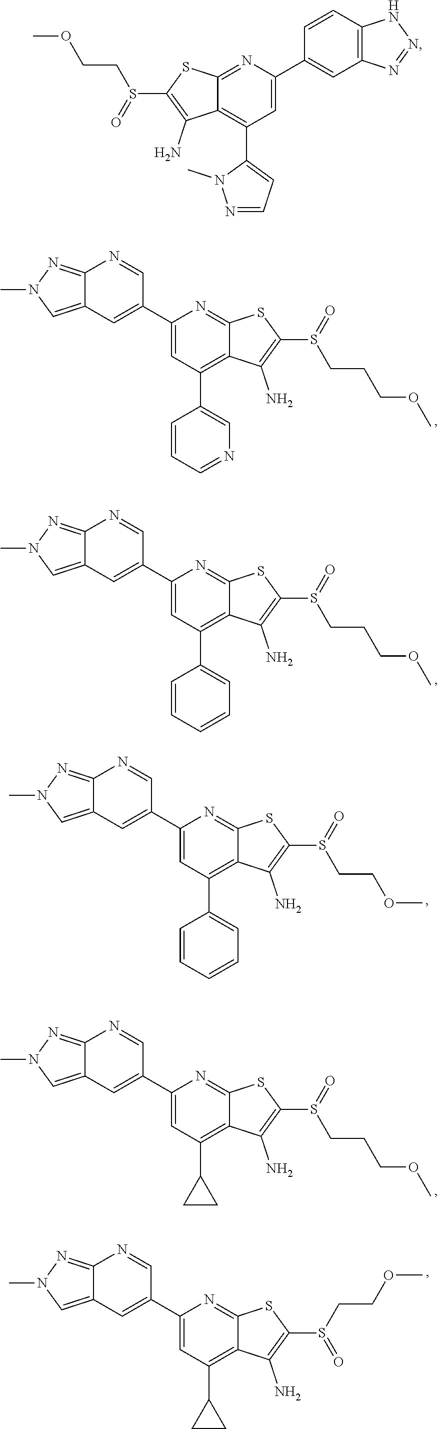

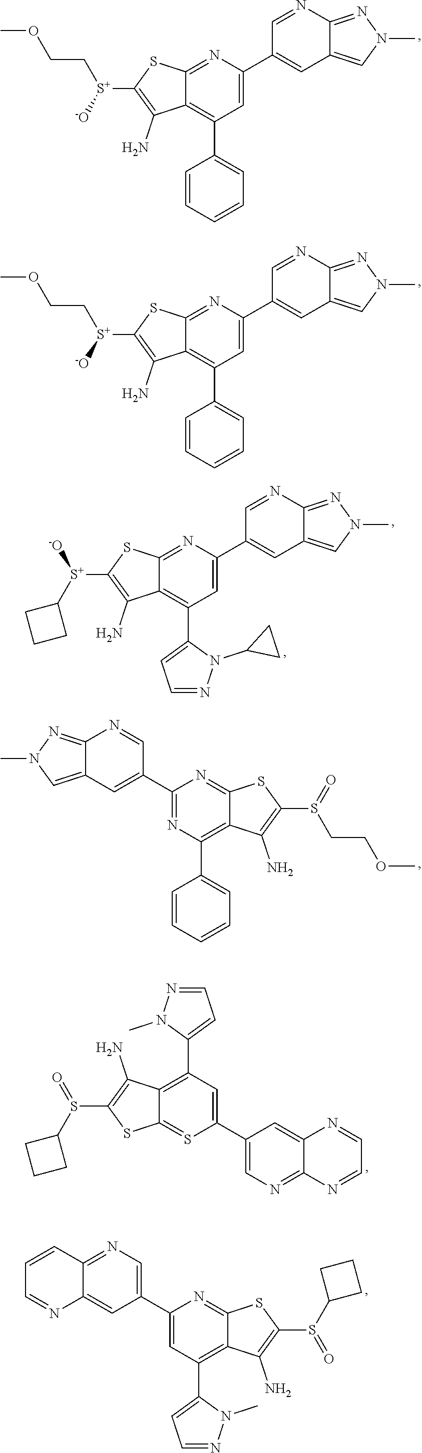

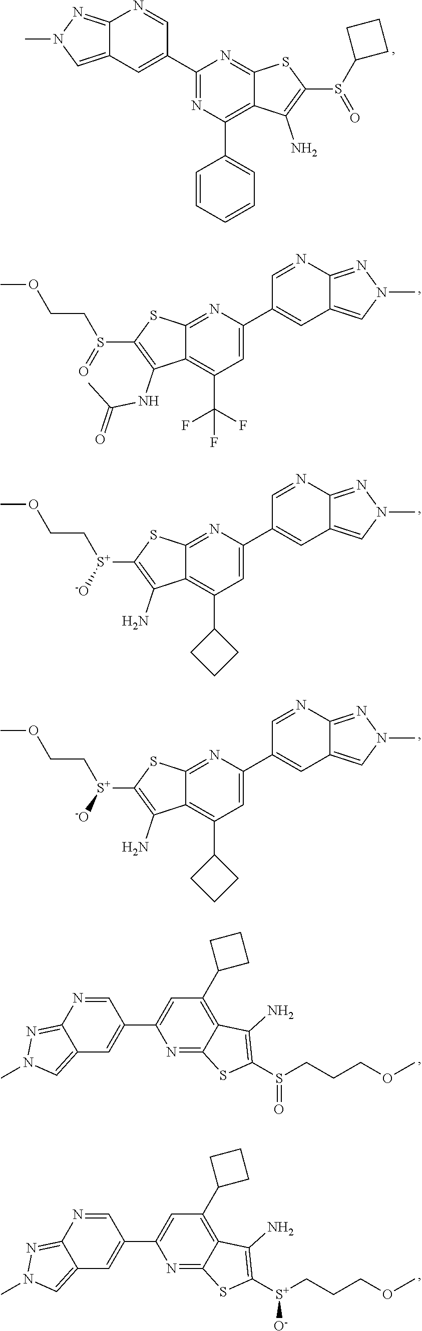

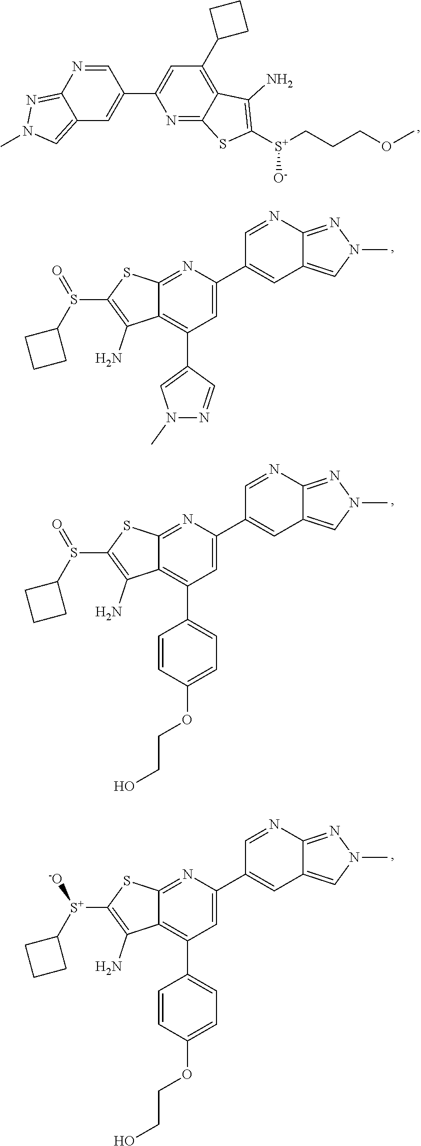

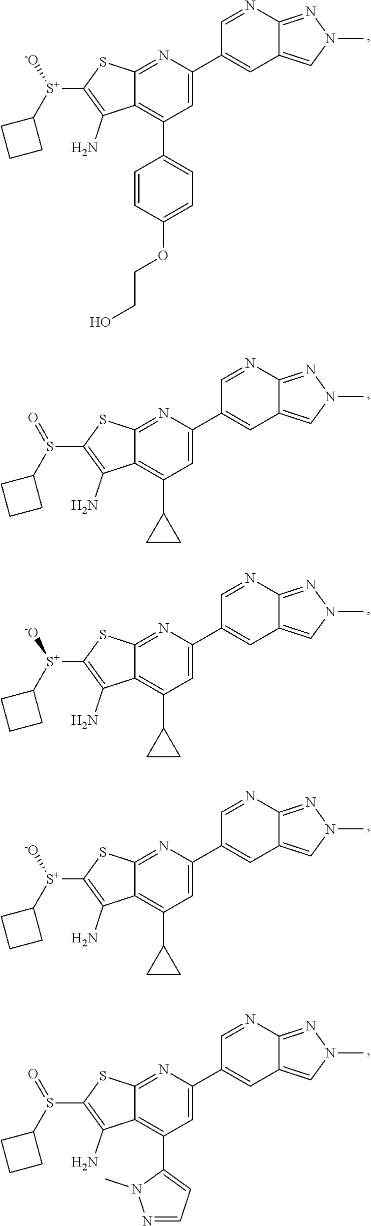

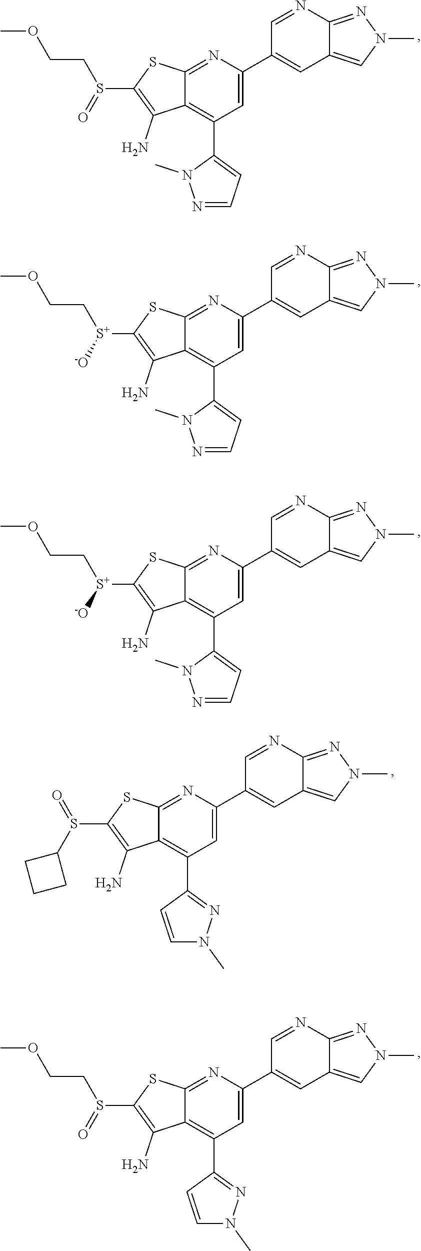

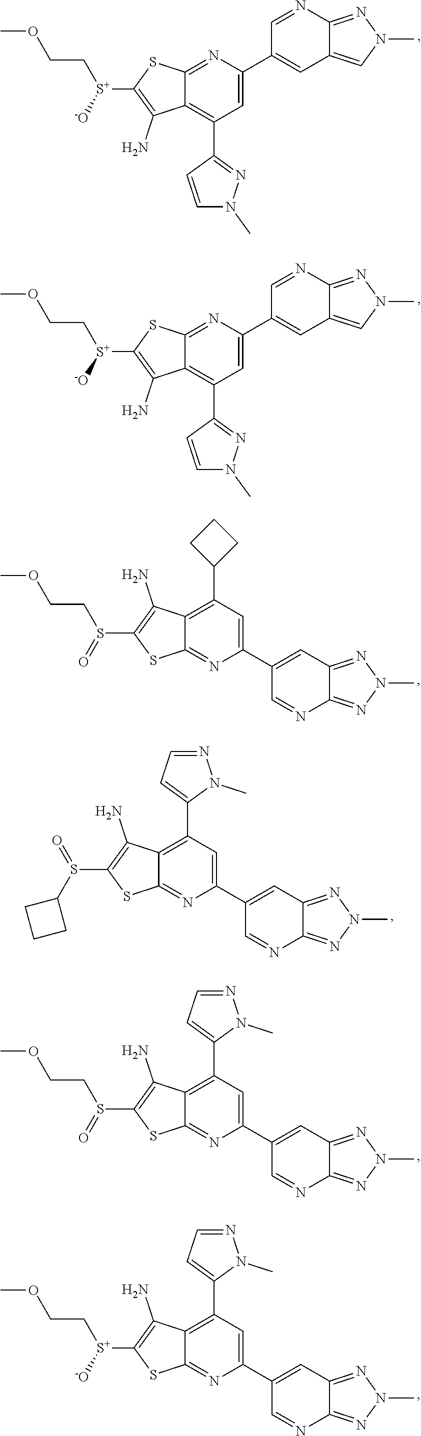

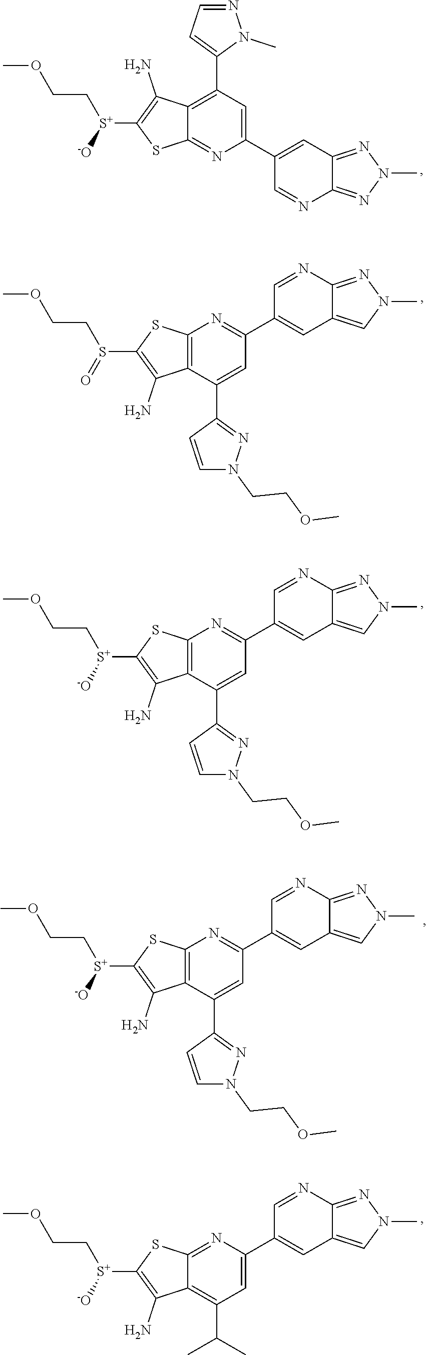

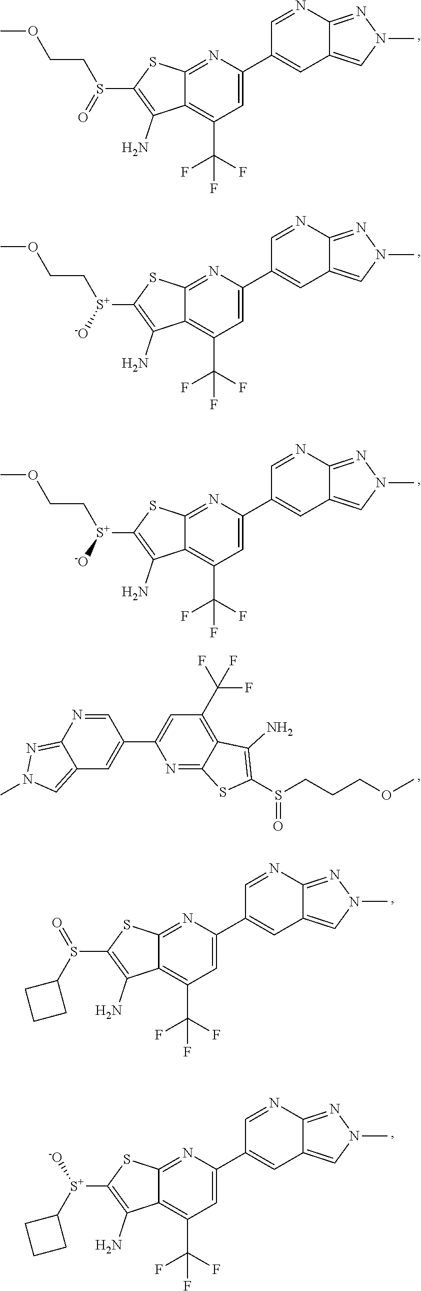

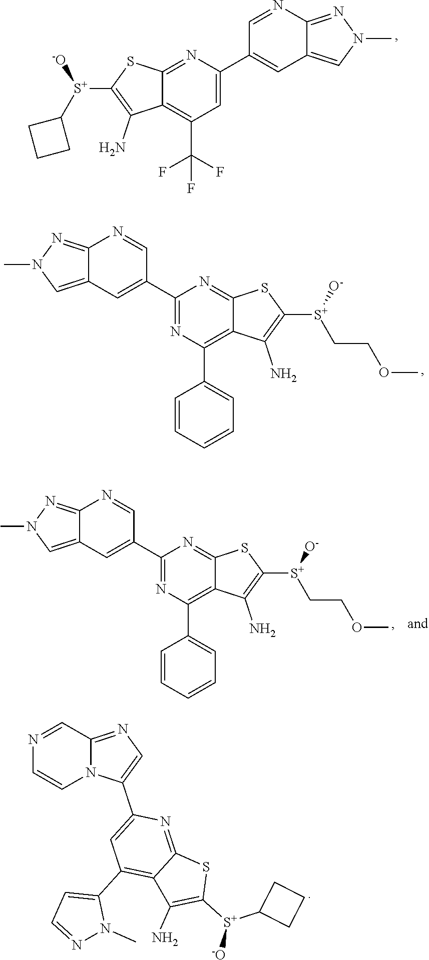

In embodiments of compounds of formula (III), R¹ is C₁-C₆ alkyl, C₃-C₆ cycloalkyl, or —(C₁-C₃ alkylene)-(C₁-C₃ alkoxy). In embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

In embodiments of compounds of formula (III), R² is NH₂ or —CN.

In embodiments of compounds of formula (III), when R⁴ is oxo and R⁷ is aryl or heteroaryl, oxo does not violate the valency of the aryl or the heteroaryl. In embodiments, when R⁷ is aryl or heteroaryl, R⁴ is not oxo. In embodiments, R⁴ is halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸.

In embodiments of compounds of formula (III), R⁶ is

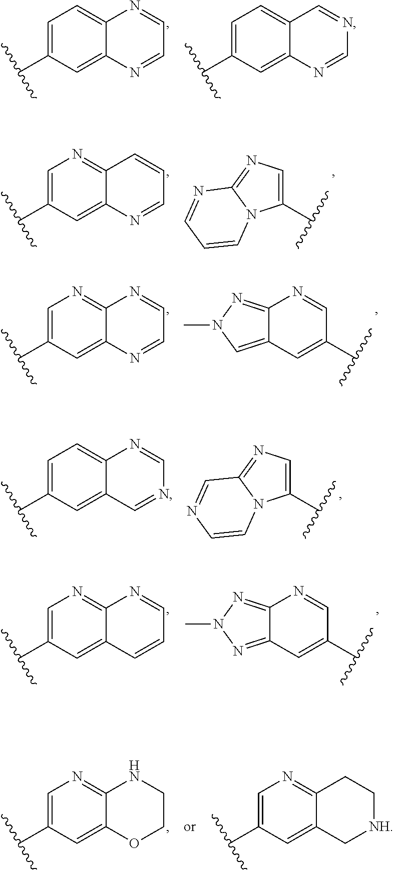

In embodiments of compounds of formula (III), R⁶ is

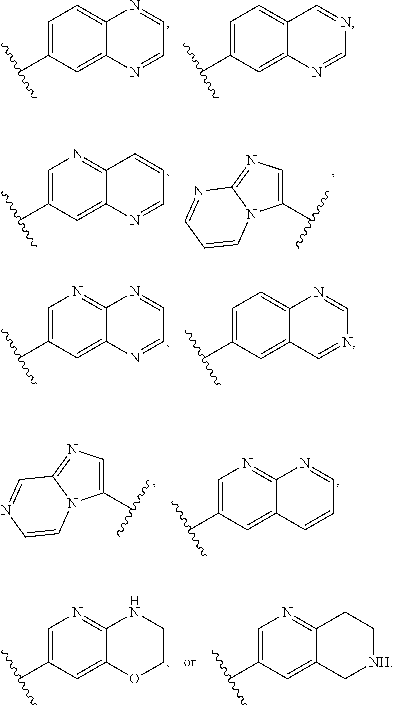

In embodiments of compounds of formula (III), R⁷ is alkyl, cycloalkyl, aryl, heterocyclyl, or heteroaryl, each of which is optionally substituted with one or more R⁴.

In embodiments of compounds of formula (III), n is 1.

In embodiments of compounds of formula (III), the compound is selected from:

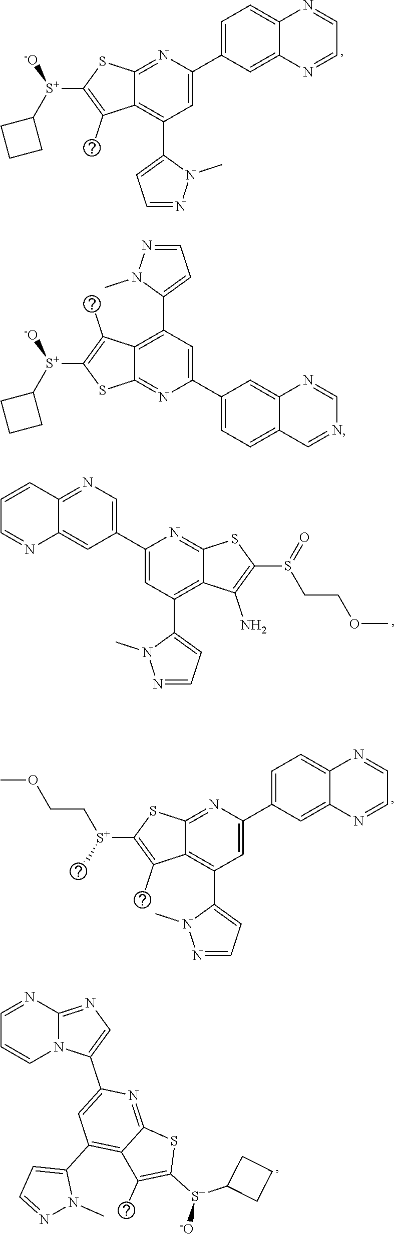

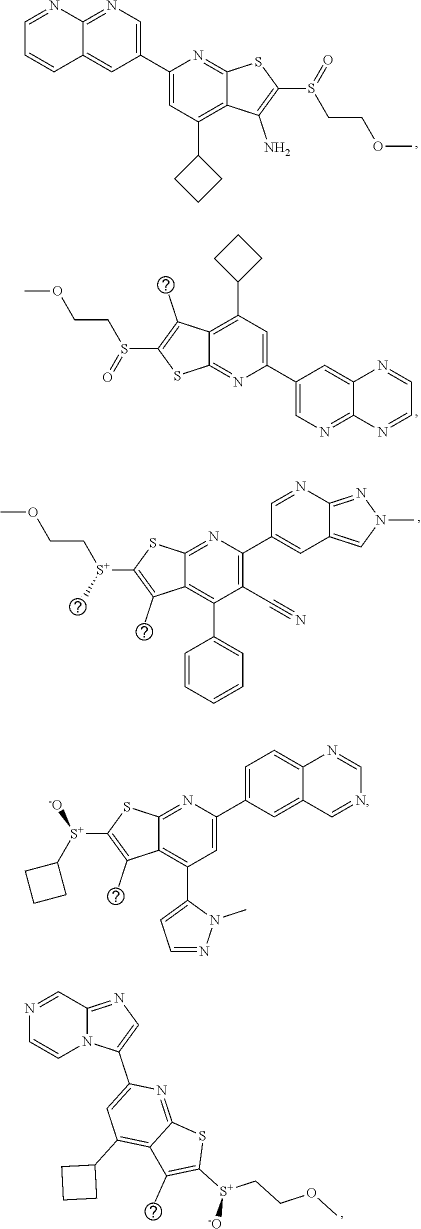

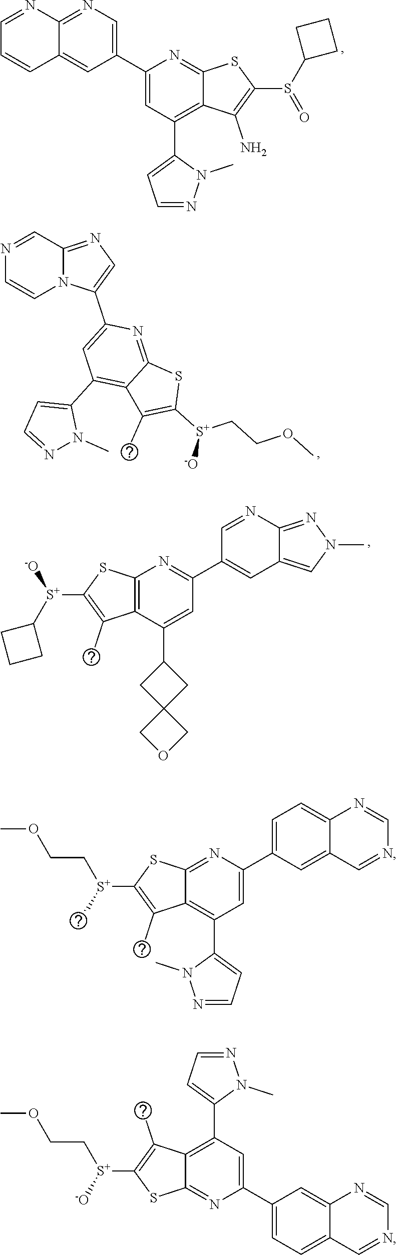

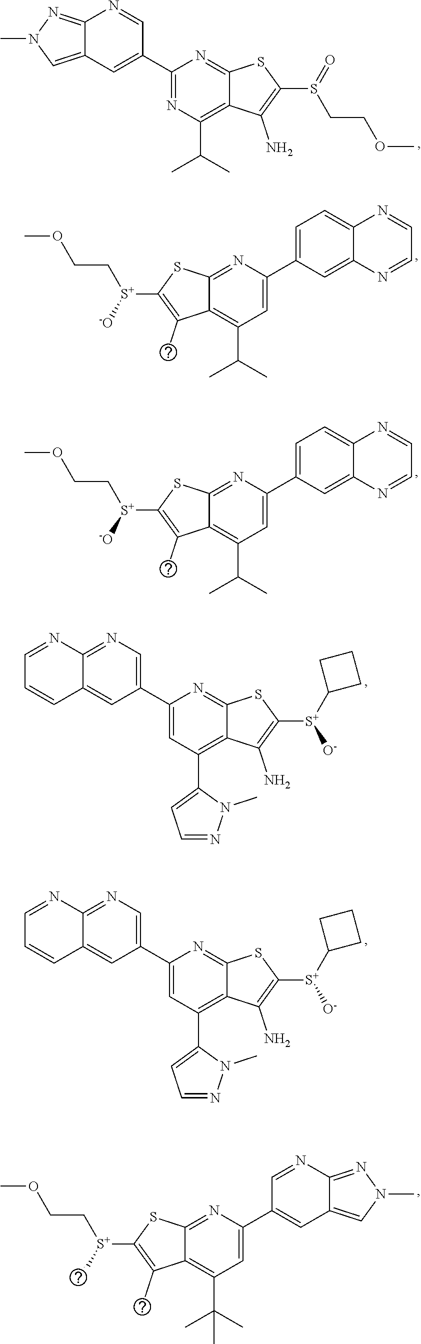

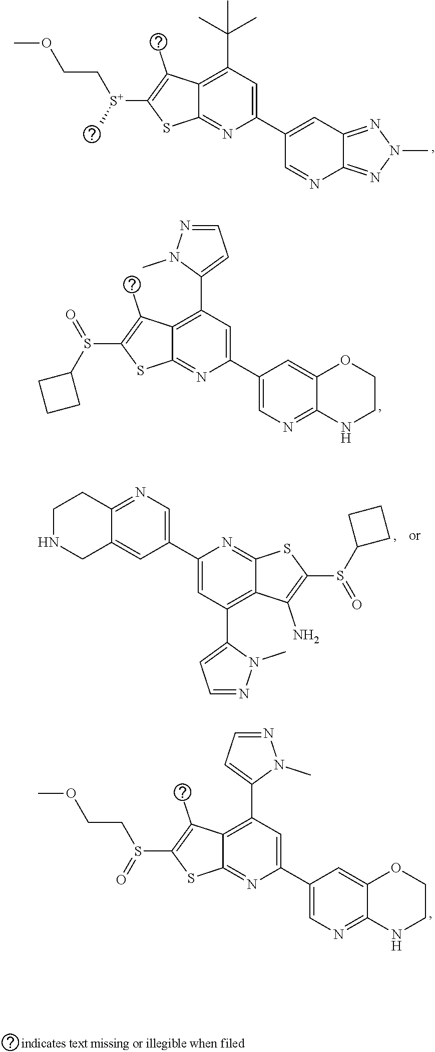

or a pharmaceutically acceptable salt, tautomer, or solvate thereof.

The present disclosure also relates to compounds of formula (IV):

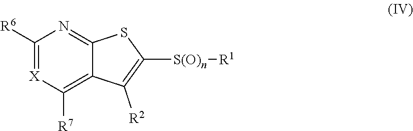

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

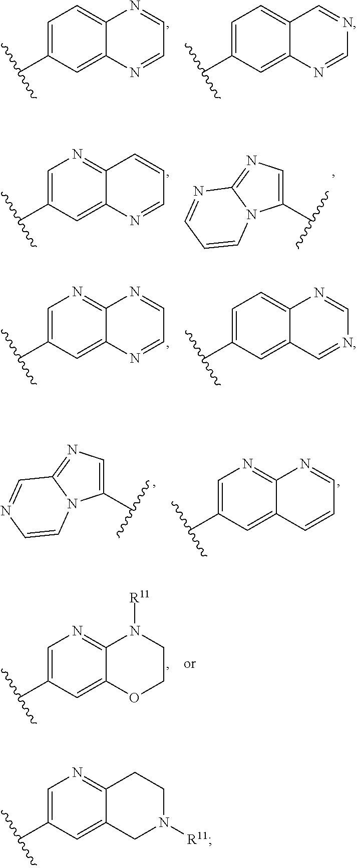

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2;
wherein the compound is not:

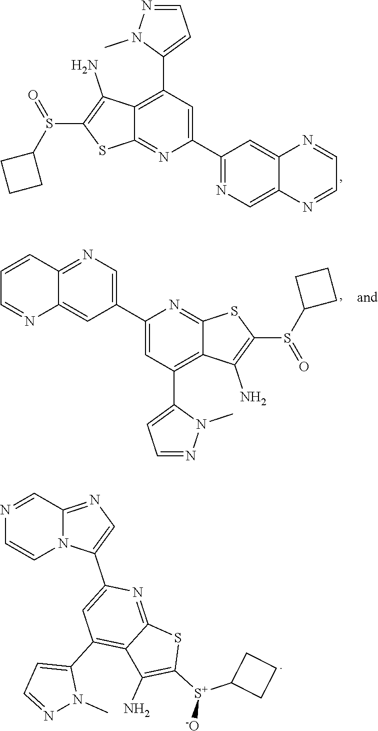

The present disclosure also relates to compounds of:

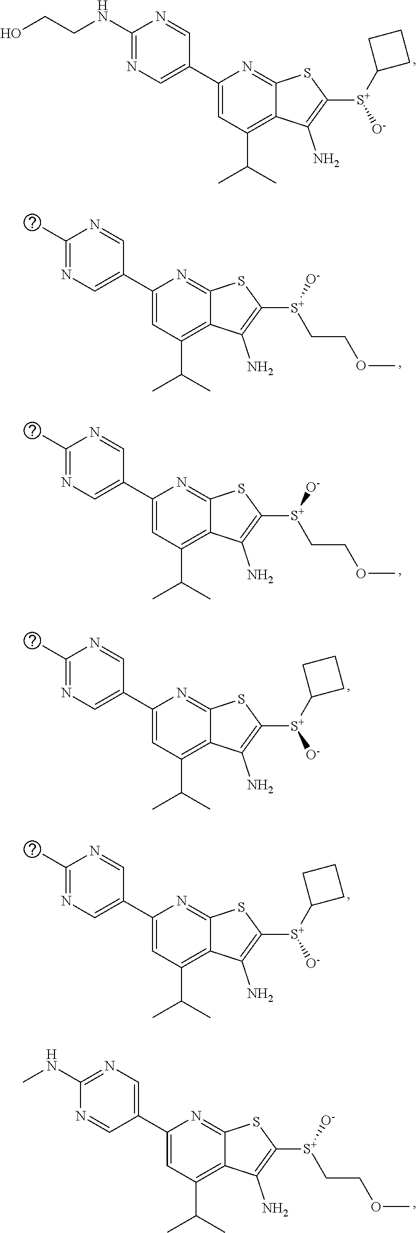

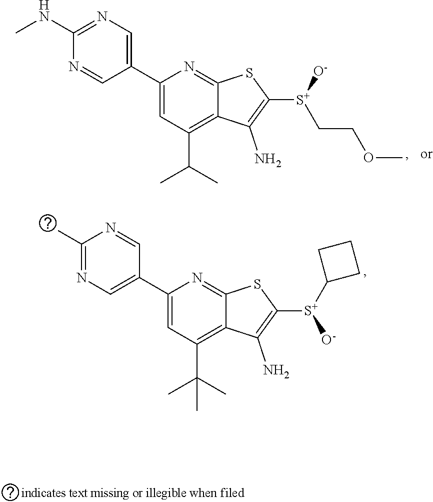

or a pharmaceutically acceptable salt, tautomer, or solvate thereof.

In embodiments of compounds of formula (IV), when R⁴ is oxo and R⁷ is aryl or heteroaryl, oxo does not violate the valency of the aryl or the heteroaryl. In embodiments, when R⁷ is aryl or heteroaryl, R⁴ is not oxo. In embodiments, R⁴ is halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸.

In embodiments of formula (I)-(IV), R¹ is C₁-C₆ alkyl, C₃-C₆ cycloalkyl, or —(C₁-C₃ alkylene)-(C₁-C₃ alkoxy). In embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3. In embodiments, R¹ is 3- to 5-membered cycloalkyl or —(C₁-C₆ alkylene)-(3- to 5-membered cycloalkyl). In embodiments, R¹ is cyclobutyl. In embodiments, R¹ is —(CH₂)₂OMe or —(CH₂)₃OMe.

In embodiments of formula (I)-(IV), R² is —NH₂ or CN. In embodiments, R² is —NH₂.

In embodiments of formula (I)-(IV), R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴. In embodiments, R⁷ is C₁-C₆ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In embodiments, R⁷ is C₁-C₆ haloalkyl, 3- to 6-membered cycloalkyl, phenyl, 5- to 10-membered heteroaryl each of which is optionally substituted with one or more R⁴. In embodiments, R⁷ is a linear or branched, non-cyclic C₁-C₆ alkyl. In embodiments, R⁷ is methyl, ethyl, n-propyl, i-propyl, n-butyl, s-butyl, or t-butyl. In embodiments, R⁷ is i-propyl, n-butyl, s-butyl, t-butyl, cyclobutyl, phenyl, pyrazoyl, or 2-oxaspiro[3.3]heptane. In embodiments, R⁷ is i-propyl, n-butyl, s-butyl, t-butyl, cyclobutyl, phenyl,

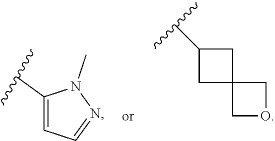

In embodiments of formula (I)-(IV), R⁴ is halogen, alkyl, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl. In embodiments, R⁴ is independently selected from methyl or ethyl. In embodiments, R⁴ is methyl.

In embodiments of formula (I)-(IV), X is CH.

In embodiments of formula (I)-(IV), n is 1.

In embodiments, 15-PGDH inhibitor of the present disclosure relates to compounds in Table 1, or a pharmaceutically acceptable salt, tautomer, or solvate thereof.

Colon 15-PGDH inhibition can be measured using an appropriate dose of the compounds of the present disclosure, at 30 min, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 15 hours, 20 hours, 24 hours, 48 hours, 72 hours, or more hours after administration, including all times between these values. In embodiments, colon 15-PGDH inhibition is measured at 30 minutes after administration. In embodiments, colon 15-PGDH inhibition is measured at 4 hours. In embodiments, the appropriate dose is 1 2, 3, 4, 5, 6, 7, 8, 9, 0 15, 20, 30, 40, 50, or more mg/kg, including all values and ranges in between these values. In embodiments, the 15-PGDH inhibitor of the present disclosure inhibit colon 15-PGDH activity in a range of from about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. See PCT/US2019/062686.

In embodiments, lung, liver, intestines, the skin, heart (or any other organ disclosed herein) 15-PGDH inhibition can be measured using an appropriate dose of the compounds of the present disclosure, at 30 min, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 15 hours, 20 hours, 24 hours, 48 hours, 72 hours, or more, including all times and ranges in between these values. In embodiments, lung 15-PGDH inhibition is measured at 30 minutes. In particular embodiments, lung 15-PGDH inhibition is measured at 4 hours. In embodiments, the appropriate dose is 1 2, 3, 4, 5, 6, 7, 8, 9, 0 15, 20, 30, 40, 50, or more mg/kg, including all values and ranges in between these values. In embodiments, the 15-PGDH inhibitor of the present disclosure inhibit lung 15-PGDH activity in a range of from about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein.

In embodiments, the 15-PGDH inhibitor of the present disclosure (e.g., having formula I-IV), is administered at 10 mg/kg in a mammal and inhibits colon 15-PGDH activity at 30 minutes in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In embodiments, the compounds of the present invention, when administered at 10 mg/kg in a mammal, inhibit colon 15-PGDH activity at 30 minutes in a range of about 65% to 100% (e.g., about 65%, about 70%, about 75%, about 80%, about 85%, about 90%, about 95%, or about 100%), and any subranges therein. In embodiments, the compounds of the present invention when administered at 10 mg/kg in a mammal can inhibit colon 15-PGDH activity at 30 minutes in a range of about 70% to 100% (e.g., about 70%, about 75%, about 80%, about 85%, about 90%, about 95%, or about 100%), and any subranges therein. In embodiments, the compounds of the present invention, when administered at 10 mg/kg in a mammal, inhibit colon 15-PGDH activity at 30 minutes in a range of about 80% to 100%, and any subranges therein. In embodiments, the compounds of the present invention, when administered at 10 mg/kg in a mammal, inhibit colon 15-PGDH activity at 30 minutes in a range of about 90% to 100%, and any subranges therein.

In embodiments, the 15-PGDH inhibitor of the present disclosure (e.g., having formula I-IV), is administered at 10 mg/kg in a mammal and inhibits colon 15-PGDH activity at 4 hours in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In embodiments, the compounds of the present invention, when administered at 10 mg/kg in a mammal, inhibit colon 15-PGDH activity at 4 hours in a range of about 65% to 100% (e.g., about 65%, about 70%, about 75%, about 80%, about 85%, about 90%, about 95%, or about 100%), and any subranges therein. In embodiments, the compounds of the present invention when administered at 10 mg/kg in a mammal can inhibit colon 15-PGDH activity at 4 hours in a range of about 70% to 100% (e.g., about 70%, about 75%, about 80%, about 85%, about 90%, about 95%, or about 100%), and any subranges therein. In embodiments, the compounds of the present invention, when administered at 10 mg/kg in a mammal, inhibit colon 15-PGDH activity at 4 hours in a range of about 80% to 100%, and any subranges therein. In embodiments, the compounds of the present invention, when administered at 10 mg/kg in a mammal, inhibit colon 15-PGDH activity at 4 hours in a range of about 80% to 98%, and any subranges therein.

In embodiments, the 15-PGDH inhibitor of the present disclosure (e.g., having formula I-IV), is administered at 10 mg/kg in a mammal and inhibits lung, liver, intestines, the skin, heart (or any other organ disclosed herein) 15-PGDH activity at 30 minutes in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In embodiments, the compounds of the present invention, when administered at 10 mg/kg in a mammal, inhibit lung 15-PGDH activity at 30 minutes in a range of about 65% to 100% (e.g., about 65%, about 70%, about 75%, about 80%, about 85%, about 90%, about 95%, or about 100%), and any subranges therein. In embodiments, the compounds of the present invention when administered at 10 mg/kg in a mammal can inhibit lung 15-PGDH activity at 30 minutes in a range of about 70% to 100% (e.g., about 70%, about 75%, about 80%, about 85%, about 90%, about 95%, or about 100%), and any subranges therein. In embodiments, the compounds of the present invention, when administered at 10 mg/kg in a mammal, inhibit lung 15-PGDH activity at 30 minutes in a range of about 80% to 100%, and any subranges therein.

In embodiments, the 15-PGDH inhibitor of the present disclosure (e.g., having formula I-IV), is administered at 10 mg/kg in a mammal and inhibits lung, liver, intestines, the skin, heart (or any other organ disclosed herein) 15-PGDH activity at 4 hours in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In embodiments, the compounds of the present invention, when administered at 10 mg/kg in a mammal, inhibit lung 15-PGDH activity at 4 hours in a range of about 65% to 100% (e.g., about 65%, about 70%, about 75%, about 80%, about 85%, about 90%, about 95%, or about 100%), and any subranges therein. In embodiments, the compounds of the present invention when administered at 10 mg/kg in a mammal can inhibit lung 15-PGDH activity at 4 hours in a range of about 70% to 100% (e.g., about 70%, about 75%, about 80%, about 85%, about 90%, about 95%, or about 100%), and any subranges therein. In embodiments, the compounds of the present invention, when administered at 10 mg/kg in a mammal, inhibit lung 15-PGDH activity at 4 hours in a range of about 80% to 100%, and any subranges therein.

In embodiments, the 15-PGDH inhibitors of the present invention (e.g., formula I-IV) have a human or mouse microsome stability T_{1/2} of greater than 50 minutes, greater than 60 minute, greater than 70 minutes, greater than 80 minutes, greater than 90 minutes, or greater than 100 minutes, including all values and ranges there between. In embodiments, the compounds of the invention has a human or mouse microsome stability T_{1/2} of greater than 110 minutes, greater than 120 minutes, greater than 130 minutes, or greater than 145 minutes, including all values and ranges therebetween. In embodiments, the 15-PGDH inhibitors of the invention have a human or mouse microsome stability T_{1/2} ranging from 65 to at least 145 (e.g., 65, 70, 80, 90, 100, 110, 120, 130, 140, 150, 160, 170, 180, 190, 200, or more, including all values and ranges therebetween). In embodiments, the compounds of the invention has a human or mouse microsome stability T_{1/2} of greater than 145 minutes.

In embodiments, the 15-PGDH inhibitors of the present invention (e.g., formula I-IV) have better human or mouse microsome stability compared to previously disclosed 15-PGDH inhibitors. See WO 2013/158649, WO 2015/065716, WO 2016/144958, WO 2016/168472, WO 2018/017582, WO 2018/102552, WO 2018/145080, WO 2018/187810, WO 2018/218251 and/or PCT/US2019/062686, the disclosures of each are hereby incorporated by reference in their entireties for all purposes. In embodiments, the 15-PGDH inhibitors of the invention have a human or mouse microsome stability T_{1/2} which is at least 15 minutes longer, at least 25 minutes longer, at least 35 minutes longer, at least 45 minutes longer, at least 55 minutes longer, at least 65 minutes longer, at least 75 minutes longer, at least 85 minutes longer, at least 95 minutes longer, at least 100 minutes longer, at least 110 minutes longer, at least 120 minutes longer than previously disclosed 15-PGDH inhibitors, including all values and ranges therebetween. In embodiments, the 15-PGDH inhibitors of the invention have a human or mouse microsome stability T_{1/2} ranging that is from 15 minutes to about 120 minutes longer than the microsome stability T_{1/2} of previously disclosed 15-PGDH inhibitors.

In embodiments, the 15-PGDH inhibitors of the present invention (e.g., formula I-IV) have a kinetic aqueous solubility in pH 7 or pH 4 citrate buffer solution greater than about 150 μM. In embodiments, the 15-PGDH inhibitors of the present invention have a kinetic aqueous solubility in pH 7 or pH 4 citrate buffer solution greater than about 160 μM. In embodiments, the 15-PGDH inhibitors of the present invention have a kinetic aqueous solubility in pH 7 or pH 4 citrate buffer solution greater than about 170 μM. In embodiments, the 15-PGDH inhibitors of the present invention have a kinetic aqueous solubility in pH 7 or pH 4 citrate buffer solution greater than about 180 μM. In embodiments, the 15-PGDH inhibitors of the present invention have a kinetic aqueous solubility in pH 7 or pH 4 citrate buffer solution greater than about 190 μM. In embodiments, the 15-PGDH inhibitors of the present invention have a kinetic aqueous solubility in pH 7 or pH 4 citrate buffer solution greater than about 200 μM.

In embodiments, the 15-PGDH inhibitors of the present invention (e.g., formula I-IV) have a kinetic aqueous solubility in pH 7 or pH 4 citrate buffer solution greater than previously disclosed 15-PGDH inhibitors. In embodiments, the kinetic aqueous solubility in pH 7 or pH 4 citrate buffer solution of the 15-PGDH inhibitors of the present invention is at least about 5% greater, about 10% greater, about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, or about 95% greater than previously disclosed 15-PGDH inhibitors, including all values and ranges therebetween.

In embodiments, the 15-PGDH inhibitors of the present invention (e.g., formula I-IV) have a high permeability by Caco-2 permeability assay. In embodiments, the 15-PGDH inhibitors of the present invention have an efflux ratio (ER) of less than about 15, less than about 14, less than about 13, less than about 12, less than about 11, less than about 10, less than about 9, less than about 8, less than about 7, or less than about 6, including all values and ranges therebetween. In embodiments, the 15-PGDH inhibitors of the present invention have an efflux ratio (ER) of less than about 10. In embodiments, the 15-PGDH inhibitors of the present invention have an efflux ratio (ER) of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In embodiments, the 15-PGDH inhibitors of the present invention have an efflux ratio (ER) in the range of about 1 to 6, including all values and ranges therebetween.

In embodiments, the 15-PGDH inhibitors of the present invention (e.g., formula I-IV) provides Cmax in the range of about 7,000 ng/mL to about 16,000 ng/mL, including all values and ranges therebetween. In embodiments, the 15-PGDH inhibitors of the present invention (e.g., formula I-IV) provides Cmax in the range of about 7,000 ng/mL to about 16,000 ng/mL, including all values and ranges therebetween, when a single dose of the 15-PGDH inhibitor is administered at 20 mg/kg. In embodiments, the 15-PGDH inhibitors of the present invention provides Cmax in the range of about 8,000 ng/mL to about 15,000 ng/mL, including all values and ranges there between. In embodiments, the 15-PGDH inhibitors of the present invention provides Cmax in the range of about 9,000 ng/mL to about 14,000 ng/mL, including all values and ranges therebetween. In embodiments, the 15-PGDH inhibitors of the present invention provides Cmax in the range of about 9,500 ng/mL to about 13,500 ng/mL, including all values and ranges there between. In embodiments, the Cmax as disclosed herein relates to a single oral dose of 20 mg/kg 15-PGDH inhibitor administered to mice.

In embodiments, the 15-PGDH inhibitors of the present invention (e.g., formula I-IV) provides AUC in the range of about 10,000 ng*h/mL to about 60,000 ng*h/mL, including all values and ranges therebetween. In embodiments, the 15-PGDH inhibitors of the present invention (e.g., formula I-IV) provides AUC in the range of about 10,000 ng*h/mL to about 60,000 ng*h/mL, including all values and ranges therebetween, when a single dose of the 15-PGDH inhibitor is administered at 20 mg/kg. In embodiments, the 15-PGDH inhibitors of the present invention provides AUC in the range of about 20,000 ng*h/mL to about 50,000 ng*h/mL, including all values and ranges there between. In embodiments, the 15-PGDH inhibitors of the present invention provides AUC in the range of about 22,000 ng*h/mL to about 45,000 ng*h/mL, including all values and ranges therebetween. In embodiments, the AUC as disclosed herein relates to a single oral dose of 20 mg/kg 15-PGDH inhibitor administered to mice.

In embodiments, the 15-PGDH inhibitors of the present invention (e.g., formula I-IV) provides clearance (Cl) in the range of about 5 ml/min/kg to about 20 ml/min/kg, including all values and ranges there between. In embodiments, the 15-PGDH inhibitors of the present invention (e.g., formula I-IV) provides clearance (Cl) in the range of about 5 ml/min/kg to about 20 ml/min/kg, including all values and ranges therebetween, when a single dose of the 15-PGDH inhibitor is administered at 5 mg/kg. In embodiments, the 15-PGDH inhibitors of the present invention provides clearance (Cl) in the range of about 6 ml/min/kg to about 19 ml/min/kg, including all values and ranges therebetween. In embodiments, the 15-PGDH inhibitors of the present invention provides clearance (Cl) in the range of about 6 ml/min/kg to about 18 ml/min/kg, including all values and ranges therebetween. In embodiments, the 15-PGDH inhibitors of the present invention provides clearance (Cl) of about 5 ml/min/kg, about 6 ml/min/kg, about 7 ml/min/kg, about 8 ml/min/kg, about 9 ml/min/kg, about 10 ml/min/kg, about 11 ml/min/kg, about 12 ml/min/kg, about 13 ml/min/kg, about 14 ml/min/kg, about 15 ml/min/kg, about 16 ml/min/kg, about 17 ml/min/kg, about 18 ml/min/kg, about 19 ml/min/kg, or about 20 ml/min/kg, including all values and ranges therebetween. In embodiments, the C₁ values as disclosed herein relates to a single IV dose of 5 mg/kg 15-PGDH inhibitor administered to mice.

The EC₅₀ for induction of PGE2 is determined using A549 cells that have been treated with IL1-β 24 hours. In embodiments, the 15-PGDH inhibitors of the present invention (e.g., formula I-IV) have an EC₅₀ for induction of PGE2 that is less than or equal to 10 nM. In embodiments, the EC₅₀ is less than or equal to 5 nM. In embodiments, the EC₅₀ is less than or equal to 4 nM. In embodiments, the EC₅₀ is less than or equal to 3 nM. In embodiments, the EC₅₀ is less than or equal to 2 nM. In embodiments, the EC₅₀ is less than or equal to 1 nM. In embodiments, the EC₅₀ is from 10 nM to about 0.01 nM including all values and subranges in between these values). In embodiments, the EC₅₀ is at least 4 times less than the previously disclosed 15-PGDH inhibitors, such as those disclosed in the publications referenced above. In embodiments, the EC₅₀ is at least 8 times less than the previously disclosed 15-PGDH inhibitors. In embodiments, the EC₅₀ is at least 10 times less than the previously disclosed 15-PGDH inhibitors. In embodiments, the EC₅₀ is at least 15 times less than the previously disclosed 15-PGDH inhibitors. In embodiments, the EC₅₀ is at least 20 times less than the previously disclosed 15-PGDH inhibitors. In embodiments, the EC₅₀ is at least 30 times less than the previously disclosed 15-PGDH inhibitors. In embodiments, the EC₅₀ is at least 40 times less than the previously disclosed 15-PGDH inhibitors. In embodiments, the EC₅₀ is at least 50 times less than the previously disclosed 15-PGDH inhibitors. In embodiments, the EC₅₀ is 10 times to 50 times less than the previously disclosed 15-PGDH inhibitors.

In certain embodiments, the 15-PGDH inhibitor having formula (I-)-(IV), can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 μM concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-renilla luciferase reporter to a level greater than 20; and va) inhibits the enzymatic activity of recombinant 15-PGDH protein at an IC₅₀ of less than 1 μM.

In embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-renilla luciferase reporter to a luciferase level greater than 20% above background; and vb) inhibits the enzymatic activity of recombinant 15-PGDH protein at an IC₅₀ of less than 1 μM.

In embodiments, the compound or 15-PGDH inhibitor can inhibit the enzymatic activity of recombinant 15-PGDH at an IC₅₀ of less than 1 μM, at an IC₅₀ of less than 250 nM, at an IC₅₀ of less than 50 nM, at an IC₅₀ of less than 10 nM, at an IC₅₀ of less than 5 nM at a recombinant, at an IC₅₀ of about 2.5 nM to about 10 nM, or less than about 2.5 nM at a 15-PGDH concentration of about 5 nM to about 10 nM.

In embodiments, the 15-PGDH inhibitor can increase the cellular levels of PGE-2 following stimulation of an A459 cell with an appropriate agent, for example IL1β.

### Therapeutic Use

The 15-PGDH inhibitors described herein can be used for the prevention or the treatment of diseases that are associated with 15-PGDH and/or decreased prostaglandin levels and/or where it desirable to increase prostaglandin levels in the subject. For example, as discussed above, it is known that prostaglandins play an important role in hair growth. Specifically, internal storage of various types (A₂, F₂ₐ, E₂) of prostaglandins in the various compartments of hair follicles or their adjacent skin environments has been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF₂ₐ and PGE₂, to cause scalp damage and alopecia (Michelet J F et. al., 2008, Exp. Dermatol, 17(10), 821-8). Thus, the compounds described herein, which have a suppressive or inhibitory activity against 15-PGDH that degrades prostaglandins, can improve scalp damage, prevent alopecia and promote hair growth and be used in a pharmaceutical composition for the prevention of alopecia and the promotion of hair growth.

In embodiments, the 15-PGDH inhibitors described herein can be used in a pharmaceutical composition for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities.

In embodiments, the 15-PGDH inhibitor can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In still other embodiments, the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension.

In embodiments, the 15-PGDH inhibitors described herein can be used in a pharmaceutical composition for the prevention or the treatment of oral, intestinal, and/or gastrointestinal injury or diseases, or inflammatory bowel disease, such as oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In the stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, that is, the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Moreover, 15-PGDH inhibitors would also be expected to protect from other form of intestinal injury that would include toxicity from radiation, toxicity from chemotherapy, and chemotherapy induced mucositis.

In the kidney, prostaglandins modulate renal blood flow and may serve to regulate urine formation by both renovascular and tubular effects. In clinical studies, PGE₁ has been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (see Porter, Am., 1989, J. Cardiol., 64: 22E-26E). In addition, U.S. Pat. No. 5,807,895 discloses a method of preventing renal dysfunction by intravenous administration of prostaglandins such as PGE₁, PGE₂ and PGI₂. Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about.77).

Thus, the 15-PGDH inhibitors described herein, which have a suppressive or inhibitory activity against 15-PGDH that degrades prostaglandins, may be effective in the prevention or the treatment of renal diseases that are associated with renal dysfunction.

The term “renal dysfunction” as used herein includes such manifestations as follows: lower than normal creatinine clearance, lower than normal free water clearance, higher than normal blood urea, nitrogen, potassium and/or creatinine levels, altered activity of kidney enzymes such as gamma glutamyl synthetase, alanine phosphatidase, N-acetyl-β-D-glucosaminidase, or β-w-microglobulin; and increase over normal levels of macroalbuminuria.

In other embodiments, the 15-PGDH inhibitors can be used to prevent, treat, or reduce the severity of a renal disorder, disease, and/or injury. Examples of renal disorders, diseases, and/or injuries that can be treated include acute kidney injury; hypotensive injury to the kidney; hypertensive renal disease; diabetic renal disease and diabetic nephropathy; renal disease from vasculitis and autoimmune diseases, including but not limited to lupus erythematosis, polyarteritis, Wegeners' Granulomatosis, mixed connective tissue disease; ischemic renal injury; acute renal failure; chronic renal failure; glomerulonephritis; nephrotic syndrome; acute tubular necrosis; nephrosclerosis; glomerulosclerosis; minimal change disease; idiopathic membranous nephropathy; membranoproliferative glomerulonephritis; Berger's disease; mesangial proliferative glomerulonephritis; chronic glomerulonephritis; focal glomerulosclerosis; renal effects of Sjogren's syndrome; renal effects of scleroderma; interstitial nephritis; and renal injury following kidney transplant to the kidney donor, transplant recipient, and/or the transplanted kidney.

In certain embodiments, the subject has been identified as having an acute kidney injury (AKI) based on the Acute Kidney Injury Network (AKIN) criteria or Risk/Injury/Failure/Loss/ESRD (RIFLE) criteria.

In some embodiments, the renal disorder, disease, and/or injury is an acute kidney injury. In other embodiments, the renal disorder, disease, and/or injury is an ischemic acute kidney injury. In one embodiment, the subject is a human who has been identified as having reduced effective arterial volume. In one embodiment, the subject has been identified as having intravascular volume depletion (e.g., due to hemorrhage, gastrointestinal loss, renal loss, skin and mucous membrane loss, nephrotic syndrome, cirrhosis, or capillary leak). In one embodiment, the subject has been identified as having reduced cardiac output (e.g., due to cardiogenic shock, pericardial disease, congestive heart failure, valvular heart disease, pulmonary disease, or sepsis). In one embodiment, the subject has been identified as having systemic vasodilation (e.g., caused by cirrhosis, anaphylaxis, or sepsis). In one embodiment, the subject has been identified as having renal vasoconstriction (e.g., caused by early sepsis, hepatorenal syndrome, acute hypercalcemia, a drug, or a radiocontrast agent).

In some embodiments, the renal disorder, disease, and/or injury is a nephrotoxic kidney injury. In one embodiment, the human subject has been exposed to a nephrotoxin. For example, the nephrotoxin can be a nephrotoxic drug selected from the group consisting of an antibiotic (e.g., an aminoglycoside), a chemotherapeutic agent (e.g., cis-platinum), a calcineurin inhibitor, amphotericin B, and a radiographic contrast agent. In another example, the nephrotoxin can be an illicit drug or a heavy metal.

In certain embodiments, the subject has undergone a trauma injury or a crush injury.

In certain embodiments, the subject will undergo or has undergone an organ transplant surgery (e.g., a kidney transplant surgery or heart transplant surgery).

In certain embodiments, the subject will undergo or has undergone a surgery complicated by hypoperfusion.

In certain embodiments, the subject will undergo or has undergone cardiothoracic surgery or a vascular surgery.

In certain embodiments, the subject will be taking or has taken medication (e.g., an anticholinergic) that interferes with normal emptying of the bladder.

In certain embodiments, the subject has benign prostatic hypertrophy or a cancer (e.g., prostate cancer, ovarian cancer, or colorectal cancer).

In certain embodiments, the subject has a kidney stone.

In certain embodiments, the subject has an obstructed urinary catheter.

In certain embodiments, the subject has taken a drug that causes or leads to crystalluria, a drug that causes or leads to myoglobinuria, or a drug that causes or leads to cystitis.

In other embodiments, the 15-PGDH inhibitor can be administered to a subject to protect the subject's kidney from injury. In some embodiments, the subject is a human subject that has been or will be exposed to an ischemic or nephrotoxic insult. In some embodiments, the human subject has been exposed to oxidative damage (e.g., by free radicals such as reactive oxygen or nitrogen species.

In some embodiments, the 15-PGDH inhibitor can be administered to a human subject to protect the human subject's kidney from kidney injury during organ transplantation, such as kidney transplantation. The 15-PGDH can be administered to the kidney transplant donor, kidney transplant recipient, and/or transplanted kidney at an effective to protect the transplant donor, transplant recipient, and/or transplanted kidney from injury. In certain embodiments, the human subject can be administered one or more doses of a 15-PGDH inhibitor before and/or after (e.g., 0.1, 0.2, 0.3, 0.4, 0.5, 1, 2, 3, 4, 5, 6, 7, 8, 9, 10, 11, 12, 24, 48, 72, 96, 168 hours, or 1 week, 2 weeks, 3 weeks or 1 month) the kidney transplantation. It will be appreciated that administration of the 15-PGDH inhibitor can protect the human subject's kidney from kidney injury during other non-kidney organ transplantation.

Prostaglandins including PGE₁, PGE₂ and PGF₂ₐ have also been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Considering that 15-PGDH inhibits the activities of prostaglandins as mentioned in the above, the inhibition of 15-PGDH activity may lead to the promotion of bone resorption and bone formation that are inhibited by 15-PGDH. Thus, the 15-PGDH inhibitors described herein can be effective for the promotion of bone resorption and bone formation by inhibiting 15-PGDH activity. 15-PGDH inhibitors can also be used to increase bone density, treat osteoporosis, promote healing of fractures, or promote healing after bone surgery or joint replacement, or to promote healing of bone to bone implants, bone to artificial implants, dental implants, and bone grafts.

In yet other embodiments, the 15-PGDH inhibitors described herein can effective for treating 15-PGDH expressing cancers. Inhibition of 15-PGDH can inhibit the growth, proliferation, and metastasis of 15-PGDH expressing cancers.

In still other embodiments, the 15-PGDH inhibitors described herein can be effective for wound healing. Among various prostaglandins, PGE₂ is known to serve as a mediator for wound healing. Therefore, when skin is injured by wounds or burns, the inhibition of 15-PGDH activity can produce the treatment effect of the wounds or the burns by PGE₂.

Additionally, as discussed above, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in organs that would include liver, colon, and bone marrow. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, peripheral nerve.

Syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In embodiments, the ischemia is associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

In embodiments, the 15-PGDH inhibitor can be administered to a preparation of hematopoietic stem cells, such as peripheral blood hematopoietic stem cells or umbilical cord stem cells of the subject, to increase the fitness of the stem cell preparation as a donor graft or to decrease the number of units of umbilical cord blood required for transplantation.

Hematopoietic stem cells are multipotent stem cells that give rise to all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al, U.S. Pat. No. 5,635,387; McGlave, et al, U.S. Pat. No. 5,460,964; Simmons, P., et al, U.S. Pat. No. 5,677,136; Tsukamoto, et al, U.S. Pat. No. 5,750,397; Schwartz, et al, U.S. Pat. No. 5,759,793; DiGuisto, et al, U.S. Pat. No. 5,681,599; Tsukamoto, et al, U.S. Pat. No. 5,716,827). Hematopoietic stem cells (HSCs) give rise to committed hematopoietic progenitor cells (HPCs) that are capable of generating the entire repertoire of mature blood cells over the lifetime of an organism.

Hematopoietic stem cells and hematopoietic progenitor cells are described herein generally as hematopoietic stem cells unless noted otherwise and can refer to cells or populations identified by the presence of the antigenic marker CD34 (CD34⁺). In embodiments, the hematopoietic stem cells can be identified by the presence of the antigenic marker CD34 and the absence of lineage (lin) markers and are therefore characterized as CD34⁺/lin⁻ cells.

The hematopoietic stem cells used in the methods described herein may be obtained from any suitable source of hematopoietic stem and progenitor cells and can be provided as a high purified population of hematopoietic stem cells or as composition that includes about 0.01% to about 100% of hematopoietic stem cells. For example, hematopoietic stem cells may be provided in compositions, such as unfractionated bone marrow (where the hematopoietic stem cells comprise less than about 1% of the bone marrow cell population), umbilical cord blood, placental blood, placenta, fetal blood, fetal liver, fetal spleen, Wharton's jelly, or mobilized peripheral blood.

Suitable sources of hematopoietic stem cells can be isolated or obtained from an organ of the body containing cells of hematopoietic origin. The isolated cells can include cells that are removed from their original environment. For example, a cell is isolated if it is separated from some or all of the components that normally accompany it in its native state. For example, an “isolated population of cells,” an “isolated source of cells,” or “isolated hematopoietic stem cells” and the like, as used herein, refer to in vitro or ex vivo separation of one or more cells from their natural cellular environment, and from association with other components of the tissue or organ, i.e., it is not significantly associated with in vivo substances.

Hematopoietic stem cells can be obtained or isolated from bone marrow of adults, which includes femurs, hip, ribs, sternum, and other bones. Bone marrow aspirates containing hematopoietic stem cells can be obtained or isolated directly from the hip using a needle and syringe. Other sources of hematopoietic stem cells include umbilical cord blood, placental blood, mobilized peripheral blood, Wharton's jelly, placenta, fetal blood, fetal liver, or fetal spleen. In particular embodiments, harvesting a sufficient quantity of hematopoietic stem cells for use in therapeutic applications may require mobilizing the stem and progenitor cells in the donor.

“Hematopoietic stem cell mobilization” refers to the release of stem cells from the bone marrow into the peripheral blood circulation for the purpose of leukapheresis, prior to stem cell transplantation. By increasing the number of stem cells harvested from the donor, the number of stem cells available for therapeutic applications can be significantly improved. Hematopoietic growth factors, e.g., granulocyte colony stimulating factor (G-CSF) or chemotherapeutic agents often are used to stimulate the mobilization. Commercial stem cell mobilization drugs exist and can be used in combination with G-CSF to mobilize sufficient quantities of hematopoietic stem and progenitor cells for transplantation into a subject. For example, G-CSF and Mozobil (Genzyme Corporation) can be administered to a donor in order to harvest a sufficient number of hematopoietic cells for transplantation. Other methods of mobilizing hematopoietic stem cells would be apparent to one having skill in the art.

In embodiments, hematopoietic stem and progenitor cells (HSPCs) are obtained from umbilical cord blood. Cord blood can be harvested according to techniques known in the art {see, e.g., U.S. Pat. Nos. 7,147,626 and 7,131,958, herein incorporated by reference for such methodologies).

In embodiments, HSPCs can be obtained from pluripotent stem cell sources, e.g., induced pluripotent stem cells (iPSCs) and embryonic stem cells (ESCs). As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell. As used herein, the term “reprogramming” refers to a method of increasing the potency of a cell to a less differentiated state. As used herein, the term “programming” refers to a method of decreasing the potency of a cell or differentiating the cell to a more differentiated state.

In embodiments, the hematopoietic stem cells can be administered or contacted ex vivo with one or more 15-PGDH inhibitors described herein to provide a therapeutic composition. In embodiments, the therapeutic compositions of the can include a population of hematopoietic stem cells treated ex vivo with a one or more 15-PGDH inhibitor. In certain embodiments, the therapeutic composition comprising the enhanced HSPCs is whole bone marrow, umbilical cord blood, or mobilized peripheral blood.

In particular embodiments, the therapeutic composition includes a population of cells, wherein the population of cells is about 95% to about 100% hematopoietic stem cells. The invention contemplates, in part, that using therapeutic compositions of highly purified hematopoietic stem cells, e.g., a composition comprising a population of cells wherein the cells comprise about 95% hematopoietic stem cells, may improve the efficiency of stem cell therapies. Currently practiced methods of transplantations typically use unfractionated mixtures of cells where hematopoietic stem cells comprise less than 1% of the total cell population.

In embodiments, the therapeutic composition comprises a population of cells, wherein the population of cells comprises less than about 0.1%, 0.5%, 1%, 2%, 5%, 10%, 15%, 20%, 25%, or 30% hematopoietic stem cells. The population of cells In embodiments comprises less than about 0.1%, 0.5%, 1%, 2%, 5%, 10%, 15%, 20%, 25%, or 30% hematopoietic stem cells. In embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% hematopoietic stem cells.

Hematopoietic stem cells in the therapeutic compositions of the invention can be autologous/autogeneic (“self) or non-autologous (“non-self,” e.g., allogeneic, syngeneic or xenogeneic) relative to a subject to which the therapeutic composition is to be administered. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Hematopoietic stem cells for use in the methods of the present invention may be depleted of mature hematopoietic cells such as T cells, B cells, NK cells, dendritic cells, monocytes, granulocytes, erythroid cells, and their committed precursors from bone marrow aspirate, umbilical cord blood, or mobilized peripheral blood (mobilized leukapheresis product). Mature, lineage committed cells are depleted by immunodepletion, for example, by labeling solid substrates with antibodies that bind to a panel of so-called “lineage” antigens: CD2, CD3, CD11b, CD14, CD15, CD16, CD79, CD56, CD123, and CD235a. A subsequent step can be performed to further purify the population of cells, in which a substrate labeled with antibodies that bind to the CD34⁺ antigen are used to isolate primitive hematopoietic stem cells. Kits are commercially available for purifying stem and progenitor cells from various cell sources and in particular embodiments, these kits are suitable for use with the methods described herein.

In embodiments, the amount of hematopoietic stem cells in the therapeutic composition is at least 0.1×10⁵ cells, at least 0.5×10⁵ cells, at least 1×10⁵ cells, at least 5×10⁵ cells, at least 10×10⁵ cells, at least 0.5×10⁶ cells, at least 0.75×10⁶ cells, at least 1×10⁶ cells, at least 1.25×10⁶ cells, at least 1.5×10⁶ cells, at least 1.75×10⁶ cells, at least 2×10⁶ cells, at least 2.5×10⁶ cells, at least 3×10⁶ cells, at least 4×10⁶ cells, at least 5×10⁶ cells, at least 10×10⁶ cells, at least 15×10⁶ cells, at least 20×10⁶ cells, at least 25×10⁶ cells, or at least 30×10⁶ cells.

In embodiments, the amount of hematopoietic stem cells in the therapeutic composition is the amount of HSPCs in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.5×10⁶ cells/kg of bodyweight, at least 0.75×10⁶ cells/kg of bodyweight, at least 1×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight.

Preparations of hematopoietic stem cells administered one or more 15-PGDH inhibitors and/or therapeutic compositions that include hematopoietic stem cells and one or more 15-PGDH inhibitor can be used for improving hematopoietic stem cell transplants and in treating ischemia or ischemia-damaged tissue, and in reducing further damage to ischemic tissue and/or repairing damage to ischemic tissue through cell recruitment, improving vascularization in ischemic tissue, improving tissue regeneration at sites of ischemia, decreasing ischemic tissue necrosis or apoptosis, and/or increasing cell survival at sites of ischemia. In particular embodiments, the preparations of 15-PGDH inhibitor treated hematopoietic stem cells and/or therapeutic compositions of 15-PGDH inhibitors and hematopoietic stem cells are useful to subjects in need of hematopoietic reconstitution, such as subjects that have undergone or are scheduled to undergo myeloablative therapy.

Subjects, which can be treated with the preparations of 15-PGDH inhibitor treated hematopoietic stem cells and/or therapeutic compositions of 15-PGDH inhibitors and hematopoietic stem cells, can include subjects that have or that have been diagnosed with various types of leukemias, anemias, lymphomas, myelomas, immune deficiency disorders, and solid tumors. A subject also includes a human who is a candidate for stem cell transplant or bone marrow transplantation, such as during the course of treatment for a malignant disease or a component of gene therapy. Subjects may also include individuals or animals that donate stem cells or bone marrow for allogeneic transplantation. In certain embodiments, a subject may have undergone myeloablative irradiation therapy or chemotherapy, or may have experienced an acute radiation or chemical insult resulting in myeloablation. In certain embodiments, a subject may have undergone irradiation therapy or chemotherapy, such as during various cancer treatments. Typical subjects include animals that exhibit aberrant amounts (lower or higher amounts than a “normal” or “healthy” subject) of one or more physiological activities that can be modulated by an agent or a stem cell or marrow transplant.

Subjects, which can be treated with the preparations of 15-PGDH inhibitor treated hematopoietic stem cells and/or therapeutic compositions of 15-PGDH inhibitors and hematopoietic stem cells, can also include subjects undergoing chemotherapy or radiation therapy for cancer, as well as subjects suffering from (e.g., afflicted with) nonmalignant blood disorders, particularly immunodeficiencies (e.g. SCID, Fanconi's anemia, severe aplastic anemia, or congenital hemoglobinopathies, or metabolic storage diseases, such as Hurler's disease, Hunter's disease, mannosidosis, among others) or cancer, particularly hematological malignancies, such as acute leukemia, chronic leukemia (myeloid or lymphoid), lymphoma (Hodgkin's or non-Hodgkin's), multiple myeloma, myelodysplastic syndrome, or non-hematological cancers such as solid tumors (including breast cancer, ovarian cancer, brain cancer, prostate cancer, lung cancer, colon cancer, skin cancer, liver cancer, or pancreatic cancer).

Subjects may also include subjects suffering from aplastic anemia, an immune disorder (severe combined immune deficiency syndrome or lupus), myelodysplasia, thalassemaia, sickle-cell disease or Wiskott-Aldrich syndrome. In embodiments, the subject suffers from a disorder that is the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. Other subjects may have disorders caused by an infection (e.g., viral infection, bacterial infection or fungal infection) which causes damage to stem or progenitor cells of the bone marrow.

In addition, subjects suffering from the following conditions can also benefit from treatment using the preparations of 15-PGDH inhibitor treated hematopoietic stem cells and/or therapeutic compositions of 15-PGDH inhibitors and hematopoietic stem cells: lymphocytopenia, lymphorrhea, lymphostasis, erythrocytopenia, erthrodegenerative disorders, erythroblastopenia, leukoerythroblastosis; erythroclasis, thalassemia, myelodysplasia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated neutropenias, childhood and adult cyclic neutropaenia; post-infective neutropaenia; myelodysplastic syndrome; neutropaenia associated with chemotherapy and radiotherapy; chronic granulomatous disease; mucopolysaccharidoses; Diamond Blackfan Anemia; Sickle cell disease; or Beta thalassemia major.

In embodiments, the preparations of 15-PGDH inhibitor treated hematopoietic stem cells and/or therapeutic compositions or 15-PGDH inhibitors and hematopoietic stem cells can be used in cell-based therapy for treating ischemic tissue or treating or ameliorating one or more symptoms associated with tissue ischemia, including, but not limited to, impaired, or loss of, organ function (including without limitation impairments or loss of brain, kidney, or heart function), cramping, claudication, numbness, tingling, weakness, pain, reduced wound healing, inflammation, skin discoloration, and gangrene.

In embodiments, the subject exhibits at least one symptom of an ischemic tissue or tissue damaged by ischemia. In particular embodiments, the subject is a human who is has or who is at risk of having an ischemic tissue or tissue damaged by ischemia, e.g., a subject that has diabetes, peripheral vascular disease, thromboangiitis obliterans, vasculitis, cardiovascular disease, coronary artery disease or heart failure, or cerebrovascular disease, cardiovascular disease, or cerebrovascular disease.

Illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In various embodiments, the invention contemplates that the therapeutic cell compositions disclosed herein can be used to treat an ischemic tissue in which it is desirable to increase the blood flow, oxygen supply, glucose supply, or supply of nutrients to the tissue.

In embodiments, the 15-PGDH inhibitor can be administered to a preparation of tissue stem cells, such as neural stem stems, mesenchymal stem cells, or stem cells that can generate other tissues, and/or a preparation of pluripotent stem cells.

In embodiments, tissue stems cells can be obtained from pluripotent stem cell sources, e.g., induced pluripotent stem cells (iPSCs) and embryonic stem cells (ESCs). As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell. As used herein, the term “reprogramming” refers to a method of increasing the potency of a cell to a less differentiated state. As used herein, the term “programming” refers to a method of decreasing the potency of a cell or differentiating the cell to a more differentiated state.

In embodiments, the tissue stem cells and/or pluripotent stem cells can be administered or contacted ex vivo with one or more 15-PGDH inhibitors described herein to provide a therapeutic composition. In embodiments, the therapeutic compositions of the can include a population of tissue stem cells treated ex vivo with a one or more 15-PGDH inhibitor.

In particular embodiments, the therapeutic composition includes a population of cells, wherein the population of cells is about 95% to about 100% tissue stem cells. The invention contemplates, in part, that using therapeutic compositions of highly purified tissue stem cells, e.g., a composition comprising a population of cells wherein the cells comprise about 95% tissue stem cells, may improve the efficiency of stem cell therapies.

In embodiments, the therapeutic composition comprises a population of cells, wherein the population of cells comprises less than about 0.1%, 0.5%, 1%, 2%, 5%, 10%, 15%, 20%, 25%, or 30% tissue stem cells. The population of cells in embodiments comprises less than about 0.1%, 0.5%, 1%, 2%, 5%, 10%, 15%, 20%, 25%, or 30% tissue stem cells. In embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% tissue stem cells.

Tissue stem cells in the therapeutic compositions of the invention can be autologous/autogeneic (“self) or non-autologous (“non-self,” e.g., allogeneic, syngeneic or xenogeneic) relative to a subject to which the therapeutic composition is to be administered. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Preparations of tissue stem cells administered one or more 15-PGDH inhibitors and/or therapeutic compositions that include tissue stem cells and one or more 15-PGDH inhibitor can be used for improving tissue stem cell transplants and in treating damaged tissue, and in reducing further tissue damage tissue and/or potentiating repair to damaged tissue through stem cell recruitment and/or increasing cell survival at sites of tissue damage.

Syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In embodiments, the 15-PGDH inhibitor can be administered to a bone marrow graft donor or a hematopoietic stem cell donor to increase the fitness of a donor bone marrow graft or a donor hematopoietic stem cell graft.

In embodiments, the 15-PGDH inhibitor can also be administered to bone marrow of a subject to increase stem cells in the subject or to increase the fitness of the marrow as a donor graft.

In embodiments, the 15-PGDH inhibitor can be administered to a subject to mitigate bone marrow graft rejection, to enhance bone marrow graft engraftment, to enhance engraftment of a hematopoietic stem cell graft, or an umbilical cord blood stem cell graft, to enhance engraftment of a hematopoietic stem cell graft, or an umbilical cord stem cell graft, and/or to decrease the number of units of umbilical cord blood required for transplantation into the subject. The administration can be, for example, following treatment of the subject or the marrow of the subject with radiation therapy, chemotherapy, or immunosuppressive therapy.

In embodiments, the 15-PGDH inhibitor can be administered to a recipient of a bone marrow transplant, of a hematopoietic stem cell transplant, or of an umbilical cord blood stem cell transplant, in order to decrease the administration of other treatments or growth factors.

In embodiments, the 15-PGDH inhibitor can be administered to a subject to enhance recovery of neutrophils following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, neutropenias from other bone marrow diseases, drug induced neutropenia, immune neutropenias, idiopathic neutropenia, and following infections with viruses that include, but are not limited to, HIV, CMV, and parvovirus.

In embodiments, the 15-PGDH inhibitor can be administered to a subject to enhance recovery of platelets following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, thrombocytopenias from other bone marrow diseases, drug induced thrombocytopenia, immune thrombocytopenia, idiopathic thrombocytopenic purpura, idiopathic thrombocytopenia, and following infections with viruses that include, but are not limited to, HIV, CMV, and parvovirus.

In embodiments, the 15-PGDH inhibitor can be administered to a subject to enhance recovery of hemoglobin following bone marrow transplantation, following umbilical cord blood transplantation, following transplantation with hematopoietic stem cells, following conventional chemotherapy, following radiation treatment, and in individuals with anemias from diseases that include but are not limited to aplastic anemia, myelodysplasia, myelofibrosis, anemia from other bone marrow diseases, drug induced anemia, immune mediated anemias, anemia of chronic disease, idiopathic anemia, and following infections with viruses that include, but are not limited to, HIV, CMV, and parvovirus.

In embodiments, the 15-PGDH inhibitor can be administered to a subject to enhance numbers of bone marrow stem cell numbers following bone marrow transplantation, following umbilical cord blood transplantation, following transplantation with hematopoietic stem cells, following conventional chemotherapy, following radiation treatment, in individuals with other bone marrow diseases, in individuals with cytopenias following viral infections, and in individuals with cytopenias.

In embodiments, the 15-PGDH inhibitor can be administered to a subject to enhance response to cytokines administered to individuals with cytopenias that include but are not limited to neutropenia, thrombocytopenia, lymphocytopenia, and anemia. Cytokines whose responses may be enhanced by SW033291 include, but are not limited to: G-CSF, GM-CSF, EPO, IL-3, IL-6, TPO, SCF, and TPO-RA (thrombopoietin receptor agonist).

In further embodiments, the 15-PGDH inhibitor can be administered to a subject or to a tissue graft of a subject to mitigate graft rejection, to enhance graft engraftment, to enhance graft engraftment following treatment of the subject or the marrow of the subject with radiation therapy, chemotherapy, or immunosuppressive therapy, to confer resistance to toxic or lethal effects of exposure to radiation, confer resistance to the toxic effect of Cytoxan, the toxic effect of fludarabine, the toxic effect of chemotherapy, or the toxic effect of immunosuppressive therapy, to decrease infection, and/or to decrease pulmonary toxicity from radiation.

In embodiments, the 15-PGDH inhibitor can be administered to a recipient of a tissue stem cell transplant, including but not limited to a transplant with hematopoietic stem cells, neural stem stems, mesenchymal stem cells, or stem cells for other tissues, so as to accelerate tissue regeneration and repair following the transplant.

In embodiments, the administration of a 15-PGDH inhibitor can be in combination with G-CSF for the purpose of increasing neutrophils.

In embodiments, the administration of a 15-PGDH inhibitor can be in combination with a hematopoietic cytokine for the purpose of increasing neutrophils.

In still other embodiments, the administration of a 15-PGDH inhibitor can be in combination with G-CSF for the purpose of increasing numbers of and/or of mobilizing peripheral blood hematopoietic stem cells.

In embodiments, the administration of a 15-PGDH inhibitor can be in combination with a hemopoietic cytokine for the purpose of increasing numbers of and/or of mobilizing peripheral blood hematopoietic stem cells.

In embodiments, the administration of a 15-PGDH inhibitor can be in combination with a second agent, including Plerixafor, for the purpose of increasing numbers of and/or of mobilizing peripheral blood hematopoietic stem cells.

In embodiments, the administration of a 15-PGDH inhibitor can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor can be in combination with a hemopoietic cytokine for the purpose of increasing numbers of and/or of mobilizing peripheral blood hematopoietic stem cells for use in hematopoietic stem cell transplantation.

In embodiments, the administration of a 15-PGDH inhibitor can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor can be in combination with G-CSF for the purpose of increasing numbers of hematopoietic stem cells in blood or bone marrow.

In embodiments, the administration of a 15-PGDH inhibitor can be in combination with a hemopoietic cytokine for the purpose of increasing numbers of hematopoietic stem cells in blood or bone marrow.

In embodiments, the 15-PGDH inhibitors can be used to treat and/or prevent fibrosis and various fibrotic diseases, disorders or conditions, and decrease fibrotic symptoms, such as collagen deposition, inflammatory cytokine expression, and inflammatory cell infiltration.

In embodiments, a method of treating or preventing a fibrotic disease, disorder or condition includes administering to a subject in need thereof a therapeutically effect amount of a 15-PGDH inhibitor such that at least one symptom or feature of a fibrotic disease, disorder or condition, or other related diseases, disorders or conditions, is reduced in intensity, severity, or frequency, or has delayed onset.

As used herein, the term “fibrotic” diseases, disorders, or conditions include diseases, disorders, or conditions characterized, in whole or in part, by the excess production of fibrous material, including excess production of fibrotic material within the extracellular matrix, or the replacement of normal tissue elements by abnormal, non-functional, and/or excessive accumulation of matrix-associated components. The fibrotic diseases, disorders, or conditions, can include acute and chronic, clinical or subclinical presentation, in which fibrogenic associated biology or pathology is evident.

Examples of fibrotic diseases, disorders and conditions include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myeloid leukemia, acute myelogenous leukemia, myelodysplastic syndrome, myeloproliferative syndrome, gynecological cancer, Kaposi's sarcoma, Hansen's disease, collagenous colitis, acute fibrosis, organ specific fibrosis, and the like.

Illustrative organ specific fibrotic disorders include, but are not limited to, pulmonary fibrosis, pulmonary hypertension, cystic fibrosis, asthma, chronic obstructive pulmonary disease, liver fibrosis, kidney fibrosis, NASH, and the like. Many fibrotic diseases, disorders or conditions have disordered and/or exaggerated deposition of extracellular matrix in affected tissues. Fibrosis may be associated with inflammation, occur as a symptom of underlying disease, and/or caused by surgical procedure or wound healing process. Unchecked fibrosis can result in destruction of the architecture of the underlying organ or tissue, commonly referred to as scarring.

In embodiments, the 15-PGDH inhibitors can be used to treat or prevent lung fibrosis. The lung fibrosis can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

Pulmonary fibrosis is characterized by progressive scarring of lung tissue accompanied by fibroblast proliferation, excessive accumulation of extracellular matrix proteins, and abnormal alveolar structure. The thickened and stiff tissue makes it difficult for lungs to work properly, leading to breathing problems, such as shortness of breath, and can ultimately be fatal. Pulmonary fibrosis may be caused by acute lung injury, viral infection, exposure to toxins, radiation, chronic disease, medications, or may be idiopathic (i.e., an undiscovered underlying cause).

The classic findings in idiopathic pulmonary fibrosis show diffuse peripheral scarring of the lungs with small bubbles (known as bullae) adjacent to the outer lining of the surface of the lung, often at the bases of the lungs. Idiopathic pulmonary fibrosis often has a slow and relentless progression. Early on, patients often complain of a dry unexplained cough. Next, shortness of breath (dyspnea) sets in and worsens over time triggered by less and less activity. Eventually, the shortness of breath becomes disabling, limiting all activity and even occurring while sitting still. In rarer cases, the fibrosis can be rapidly progressive, with dyspnea and disability occurring in weeks to months of onset of the disease. This form of pulmonary fibrosis has been referred to as Hamman-Rich syndrome.

Pulmonary hypertension is marked by an increase in the blood pressure of the lung vasculature, including the pulmonary artery, pulmonary vein, and/or pulmonary capillaries. Abnormally high pressure strains the right ventricle of the heart, causing it to expand. Over time, the right ventricle can weaken and lose its ability to pump enough blood to the lungs, leading to the development of heart failure. Pulmonary hypertension can occur as a result of other medical conditions, such as chronic liver disease and liver cirrhosis; rheumatic disorders such as scleroderma or systemic lupus erythematosus (lupus); and lung conditions including tumors, emphysema, chronic obstructive pulmonary disease (COPD), and pulmonary fibrosis. Pulmonary fibrosis may lead to narrowing of pulmonary vasculature resulting in pulmonary hypertension.

Chronic Obstructive Pulmonary Disease (COPD) is a common lung disease that is often associated with chronic bronchitis or emphysema. Symptoms can often include cough, mucus build up, fatigue, wheezing, and respiratory infection.

Chronic bronchitis and emphysema are diseases of the lungs in which the airways become narrowed. This leads to a limitation of the flow of air to and from the lungs, causing shortness of breath (dyspnea). In clinical practice, COPD is defined by its characteristically low airflow on lung function tests.

Lung damage and inflammation in the large airways results in chronic bronchitis. In the airways of the lung, the hallmark of chronic bronchitis is an increased number (hyperplasia) and increased size (hypertrophy) of the goblet cells and mucous glands of the airway. As a result, there is more mucus than usual in the airways, contributing to narrowing of the airways and causing a cough with sputum. Microscopically there is infiltration of the airway walls with inflammatory cells. Inflammation is followed by scarring and remodeling that thickens the walls and also results in narrowing of the airways. As chronic bronchitis progresses, there is squamous metaplasia (an abnormal change in the tissue lining the inside of the airway) and fibrosis (further thickening and scarring of the airway wall). The consequence of these changes is a limitation of airflow and difficulty breathing.

Asthma is a chronic lung disease characterized by inflammation and constriction of the airways. Asthma causes recurring periods of wheezing, tightness of the chest, shortness of breath, and coughing. Swelling and overproduction of mucus can cause further airway constriction and worsening of symptoms. There is evidence that increased matrix degradation may occur in asthma, and this may contribute to mechanical changes in the airways in asthma (Roberts et al (1995) Chest 107:111 S-117S, incorporated herein by reference in its entirety. Treatment of extracellular matrix degradation may ameliorate symptoms of asthma.

Cystic fibrosis is a recessive multi-system genetic disease characterized by abnormal transport of chloride and sodium across epithelium, leading to thick, viscous secretions in the lungs, pancreas, liver, intestine and reproductive tract. Cystic fibrosis is caused by a mutation in the gene for the protein cystic fibrosis transmembrane conductance regulator (CFTR). Lung disease results from clogging of the airways due to mucus build-up, decreased mucociliary clearance, and resulting inflammation, which can cause fibrotic injury and structural changes to the lungs. The fibrotic lung damage progresses over time leading some cystic fibrosis patients to require lung transplant.

Common symptoms of subjects suffering from cystic fibrosis include, but are not limited to, accumulation of thick mucus, copious phlegm production, frequent chest infections, frequent coughing, frequent shortness of breath, inflammation, decreased ability to exercise, opportunistic infections of the lung and sinus (including but not limited to Staphylococcus aureus, Haemophilus influenzae, Mycobacterium avium, and Pseudomonas aeruginosa), pneumonia, tuberculosis, bronchiectasis, hemoptysis, pulmonary hypertension (and resulting heart failure), hypoxia, respiratory failure, allergic bronchopulmonary aspergillosis, mucus in the paranasal sinuses, sinus infection, facial pain, fever, excessive nasal drainage, development of nasal polyps, cardiorespiratory complications, CF-related diabetes, rectal prolapse, pancreatitis, malabsorption, intestinal blockage, exocrine pancreatic insufficiency, bile duct blockage, and liver cirrhosis.

In embodiments, the 15-PGDH inhibitors can be used to treat or prevent fibrotic diseases, disorders or conditions caused by post-surgical adhesion formation. Post-surgical adhesion formation is a common complication of surgery. The formation of adhesions, from mechanical damage, ischemia, and infections, can increase morbidity and mortality following surgery. Although refined surgical procedures can reduce the magnitude of adhesion formation, adhesions are rarely eviscerated and an effective adjunctive therapy is needed. Reducing the fibrosis associated with this process could reduce pain, obstruction and other complications of surgery and promote healing and recovery.

Wounds (i.e., lacerations, openings) in mammalian tissue result in tissue disruption and coagulation of the microvasculature at the wound face. Repair of such tissue represents an orderly, controlled cellular response to injury. Soft tissue wounds, regardless of size, heal in a similar manner. Tissue growth and repair are biologic systems wherein cellular proliferation and angiogenesis occur in the presence of an oxygen gradient. The sequential morphological and structural changes which occur during tissue repair have been characterized in detail and have in some instances been quantified (see e.g., Hunt, T. K., et al., “Coagulation and macrophage stimulation of angiogenesis and wound healing,” in The Surgical Wound, pp. 1-18, ed. F. Dineen & G. Hildrick-Smith (Lea & Febiger, Philadelphia: 1981)). The cellular morphology consists of three distinct zones. The central avascular wound space is oxygen deficient, acidotic and hypercarbic, and has high lactate levels. Adjacent to the wound space is a gradient zone of local anemia (ischemia) which is populated by dividing fibroblasts. Behind the leading zone is an area of active collagen synthesis characterized by mature fibroblasts and numerous newly-formed capillaries (i.e., neovascularization). U.S. Pat. Nos. 5,015,629 and 7,022,675 (each incorporated by reference herein) disclose methods and compositions for increasing the rate of wound repair.

In embodiments, the 15-PGDH inhibitors can used for reducing or preventing scar formation in a subject by administering to a subject in need of treatment. Scar formation is a natural part of the healing process. Disorderly collagen synthesis and deposition in a wound can result in excessive, thick, or raised scar formation. Generally, the larger the wound, the longer it takes to heal and the greater the chance of a problematic scar.

In embodiments, the 15-PGDH inhibitors can be used to reduce or prevent scar formation on skin or scleroderma. There are several types of scars on skin. Hypertrophic scars are raised, pinkish-red areas located inside the borders of the original injury. They are often described as itchy. In some cases, hypertrophic scars shrink and fade on their own. Keloids are raised, deep-red areas that tend to cover much more area than that of the original injury. Even when surgically removed, keloids tend to recur. Atrophic scars are skin depressions, like those that sometimes form from severe acne. They are caused by inflammation that destroys the collagen during the rebuilding process, leaving an area of indentation.

In embodiments, the 15-PGDH inhibitors can be used to treat or prevent systemic sclerosis. Systemic sclerosis is a systemic connective tissue disease characterized by alterations of the microvasculature, disturbances of the immune system and by massive deposition of collagen and other matrix substances in the connective tissue. Systemic sclerosis is a clinically heterogeneous generalized disorder which affects the connective tissue of the skin and internal organs such as gastrointestinal tract, lungs, heart and kidneys. Reduction of fibrosis resulting from systemic sclerosis may ameliorate symptoms and/or prevent further complications in affected tissues.

In embodiments, the 15-PGDH inhibitors can be used to treat or prevent liver fibrosis. Liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, α-1-antitrypsin deficiency, primary biliary cirrhosis, drug reaction and exposure to toxins.

Nonalcoholic steatohepatitis (NASH) is a common liver disease. It resembles alcoholic liver disease but occurs in people who drink little or no alcohol. The major feature in NASH is fat in the liver, along with inflammation and damage. Nevertheless, NASH can be severe and can lead to cirrhosis, in which the liver is permanently damaged and scarred and no longer able to work properly.

NASH is usually a silent disease with few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases may even begin to reverse on its own without specific therapy. Or NASH can slowly worsen, causing scarring or fibrosis to appear and accumulate in the liver. As fibrosis worsens, cirrhosis develops in which the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

In embodiments, the 15-PGDH inhibitors can be used to treat or prevent kidney fibrosis. Kidney fibrosis can result from dialysis following kidney failure, catheter placement, a nephropathy, glomerulosclerosis, glomerulonephritis, chronic renal insufficiency, acute kidney injury, end stage renal disease or renal failure.

Kidney (renal) fibrosis results from excessive formation of fibrous connective tissue in the kidney. Kidney fibrosis causes significant morbidity and mortality and leads to a need for dialysis or kidney transplantation. Fibrosis can occur in either the filtering or reabsorptive component of the nephron, the functional unit of the kidney. A number of factors may contribute to kidney scarring, particularly derangements of physiology involved in the autoregulation of glomerular filtration. This in turn leads to replacement of normal structures with accumulated extracellular matrix. A spectrum of changes in the physiology of individual cells leads to the production of numerous peptide and non-peptide fibrogens that stimulate alterations in the balance between extracellular matrix synthesis and degradation to favor scarring.

In embodiments, the symptoms of fibrosis of a tissue organ can comprise inflammation. In these embodiments, a therapeutically effective amount of the 15-PGDH inhibitor administered to the subject in need thereof can be an amount effective to decrease or reduce inflammatory cell count in the tissue or organ. A relevant sample can be obtained from the subject to determine the decrease or reduction in inflammatory cell count. In a non-limiting embodiment, the beneficial effect may be assessed by demonstrating a reduction in neutrophil count in BAL fluid from the subject with cystic fibrosis. The excessive recruitment of neutrophils into the airways of patients with CF is a significant predictor of lung disease severity in CF and therefore is an important therapeutic target. Methods for measuring such cell counts are well known in the art, including but not limited to FACS techniques. In embodiments, the method may comprise reducing neutrophil cell count in BAL fluid from the subject compared to control. Any suitable control can be used for comparison, such as cystic fibrosis subjects not treated the 15-PGDH inhibitors. In embodiments, a decrease in inflammatory cell count, such as neutrophil count, provides a clinical benefit to the subject. In various embodiments, the reduction in inflammatory cell count is at least 5%, 10%, 15%, 20%, 25%, 50%, or more compared to control.

In another embodiment, the beneficial effect of the 15-PGDH inhibitors may be assessed by a reduction in one or more inflammatory biomarkers in a relevant sample from the subject. In various non-limiting embodiments, the inflammatory biomarker may comprise or consist of one or more of cytokines or inflammatory cytokines associated with fibrosis. Such cytokines can include, for example, IL1β, MIP2 (e.g., CCL3 or CCL4), IFNδ, TGFβ, TNFα, IL-6, MCP-1, IL2, and IL-10 in BAL fluid. Methods for measuring the amount of such biomarkers are well known in the art, including but not limited to ELISAs. Thus, in this embodiment, the methods may further comprise the reducing an amount of one or more inflammatory biomarkers in a sample from the subject compared to control.

In embodiments, the 15-PGDH inhibitors can be used in a method for decreasing or reducing collagen secretion or collagen deposition in a tissue or organ, such as the lung, the liver, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult are organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

15-PGDH inhibitors used to treat the fibrotic disease, disorder or condition and/or reduce collagen deposition can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%. Additionally, in a model organism, PGE₂ signaling stimulates liver regeneration and increase survival after exposure to hepatoxic agents, such as acetaminophen. Hence, 15-PGDH inhibitors described herein may be utilized to increase liver regeneration after liver resection, in other settings that include after liver surgery, after live liver donation, or after receiving a liver transplant or to increase liver regeneration and increase survival after exposures to hepatoxic agents, including but not limited to acetaminophen and similar compounds.

PGE1 analogues have also been used in the treatment of erectile dysfunction. Accordingly, in embodiments, 15-PGDH inhibitors described herein can used either alone or combination with a prostaglandin for the treatment of erectile dysfunction.

Other embodiments described herein relate to the use of 15-PGDH inhibitors in combination with corticosteroids to treat inflammation and/or reduce aberrant activity of the immune system in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory and/or immune disorders by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects. Additional embodiments herein relate to the use of 15-PGDH inhibitors in combination with TNF alpha inhibitors to treat inflammation and/or reduce aberrant activity of the immune system in a subject in need thereof.

In embodiments, the 15-PGDH inhibitors can be administered in combination with corticosteroids and/or TNF inhibitors to treat intestinal, gastrointestinal, or bowel disorders. The intestinal, gastrointestinal, or bowel disorders treated can include oral ulcers, gum disease, gastritis, colitis, ulcerative colitis, gastric ulcers, inflammatory bowel disease, and Crohn's disease. As described below, it was found that that inhibitors of short-chain dehydrogenase activity, such as 15-PGDH inhibitors, can be administered to a subject in need thereof alone or in combination with corticosteroids to treat intestinal, gastrointestinal, or bowel disorders, such as oral ulcers, gum disease, gastritis, colitis, ulcerative colitis, gastric ulcers, inflammatory bowel disease, and Crohn's disease.

The 15-PGDH inhibitors described herein can be used in a pharmaceutical composition for the prevention or the treatment of oral, intestinal, and/or gastrointestinal injury or diseases, or inflammatory bowel disease (IBD), such as Crohn's disease, oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In the stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, that is, the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Additionally, corticosteroids and TNF alpha antagonists are both used in the treatment of ulcerative colitis and IBD patients. In mouse models, 15-PGDH inhibitors speed healing of ulcerative colitis. We have found that administering corticosteroids to mice elevates levels of colon 15-PGDH, an effect that should reduce the therapeutic effectiveness of corticosteroids in colitis treatment. This suggests that combining a corticosteroid with a 15-PGDH inhibitor should be more effective in colitis (and IBD) treatment than using either agent alone.

Similarly, we have shown that TNF-alpha suppresses colon 15-PGDH expression. This suggests that TNF-alpha antagonists will increase colon 15-PGDH expression, an effect that should reduce the therapeutic effectiveness of corticosteroids in colitis treatment. This suggests that combining a TNF-α antagonist, e.g., the chimeric antibody REMICADE (infliximab), with a 15-PGDH inhibitor should be more effective in colitis (and IBD) treatment than using either agent alone.

In embodiments, the 15-PGDH inhibitors and corticosteroids or 15-PGDH inhibitors and TNF inhibitors can be provided in a topical composition or formulation that is used to treat inflammation and/or aberrant immune system activity associated with medical conditions, such as atopic dermatitis, psoriasis, eczematous dermatitis, nummular dermatitis, irritant contact dermatitis, allergic contact dermatitis (such as poison ivy exposure, poison oak exposure, and poison sumac exposure), seborrheic dermatitis, stasis dermatitis, and other steroid responsive dermatoses.

In embodiments, the 15-PGDH inhibitors and corticosteroids or 15-PGDH inhibitors and TNF inhibitors provided in a topical composition can be used to treat, for example, acne vulgaris, alopecia, alopecia greata, vitiligo, eczema, xerotic eczema, keratosis pilaris, lichen planus, lichen sclerosus, lichen striatus, lichen simplex chronicus, prurigo nodularis, discoid lupus erythematosus, lymphocytic infiltrate of Jessner/Kanof, lymphacytoma cutis, pyoderma gangrenosum, pruritis ani, sarcoidosis, chondrodermatitis nodularis helices, and other inflammatory dermatological disorders.

Medical conditions treated by the 15-PGDH inhibitors and corticosteroids or 15-PGDH inhibitors and TNF inhibitors can also include, for example, keloids, hypertrophic scars, pretibial myxedema and other infiltrative dermatological disorders. Additional medical conditions include, for example, granuloma annulare, necrobiosis lipoidica diabeticorum, sarcoidosis, and other noninfectious granulomas.

In still other embodiments, the 15-PGDH inhibitors described herein can be administered in combination with corticosteroids or TNF inhibitors for wound healing, tissue regeneration, and/or tissue repair. Among various prostaglandins, PGE₂ is known to serve as a mediator for wound healing. Therefore, subjects who are receiving steroids, including those healing of wounds from undergoing surgery, can be administered a 15-PGDH inhibitor to enhance PGE₂ and promote would healing.

Additionally, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in subjects receiving corticosteroid treatment. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, and peripheral nerve.

In embodiments, the 15-PGDH inhibitor can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. Therapeutic effects of the 15-PGDH inhibitors when used as a glucocorticoid sensitizer include any, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune functions, easier responses for the subject or patient when steroid administration is tapered or withdrawn, or after prolonged administration of corticosteroids, decreased risks for developing corticosteroid-related adverse events such as opportunistic infections, bone loss, pathologic fracture, diabetes, cataract, and combinations thereof.

In embodiments, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. The glucocorticoid insensitivity related conditions can include a range of immune-inflammatory disorders/diseases treated with steroids when the therapy fails to achieve disease control or is not effective or intolerant or dependent to corticosteroids, and combinations thereof.

In embodiments, the 15-PGDH inhibitor and corticosteroid or the 15-PGDH inhibitor and TNF inhibitor can be administered to a subject that exhibits one or more glucocorticoid insensitivity related diseases, disorders, or conditions selected from the group consisting of glucocorticoid resistant asthma, refractory rheumatoid arthritis, refractory inflammatory bowel disease, chronic obstructive pulmonary disease, acute respiratory distress syndrome, interstitial pulmonary fibrosis, cystic fibrosis, refractory ulcerative colitis, children with severe Crohn's disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, graft-versus-host disease, inflammatory diseases, autoimmune diseases, hyperproliferative diseases, lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitus, malignancies, leukemia, myelomas, lymphomas, acute adrenal insufficiency, rheumatic fever, granulomatous disease, immune proliferation/apoptosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, bacterial infections, viral infections, rickettsial infections, parasitic infections, type II diabetes, obesity, metabolic syndrome, depression, schizophrenia, mood disorders, Cushing's syndrome, anxiety, sleep disorders, memory and learning enhancement, glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis, ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, Pneumocystis carinii pneumonia (PCP) associated with hypoxemia occurring in an HIV(+) individual who is also under treatment with appropriate anti-PCP antibiotics, a diuresis or remission of proteinuria in nephrotic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

### Pharmaceutical Compositions

The 15-PGDH inhibitors described herein can be provided in a pharmaceutical composition or cosmetic composition depending on the pathological or cosmetic condition or disorder being treated. A pharmaceutical composition containing the 15-PGDH inhibitors described herein as an active ingredient may be manufactured by mixing the derivative with a pharmaceutically acceptable carrier(s) or an excipient(s) or diluting the 15-PGDH inhibitors with a diluent in accordance with conventional methods. The pharmaceutical composition may further contain fillers, anti-cohesives, lubricants, wetting agents, flavoring agents, emulsifying agents, preservatives and the like. The pharmaceutical composition may be formulated into a suitable formulation in accordance with the methods known to those skilled in the art so that it can provide an immediate, controlled or sustained release of the 15-PGDH inhibitors after being administered into a mammal.

In embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In embodiments, the 15-PGDH inhibitors described herein may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HCl, lidocaine HCl and the like. The relaxants may be sodium chloride, glucose and the like.

In embodiments, the 15-PGDH inhibitors described herein may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. In embodiments, the 15-PGDH inhibitors described herein can be administered orally, intravenously, or intraperitoneally. The dosage can be a pharmaceutically effective amount. The pharmaceutically effective amount can be an amount of the 15-PGDH inhibitor to treat or improve alopecia, cardiovascular disease, gastrointestinal disease, wounds, and renal disease. The pharmaceutically effective amount of the compound will be appropriately determined depending on the kind and the severity of the disease to be treated, age, sex, body weight and the physical condition of the patients to be treated, administration route, duration of therapy and the like. Generally, the effective amount of the compound may be in the range of about 1 to 1,000 mg in the oral administration, about 0.1 to 500 mg in the intravenous administration, about 5 to 1,000 mg in the rectal administration. Generally, the daily dosage for adults is in the range of about 0.1 to 5,000 mg, preferably about to 1,000 mg but cannot be determined uniformly because it depends on age, sex, body weight and the physical condition of the patients to be treated. The formulation may be administered once a day or several times a day with a divided dose.

Cosmetic compositions containing the 15-PGDH inhibitor can include any substance or preparation intended to be brought into contact with the various superficial parts of the human body (epidermis, body hair and hair system, nails, lips and external genital organs) or with the teeth or the buccal mucous membranes for the purpose, exclusively or mainly, of cleansing them, of giving them a fragrance, of modifying their appearance and/or of correcting body odors and/or protecting them or of maintaining them in good condition.

The cosmetic composition can comprise a cosmetically acceptable medium that may be water or a mixture of water and at least one solvent selected from among hydrophilic organic solvents, lipophilic organic solvents, amphiphilic organic solvents, and mixtures thereof.

For topical application, the cosmetic composition can be administered in the form of aqueous, alcoholic, aqueous-alcoholic or oily solutions or suspensions, or of a dispersion of the lotion or serum type, of emulsions that have a liquid or semi-liquid consistency or are pasty, obtained by dispersion of a fatty phase in an aqueous phase (0/W) or vice versa (W/O) or multiple emulsions, of a free or compacted powder to be used as it is or to be incorporated into a physiologically acceptable medium, or else of microcapsules or microparticles, or of vesicular dispersions of ionic and/or nonionic type. It may thus be in the form of a salve, a tincture, milks, a cream, an ointment, a powder, a patch, an impregnated pad, a solution, an emulsion or a vesicular dispersion, a lotion, aqueous or anhydrous gels, a spray, a suspension, a shampoo, an aerosol or a foam. It may be anhydrous or aqueous. It may also comprise solid preparations constituting soaps or cleansing cakes.

The cosmetic compositions may in particular comprise a hair care composition, and in particular a shampoo, a setting lotion, a treating lotion, a styling cream or gel, restructuring lotions for the hair, a mask, etc. The cosmetic compositions can be a cream, a hair lotion, a shampoo or a conditioner. These can be used in particular in treatments using an application that may or may not be followed by rinsing, or else in the form of a shampoo. A composition in the form of a foam, or else in the form of spray or an aerosol, then comprising propellant under pressure, is also intended. It can thus be in the form of a lotion, serum, milk, cream, gel, salve, ointment, powder, balm, patch, impregnated pad, cake or foam.

In particular, the compositions for application to the scalp or the hair can be in the form of a hair care lotion, for example for daily or twice-weekly application, of a shampoo or of a hair conditioner, in particular for twice-weekly or weekly application, of a liquid or solid soap for cleansing the scalp, for daily application, of a hairstyle shaping product (lacquer, hair setting product or styling gel), of a treatment mask, or of a foaming gel or cream for cleansing the hair. These may also be in the form of a hair dye or mascara to be applied with a brush or a comb.

Moreover, for topical application to the eyelashes or body hair, the compositions may be in the form of a pigmented or unpigmented mascara, to be applied with a brush to the eyelashes or alternatively to beard or moustache hair. For a composition administration by injection, the composition may be in the form of an aqueous lotion or an oily suspension. For oral use, the composition may be in the form of capsules, granules, oral syrups or tablets. According to a particular embodiment, the composition is in the form of a hair cream or hair lotion, a shampoo, a hair conditioner or a mascara for the hair or for the eyelashes.

In a known manner, the cosmetic compositions may also contain adjuvants that are normal in the cosmetics field, such as hydrophilic or lipophilic gelling agents, hydrophilic or lipophilic additives, preservatives, antioxidants, solvents, fragrances, fillers, UV-screening agents, odor absorbers and dyestuffs. The amounts of these various adjuvants are those conventionally used in the cosmetics field, and are for example from 0.1% to 20%, in particular less than or equal to 10%, of the total weight of the composition. According to their nature, these adjuvants can be introduced into the fatty phase, into the aqueous phase and/or into the lipid spherules.

In embodiments, the 15-PGDH inhibitor can be administered in a combinatorial therapy or combination therapy that includes administration of a 15-PGDH inhibitor with one or more additional active agents. The phrase “combinatorial therapy” or “combination therapy” embraces the administration of the 15-PGDH inhibitor, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

In embodiments, the additional active agent can be chosen in particular from lipoxygenase inhibitors as described in EP 648488, the bradykinin inhibitors described in particular in EP 845700, prostaglandins and their derivatives, in particular those described in WO 98/33497, WO 95/11003, JP 97-100091, JP 96-134242, the agonists or antagonists of the receptors for prostaglandins, and the nonprostanoic analogues of prostaglandins as described in EP 1175891 and EP 1175890, WO 01/74307, WO 01/74313, WO 01/74314, WO 01/74315 or WO 01/72268.

In embodiments, the 15-PGDH inhibitors can be administered in combination with active agents, such as vasodilators, prostanoid agonists, antiandrogens, cyclosporins and their analogues, antimicrobials, triterpenes, alone or as a mixture. The vasodilators can include potassium channel agonists including minoxidil and its derivatives, aminexil and the compounds described in U.S. Pat. Nos. 3,382,247, 5,756,092, 5,772,990, 5,760,043, 5,466,694, 5,438,058, 4,973,474, chromakalin and diazoxide. The antiandrogens can include 5 α-reductase inhibitors such as finasteride and the compounds described in U.S. Pat. No. 5,516,779, cyprosterone acetate, azelaic acid, its salts and its derivatives, and the compounds described in U.S. Pat. No. 5,480,913, flutamide and the compounds described in U.S. Pat. Nos. 5,411,981, 5,565,467 and 4,910,226. The antimicrobial compounds can include selenium derivatives, ketoconazole, triclocarban, triclosan, zinc pyrithione, itraconazole, pyridine acid, hinokitiol, mipirocine, and the compounds described in EP 680745, clinycine hydrochloride, benzoyl or benzyl peroxide and minocycline. The anti-inflammatory agents can include inhibitors specific for Cox-2 such as for example NS-398 and DuP-697 (B. Batistini et al., DN&P 1994; 7(8):501-511) and/or inhibitors of lipoxygenases, in particular 5-lipoxygenase, such as for example zileuton (F. J. Alvarez & R. T. Slade, Pharmaceutical Res. 1992; 9(11):1465-1473).

Other active compounds, which can be present in pharmaceutical and/or cosmetic compositions can include aminexil and its derivatives, 60-[(9Z,12Z)octadec-9,12-dienoyl]hexapyranose, benzalkonium chloride, benzethonium chloride, phenol, oestradiol, chlorpheniramine maleate, chlorophyllin derivatives, cholesterol, cysteine, methionine, benzyl nicotinate, menthol, peppermint oil, calcium panthotenate, panthenol, resorcinol, protein kinase C inhibitors, prostaglandin H synthase 1 or COX-1 activators, or COX-2 activators, glycosidase inhibitors, glycosaminoglycanase inhibitors, pyroglutamic acid esters, hexosaccharidic or acylhexosaccharidic acids, substituted ethylenearyls, N-acylated amino acids, flavonoids, derivatives and analogues of ascomycin, histamine antagonists, triterpenes, such as ursolic acid and the compounds described in U.S. Pat. Nos. 5,529,769, 5,468,888, 5,631,282, saponins, proteoglycanase inhibitors, agonists and antagonists of oestrogens, pseudopterins, cytokines and growth factor promoters, IL-1 or IL-6 inhibitors, IL-10 promoters, TNF inhibitors, vitamins, such as vitamin D, analogues of vitamin B12 and panthotenol, hydroxy acids, benzophenones, esterified fatty acids, and hydantoin.

Pharmaceutical and/or cosmetic compositions including the 15-PGDH inhibitor described herein can additionally contain, for example, at least one compound chosen from prostaglandins, in particular prostaglandin PGE₁, PGE₂, their salts, their esters, their analogues and their derivatives, in particular those described in WO 98/33497, WO 95/11003, JP 97-100091, JP 96-134242, in particular agonists of the prostaglandin receptors. It may in particular contain at least one compound such as the agonists (in acid form or in the form of a precursor, in particular in ester form) of the prostaglandin Fla receptor, such as for example latanoprost, fluprostenol, cloprostenol, bimatoprost, unoprostone, the agonists (and their precursors, in particular the esters such as travoprost) of the prostaglandin E₂ receptors such as 17-phenyl PGE₂, viprostol, butaprost, misoprostol, sulprostone, 16,16-dimethyl PGE₂, 11-deoxy PGE₁, 1-deoxy PGE₁, the agonists and their precursors, in particular esters, of the prostacycline (IP) receptor such as cicaprost, iloprost, isocarbacycline, beraprost, eprostenol, treprostinil, the agonists and their precursors, in particular the esters, of the prostaglandin D₂ receptor such as BW245C ((4S)-(3-[(3R,S)-3-cyclohexyl-3-isopropyl]-2,5-dioxo)-4-imidazolidinehept-anoic acid), BW246C ((4R)-(3-[(3R,S)-3-cyclohexyl-3-isopropyl]-2,5-dioxo)-4-imidazolidinehept-anoic acid), the agonists and their precursors, in particular the esters, of the receptor for the thromboxanes A2 (TP) such as I—BOP ([1S-[1a,2a(Z), 3b(1E,3S),4a]]-7-[3-[3-hydroxy-4-[4-(iodophenoxy)-1-butenyl]-7-oxabicyclo-[2.2.1]hept-2-yl]-5-heptenoic acid).

Advantageously, the composition can include at least one 15-PGDH inhibitor as defined above and at least one prostaglandin or one prostaglandin derivative such as for example the prostaglandins of series 2 including in particular PGF_{2α} and PGE₂ in saline form or in the form of precursors, in particular of the esters (example isopropyl esters), their derivatives such as 16,16-dimethyl PGE₂, 17-phenyl PGE₂ and 16,16-dimethyl PGF_{2α} 17-phenyl PGF_{2α}, prostaglandins of series 1 such as 11-deoxyprostaglandin E1, 1-deoxyprostaglandin E1 in saline or ester form, is their analogues, in particular latanoprost, travoprost, fluprostenol, unoprostone, bimatoprost, cloprostenol, viprostol, butaprost, misoprostol, their salts or their esters.

The invention is further illustrated by the following examples, which is not intended to limit the scope of the claims.

### EXAMPLES

### Example A. Analysis of 15-PGDH Inhibitors of the Present Invention

This Example provides data on 15-PGDH inhibitors using an assay described in U.S. Pat. No. 9,790,233, which is herein incorporated by reference in its entirety. The data categorizes the IC₅₀ of each compound for inhibiting enzymatic activity of recombinant 15-PGDH in an in vitro assay as follows: <2.5 nM (***), ≥2.5 nM and ≤10 nM (**), or >10 nM (*). The Recombinant 15-PGDH is human unless otherwise specified. Additionally, the example provides kinetic aqueous solubility data for selected analogues in pH 7 citrate buffer solution.

TABLE 1

| Compound | | PGDH | Kinetic Aq |
|---|---|---|---|
| No. | Structure | Assay-IC₅₀ | solubility (μM pH 7) |
| 1 | 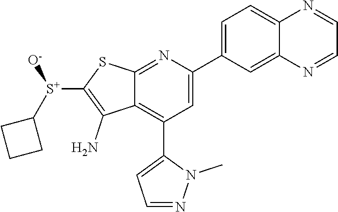 [SMILES: Cn1nccc1-c1cc(-c2ccc3nccnc3c2)nc2sc([S@+]([O-])C3CCC3)c(N)c12] | *** | NA |
| 2 | 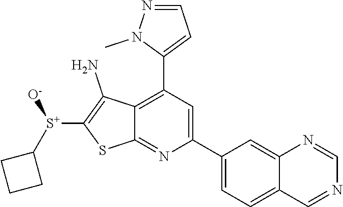 [SMILES: Cn1nccc1-c1cc(-c2ccc3cncnc3c2)nc2sc([S@+]([O-])C3CCC3)c(N)c12] | *** | NA |
| 3 | 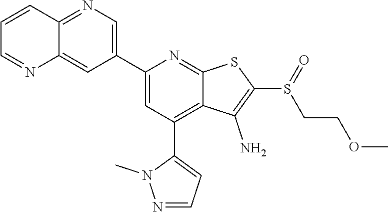 [SMILES: COCCS(=O)c1sc2nc(-c3cnc4cccnc4c3)cc(-c3ccnn3C)c2c1N] | *** | NA |
| 4 | 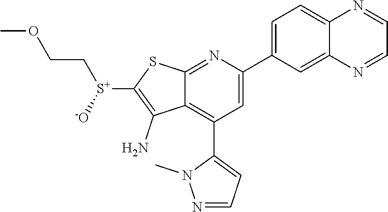 | *** | NA |
| 5 | 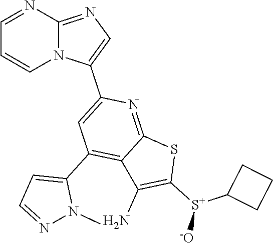 | ** | NA |
| 6 | 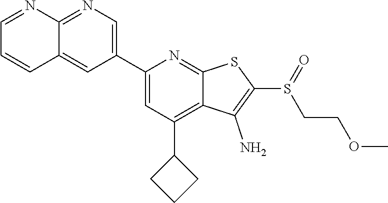 [SMILES: COCCS(=O)c1sc2nc(-c3cnc4ncccc4c3)cc(C3CCC3)c2c1N] | *** | NA |
| 7 | 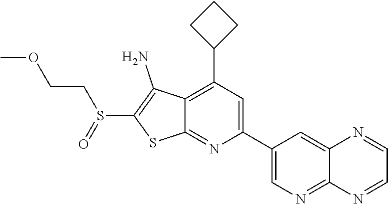 [SMILES: COCCS(=O)c1sc2nc(-c3cnc4nccnc4c3)cc(C3CCC3)c2c1N] | *** | NA |
| 8 | 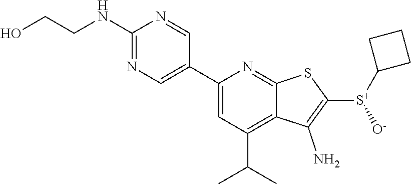 [SMILES: CC(C)c1cc(-c2cnc(NCCO)nc2)nc2sc([S@@+]([O-])C3CCC3)c(N)c12] | * | NA |
| 9 | 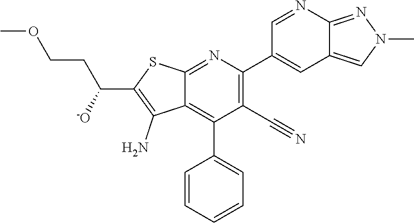 | *** | 169 |
| 10 | 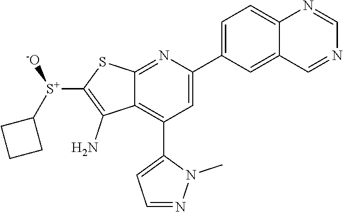 | *** | NA |
| 11 | 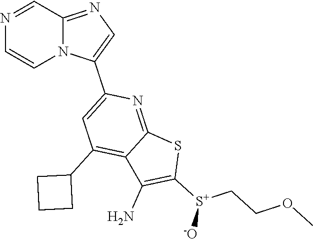 | *** | NA |
| 12 | 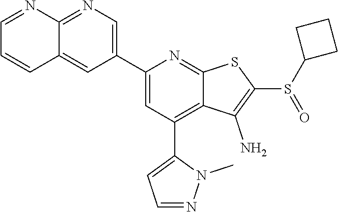 [SMILES: Cn1nccc1-c1cc(-c2cnc3ncccc3c2)nc2sc(S(=O)C3CCC3)c(N)c12] | *** | NA |
| 13 | 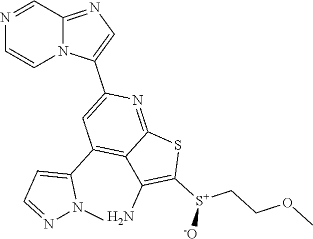 | *** | NA |
| 14 | 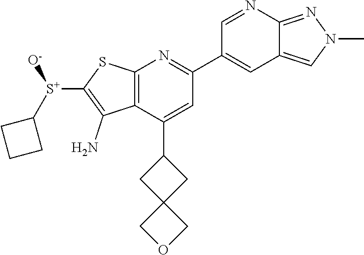 [SMILES: Cn1cc2cc(-c3cc(C4CC5(COC5)C4)c4c(N)c([S@+]([O-])C5CCC5)sc4n3)cnc2n1] | *** | 193 |
| 15 | 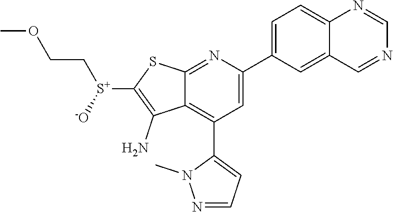 | *** | 188 |
| 16 | 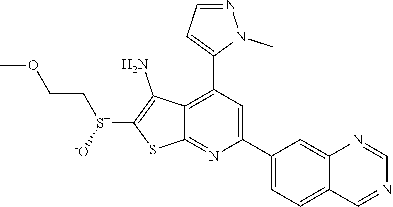 | *** | 195 |
| 17 | 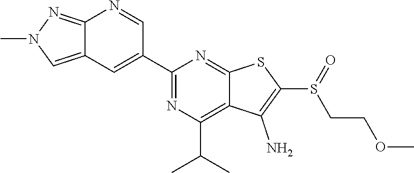 | *** | 187 |
| 18 | 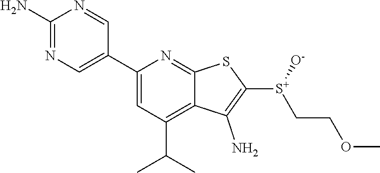 [SMILES: COCC[S@@+]([O-])c1sc2nc(-c3cnc(N)nc3)cc(C(C)C)c2c1N] | *** | 196 |
| 19 | 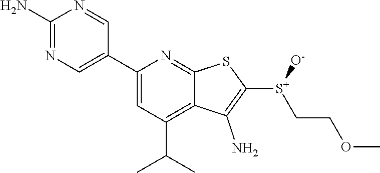 [SMILES: COCC[S@+]([O-])c1sc2nc(-c3cnc(N)nc3)cc(C(C)C)c2c1N] | * | NA |
| 20 | 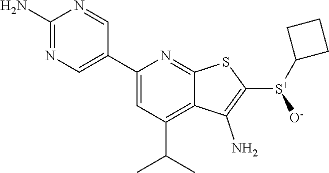 [SMILES: CC(C)c1cc(-c2cnc(N)nc2)nc2sc([S@+]([O-])C3CCC3)c(N)c12] | *** | 181 |
| 21 | 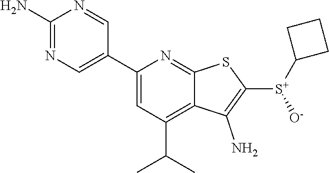 [SMILES: CC(C)c1cc(-c2cnc(N)nc2)nc2sc([S@@+]([O-])C3CCC3)c(N)c12] | * | NA |
| 22 | 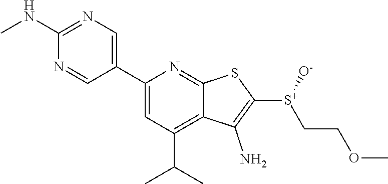 | *** | 171 |
| 23 | 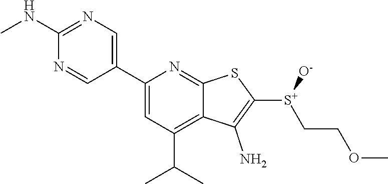 | * | NA |
| 24 | 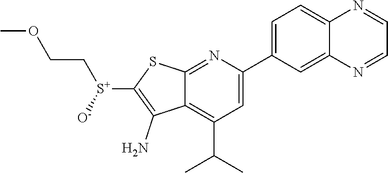 [SMILES: COCC[S@@+]([O-])c1sc2nc(-c3ccc4nccnc4c3)cc(C(C)C)c2c1N] | *** | 188 |
| 25 | 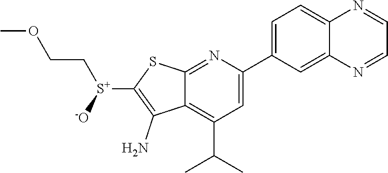 | * | NA |
| 26 | 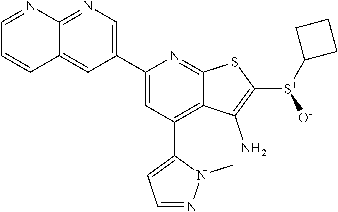 [SMILES: Cn1nccc1-c1cc(-c2cnc3ncccc3c2)nc2sc([S@+]([O-])C3CCC3)c(N)c12] | *** | NA |
| 27 | 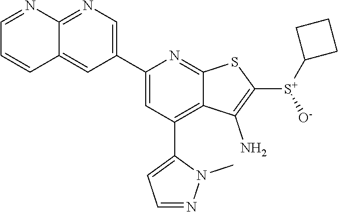 [SMILES: Cn1nccc1-c1cc(-c2cnc3ncccc3c2)nc2sc([S@@+]([O-])C3CCC3)c(N)c12] | * | NA |
| 28 | 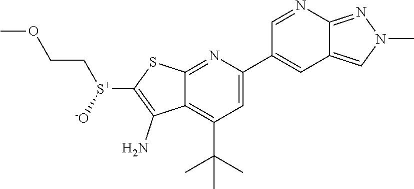 | *** | >200 |
| 29 | 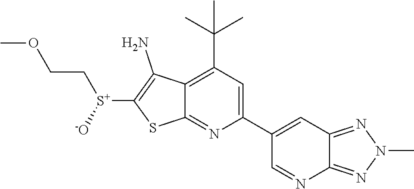 | *** | 188 |
| 30 | 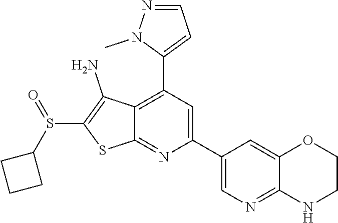 [SMILES: Cn1nccc1-c1cc(-c2cnc3c(c2)OCCN3)nc2sc(S(=O)C3CCC3)c(N)c12] | *** | NA |
| 31 | 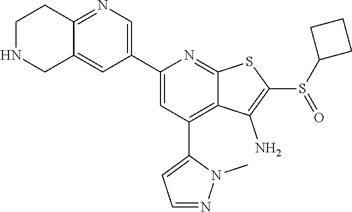 [SMILES: Cn1nccc1-c1cc(-c2cnc3c(c2)CNCC3)nc2sc(S(=O)C3CCC3)c(N)c12] | ** | NA |
| 32 | 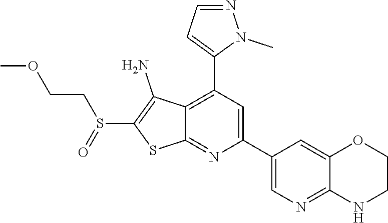 [SMILES: COCCS(=O)c1sc2nc(-c3cnc4c(c3)OCCN4)cc(-c3ccnn3C)c2c1N] | *** | NA |
| 33 | 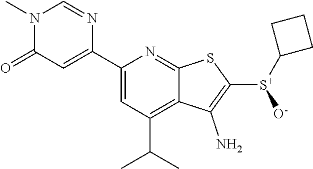 [SMILES: CC(C)c1cc(-c2cc(=O)n(C)cn2)nc2sc([S@+]([O-])C3CCC3)c(N)c12] | *** | 195 |
| 34 | 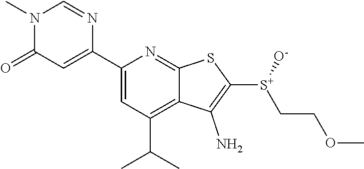 | *** | 163 |
| 35 | 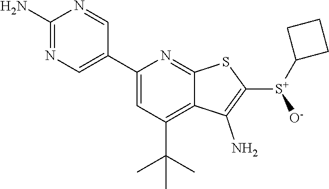 [SMILES: CC(C)(C)c1cc(-c2cnc(N)nc2)nc2sc([S@+]([O-])C3CCC3)c(N)c12] | *** | 196 |
| A | 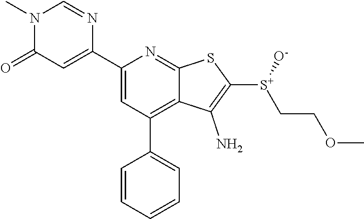 | *** | 64 |

NA = not available

### Example B. Biological Assays

Human microsome stability (HLM), mouse microsome stability (MLM), hERG IC50, Caco-2 permeability, CYP inhibition, and pharmacokinetic (PK) properties were determined on selected compounds of the disclosure. PK studies were performed with single oral 20 mg/kg dose to mice to obtain Cmax and AUC and with single IV 5 mg/kg dose to mice to obtain clearance (Cl). See Table 2.

### In Vitro Microsome Metabolic Stability Assay

Pooled liver microsomes (Human and CD-1 mouse) were purchased from Corning or XenoTech LLC and stored at −80° C. freezer before use. NADPH cofactor system—β-Nicotinamide adenine dinucleotide phosphate reduced form, tetrasodium salt, NADPH·4Na(NADPH) (Vendor: Chem-impex international, Cat.No. 00616) was used. Control compounds were Testosterone, Diclofenac, Propafenone.

### Test Compounds and Reagents

Stock solution—Test compound was 10 mM in DMSO (Dimethyl Sulfoxide).

Working solution—Dilute 5 μL of compound or control from stock solution (10 mM) with 495 μL 100% Acetonitrile (Conc.: 100 μM, 99% ACN, 1% DMSO; final concentration in reaction system: 1 μM, 0.99% ACN, 0.01% DMSO).

Potassium Phosphate Buffer 100 mM (pH 7.4±0.1)

NADPH Cofactor: The appropriate amount of NADPH powder was weighed, and diluted into MgCl₂ solution (work solution concentration: 10 mM NADPH and 10 mM MgCl₂; final concentration in reaction system: 1 mM NADPH and 1 mM MgCl₂).

Liver microsome preparation (0.5 mg/mL): Pipette appropriate volume of microsome (20 mg/mL) to 100 mM buffer solution (Conc.: 0.56 mg/mL, Final concentration in reaction system: 0.5 mg/mL).

### Assay Procedure

Automatic Workstation was used for all liquid handling and incubation. Duplicate points for each test condition (n=2) was obtained.

1) Pre-warmed empty ‘Incubation’ plates T60 and NCF60 for 10 min minutes.

2) Diluted liver microsomes to 0.56 mg/mL in 100 mM phosphate buffer.

3) Transferred 445 μL microsome working solutions (0.56 mg/mL) into pre-warmed ‘Incubation’ plates T60 and NCF60, then pre-incubated ‘Incubation’ plates T60 and NCF60 for 10 min at 37° C. with constant shaking. Transferred 54 μL liver microsomes to blank plate and add 6 μL NAPDH cofactor to blank plate, and then add 180 μL quenching solution to blank plate.

4) Added 5 μL compound working solution (100 μM) into ‘incubation’ plates (T60 and NCF60) containing microsomes and mix 3 times thoroughly.

5) For the NCF60 plate, added 50 μL of buffer and mix 3 times thoroughly. Started timing; plate was incubated at 37° C. for 60 min while shaking.

6) In ‘Quenching’ plate TO, added 180 μL quenching solution and 6 μL NAPDH cofactor. Ensured the plate is chilled to prevent evaporation.

7) For the T60 plate, mixed 3 times thoroughly, and immediately removed 54 μL mixture for the 0-min time point to ‘Quenching’ plate. Then added 44 μL NAPDH cofactor to incubation plate (T60). Started timing; plate will be incubated at 37° C. for 60 min while shaking.

8) At 5, 10, 20, 30, and 60 min, add 180 μL quenching solution to ‘Quenching’ plates, mixed once, and serially transfer 60 μL sample from T60 plate per time point to ‘Quenching’ plates.

9) For NCF60: mix once, and transferred 60 μL sample from the NCF60 incubation to ‘Quenching’ plate containing quenching solution at the 60-min time point.

10) All sampling plates are shaken for 10 min, then centrifuged at 4000 rpm for 20 minutes at 4° C.

11) Transferred 80 μL supernatant into 240 μL HPLC water, and mix by plate shaker for 10 min.

12) Each bioanalysis plate was sealed and shaken for 10 minutes prior to BA analysis.

All samples were injected and analyzed using LC-MS/MS. In the determination of the in vitro elimination constant, ke, of the control and compounds, the analyte/internal standard peak area ratios were converted to percentage remaining (% Remaining) with the following equation:

$$\% Remaining = \frac{Peak area ratio of analyte to IS at each timepoint}{Peak area ratio of analyte to IS at t = 0} \times 100$$

$${C}_{t} = {C}_{0} \cdot \text{?}$$

$$when {C}_{t} = \frac{1}{2} {C}_{0} ,$$

$${T}_{1 / 2} = \frac{Ln 2}{\text{?}} = \frac{0.693}{\text{?}}$$

$$\text{?} = \frac{0.693}{In vitro {T}_{1 / 2}} \cdot \frac{1}{mg / mL microsomal protein in reaction system}$$

$$\text{?} = \text{?} \cdot \frac{mg microsomes}{g liver} \cdot \frac{g liver}{kg body weight}$$

$$\text{?} \text{indicates text missing or illegible when filed}$$

Liver wt: 20 g/kg and 88 g/kg for human and mouse, respectively. Used 45 mg/g for 5 species (mg microsomal protein/g liver weight) to calculate the liver clearance:

hERG Test on Manual Patch-Clamp System

Stable CHO-K1 cells expressing hERG channels (from Sophion Biosciences) were used.

### Compound Preparation

Test compounds were dissolved in 100% DMSO to make stock solutions for each concentration, transferred into compound plates, and then diluted into extracellular solution to achieve final concentration for testing. Visual check for precipitation was conducted before testing. If an ECS working solution was not clear, the solution was not used in the test. As a remedial step, the final DMSO concentration in ECS was increased up to 0.3% to improve the solubility. If the solution is still not clear, the test with the concentration was cancelled. Final DMSO concentration was not more than 0.30% for all concentrations of compounds, vehicle (negative) control, and Amitriptyline (positive) control.

### Electrophysiology

hERG current was recorded at room temperature using the whole-cell patch clamp techniques. As to Axon system, output signals from the amplifier were digitized using a DigiData 1440 A/D D/A board. The recording was controlled with Pclamp10 software. For HEKA system, the recording was controlled with PatchMaster software. The recorded cell was continuously perfused with bath solution from a perfusion system (˜1 ml/min) mounted on the stage of an inverted or upright microscope. The perfusion tip was manually positioned under microscope. Micropipettes were pulled and heat-polished from borosilicate glass capillaries (GC150tF-10, Harvard Apparatus Co. UK) with a programmable micropipette puller. The pipette tip resistance was between 2˜5 MΩ.

### Solutions

External solution (mM): HEPES 10, NaCl 145, KCl 4, CaCl₂ 2, MgCl₂ 1, Glucose 10. pH to 7.4 with 1N NaOH, osmolarity to 290-320 mOsm. Filtered and kept at 4° C. Once prepared, the ECS was used within one month. Internal solution (mM): KOH 31.25, KCl 120, CaCl₂ 5.374, MgCl₂ 1.75, EGTA 10, HEPES 10, Na₂-ATP 4, pH to 7.2 with 1N KOH, osmolarity to 280-310 mOsm. Filtered and kept at −20° C. The solution was stored up to a maximum of three months.

### Voltage Command Protocol

From the holding potential of −80 mV, the voltage was firstly stepped to +60 mV for 850 ms to open hERG channels. After that, the voltage was stepped back down to −50 mV for 1275 ms, causing a “rebound” or tail current, which was measured and collected for data analysis. Finally, the voltage was stepped back to the holding potential (−80 mV). This voltage command protocol was repeated every 15 s continuously during the test (vehicle control, test compound, and washout). For quality control, the minimum seal resistance was 500 MOhms, and the minimum specific hERG current (pre-compound) was 0.4 nA.

### Compound Application

During the initial recording period, the peak current amplitude was monitored until stable (<5% change) for 5 sweeps. Once stabilized, drug perfusion started with the lowest concentration and continued until the peak current was again stable for 5 sweeps, or 5 minutes if peak current remains no change. If required, higher drug concentration was then applied, otherwise the experiment is terminated and the cell dish was discarded.

### Data Analysis

Data was analyzed and fitted using Clampfit or Patchmaster and Prism. Inhibition percentage values for each test compound concentration was calculated from recorded current responses: (1−current measured under compound perfusion/current measured with vehicle perfusion)×100%.

For three or more concentrations testing, IC₅₀ values will be determined from Dose-Response curves that are obtained with Logistic fitting:

$$y = [ \begin{matrix}\underline{max - min} \\ 1 + \text{?}\end{matrix} ] + min$$

$$\text{?} \text{indicates text missing or illegible when filed}$$

where y=I/I_{control}; max=100%; min=0%; [drug]=concentration of compound; n_{H}=Hill coefficient, and IC₅₀=concentration of compound at 50% inhibition.

### Caco-2 Permeability Test

Caco-2 Cells (obtained from ATCC) were seeded onto PET membranes of 96-well Insert Plates and cultured for 21-28 days before being used in the transport assays. The integrity of the monolayer was verified by performing Lucifer yellow rejection assay. The quality of the monolayer was verified by measuring the unidirectional (A to B) permeability of nadolol (low permeability marker), metoprolol (high permeability marker) and bi-directional permeability of digoxin (a P-glycoprotein substrate marker) in duplicate wells. Nadolol and metoprolol were tested at 2.0 μM, and digoxin was tested at 10.0 μM.

Standard assay conditions for test compounds were as follows:
Test concentration: 2.0 μM (DMSO≤1%);
Replicate: n=2;
Directions: A to B and B to A directions;
Incubation time: 2 hours;
Transport buffer: HBSS containing 10 mM HEPES, pH 7.40±0.05; and
Incubation condition: 37±1° C., 5% CO₂, relatively saturated humidity

Dosing solutions were removed and mixed with transport buffer and Stop Solution containing an appropriate internal standard (IS) as T0 samples. After incubation, sample solutions were removed from both donor and receiver wells and mixed with Stop Solution immediately. All samples including T0 samples, donor samples and receiver samples were analyzed using LC/MS/MS. Concentrations of test compounds were expressed as peak area ratio of analytes to IS without a standard curve.

The A to B and B to A apparent permeability coefficients (Pₐₚₚ), % Solution Recovery, and Efflux ratio (ER) were determined.

### Microsomal CYP Inhibition

CYP450 enzymatic activities was determined using 5in1 marker substrate cocktail. For each reaction, enzyme activities in the presence of the test compound at 8 concentrations (0, 0.05, 0.15, 0.5, 1.5, 5.0, 15.0, or 50.0 μM) were measured in singlet (n=1). A known inhibitor for each isoform, tested at a single concentration (3.0 μM) in duplicate (n=2), was included as positive control.

Incubation mixture containing pooled human liver microsomes (Corning, Xenotech, or other qualified vendors; pooled from multiple donors) at 0.2 mg/ml, marker substrates and standard inhibitors (listed in the following table) or the test compound was warm up at 37° C. for 10 minutes. The reactions were initiated by the addition of the NADPH (1.0 mM).

| | | Substrate | | Inhibitor |
|---|---|---|---|---|
| | | Final | | Final |
| CYP | Marker | Conc. | Standard | Conc. |
| isoform | Substrate | (μM) | Inhibitor | (μM) |
| 1A2 | Phenacetin | 10.0 | α-Naphthoflavone | 3.0 |
| 2C9 | Diclofenac | 5.0 | sulfaphenazole | 3.0 |
| 2C19 | S-Mephenytoin | 30.0 | (+)-N-3- | 3.0 |
| | | | benzylnirvanol | |
| 2D6 | Dextromethorphan | 5.0 | quinidine | 3.0 |
| 3A4 | Midazolam | 2.0 | ketoconazole | 3.0 |

After the mixture was incubated at 37° C. for 10 minutes, ice cold acetonitrile containing the internal standard (IS) was added to terminate the reactions.

The metabolites generated from the marker substrates were measured by LC-MS/MS and were assessed based on peak area ratios of the analyte to IS.

The remaining activity (expressed as % of control activity) were calculated; IC50 values of the test compound were determined using SigmaPlot or XLfit with 3- or 4-parameter logistic sigmoidal equation.

### Representative PK Study

The pharmacokinetic (PK) profile and parameters of compounds when administered as an intravenous (IV) bolus dose of 5 mg/kg and oral gavage dose of 20 mg/kg in male C₅₇BL/6J mice was determined. Plasma levels of compound were quantitated for up to 24 hours for each dose route. Dosing can vary depending on the study.

Formulation: For IV dosing, compound was dissolved in 10% (w/v) Captisol in 10 mM citrate buffer, pH 3.5 at a concentration of 1 mg/kg. For oral dosing, compound was suspended in 0.5% (w/v) CMC-Na with 0.2% (v/v) Tween 80 at a concentration of 2 mg/kg.

Appropriate amount of test compound were accurately weighed and mixed with appropriate volume of vehicle to get a clear solution. Vortexing or sonication in water bath may also be need. Animals were dosed within four hours after the formulation was prepared.

Two formulation samples were removed from each of the formulation solutions, transferred into 1.5 mL of polypropylene microcentrifuge tubes and ran dose validation by LC/UV or LC-MS/MS.

For suspension formulations, samples were removed from the top, middle and bottom of each preparation, transferred into 1.5 mL of polypropylene microcentrifuge tubes and ran dose validation by LC/UV or LC-MS/MS. Formulation can vary depending on the study.

### Administration

For both IV and oral routes of dosing, the dose formulation were administered following facility SOPs. The dose volume were determined by the animals' body weight collected on the morning of dosing day.

### Blood Sample Collection and Plasma Processing

At each time point, about 0.03 mL blood was performed from saphenous vein of each animal. All blood samples were transferred into pre-chilled commercial EDTA-K2 tubes and placed on wet ice until centrifugation.

Blood samples were processed for plasma by centrifugation at approximately 4° C., 3,200 g for 10 min. Plasma was collected and transferred into pre-labeled 96 well plate or polypropylene tubes, quickly frozen over dry ice and kept at −70±10° C. until LC/MS/MS analysis.

### Data Analysis

Plasma concentration versus time data were analyzed by non-compartmental approaches using the Phoenix WinNonlin 6.3 software program. Cl, Vdss, C0, Cmax, Tmax, T½, AUC(0-t), AUC(0-inf), MRT(0-t), MRT(0-inf), % F and graphs of plasma concentration versus time profile were determined.

TABLE 2

| Compound | | | hERG | | |
|---|---|---|---|---|---|
| No. from | HLM/MLM | Caco | (IC₅₀, | CYP inhibition | PK |
| Table 1 | (T1/2, min) | summary | μM) | (μM) | summary |
| 9 | NA | NA | 13 | NA | NA |
| 14 | >145/>145 | ER = 88, low | >100 | 2C9:21; others >50 | NA |
| | | permeability | | | |
| 15 | 100/107 | ER = 3, high | 96 | 2C9: 30, 3A4: 11; | NA |
| | | permeability | | others >50 | |
| 16 | 14/17 | ER = 2.6, | 20 | 2C9: 5; 3A4: 2; 2C19: | NA |
| | | high | | 19; lA2and 2D6 >50 | |
| | | permeability | | | |
| 18 | >145/132 | ER = 4, high | 15 | >50 vs 5 isoforms | NA |
| | | permeability | | | |
| 20 | >145/69 | ER = 1; high | 35 | 3A4M: 23; >50 4 | NA |
| | | permeability | | isoforms | |
| 22 | >145/125 | ER = 2; high | 43 | >50 5 isoforms | NA |
| | | permeability | | | |
| 24 | 66/52 | ER = 0.8. | 11 | 1A2: 18, 3A4M: 39. | NA |
| | | High | | others >50 | |
| | | permeability | | | |
| 28 | >145/>145 | ER = 5; high | 17 | 2C9: 11; 2C19: 15, | NA |
| | | permeability | | others >50 pM | |
| 29 | 100/86 | ER = l; high | 11 | 2C9: 4; 2C19:1; 2D6: | NA |
| | | permeability | | 29; 3A4M: 39; 1A2: | |
| | | | | >50 | |
| 33 | >145/>145 | ER = 2; high | >100 | >50 5 isoforms | Cmax = 9913 |
| | | permeability | | | ng/mL; |
| | | | | | AUC = 24570 |
| | | | | | ng * h/mL; |
| | | | | | Cl = 14 |
| | | | | | ml/min/kg |
| 34 | >145/>145 | ER = 5; high | >100 | >50 5 isoforms | Cmax = 13365 |
| | | permeability | | | ng/mL; |
| | | | | | AUC = 42600 |
| | | | | | ng * h/mL; |
| | | | | | Cl = 7 |
| | | | | | ml/min/kg |
| 35 | 114/42 | ER = 1; high | 14 | 2C9: 16; 3A4: 20; | NA |
| | | permeability | | 1A2: 39, 2C19 and | |
| | | | | 2D6: >50 | |

NA = not available

The Applicants discovered the IC50 values, alone, are not predictive of in vivo efficacy, such as in disease models. Additional pharmacokinetic and pharmacodynamic properties, human or mouse microsome stability, Caco, and hERG IC50 are important to in vivo efficacy. Without being bound by theory, in embodiments, clinical candidates should have at least two of the following characteristics: (i) a human microsome stability ≥60 minutes; (ii) hERG IC50>15 μM; (iii) high permeability by Caco permeability study; or (iv) efflux ratio (ER) of <10. In embodiments, clinical candidates have at least two of the following characteristics: (i) a human microsome stability ≥100 minutes; (ii) hERG IC50>35 μM; (iii) high permeability by Caco permeability study; or (iv) efflux ratio (ER) of <10. In embodiments, clinical candidates should have at least two of the following characteristics: (i) a human microsome stability ≥100 minutes; (ii) hERG IC50>50 μM; (iii) high permeability by Caco permeability study; or (iv) efflux ratio (ER) of <10. In embodiments, clinical candidates should have at least two of the following characteristics: (i) a human microsome stability ≥120 minutes; (ii) hERG IC50>75 μM; (iii) high permeability by Caco permeability study; or (iv) efflux ratio (ER) of <10. In embodiments, clinical candidates should have at least two of the following characteristics: (i) a human microsome stability ≥145 minutes; (ii) hERG IC50>100 μM; (iii) high permeability by Caco permeability study; or (iv) efflux ratio (ER) of <10. In embodiments, clinical candidates should have (i) a human microsome stability ≥145 minutes; (ii) hERG IC50>100 μM; (iii) high permeability by Caco permeability study; and (iv) efflux ratio (ER) of <10.

### Synthesis

### Example 1: Synthesis of 4-isopropyl-6-((2-methoxyethyl)sulfinyl)-2-(2-methyl-2H-pyrazolo[3,4-b]pyridin-5-yl)thieno[2,3-d]pyrimidin-5-amine (Compound 17, Table 1)

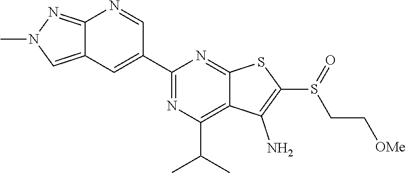 [SMILES: COCCS(=O)c1sc2nc(-c3cnc4nn(C)cc4c3)nc(C(C)C)c2c1N]

### Example 1A: 4-hydroxy-6-isopropyl-2-(2-methyl-2H-pyrazolo[3,4-b]pyridin-5-yl)pyrimidine-5-carbonitrile

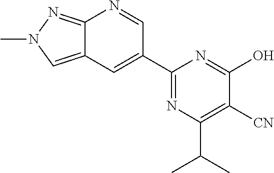 [SMILES: CC(C)c1nc(-c2cnc3nn(C)cc3c2)nc(O)c1C#N]

To the solution of methyl 2-cyano-4-methylpent-2-enoate (1.5 mmol, 232 mg) in EtOH (3 mL) was added 2-methyl-2H-pyrazolo[3,4-b]pyridine-5-carboximidamide hydrochloride (1.5 mmol, 320 mg, 1.0 equiv) and potassium carbonate (3.0 mmol, 414 mg, 2.0 equiv). The reaction mixture was stirred at 80° C. for 3 h. Once completed, the reaction was acidified with conc. HCl, diluted with EtOAc and water. The organic phase was separated and aqueous layer was extracted twice with EtOAc. The combined extractions were dried over magnesium sulfate, filtered and concentrated under reduced pressure to give crude product which was used in the next step without further purification. ESI-MS (m/z): 295.1 [M+H]⁺.

### Example 1B: 4-chloro-6-isopropyl-2-(2-methyl-2H-pyrazolo[3,4-b]pyridin-5-yl)pyrimidine-5-carbonitrile

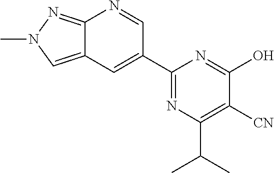 [SMILES: CC(C)c1nc(-c2cnc3nn(C)cc3c2)nc(O)c1C#N]

The reaction mixture of 4-hydroxy-6-isopropyl-2-(2-methyl-2H-pyrazolo[3,4-b]pyridin-5-yl)pyrimidine-5-carbonitrile in POCl₃ (1 mL) was stirred at 100° C. for 20 min. Once completed (the reaction progress was monitored by LCMS) the reaction mixture was cooled to room temperature diluted with EtOAc and water. The organic phase was separated and aqueous layer was extracted twice with EtOAc. The combined extractions were dried over magnesium sulfate, filtered and concentrated under reduced pressure. The crude product was purified by flash chromatography to give desire compound. ESI-MS (m/z): 313.1 [M+H]⁺.

### Example 1C: 1-((chloromethyl)sulfinyl)-2-methoxyethane

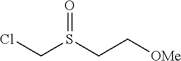

To a solution of (chloromethyl)(2-methoxyethyl)sulfane (500 mg, 3.57 mmol, 1.0 equiv) in 25 mL of DCM was added mCPBA (678 mg, 1.1 mmol, 1.0 equiv) and the reaction mixture was stirred at room temperature. After 1 h, the reaction was diluted with EtOAc and saturated NaHCO₃ solution. The organic phase was separated, washed with saturated NaHCO₃ solution, dried over magnesium sulfate, filtered and concentrated under reduced pressure to give crude product, which was purified by CombiFlash purification system to give pure ((chloromethyl)sulfinyl)cyclobutane in 29% yield. ¹H NMR (400 MHz, Chloroform-d) δ 4.64 (d, J=10.8 Hz, 1H), 4.44 (d, J=10.9 Hz, 1H), 3.95-3.76 (m, 2H), 3.40 (s, 3H), 3.21 (m, 1H), 3.05 (m, 1H).

### Example 1D: 4-isopropyl-6-((((2-methoxyethyl)sulfinyl)methyl)thio)-2-(2-methyl-2H-pyrazolo[3,4-b]pyridin-5-yl)pyrimidine-5-carbonitrile

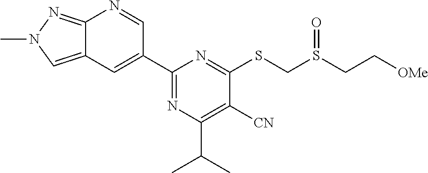 [SMILES: COCCS(=O)CSc1nc(-c2cnc3nn(C)cc3c2)nc(C(C)C)c1C#N]

To the solution of 4-chloro-6-isopropyl-2-(2-methyl-2H-pyrazolo[3,4-b]pyridin-5-yl)pyrimidine-5-carbonitrile (40 mg, 0.128 mmol) in DMF (500 μL) was added sodium sulfide (12 mg, 0.15 mmol, 1.2 equiv) and the reaction mixture was stirred at 100° C. for 20 min. The progress of the reaction was followed by LCMS. Once complete, two drops of conc. HCl was added and the reaction mixture was stirred in the hood for 10 min. ESI-MS (m/z): 311.0 [M+H]⁺. The reaction mixture was diluted with CH₃CN (1 mL) and Et₃N (0.38 mmol, 39 mg) was added followed by 1-((chloromethyl)sulfinyl)-2-methoxyethane (0.38 mmol, 60 mg). The reaction mixture was stirred at 80° C. for 2 h. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give product in 62%. ESI-MS (m/z): 431.1 [M+H]⁺.

### Example 1: Synthesis of Compound 17

To the solution of 4-isopropyl-6-((((2-methoxyethyl)sulfinyl)methyl)thio)-2-(2-methyl-2H-pyrazolo[3,4-b]pyridin-5-yl)pyrimidine carbonitrile (20 mg, 0.046 mmol) in DMF (0.5 mL) was added KOH (0.023 mmol, 2.6 mg, in 26 μl of water). The reaction mixture was stirred at room temperature for 20 min (the reaction was monitored by TLC). Once complete, the reaction was diluted with EtOAc and washed with 5% aq. solution of acetic acid. The organic phase was separated and aqueous layer was extracted twice with EtOAc, dried over magnesium sulfate, filtered and concentrated under reduced pressure to give crude product, which was purified by flash chromatography in 42% isolated yield. ¹H NMR (400 MHz, Methylene Chloride-d₂) δ 9.83 (d, J=2.1 Hz, 1H), 9.24 (d, J=2.1 Hz, 1H), 8.10 (s, 1H), 5.14 (s, 2H), 4.28 (s, 3H), 3.86 (ddd, J=10.3, 7.5, 4.0 Hz, 1H), 3.76-3.63 (m, 2H), 3.59 (ddd, J=13.0, 6.4, 4.0 Hz, 1H), 3.39 (s, 3H), 3.27 (ddd, J=13.0, 7.5, 4.2 Hz, 1H), 1.53 (dd, J=6.7, 3.1 Hz, 6H). ESI-MS (m/z): 431.1 [M+H]⁺.

### Example 2: Synthesis of (R)-2-(cyclobutylsulfinyl)-6-(2-methyl-2H-pyrazolo[3,4-b]pyridin-5-yl)-4-(2-oxaspiro[3.3]heptan-6-yl)thieno[2,3-b]pyridin-3-amine (Compound 14)

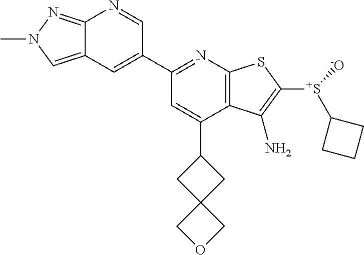

### Example 2A: Synthesis of 1-(2-methyl-2H-pyrazolo[3,4-b]pyridin-5-yl)ethenone

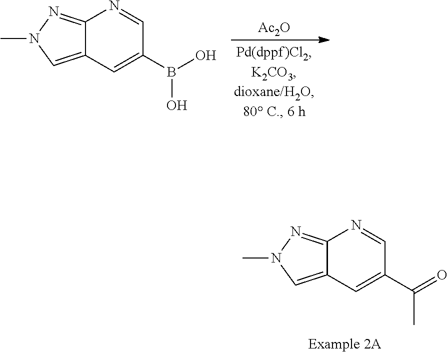

To a solution of (2-methyl-2H-pyrazolo[3,4-b]pyridin-5-yl)boronic acid (10 g, 56.5 mmol) and acetic anhydride (28.8 g, 282 mmol, 26.6 mL) in dioxane (200 mL) and H₂O (20 mL) was added Pd(dppf)Cl₂·CH₂Cl₂ (4.61 g, 5.65 mmol) and K₂CO₃ (23.4 g, 169 mmol). The mixture was stirred at 80° C. for 6 hours. The solution was poured into water (300 mL) and extracted with ethyl acetate (500 mL*2). The organic layer was concentrated. The residue was purified by column chromatography (SiO₂, Petroleum ether: Ethyl acetate=2:1˜0:1) to give the target compound (1.6 g, 16% yield) as yellow solid.

### Example 2B: Synthesis of 2-bromo-1-(2-methyl-2H-pyrazolo[3,4-b]pyridin-5-yl)ethanone

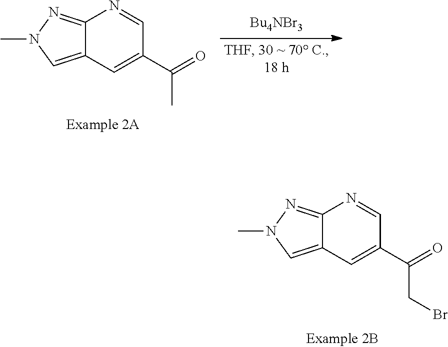

To a solution of Example 2A (1.5 g, 8.56 mmol) in THF (25 mL) was added tetrabutylammonium tribromide (2.89 g, 5.99 mmol). The mixture was stirred at 30° C. for 3 hours, then the reaction mixture was stirred at 70° C. for 15 hours. The reaction mixture was filtered and the filter cake was washed with ethyl acetate (10 mL*2). The filtrate was concentrated under reduced pressure to give the target compound (1.5 g, 68% yield) as a yellow solid.

### Example 2C: Synthesis of 1-(2-methyl-2H-pyrazolo[3,4-b]pyridin-5-yl)-2-(triphenylphosphoranylidene)ethanone

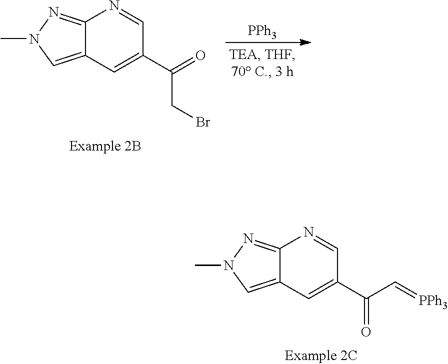

To a solution of Example 2B (1.3 g, 5.12 mmol) and triphenylphosphine (1.34 g, 5.12 mmol) in THF (15 mL) was added TEA (1.04 g, 10.2 mmol, 1.4 mL). The mixture was stirred at 70° C. for 3 hours. The reaction mixture was concentrated under reduced pressure to give a residue. The residue was triturated with toluene 20 mL to give the target compound (2.3 g, crude) as a red solid.

### Example 2D: Synthesis of 2-oxaspiro[3.3]heptan-6-ylmethanol

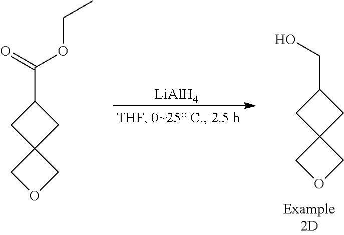

To a solution of ethyl 2-oxaspiro[3.3]heptane-6-carboxylate (1.7 g, 9.99 mmol) in THF (15 mL) was added LiAlH₄ (417 mg, 10.9 mmol) in THF (5 mL) at 25° C. over 0.5 hour. The mixture was stirred at 25° C. for 2 hours. The reaction mixture was quenched by addition of saturated ammonium chloride (20 mL) at 0° C., and extracted with ethyl acetate 150 mL (50 mL*3). The combined organic layers were washed with saturated sodium chloride solution (20 mL*3), dried over Na₂SO₄, filtered and concentrated under reduced pressure to give the target compound (1.1 g, 85% yield) as a yellow oil. ¹H NMR (400 MHz, CDCl₃) δ 4.71 (s, 2H), 4.62 (s, 2H), 3.54 (s, 2H), 2.34-2.30 (m, 3H), 2.01-1.97 (m, 2H).

### Example 2E: Synthesis of 2-oxaspiro[3.3]heptane-6-carbaldehyde

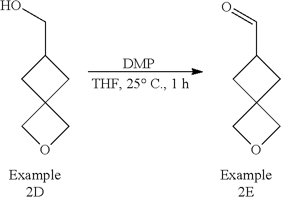

To a solution of Example 2D (1.08 g, 8.43 mmol) in DCM (20 mL) was added DMP (4.29 g, 10.1 mmol, 3.1 mL) at 0° C. The mixture was stirred at 25° C. for 1 hour. The reaction mixture was concentrated under pressure to give the target compound (450 mg, 42% yield) as yellow oil.

### Example 2F: Synthesis of 1-(2-methyl-2H-pyrazolo[3,4-b]pyridin-5-yl)-3-(2-oxaspiro[3.3]heptan-6-yl)prop-2-en-1-one

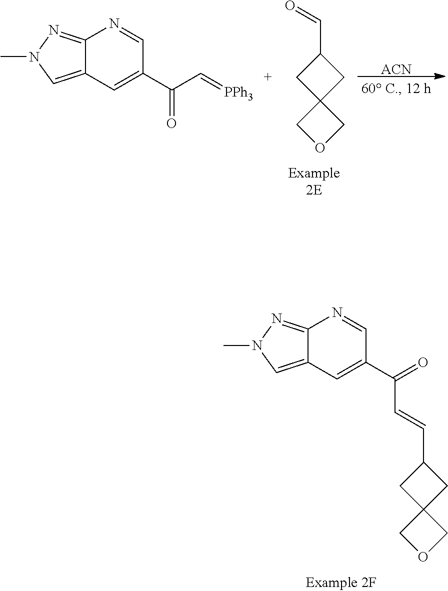

To a solution of Example 2E (369 mg, 2.93 mmol) in acetonitrile (20 mL) was added Example 2C (1.28 g, 2.93 mmol). The mixture was heated to 60° C. and stirred for 12 hours. After cooling, the reaction mixture was concentrated under reduced pressure to give a residue. The residue was purified by column chromatography (SiO₂, Petroleum ether: Ethyl acetate=5:1 to 0:1 to Ethyl acetate:Methanol=30:1 to 0:1) to give the target compound (350 mg, 42% yield) as a white solid.

### Example 2G: Synthesis of 6-(2-methylpyrazolo[3,4-b]pyridin-5-yl)-4-(2-oxaspiro[3.3]heptan-6-yl)-2-sulfanyl-3,4-dihydropyridine-3-carbonitrile

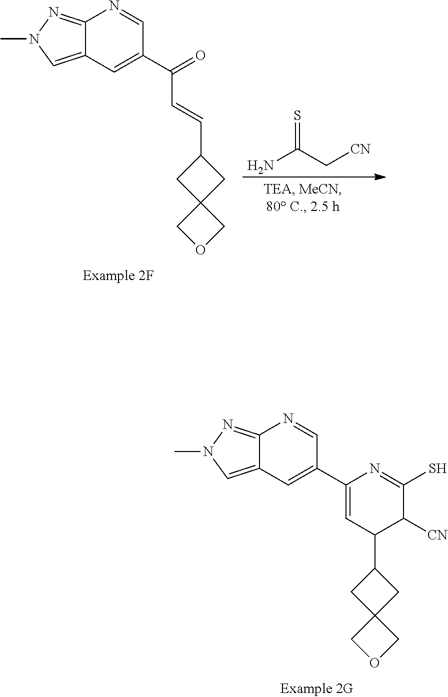

To a solution of Example 2F (300 mg, 1.06 mmol) and 2-cyanothioacetamide (424 mg, 4.24 mmol) was added TEA (321 mg, 3.18 mmol, 0.4 mL) in ACN (10 mL). The mixture was stirred at 80° C. for 2.5 hours. The reaction mixture was concentrated under reduced pressure to give the target compound (386 mg, crude) as a yellow oil.

### Example 2H: Synthesis of 6-(2-methylpyrazolo[3,4-b]pyridin-5-yl)-4-(2-oxaspiro[3.3]heptan-6-yl)-2-sulfanyl-pyridine-3-carbonitrile

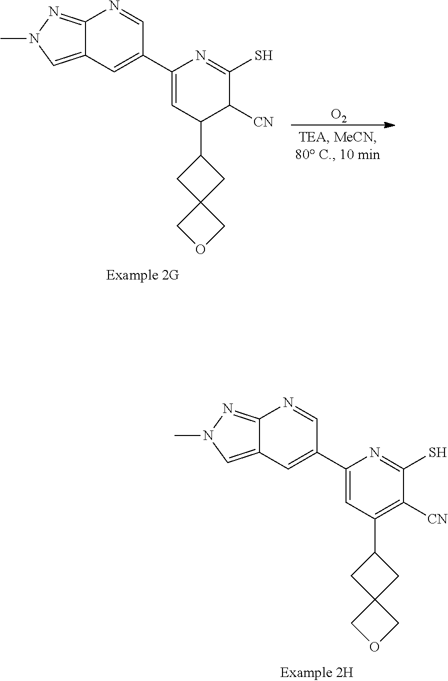 [SMILES: Cn1cc2cc(C3=CC(C4CC5(COC5)C4)C(C#N)C(S)=N3)cnc2n1 ; "O2" ; Cn1cc2cc(-c3cc(C4CC5(COC5)C4)c(C#N)c(S)n3)cnc2n1]

To a solution of Example 2G (386 mg, 1.06 mmol) was added TEA (213 mg, 2.11 mmol, 0.3 mL) in ACN (10 mL) under 02. The mixture was stirred at 80° C. for 10 min. The reaction mixture was concentrated under reduced pressure to give the target compound (383 mg, crude) as a yellow oil.

### Example 21: Synthesis of (R)-2-(((cyclobutylsulfinyl)methyl)thio)-6-(2-methyl-2H-pyrazolo[3,4-b]pyridin-5-yl)-4-(2-oxaspiro[3.3]heptan-6-yl)nicotinonitrile

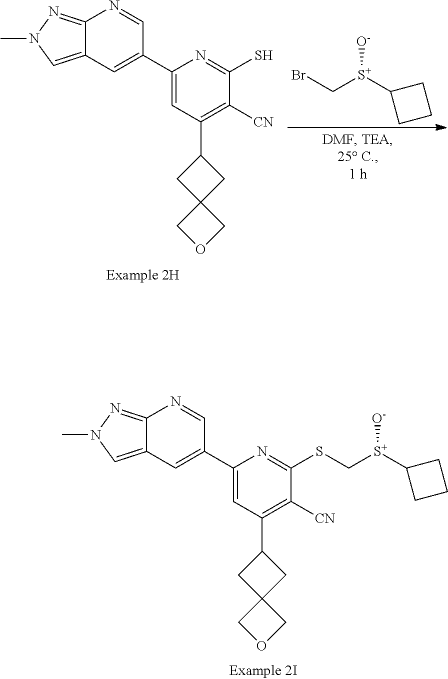 [SMILES: Cn1cc2cc(-c3cc(C4CC5(COC5)C4)c(C#N)c(S)n3)cnc2n1 ; [O-][S@@+](CBr)C1CCC1 ; Cn1cc2cc(-c3cc(C4CC5(COC5)C4)c(C#N)c(SC[S@+]([O-])C4CCC4)n3)cnc2n1]

To a solution of Example 2H (383 mg, 1.05 mmol) in DMF (5 mL) was added triethylamine (2.0 eq) and (R)-((bromomethyl)sulfinyl)cyclobutane (207 mg, 1.05 mmol). The mixture was stirred at 25° C. for 15 minutes. The mixture was concentrated and the crude product was purified by prep-HPLC (column: Phenomenex Synergi C18 150*25*10 um; mobile phase: [water (0.1% TFA)-ACN]) to give the target compound (200 mg, 39% yield) as a white solid. ¹H NMR (400 MHz, CDCl₃) δ 9.26 (d, J=2.4 Hz, 1H), 8.97 (d, J=2.4 Hz, 1H), 8.14 (s, 1H), 7.51 (s, 1H), 4.90 (s, 2H), 4.70-4.66 (m, 3H), 4.32 (s, 3H), 4.07 (d, J=12.8 Hz, 1H), 3.80-3.74 (m, 1H), 3.68-3.66 (m, 1H), 2.94-2.89 (m, 2H), 2.80-2.74 (m, 1H), 2.50-2.44 (m, 3H), 2.32-2.27 (m, 1H), 2.13-2.10 (m, 2H), 2.02-1.99 (m, 1H).

### Example 2: Synthesis of Compound 14

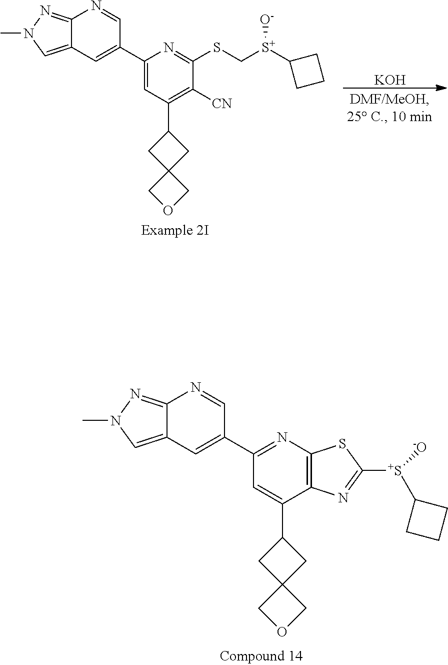

Example 2: To a solution of Example 21 (190 mg, 396 mol, 1 equiv) in methanol and N, N-dimethylformamide was added potassium hydroxide solution (5%, 0.6 equiv). The mixture was stirred at 25° C. for 10 minutes. The mixture was neutralized with aqueous acetic acid (10%) and concentrated. The residue was purified by reversed-phase HPLC to give the target compound (135 mg, 70% yield, 98% purity) as a yellow solid. Optical Rotation determination showed the Specific Rotation was +75.984°; LCMS: (ES+) m/z (M+H)+=480.2. ¹H NMR (400 MHz, CDCl₃) δ 9.29 (d, J=2.0 Hz, 1H), 8.56 (d, J=2.4 Hz, 1H), 7.95 (s, 1H), 7.49 (s, 1H), 5.09 (s, 2H), 4.91 (s, 2H), 4.67 (d, J=6.8 Hz, 1H), 4.63 (d, J=6.4 Hz, 1H), 4.29 (s, 3H), 4.09-4.05 (m, 1H), 3.94-3.91 (m, 1H), 2.87-2.82 (m, 3H), 2.75-2.68 (m, 1H), 2.40-2.37 (m, 3H), 2.32-2.27 (m, 1H), 2.13-2.05 (m, 2H).

### Example 3: Synthesis of (R)-3-amino-2-((2-methoxyethyl)sulfinyl)-6-(2-methyl-2H-pyrazolo[3,4-b]pyridin-5-yl)-4-phenylthieno[2,3-b]pyridine-5-carbonitrile (Compound 9)

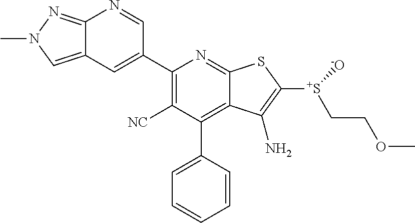

### Example 3A: Synthesis of 2-amino-6-chloro-4-phenylpyridine-3,5-dicarbonitrile

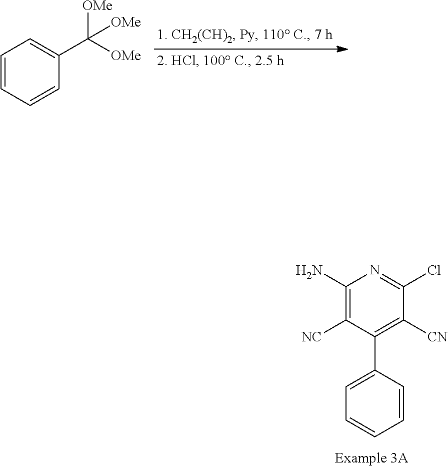 [SMILES: COC(OC)(OC)c1ccccc1 ; N#Cc1c(N)nc(Cl)c(C#N)c1-c1ccccc1]

To a solution of trimethoxymethylbenzene (15 g, 82.3 mmol, 14.1 mL) in pyridine (40 mL) was added propanedinitrile (10.9 g, 165 mmol, 10.4 mL). The mixture was stirred at 110° C. for 7 hours. After cooling, HCl (12 M, 82.4 mL) was added and the mixture was stirred at 100° C. for another 2.5 hours. The reaction mixture was cooled and filtered. The filter cake was collected and used as-is in the next step. The target compound (7.7 g, 37% yield) was obtained as a yellow solid.

### Example 3B: Synthesis of 2-amino-6-(2-methyl-2H-pyrazolo[3,4-b]pyridin-5-yl)-4-phenylpyridine-3,5-dicarbonitrile

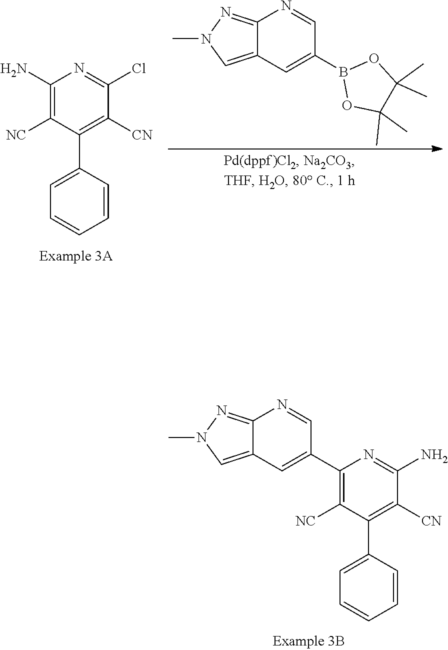

To a solution of Example 3A (6.7 g, 26.3 mmol, 1.0 eq) in tetrahydrofuran and water was added sodium carbonate (2.0 eq). 1,1′-bis(diphenylphosphino)ferrocene-palladium(II) dichloride dichloromethane complex (0.05 eq) and 2-methyl-5-(4,4,5,5-tetramethyl-1,3,2-dioxaborolan-2-yl)-2H-pyrazolo[3,4-b]pyridine (10.2 g, 39.5 mmol) were added. The reaction was stirred at 100° C. for 3 hours under nitrogen. The mixture was concentrated and water was added. The mixture was extracted with dichloromethane and the combined organic phases were concentrated. The residue was purified by column chromatography to provide the target compound (2.2 g, 24% yield) as a yellow solid. ¹H NMR (400 MHz, DMSO-d6) δ 8.98 (d, J=2.0 Hz, 1H), 8.77 (d, J=2.4 Hz, 1H), 8.64 (s, 1H), 8.48 (s, 2H), 7.68-7.59 (m, 5H), 4.26 (s, 3H).

### Example 3C: Synthesis of 2-chloro-6-(2-methyl-2H-pyrazolo[3,4-b]pyridin-5-yl)-4-phenylpyridine-3,5-dicarbonitrile

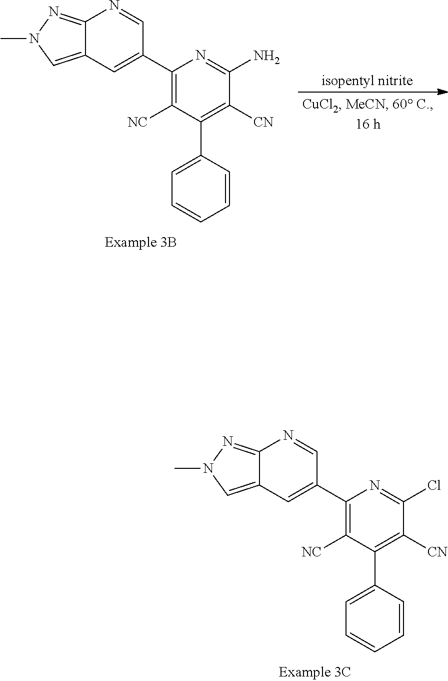

To a solution of Example 3B (2.2 g, 6.26 mmol) in MeCN (40 mL) was added CuCl₂ (1.68 g, 12.5 mmol) and isopentyl nitrite (1.47 g, 12.5 mmol, 1.69 mL). The mixture was stirred at 60° C. for 16 hours. To the reaction mixture was added 1 M HCl (30 mL) and the mixture was filtered. The filtrate was concentrated under reduced pressure to give a residue. The residue was purified by column chromatography (SiO₂, Petroleum ether: Ethyl acetate=5:1 to 0:1 to Ethyl acetate:MeOH=50:1). The target compound (0.2 g, 9% yield) was obtained as a yellow solid. ¹H NMR (400 MHz, MeOD) δ 8.31 (s, 1H), 8.28 (d, J=1.8 Hz, 1H), 8.06-8.01 (m, 1H), 7.69-7.57 (m, 5H), 4.27 (s, 3H).

### Example 3D: Synthesis of 2-mercapto-6-(2-methyl-2H-pyrazolo[3,4-b]pyridin-5-yl)-4-phenylpyridine-3,5-dicarbonitrile

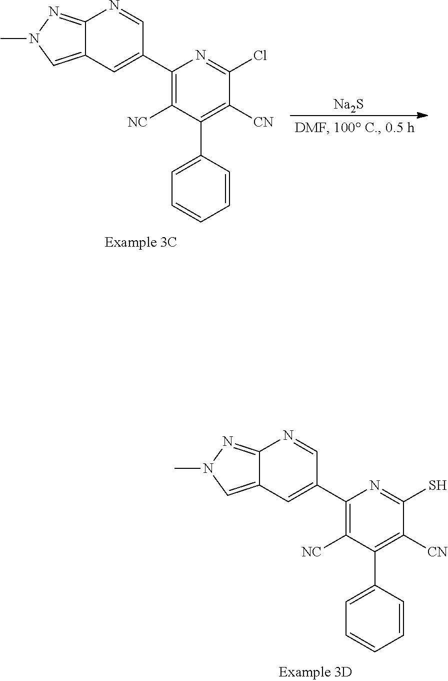

To a solution of Example 3C (0.17 g, 458 μmol) in dimethyl formamide (2 mL) was added Na₂S (42.9 mg, 550 μmol). The mixture was stirred at 100° C. for 0.5 hours. The mixture was concentrated directly to give the target compound (0.17 g, crude) as a yellow oil.

### Example 3E: Synthesis of (R)-2-((((2-methoxyethyl)sulfinyl)methyl)thio)-6-(2-methyl-2H-pyrazolo[3,4-b]pyridin-5-yl)-4-phenylpyridine-3,5-dicarbonitrile

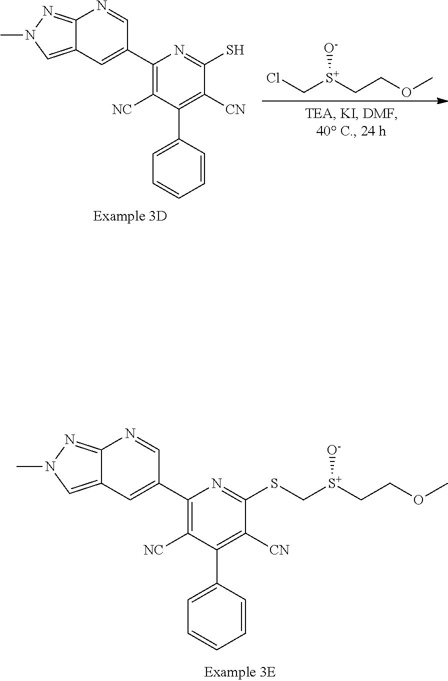

Example 3E was prepared by the procedure used for Example 21 starting from Example 3D (170 mg, 461 μmol), KI (153 mg, 923 μmol) and (R)-1-((chloromethyl)sulfinyl) methoxyethane (72.3 mg, 461 μmol) to give the target compound (170 mg, 75%) as a yellow solid.

### Example 3: Synthesis of Compound 9

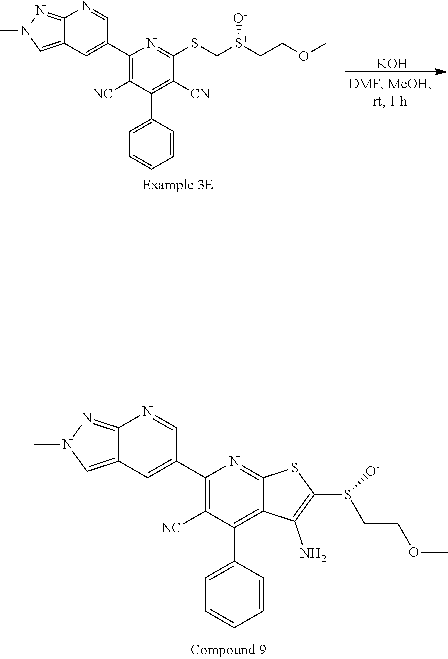

Compound 9 was prepared by the procedure used for Example 2 starting from Example 3E (170 mg, 348 μmol) to give the target compound (10.2 mg, 6% yield, 98.7% purity) as a yellow solid. Optical Rotation determination showed the Specific Rotation was +53.216°; LCMS: (ES+) m/z (M+H)+=489.1. ¹H NMR (400 MHz, CDCl₃) δ 9.21 (d, J=2.4 Hz, 1H), 8.67 (d, J=2.4 Hz, 1H), 8.00 (s, 1H), 7.60-7.53 (m, 3H), 7.48-7.41 (m, 2H), 4.44 (s, 2H), 4.24 (s, 3H), 3.82-3.7 (m, 1H), 3.66-3.59 (m, 1H), 3.55-3.47 (m, 1H), 3.31 (s, 3H), 3.23-3.16 (m, 1H).

### Example 4: Synthesis of (R)-4-cyclobutyl-6-(imidazo[1,2-a]pyrazin-3-yl)-2-((2-methoxyethyl)sulfinyl)thieno[2,3-b]pyridin-3-amine (Compound 11)

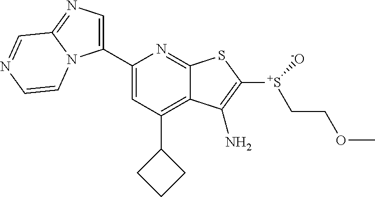

### Example 4A: Synthesis of 3-(1-ethoxyvinyl)imidazo[1,2-a]pyrazine

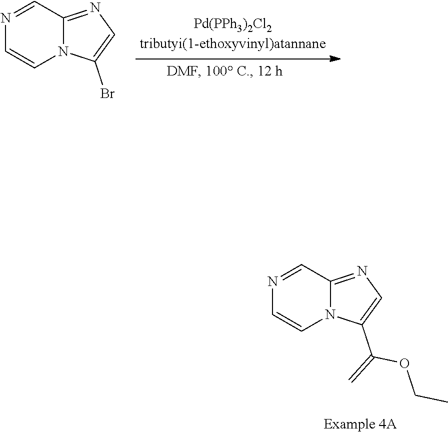

To a solution of 3-bromoimidazo[1,2-a]pyrazine (2.7 g, 13.6 mmol) in DMF (36 mL) was added tributyl(1-ethoxyvinyl)tin (1 eq.) and Pd(PPh₃)₂Cl₂ (0.05 eq.) under N₂. The mixture was stirred at 100° C. for 12 hours. The mixture was diluted with ethyl acetate (100 mL) and treated with aqueous potassium fluoride solution (12 g of KF in 20 mL of water). The solution was stirred at 25° C. for 0.5 hour. The solution was filtered. The filtrate was diluted with H₂O (80 mL) and extracted with ethyl acetate (100 mL×2). The combined organic layers were washed with brine (50 mL×2), dried over Na₂SO₄, filtered and concentrated under reduced pressure to give a residue. The target compound was obtained (2.3 g, crude) as a brown solid.

### Example 4B: Synthesis of 1-(imidazo[1,2-a]pyrazin-3-yl)ethanone

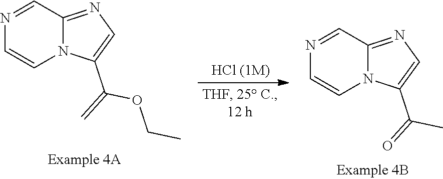

To a solution of Example 4A (2.3 g, 12.2 mmol) in THF (50 mL) was added HCl (1 M, 19.5 mL). The mixture was stirred at 25° C. for 12 hours. The reaction mixture was quenched by addition of saturated NaHCO₃ (60 mL) at 25° C., and then extracted with dichloromethane (100 mL*2). The combined organic layers were dried over Na₂SO₄, filtered and concentrated under reduced pressure to give a residue. The crude product was triturated with DMF (30 mL) at 25° C. for 5 min. The target compound (1.3 g, 66%) was obtained as a brown solid.

### Example 4C: Synthesis of (E)-3-cyclobutyl-1-(imidazo[1,2-a]pyrazin-3-yl)prop-2-en-1-one

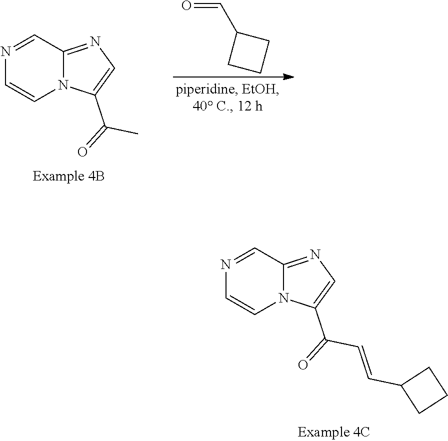 [SMILES: CC(=O)c1cnc2cnccn12 ; O=CC1CCC1 ; O=C(/C=C/C1CCC1)c1cnc2cnccn12]

To a solution of Example 4B (900 mg, 5.58 mmol) and cyclobutanecarbaldehyde (1 eq.) in ethyl alcohol (15 mL) was added piperidine (2 eq.). The mixture was stirred at 40° C. for 12 hours. The reaction mixture was concentrated under reduced pressure to give a residue. The residue was purified by column chromatography (SiO₂, Petroleum ether:Ethyl acetate=5:1 to 0:1 to Ethyl acetate:Methanol=30:1 to 0:1). The target compound (600 mg, 47%) was obtained as a brown solid.

### Example 4D: Synthesis of 4-cyclobutyl-6-(imidazo[1,2-a]pyrazin-3-yl)-2-mercaptonicotinonitrile

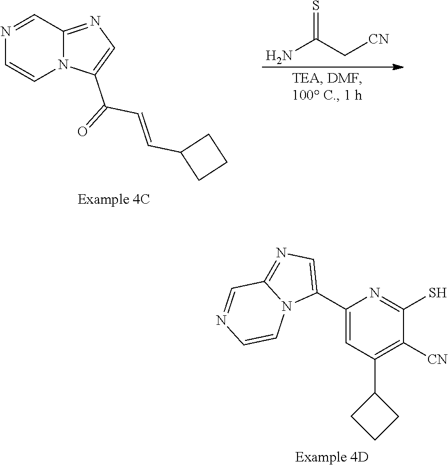 [SMILES: O=C(/C=C/C1CCC1)c1cnc2cnccn12 ; N#CCC(N)=S ; N#Cc1c(C2CCC2)cc(-c2cnc3cnccn23)nc1S]

To a solution of Example 4C (400 mg, 1.36 mmol) and 2-cyanothioacetamide (204 mg, 2.04 mmol) in MeCN (4 mL) was added TEA (0.4 mL). The mixture was stirred at 100° C. for 1 hour. The reaction mixture was concentrated under reduced pressure to give a residue which was used in the next step without further purification.

### Example 4E: Synthesis of (R)-4-cyclobutyl-6-(imidazo[1,2-a]pyrazin-3-yl)-2-((((2-methoxyethyl)sulfinyl)methyl)thio)nicotinonitrile

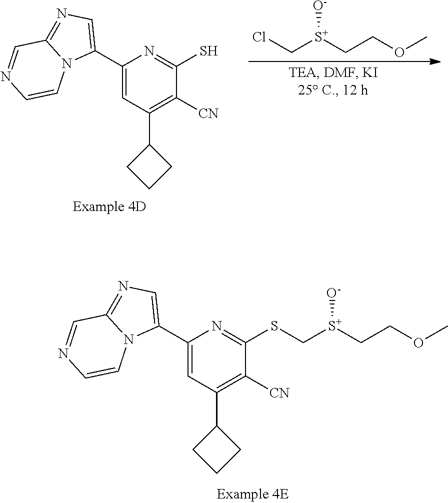

Example 4E was prepared by the procedure used for Example 21 starting from Example 4D (400 mg, 1.30 mmol) and (R)-1-((chloromethyl)sulfinyl)-2-methoxyethane (245 mg, 1.56 mmol) to give the target compound (200 mg, 73%) as a brown solid. ¹H NMR (400 MHz, CDCl₃) δ 9.52 (dd, J1=4.8 Hz, J1=1.2 Hz, 1H), 9.26 (d, J=1.2 Hz, 1H), 8.35 (s, 1H), 8.20 (d, J=4.4 Hz, 1H), 7.50 (s, 1H), 4.85-4.78 (m, 1H), 4.70-4.64 (m, 1H), 4.04-3.96 (m, 1H), 3.92-3.80 (m, 2H), 3.43 (s, 3H), 3.28-3.19 (m, 1H), 3.15-3.07 (m, 1H), 2.65-2.55 (m, 2H), 2.36-2.16 (m, 3H), 2.03-1.95 (m, 1H).

### Example 4: Synthesis of Compound 11

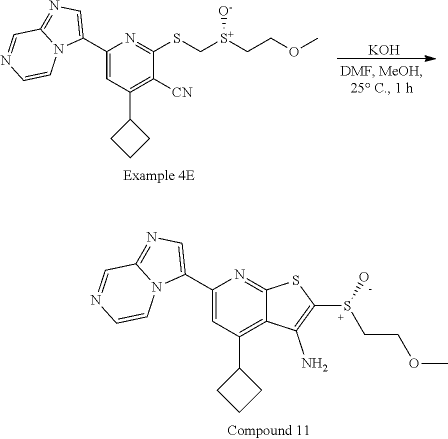

Compound 11 was prepared by the procedure used for Example 2 starting from Example 4E (270 mg, 631 μmol) to give the target compound (171.9 mg, 63% yield, 98.9% purity) as a yellow solid. Optical Rotation determination showed the Specific Rotation was +148.851°; LCMS: (ES+) m/z (M+H)+=428.2. ¹H NMR (400 MHz, CDCl₃) δ 9.81 (dd, J1=4.4 Hz, J2=1.6 Hz, 1H), 9.23 (d, J=1.6 Hz, 1H), 8.43 (s, 1H), 8.12 (d, J=4.8 Hz, 1H), 7.68 (d, J=0.4 Hz, 1H), 5.14 (s, 2H), 4.28-4.16 (m, 1H), 3.95-3.88 (m, 1H), 3.77-3.69 (m, 1H), 3.69-3.61 (m, 1H), 3.44 (s, 3H), 3.35-3.25 (m, 1H), 2.61-2.38 (m, 4H), 2.28-2.14 (m, 1H), 2.11-2.00 (m, 1H).

### Example 5: Synthesis of (R)-6-(imidazo[1,2-a]pyrazin-3-yl)-2-((2-methoxyethyl)sulfinyl)-4-(1-methyl-1H-pyrazol-5-yl)thieno[2,3-b]pyridin-3-amine (Compound 13)

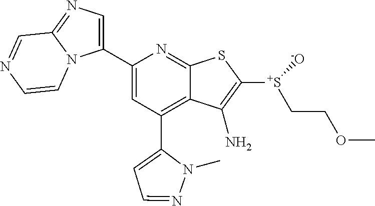

### Example 5A: (R)-6-(imidazo[1,2-a]pyrazin-3-yl)-2-((((2-methoxyethyl)sulfinyl)methyl)thio)-4-(1-methyl-1H-pyrazol-5-yl)nicotinonitrile

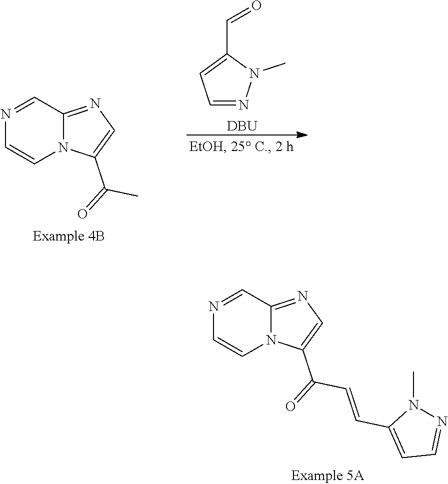

To a solution of Example 4B (247 mg, 1.5 mmol) and 2-methylpyrazole-3-carbaldehyde (253 mg, 2.3 mmol) in EtOH (2 mL) was added DBU (467 mg, 3.1 mmol). The mixture was stirred at 25° C. for 2 hours. The reaction mixture was filtered and concentrated under reduced pressure to give Example 5A (400 mg, crude) as a yellow solid.

### Example 5B

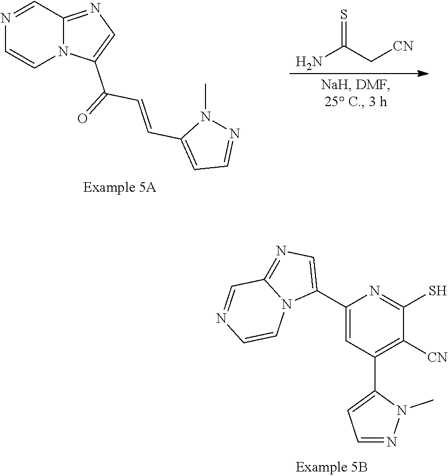 [SMILES: Cn1nccc1/C=C/C(=O)c1cnc2cnccn12 ; N#CCC(N)=S ; Cn1nccc1-c1cc(-c2cnc3cnccn23)nc(S)c1C#N]

To a solution of Example 5A (50 mg, 0.20 mmol) and 2-cyanothioacetamide (40 mg, 0.39 mmol) in DMF (1 mL) was added NaH (24 mg, 0.59 mmol, 60% purity). The mixture was stirred at 25° C. for 3 hours. The reaction mixture was quenched by addition MeOH (1 mL) at 25° C., and then concentrated under reduced pressure to give the target compound (65 mg, crude) as a yellow liquid which was used in the next step without further purification.

### Example 5C

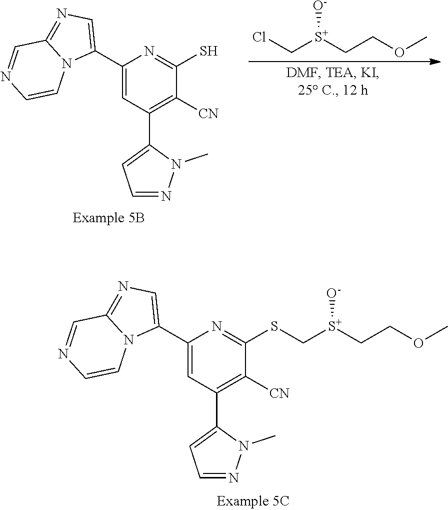

To a solution of Example 5B (70 mg, 0.20 mmol) and (R)-1-((chloromethyl)sulfinyl)-2-methoxyethane (33 mg, 0.20 mmol) in DMF (0.2 mL) was added KI (70 mg, 0.40 mmol) and TEA (43 mg, 0.40 mmol). The mixture was stirred at 25° C. for 12 hours. The reaction mixture was filtered and concentrated under reduced pressure to give a residue. The residue was purified by prep-HPLC (TFA condition; column: Phenomenex Gemini-NX C18 75*30 mm*3 um; mobile phase: [water (0.1% TFA)-ACN]; B %: 28%-38%, 7 min) to give the target compound (60 mg, 63% yield) as a yellow solid.

### Example 5: Synthesis of Compound 13

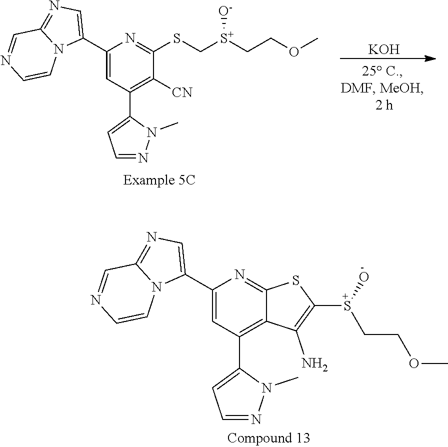

Compound 13 was prepared by the procedure used for Example 2 starting from Example 5C (50 mg, 0.11 mmol) to give the target compound (17 mg, 33% yield, 98% purity) as a yellow solid. Optical Rotation determination showed the Specific Rotation was +129.967°; LCMS: (ES⁺) m/z (M+H)⁺=454.1. ¹H NMR (400 MHz, CDCl₃) δ=9.77 (m, 1H), 9.18 (d, J=1.6 Hz, 1H), 8.32 (s, 1H), 8.10 (d, J=4.8 Hz, 1H), 7.62 (d, J=1.8 Hz, 1H), 7.60 (s, 1H), 6.45 (m, 1H), 4.57-4.39 (m, 2H), 3.82-3.79 (m, 1H), 3.79 (s, 3H), 3.69-3.61 (m, 1H), 3.58-3.50 (m, 1H), 3.34 (s, 3H), 3.24-3.18 (m, 1H).

### Example 6: Synthesis of 2-[(R)-cyclobutanesulfinyl]-6-{imidazo[1,2-a]pyrimidin-3-yl}-4-(1-methyl-1H-pyrazol-5-yl)thieno[2,3-b]pyridin-3-amine (Compound 5)

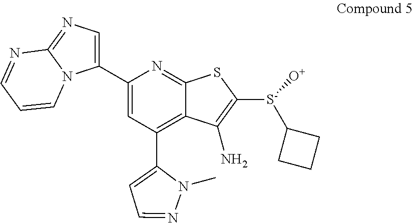

Compound 5 was prepared following the procedure used for Example 5, starting from Example 5B and (R)-((bromomethyl)sulfinyl)cyclobutane. Compound 5 was isolated as a yellow solid. Optical Rotation determination showed the Specific Rotation was +47.325°; LCMS: (ES⁺) m/z (M+H)⁺=450.2. ¹H NMR (400 MHz, CDCl₃) δ 8.63-8.62 (m, 1H), 8.53-8.51 (m, 1H), 8.38 (s, 1H), 8.30 (s, 1H), 7.67-7.66 (d, J=1.2 Hz, 1H), 6.97-6.95 (m, 1H), 6.50 (s, 1H), 4.61-4.51 (m, 2H), 4.00-3.92 (m, 1H), 3.79 (s, 3H), 2.89-2.80 (m, 1H), 2.41-2.37 (m, 2H), 2.29-2.28 (m, 1H), 2.13-2.09 (m, 2H).

### Example 7: 4-cyclobutyl-2-(2-methoxyethanesulfinyl)-6-{pyrido[2,3-b]pyrazin-7-yl}thieno[2,3-b]pyridin-3-amine (Compound 7)

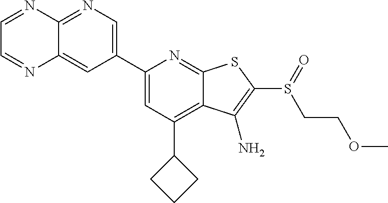 [SMILES: COCCS(=O)c1sc2nc(-c3cnc4nccnc4c3)cc(C3CCC3)c2c1N]

### Example 7A: 4-cyclobutyl-2-((((2-methoxyethyl)thio)methyl)thio)-6-(pyrido[2,3-b]pyrazin-7-yl)nicotinonitrile

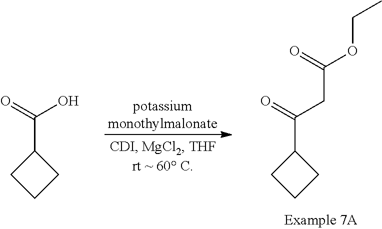

To a solution of cyclobutanecarboxylic acid (20 g, 199 mmol) in tetrahydrofuran (300 mL) was added CDI (34.01 g, 209 mmol). The mixture was stirred at 60° C. for 1 hour. After cooling to 25° C., magnesium chloride (22.82 g, 239 mmol) and potassium monoethylmalonate (37.40 g, 219 mmol) was added to the mixture and the reaction stirred at 60° C. for 1 hour. The reaction mixture was filtered and concentrated under reduced pressure to give a residue. The residue was purified by column chromatography (SiO₂, Petroleum ether/Ethyl acetate=I/O to 100:1) to give the target compound (30 g, 88% yield) as a yellow liquid. ¹H NMR (400 MHz, CDCl₃) δ 4.15-4.09 (m, 2H), 3.39-3.28 (m, 3H), 2.25-2.16 (m, 2H), 2.11-2.05 (m, 2H), 1.97-1.85 (m, 1H), 1.81-1.72 (m, 1H), 1.21 (t, J=7.2 Hz, 3H).

### Example 7B

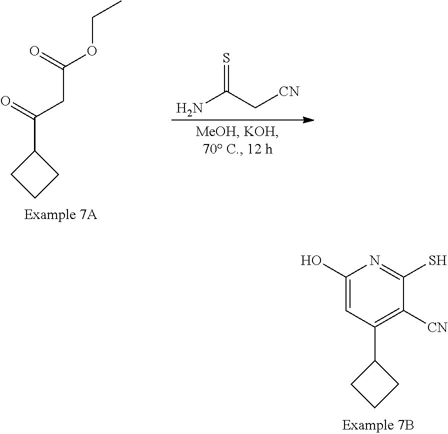

To a solution of Example 7A (10 g, 58.75 mmol) in methyl alcohol (100 mL) was added potassium hydroxide (4.94 g, 88.13 mmol) and 2-cyanothioacetamide (8.83 g, 88.13 mmol). The mixture was stirred at 70° C. for 12 hours. The reaction mixture was filtered to give crude Example 7B (12 g) as a yellow solid.

### Example 7C

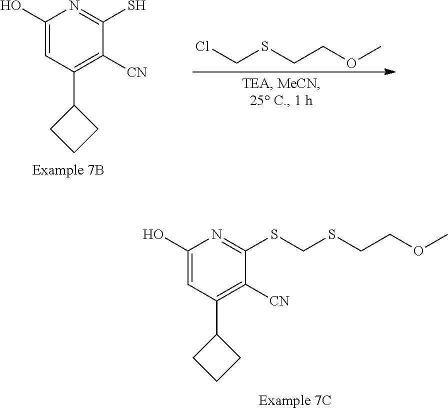

To a solution of Example 7B (5 g, 24.2 mmol) in acetonitrile (50 mL) was added triethylamine (7.36 g, 72.7 mmol) and (chloromethyl)(2-methoxyethyl)sulfane (2.73 g, 19.4 mmol). The mixture was stirred at 25° C. for 1 hour. The reaction mixture was concentrated under reduced pressure to give crude Example 7C (7.5 g) as a yellow oil.

### Example 7D

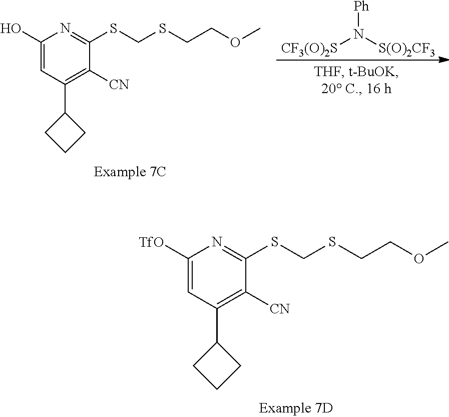

To a solution of Example 7C (7.5 g, 24.2 mmol) in tetrahydrofuran (100 mL) was added potassium tert-butoxide (5.42 g, 48.3 mmol) and 1,1,1-trifluoro-N-phenyl-N-(trifluoromethylsulfonyl)methanesulfonamide (12.95 g, 36.2 mmol). The mixture was stirred at 20° C. for 16 hours. The reaction mixture was concentrated under reduced pressure to give a residue. The residue was purified by column chromatography (SiO₂, Petroleum ether/Ethyl acetate=I/O to 100/1) to give the target compound (10 g) which was used without further purification in the next step.

### Example 7E

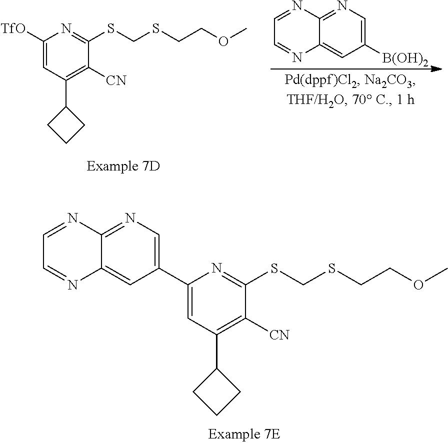

To a solution of Example 7D (1.0 g, 2.26 mmol) in tetrahydrofuran (10 mL) and water (5 mL) was added sodium carbonate (2.0 eq). Then 1,1′-bis(diphenylphosphino)ferrocene-palladium(II) dichloride dichloromethane complex (0.05 eq) and pyrido[2,3-b]pyrazin-7-ylboronic acid (790 mg, 4.52 mmol) was added into the mixture. The reaction was stirred at 100° C. for 3 hours under nitrogen. The mixture was concentrated, diluted with water (30 mL), and extracted with dichloromethane (30 mL*3). The organic phase was concentrated. The residue was purified by column chromatography (SiO₂, Petroleum ether/Ethyl acetate=10:1 to 1:1) to give the target compound (0.2 g, 20% yield) as a yellow solid. ¹H NMR (400 MHz, CDCl₃) δ 10.01 (d, J=2.4 Hz, 1H), 9.08 (d, J=1.6 Hz, 1H), 9.06 (d, J=2.4 Hz, 1H), 9.00 (d, J=1.6 Hz, 1H), 7.85 (s, 1H), 4.61 (s, 2H), 4.38-4.33 (m, 1H), 3.57 (t, J=6.4 Hz, 2H), 3.40 (s, 3H), 2.97 (t, J=6.4 Hz, 2H), 2.58-2.41 (m, 4H), 2.26-2.15 (m, 1H), 2.09-2.01 (m, 1H).

### Example 7F

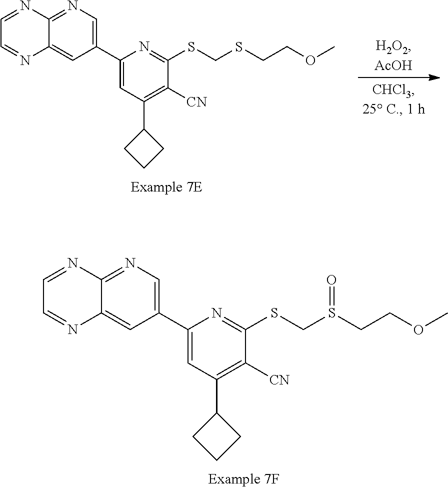 [SMILES: COCCSCSc1nc(-c2cnc3nccnc3c2)cc(C2CCC2)c1C#N ; COCCS(=O)CSc1nc(-c2cnc3nccnc3c2)cc(C2CCC2)c1C#N]

To a solution of Example 7E (0.14 g, 330 μmol) in chloroform (2 mL) was added acetic acid (25 eq) and hydrogen peroxide (75 mg, 661 mol, 63 μL, 30% purity). The mixture was stirred at 20° C. or 1 hour. The mixture was basified with saturated sodium bicarbonate solution to pH=7 and extracted with dichloromethane (10 mL*3). The combined organic phases were concentrated to give the target compound (0.12 g, 82% yield) as a yellow solid.

### Example 7: Synthesis of Compound 7

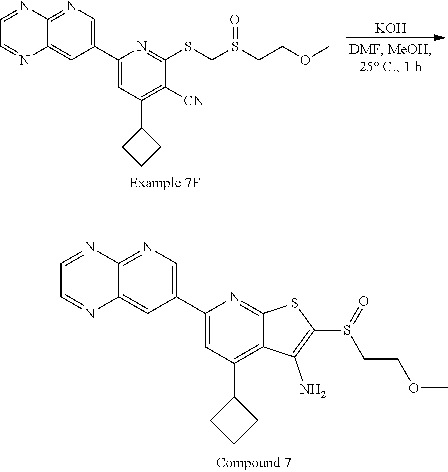

Compound 7 was prepared by the procedure used for Example 2 starting from Example 7F (0.1 g, 227 μmol) to give the target compound (6.0 mg, 5% yield) as a yellow solid. LCMS: (ES⁺) m/z (M+H)⁺=440.2. ¹H NMR (400 MHz, CDCl₃) δ 10.01 (d, J=2.4 Hz, 1H), 9.12-9.09 (m, 2H), 9.02 (d, J=2.0 Hz, 1H), 7.89 (s, 1H), 5.17 (s, 2H), 4.29-4.27 (m, 1H), 3.93-3.88 (m, 1H), 3.74-3.70 (m, 1H), 3.67-3.63 (m, 1H), 3.43 (s, 3H), 3.35-3.29 (m, 1H), 2.59-2.53 (m, 2H), 2.51-2.44 (m, 2H), 2.26-2.18 (m, 1H), 2.09-2.05 (m, 1H).

### Example 8: Synthesis of (R)-2-(cyclobutylsulfinyl)-4-(1-methyl-1H-pyrazol-5-yl)-6-(quinoxalin-6-yl)thieno[2,3-b]pyridin-3-amine (Compound 1)

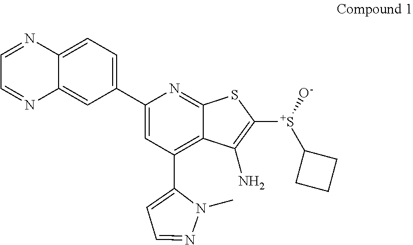 [SMILES: Cn1nccc1-c1cc(-c2ccc3nccnc3c2)nc2sc([S@+]([O-])C3CCC3)c(N)c12]

Compound 1 was prepared by the procedure used for Example 4 starting from 3-bromoimidazo[1,2-a]pyrazine and using (R)-((bromomethyl)sulfinyl)cyclobutane. The target compound was isolated as a yellow solid. Optical Rotation determination showed the Specific Rotation was +165.379°; LCMS: (ES⁺) m/z (M+H)⁺=461.2. ¹H NMR (400 MHz, DMSO-d6) δ=9.06-8.94 (m, 3H), 8.77 (d, J=8.8 Hz, 1H), 8.34 (s, 1H), 8.24 (d, J=8.8 Hz, 1H), 7.71 (s, 1H), 6.68 (s, 1H), 5.20-4.78 (m, 2H), 3.90 (m, J=8.0 Hz, 1H), 3.75 (s, 3H), 2.77-2.60 (m, 1H), 2.29-2.11 (m, 3H), 2.09-1.87 (m, 2H).

### Example 9: Synthesis of 2-[(R)-2-methoxyethanesulfinyl]-4-(1-methyl-1H-pyrazol-5-yl)-6-(quinoxalin-6-yl)thieno[2,3-b]pyridin-3-amine (Compound 4)

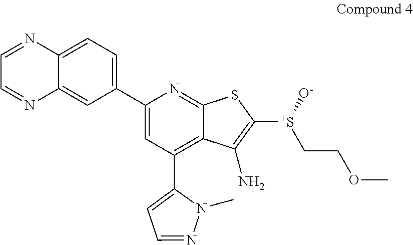 [SMILES: COCC[S@@+]([O-])c1sc2nc(-c3ccc4nccnc4c3)cc(-c3ccnn3C)c2c1N]

Compound 4 was prepared in a manner analogous to that used for Example 8 using (R)-1-((chloromethyl)sulfinyl)-2-methoxyethane. The target compound was isolated as a yellow solid. Optical Rotation determination showed the Specific Rotation was +86.501°; LCMS: (ES⁺) m/z (M+H)⁺=465.2. ¹H NMR (400 MHz, DMSO-d6) δ=9.09-8.95 (m, 3H), 8.79 (d, J=9.2 Hz, 1H), 8.37 (s, 1H), 8.25 (d, J=8.8 Hz, 1H), 7.72 (m, 1H), 6.69 (s, 1H), 5.18-4.94 (m, 2H), 3.76 (m, 4H), 3.69-3.61 (m, 1H), 3.41 (m, 1H), 3.29 (s, 3H), 3.28-3.21 (m, 1H).

### Example 10: Synthesis of 2-[(R)-cyclobutanesulfinyl]-4-(1-methyl-1H-pyrazol-5-yl)-6-(quinazolin-6-yl)thieno[2,3-b]pyridin-3-amine (Compound 10)

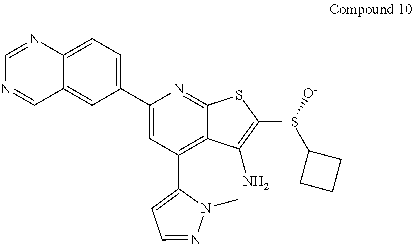 [SMILES: Cn1nccc1-c1cc(-c2ccc3ncncc3c2)nc2sc([S@+]([O-])C3CCC3)c(N)c12]

Compound 10 was prepared by the procedure used for Example 4 starting from 6-bromoquinazoline and using (R)-((bromomethyl)sulfinyl)cyclobutane. The target compound was isolated as a yellow solid. Optical Rotation determination showed the Specific Rotation was +171.791°; LCMS: (ES⁺) m/z (M+H)⁺=461.2. ¹H NMR (400 MHz, CDCl₃) δ 9.53 (s, 1H), 9.38 (s, 1H), 8.74-8.70 (m, 2H), 8.19 (d, J=9.2 Hz, 1H), 7.79 (s, 1H), 7.69 (s, 1H), 6.53 (d, J=10.0 Hz, 1H), 4.62-4.52 (m, 2H), 4.00-3.92 (s, 1H), 3.78 (s, 3H), 2.88-2.79 (m, 1H), 2.47-2.37 (m, 2H), 2.31-2.26 (m, 1H), 2.12-2.01 (m, 2H).

### Example 11: Synthesis of 2-[(R)-2-methoxyethanesulfinyl]-4-(1-methyl-1H-pyrazol-5-yl)-6-(quinazolin-6-yl)thieno[2,3-b]pyridin-3-amine (Compound 15)

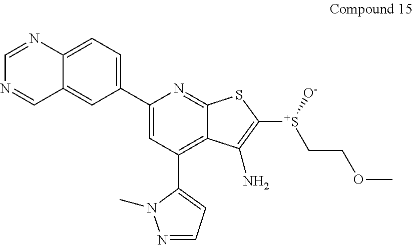 [SMILES: COCC[S@@+]([O-])c1sc2nc(-c3ccc4ncncc4c3)cc(-c3ccnn3C)c2c1N]

Compound 15 was prepared in a manner analogous to that used for Example 10 using (R)-1-((chloromethyl)sulfinyl)-2-methoxyethane. The target compound was isolated as a yellow solid. Optical Rotation determination showed the Specific Rotation was +55.805°; LCMS: (ES+) m/z (M+H)+=465.1. ¹H NMR (400 MHz, CDCl₃) δ 9.54 (s, 1H), 9.39 (s, 1H), 8.75-8.71 (m, 2H), 8.21-8.18 (m, 1H), 7.81 (s, 1H), 7.69 (s, 1H), 6.53 (d, J=8.4 Hz, 1H), 4.60-4.52 (m, 2H), 3.89-3.87 (m, 1H), 3.79 (s, 3H), 3.73 (s, 1H), 3.63-3.61 (m, 1H), 3.41 (s, 3H), 3.31-3.29 (m, 1H).

### Example 12: Synthesis of 2-[(R)-cyclobutanesulfinyl]-4-(1-methyl-1H-pyrazol-5-yl)-6-(quinazolin-7-yl)thieno[2,3-b]pyridin-3-amine (Compound 2)

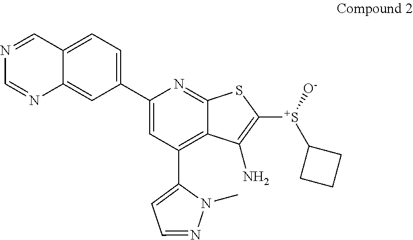 [SMILES: Cn1nccc1-c1cc(-c2ccc3cncnc3c2)nc2sc([S@+]([O-])C3CCC3)c(N)c12]

Compound 2 was prepared by the procedure used for Example 4 starting from 7-bromoquinazoline and using (R)-((bromomethyl)sulfinyl)cyclobutane. The target compound was isolated as a yellow solid. Optical Rotation determination showed the Specific Rotation was +127.389°; LCMS: (ES⁺) m/z (M+H)⁺=461.2. ¹H NMR (400 MHz, CDCl₃) δ 9.48 (s, 1H), 9.40 (s, 1H), 8.69 (s, 1H), 8.58-8.55 (m, 1H), 8.09 (d, J=8.4 Hz, 1H), 7.84 (s, 1H), 7.69 (d, J=1.6 Hz, 1H), 6.52 (s, 1H), 4.64-4.54 (m, 2H), 3.97 (t, J=8.0 Hz, 1H), 3.79 (s, 3H), 2.87-2.81 (m, 1H), 2.45-2.34 (m, 2H), 2.29-2.25 (m, 1H), 2.13-2.07 (m, 2H).

### Example 13: Synthesis of 2-[(R)-2-methoxyethanesulfinyl]-4-(1-methyl-1H-pyrazol-5-yl)-6-(quinazolin-7-yl)thieno[2,3-b]pyridin-3-amine (Compound 16)

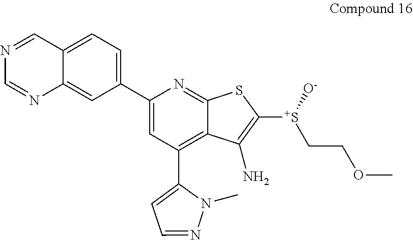 [SMILES: COCC[S@@+]([O-])c1sc2nc(-c3ccc4cncnc4c3)cc(-c3ccnn3C)c2c1N]

Compound 16 was prepared in a manner analogous to that used for Example 12 using (R)-1-((chloromethyl)sulfinyl)-2-methoxyethane. The target compound was isolated as a yellow solid. Optical Rotation determination showed the Specific Rotation was +57.551°; LCMS: (ES⁺) m/z (M+H)⁺=465.2. ¹H NMR (400 MHz, CDCl₃) δ 9.47 (s, 1H), 9.39 (s, 1H), 8.69 (s, 1H), 8.57 (d, J=8.4 Hz, 1H), 8.09 (d, J=8.8 Hz, 1H), 7.85 (s, 1H), 7.70 (s, 1H), 6.53 (s, 1H), 4.62-4.54 (m, 2H), 3.89-3.86 (m, 1H), 3.80 (s, 3H), 3.72 (s, 1H), 3.63-3.60 (m, 1H), 3.41 (s, 3H), 3.32-3.29 (m, 1H).

### Example 14: Syntheses of 2-(cyclobutanesulfinyl)-4-(1-methyl-1H-pyrazol-5-yl)-6-(1,8-naphthyridin-3-yl)thieno[2,3-b]pyridin-3-amine and its enantiomers (Compounds 12, 26, and 27)

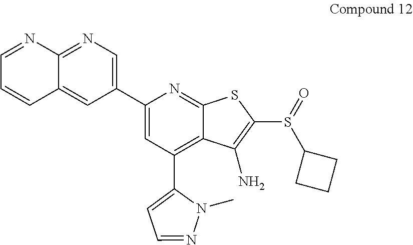 [SMILES: Cn1nccc1-c1cc(-c2cnc3ncccc3c2)nc2sc(S(=O)C3CCC3)c(N)c12]

Compounds 12, 26, and 27 were prepared in a manner analogous to that used for Example 7. The target compound was isolated as a yellow solid. LCMS: (ES+) m/z (M+H)+=461.2. ¹H NMR (400 MHz, DMSO-d6) 9.91 (d, J=2.8 Hz, 1H), 9.34 (d, J=2.4 Hz, 1H), 9.14 (dd, J1=4.4 Hz, J2=2.0 Hz, 1H), 8.61 (dd, J1=4.0 Hz, J2=2.0 Hz, 1H), 8.30 (s, 1H), 7.74-7.70 (m, 2H), 6.69 (s, 1H), 5.11-4.96 (m, 2H), 3.90 (q, J=8.0 Hz, 1H), 3.74 (s, 3H), 2.71-2.62 (m, 1H), 2.26-2.14 (m, 3H), 2.07-1.99 (m, 1H), 1.90-1.80 (m, 1H).

The enantiomers were separated by SFC (column: DAICEL CHIRALPAK AS (250 mm*30 mm, 10 um); mobile phase: [0.1% NH₃H₂O EtOH]; B %: 55%-55%, 4.0 min; 50 min) to give the (+) enantiomer (20 mg, 38% yield, 98% purity, 99% ee) and the (−) enantiomer (21 mg, 40% yield, 99% purity, 97% ee) as yellow solids. Optical Rotation determination showed the Specific Rotations were +175.541° and −130.767°.

### Example 15: Synthesis of 4-cyclobutyl-2-(2-methoxyethanesulfinyl)-6-(1,8-naphthyridin-3-yl)thieno[2,3-b]pyridin-3-amine (Compound 6)

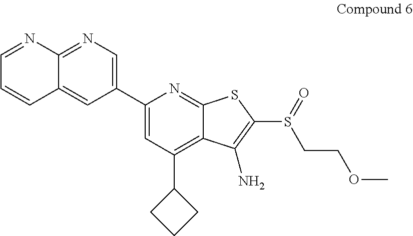 [SMILES: COCCS(=O)c1sc2nc(-c3cnc4ncccc4c3)cc(C3CCC3)c2c1N]

Compound 6 was prepared in a manner analogous to that used for Example 7. The target compound was isolated as a yellow solid. LCMS: (ES⁺) m/z (M+H)⁺=439.1. ¹H NMR (400 MHz, CDCl₃) 9.83 (d, J=2.4 Hz, 1H), 9.17-9.16 (m, 1H), 8.91 (d, J=2.4 Hz, 1H), 8.35-8.32 m, 1H), 7.5 (s, 1H), 7.58-7.53 (m, 1H), 5.15 (s, 2H), 4.30-4.21 (m, 1H), 3.93-3.87 (m, 1H), 3.74-3.69 (m, 1H), 3.66-3.60 (m, 1H), 3.42 (s, 3H), 3.30-3.27 (m, 1H), 2.60-2.40 (m, 4H), 2.27-2.15 (m, 1H), 2.10-2.02 (m, 1H).

### Example 16: Synthesis of 2-(2-methoxyethanesulfinyl)-4-(1-methyl-1H-pyrazol-5-yl)-6-(1,5-naphthyridin-3-yl)thieno[2,3-b]pyridin-3-amine (Compound 3)

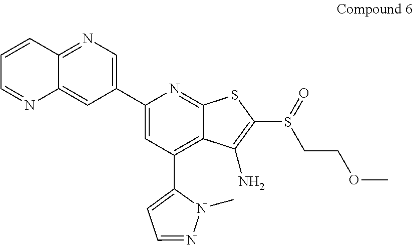 [SMILES: COCCS(=O)c1sc2nc(-c3cnc4cccnc4c3)cc(-c3ccnn3C)c2c1N]

Compound 6 was prepared in a manner analogous to that used for Example 7. The target compound was isolated as a yellow solid. LCMS: (ES+) m/z (M+H)+=465.2. ¹H NMR (400 MHz, CDCl₃) δ 9.82 (d, J=2.0 Hz, 1H), 9.09-9.06 (m, 1H), 9.04-9.01 (m, 1H), 8.52-8.48 (m, 1H), 7.85 (s, 1H), 7.75-7.70 (m, 2H), 6.59-6.53 (m, 1H), 4.60 (d, J=26.4 Hz, 2H), 3.95-3.88 (m, 1H), 3.82 (s, 3H), 3.79-3.71 (m, 1H), 3.69-3.60 (m, 1H), 3.43 (s, 3H), 3.36-3.28 (m, 1H).

### Example 17: Syntheses of 5-{3-amino-2-[2-methoxyethanesulfinyl]-4-(propan-2-yl)thieno[2,3-b]pyridin-6-yl}pyrimidin-2-amine and its enantiomers (Compounds 18 and 19)

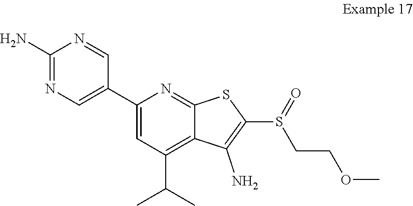 [SMILES: COCCS(=O)c1sc2nc(-c3cnc(N)nc3)cc(C(C)C)c2c1N]

Example 17 was prepared in a manner analogous to that used for Example 7. The target compound was isolated as a yellow solid. LCMS: (ES⁺) m/z (M+H)⁺=392.1. ¹H NMR (400 MHz, CDCl₃) δ 9.02 (s, 2H), 7.50 (s, 1H), 5.35 (s, 2H), 5.09 (s, 2H), 3.94-3.87 (m, 1H), 3.81-3.61 (m, 3H), 3.43 (s, 3H), 3.35-3.27 (m, 1H), 1.48 (dd, J₁=6.8 Hz, J₂=4.0 Hz, 6H).

The enantiomers (Compounds 18 and 19) were separated by SFC (column: DAICEL CHIRALPAK IC (250 mm*30 mm, 10 um); mobile phase: [0.1% NH₃H₂O MEOH]; B %: 60%-60%, 3.8 min; 99 min) to give the (+) enantiomer (76.0 mg, 42% yield, 98.7% purity, 98.7% ee) and the (−) enantiomer (61.4 mg, 34% yield, 98.4% purity, 93.9% ee) as yellow solids. Optical Rotation determination showed the Specific Rotations were +159.997° and −134.476°.

### Example 18: Syntheses of 5-{3-amino-2-[(cyclobutanesulfinyl]-4-(propan-2-yl)thieno[2,3-b]pyridin-6-yl}pyrimidin-2-amine and its Enantiomers (Compounds 20 and 21)

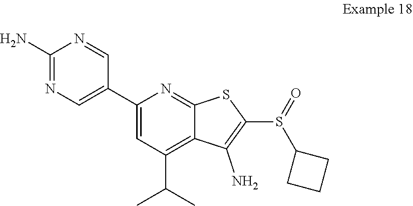 [SMILES: CC(C)c1cc(-c2cnc(N)nc2)nc2sc(S(=O)C3CCC3)c(N)c12]

Example 18 was prepared in a manner analogous to that used for Example 7. The target compound was isolated as a yellow solid. LCMS: (ES+) m/z (M+H)+=388.1. ¹H NMR (400 MHz, CDCl₃) δ 8.99 (m, 2H), 7.47 (s, 1H), 5.28 (s, 2H), 3.99-3.95 (m, 1H), 3.81-3.76 (m, 1H), 2.89-2.80 (m, 1H), 2.41-2.37 (m, 2H), 2.27-2.25 (m, 1H), 2.12-2.06 (m, 2H), 1.47-1.44 (m, 6H).

The enantiomers (Compounds 20 and 21) were separated by SFC (column: DAICEL CHIRALPAK AS (250 mm*30 mm, 10 um); mobile phase: [0.1% NH3H2O ETOH]; B %: 60%-60%, 6.4; 150 min) to give the (+) enantiomer (40.7 mg, 35% yield, 99% purity, 100% ee) and the (−) enantiomer (108.7 mg, 93% yield, 99% purity, 100% ee) as yellow solids. Optical Rotation determination showed the Specific Rotations were +73.213° and −51.454°.

### Example 19: Syntheses of 5-{3-amino-2-[(R)-2-methoxyethanesulfinyl]-4-(propan-2-yl)thieno[2,3-b]pyridin-6-yl}-N-methylpyrimidin-2-amine and its Enantiomers (Compounds 22 and 23)

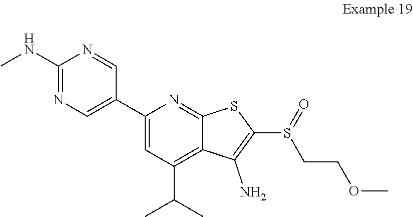

Example 19 was prepared in a manner analogous to that used for Example 7. The target compound was isolated as a yellow solid. LCMS: (ES⁺) m/z (M+H)⁺=406.2.

¹H NMR (400 MHz, CDCl₃) δ 8.99 (m, 2H), 7.47 (s, 1H), 5.40 (s, 1H), 5.06 (s, 2H), 3.90-3.88 (m, 1H), 3.74-3.61 (m, 3H), 3.42 (s, 3H), 3.30-3.29 (m, 1H), 3.10 (d, J=4.8 Hz, 3H), 1.45 (dd, J₁=6.8 Hz; J₂=3.6 Hz, 6H).

The enantiomers (Compounds 22 and 23) were separated by SFC (column: DAICEL CHIRALCEL OD (250 mm*30 mm, 10 um); mobile phase: [0.1% NH₃H₂O MeOH]; B %: 40%-40%, 2.1 min; 110 min) to give the (+) enantiomer (36.6 mg, 48% yield, 99% purity, 100% ee) and the (−) enantiomer (15.7 mg, 20% yield, 99% purity, 97% ee) as yellow solids. Optical Rotation determination showed the Specific Rotations were +26.829° and −43.948°.

### Example 20: Synthesis of 4-tert-butyl-2-[(R)-2-methoxyethanesulfinyl]-6-{2-methyl-2H-pyrazolo[3,4-b]pyridin-5-yl}thieno[2,3-b]pyridin-3-amine (Compound 28)

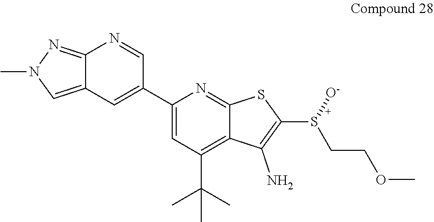

Compound 28 was prepared in a manner analogous to that used for Example 2. The target compound was isolated as a yellow solid. Optical Rotation determination showed the Specific Rotation was +52.958°; LCMS: (ES+) m/z (M+H)+=444.1. ¹H NMR (500 MHz, DMSO-d6) δ 9.37 (d, J=2.0 Hz, 1H), 8.99 (d, J=2.0 Hz, 1H), 8.57 (s, 1H), 7.94 (s, 1H), 5.53 (s, 2H), 4.25 (s, 3H), 3.77-3.74 (m, 1H), 3.63-3.61 (m, 1H), 3.46-3.44 (m, 1H), 3.34-3.29 (m, 4H), 1.65 (s, 9H).

### Example 21: 4-tert-butyl-2-[(R)-2-methoxyethanesulfinyl]-6-{2-methyl-2H-[1,2,3]triazolo[4,5-b]pyridin-6-yl}thieno[2,3-b]pyridin-3-amine (Compound 29)

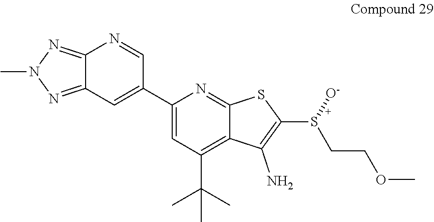

Compound 29 was prepared in a manner analogous to that used for Example 2. The target compound was isolated as a yellow solid. Optical Rotation determination showed the Specific Rotation was +60.649°; LCMS: (ES+) m/z (M+H)+=445.1. ¹H NMR (500 MHz, DMSO-d6) δ 9.56 (s, 1H), 9.16 (d, J=2.0 Hz, 1H), 8.02 (s, 1H), 5.55 (s, 2H), 4.61 (s, 3H), 3.79-3.77 (m, 1H), 3.64-3.62 (m, 1H), 3.46-3.44 (m, 1H), 3.33-3.21 (m, 4H), 1.66 (s, 9H).

### Example 22: Synthesis of 5-{3-amino-4-tert-butyl-2-[(R)-cyclobutanesulfinyl]thieno[2,3-b]pyridin-6-yl}pyrimidin-2-amine (Compound 35)

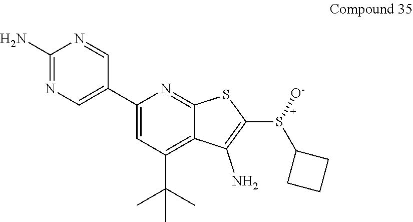

Compound 35 was prepared in a manner analogous to that used for Example 4. The target compound was isolated as a yellow solid. Optical Rotation determination showed the Specific Rotation was +141.610°; LCMS: (ES⁺) m/z (M+H)⁺=402.1. ¹H NMR (400 MHz, CDCl₃) δ 9.00 (s, 2H), 7.59 (s, 1H), 5.38 (s, 2H), 5.29 (s, 2H), 4.09-4.01 (m, 1H), 2.88-2.80 (m, 1H), 2.43-2.38 (m, 2H), 2.26-2.25 (m, 1H), 2.12-2.08 (m, 2H), 1.66 (s, 9H).

### Example 23: Synthesis of 6-{3-amino-2-[(R)-cyclobutanesulfinyl]-4-(propan-2-yl)thieno[2,3-b]pyridin-6-yl}-3-methyl-3,4-dihydropyrimidin-4-one (Compound 33)

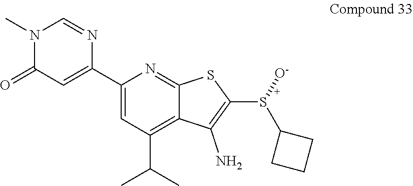

Compound 33 was prepared in a manner analogous to that used for Example 4. The target compound was isolated as a yellow solid. Optical Rotation determination showed the Specific Rotation was +84.623°; LCMS: (ES⁺) m/z (M+H)⁺=403.2. ¹H NMR (400 MHz, CDCl₃) δ 8.24 (s, 1H), 8.20 (s, 1H), 7.57 (s, 1H), 5.17 (s, 2H), 4.04-3.94 (m, 1H), 3.86-3.77 (m, 1H), 3.60 (s, 3H), 2.90-2.79 (m, 1H), 2.49-2.34 (m, 2H), 2.32-2.22 (m, 1H), 2.18-2.05 (m, 2H), 1.48 (dd, =10.4 Hz, J₂=6.8 Hz, 6H).

For example, Compound 33 can prepared in a manner analogous to that used for Example 4, using 6-bromo-3-methylpyrimidin-4(3H)-one in place of 3-bromoimidazo[1,2-a]pyrazine, isobutyraldehyde in place of cyclobutanecarbaldehyde, and (R)-((bromomethyl)sulfinyl)cyclobutane in place of (R)-1-((chloromethyl)sulfinyl)-2-methoxyethane. 6-bromo-3-methylpyrimidin-4(3H)-one is available from commercial sources (e.g., AstaTech Catalog No. AC9854) or can be prepared by methylation of 6-bromopyrimidin-4(3H)-one, as described in Example 140 (Step A) of International Publication No. WO 2014/081617.

### Example 24: Synthesis of 6-{3-amino-2-[(R)-2-methoxyethanesulfinyl]-4-(propan-2-yl)thieno[2,3-b]pyridin-6-yl}-3-methyl-3,4-dihydropyrimidin-4-one (Compound 34)

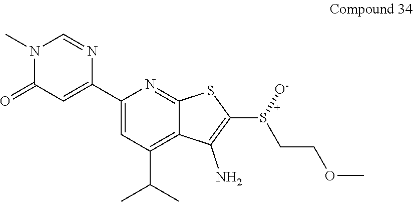

Compound 34 was prepared in a manner analogous to that used for Example 23, using (R)-1-((chloromethyl)sulfinyl)-2-methoxyethane. The target compound was isolated as a yellow solid. Optical Rotation determination showed the Specific Rotation was +48.960°; LCMS: (ES+) m/z (M+H)+=407.2. ¹H NMR (400 MHz, CDCl₃) δ 8.24 (s, 1H), 8.21 (s, 1H), 7.59 (s, 1H), 5.13 (s, 2H), 3.94-3.87 (m, 1H), 3.84-3.76 (m, 1H), 3.75-3.69 (m, 1H), 3.68-3.62 (m, 1H), 3.61 (s, 3H), 3.43 (s, 3H), 3.36-3.28 (m, 1H), 1.50 (t, J=6.8 Hz, 6H).

### Example 25: Synthesis of 2-(cyclobutanesulfinyl)-4-(1-methyl-1H-pyrazol-5-yl)-6-{2H,3H,4H-pyrido[3,2-b][1,4]oxazin-7-yl}thieno[2,3-b]pyridin-3-amine (Compound 30)

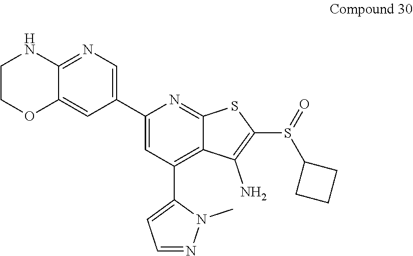 [SMILES: Cn1nccc1-c1cc(-c2cnc3c(c2)OCCN3)nc2sc(S(=O)C3CCC3)c(N)c12]

Compound 30 was prepared in a manner analogous to that used for Example 7. The target compound was isolated as a yellow solid. LCMS: (ES⁺) m/z (M+H)⁺=467.1. ¹H NMR (500 MHz, CDCl₃) δ 8.38 (s, 1H), 7.77 (s, 1H), 7.64 (s, 1H), 7.46 (s, 1H), 6.46 (s, 1H), 5.19 (s, 1H), 4.51-4.27 (m, 2H) 4.26-4.25 (m, 2H), 3.94-3.92 (m, 1H), 3.73 (s, 3H), 3.64-3.62 (m, 2H), 2.80-2.78 (m, 1H), 2.37-2.33 (m, 2H), 2.23-2.22 (m, 1H), 2.09-2.05 (m, 2H).

### Example 26: Synthesis of 2-(cyclobutanesulfinyl)-4-(1-methyl-1H-pyrazol-5-yl)-6-(5,6,7,8-tetrahydro-1,6-naphthyridin-3-yl)thieno[2,3-b]pyridin-3-amine (Compound 31)

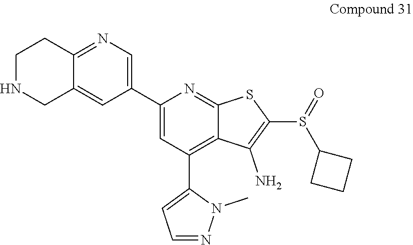 [SMILES: Cn1nccc1-c1cc(-c2cnc3c(c2)CNCC3)nc2sc(S(=O)C3CCC3)c(N)c12]

### Example 26A: tert-butyl 3-nitro-7,8-dihydro-1,6-naphthyridine-6(5H)-carboxylate

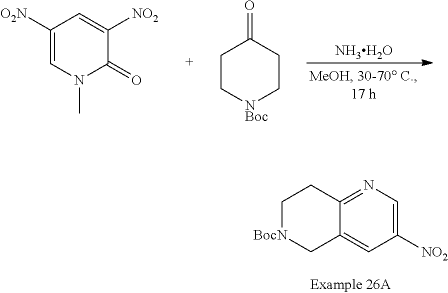 [SMILES: Cn1cc([N+](=O)[O-])cc([N+](=O)[O-])c1=O ; CC(C)(C)OC(=O)N1CCC(=O)CC1 ; "NH3·H2O" ; CC(C)(C)OC(=O)N1CCc2ncc([N+](=O)[O-])cc2C1]

1-Methyl-3,5-dinitro-pyridin-2-one (2.0 g, 10.0 mmol,) and tert-butyl-4-oxopiperidine-1-carboxylate (2.2 g, 11.0 mmol) were suspended in MeOH (20 mL) and the resulting mixture treated with NH₃H₂O (4.55 g, 39.0 mmol, 5 mL, 30% purity). The resulting mixture was heated at 70° C. for 5 hours then left to stand at 30° C. for 12 hours. The mixture was concentrated to remove the solvent. The reaction mixture was partitioned between water (30 mL) and DCM (90 mL). The organic phase was separated, washed with brine (20 mL*3), dried over Na₂SO₄, filtered and concentrated under reduced pressure to give a residue. The residue was purified by column chromatography (SiO₂, Petroleum ether/Ethyl acetate=5/1 to 1/1) to give the target compound (2 g, 71% yield) as a yellow solid. ¹H NMR (400 MHz, CDCl₃) δ=9.25 (d, J=2.0 Hz, 1H), 8.28-8.18 (m, 1H), 4.72 (s, 2H), 3.81 (t, J=6.4 Hz, 2H), 3.12 (t, J=5.8 Hz, 2H), 1.51 (s, 9H).

### Example 26B: tert-butyl 3-amino-7,8-dihydro-1,6-naphthyridine-6(5H)-carboxylate

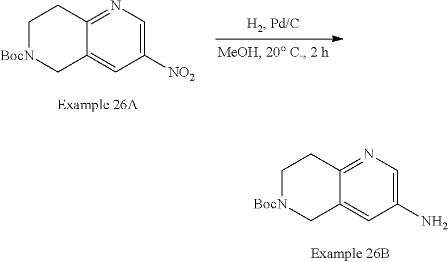 [SMILES: CC(C)(C)OC(=O)N1CCc2ncc([N+](=O)[O-])cc2C1 ; CC(C)(C)OC(=O)N1CCc2ncc(N)cc2C1]

To a solution of Example 26A (0.2 g, 716 μmol) in MeOH (5 mL) was added Pd/C (0.1 g, 10% purity) under N₂. The suspension was degassed under vacuum and purged with H₂ several times. The mixture was stirred under H₂ (15 psi) at 20° C. for 2 hours. The mixture was filtered to remove the solid. Then the filtrate was concentrated to remove the solvent to give the target compound (180 mg, crude) as a colorless oil.

### Example 26C: tert-butyl 3-bromo-7,8-dihydro-1,6-naphthyridine-6(5H)-carboxylate

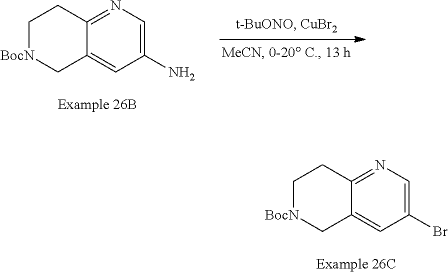 [SMILES: CC(C)(C)OC(=O)N1CCc2ncc(N)cc2C1 ; CC(C)(C)OC(=O)N1CCc2ncc(Br)cc2C1]

CuBr₂ (241.9 mg, 1.08 mmol) was added to a solution of Example 26B (180 mg, 722 μmol) in MeCN (6 mL) at 20° C., followed by dropwise addition of t-butyl nitrite (89.3 mg, 866 μmol) at 0° C. The reaction was stirred at 0° C. for 1 hour, then at 20° C. for 12 hours. The mixture was poured into 30 mL water, filtered and extracted three times each with 30 mL of EA. The combined organic phases were washed twice with 30 mL brine each time, dried over Na₂SO₄, filtered and concentrated to remove the solvent to give the target compound (200 mg, 88% yield) as a brown oil.

### Example 26D: tert-butyl 3-(4,4,5,5-tetramethyl-1,3,2-dioxaborolan-2-yl)-7,8-dihydro-1,6-naphthyridine-6(5H)-carboxylate

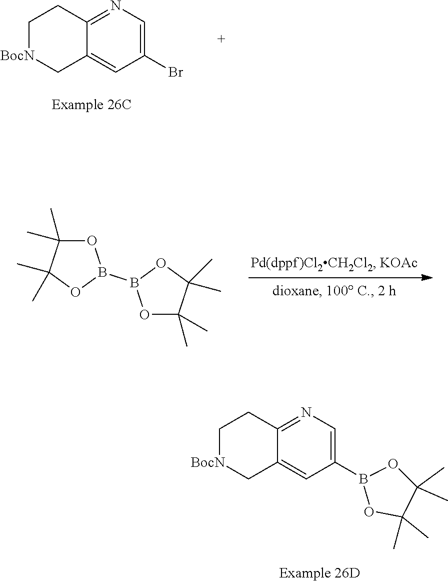

To a solution of Example 26C (150 mg, 479 mol), 4,4,4′,4′,5,5,5′,5′-octamethyl-2,2′-bi(1,3,2-dioxaborolane) (182 mg, 718 mol) and KOAc (141 mg, 1.44 mmol) in dioxane (3 mL) was added Pd(dppf)Cl₂.CH₂Cl₂ (78.2 mg, 95.8 μmol). The mixture was stirred under N₂ at 100° C. for 2 hours. The resulting dioxane solution was used as-is in the next reaction.

### Example 26F: tert-butyl 3-(5-cyano-6-(((cyclobutylthio)methyl)thio)-4-(1-methyl-1H-pyrazol-5-yl)pyridin-2-yl)-7,8-dihydro-1,6-naphthyridine-6(5H)-carboxylate

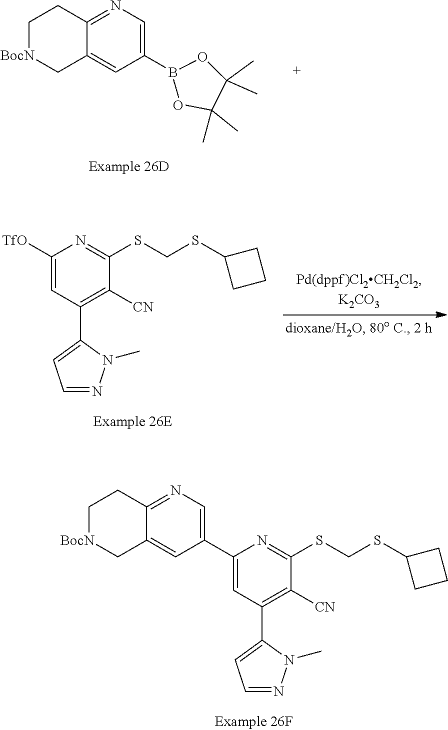 [SMILES: CC(C)(C)OC(=O)N1CCc2ncc(B3OC(C)(C)C(C)(C)O3)cc2C1 ; Cn1nccc1-c1cc(OS(=O)(=O)C(F)(F)F)nc(SCSC2CCC2)c1C#N ; Cn1nccc1-c1cc(-c2cnc3c(c2)CN(C(=O)OC(C)(C)C)CC3)nc(SCSC2CCC2)c1C#N]

Example 26E was prepared in a manner to that used for the preparation of Example 7D. A mixture of Example 26D (170 mg, 472 μmol), Example 26E (219 mg, 472 μmol), Pd(dppf)Cl₂.CH₂Cl₂ (38.5 mg, 47.2 μmol), K₂CO₃ (130 mg, 944 μmol) in dioxane (3 mL) and H₂O (1 mL) was degassed and purged with N₂ 3 times. The mixture was stirred at 80° C. for 2 hours under N₂. The reaction mixture was partitioned between water (30 mL) and EA (100 mL). The organic phase was separated, washed with brine (30 mL*2), dried over Na₂SO₄, filtered and concentrated under reduced pressure to give a residue. The residue was purified by column chromatography (SiO₂, Petroleum ether/Ethyl acetate=I/O to 1/1) to give the target compound (90 mg, 35% yield) as a yellow solid.

### Example 26G: tert-butyl 3-(5-cyano-6-(((cyclobutylsulfinyl)methyl)thio)-4-(1-methyl-1H-pyrazol-5-yl)pyridin-2-yl)-7,8-dihydro-1,6-naphthyridine-6(5H)-carboxylate

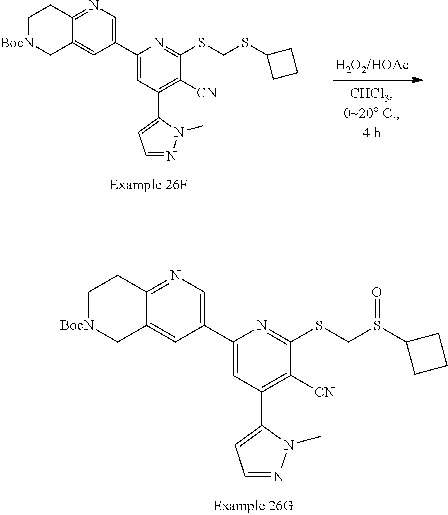 [SMILES: Cn1nccc1-c1cc(-c2cnc3c(c2)CN(C(=O)OC(C)(C)C)CC3)nc(SCSC2CCC2)c1C#N ; Cn1nccc1-c1cc(-c2cnc3c(c2)CN(C(=O)OC(C)(C)C)CC3)nc(SCS(=O)C2CCC2)c1C#N]

To a solution of Example 26F (80 mg, 146 μmol) in CHCl₃ (3 mL) was added HOAc (175 mg, 2.92 mmol) and H₂O₂ (82.6 mg, 729 μmol, 30% purity) at 0° C. The mixture was stirred at 20° C. for 4 hours. The mixture was quenched by adding 10 mL NaHCO₃ solution and 20 mL saturated Na₂SO₃ solution. The reaction mixture was partitioned between water (10 mL) and DCM (50 mL). The organic phase was separated, washed with brine (20 mL*2), dried over Na₂SO₄, filtered and concentrated under reduced pressure to give the target compound (95 mg, crude) as a yellow solid.

### Example 26H: tert-butyl 3-(3-amino-2-(cyclobutylsulfinyl)-4-(1-methyl-1H-pyrazol-5-yl)thieno[2,3-b]pyridin-6-yl)-7,8-dihydro-1,6-naphthyridine-6(5H)-carboxylate

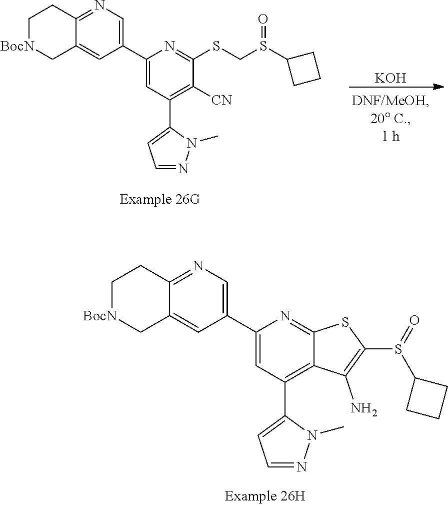

To a solution of Example 26G (90 mg, 159 μmol) in DMF (3 mL) and MeOH (3 mL) was added KOH (17.9 mg, 319 μmol). The mixture was stirred at 20° C. for 1 hour. The mixture was quenched by adding 10 mL of water. The yellow solid was filtered and used for the next step with no purification.

### Example 26: Synthesis of Compound 31

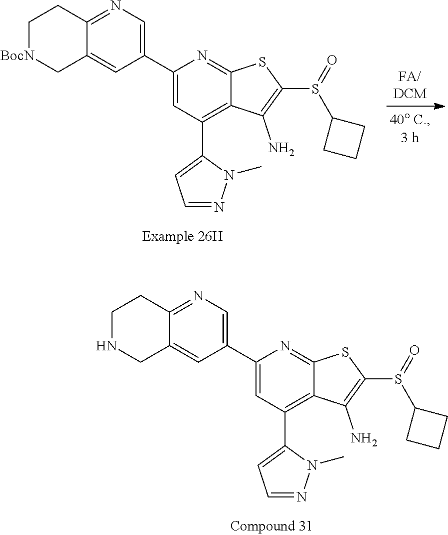 [SMILES: Cn1nccc1-c1cc(-c2cnc3c(c2)CN(C(=O)OC(C)(C)C)CC3)nc2sc(S(=O)C3CCC3)c(N)c12 ; Cn1nccc1-c1cc(-c2cnc3c(c2)CNCC3)nc2sc(S(=O)C3CCC3)c(N)c12]

To a solution of Example 26H (80 mg, 142 μmol) in DCM (4 mL) was added formic acid (4.88 g, 4.00 mL). The mixture was stirred at 40° C. for 3 hours. The mixture was poured into 200 mL saturated NaHCO₃ solution. The mixture was extracted with DCM (50 mL*3). The combined organic layers were washed with brine (30 mL*2), dried over Na₂SO₄ and concentrated to remove the solvent. The residue was purified by prep-HPLC (column: Agela DuraShell C18 150*25 mm*5 um; mobile phase: [water (0.05% NH₃H₂O+10 mM NH₄HCO₃)-ACM; B %: 21%-51%, 10 min) to give the target compound (9 mg, 13% yield) as a yellow solid. LCMS: (ES⁺) m/z (M+H)⁺=465.1. ¹H NMR (400 MHz, CDCl₃) δ=9.06 (s, 1H), 8.14 (s, 1H), 7.68 (d, J=1.6 Hz, 1H), 7.61 (s, 1H), 6.50 (d, J=6.4 Hz, 1H), 4.63-4.45 (m, 2H), 4.19 (s, 2H), 3.98-3.90 (m, 1H), 3.76 (s, 3H), 3.37-3.30 (m, 2H), 3.13-3.06 (m, 2H), 2.93-2.74 (m, 1H), 2.44-2.33 (m, 2H), 2.29-2.21 (m, 1H), 2.15-2.05 (m, 2H).

### Example 27: 2-(2-methoxyethanesulfinyl)-4-(1-methyl-1H-pyrazol-5-yl)-6-{2H,3H,4H-pyrido[3,2-b][1,4]oxazin-7-yl}thieno[2,3-b]pyridin-3-amine (Compound 32)

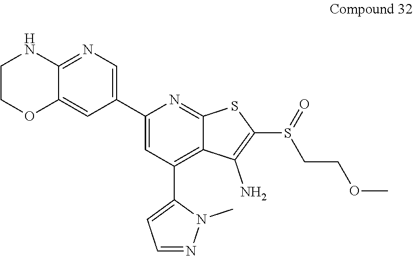 [SMILES: COCCS(=O)c1sc2nc(-c3cnc4c(c3)OCCN4)cc(-c3ccnn3C)c2c1N]

Compound 32 was prepared in a manner analogous to that used for Example 7. The target compound was isolated as a yellow solid. LCMS: (ES+) m/z (M+H)+=471.1. ¹H NMR (500 MHz, CDCl₃) δ 8.39 (s, 1H), 7.78 (s, 1H), 7.65 (s, 1H), 7.48 (s, 1H), 6.47-6.46 (m, 1H), 5.18 (s, 1H), 4.49-4.43 (m, 2H) 4.27-4.26 (m, 2H), 4.26-4.25 (m, 1H), 4.26-3.86 (m, 3H), 3.74 (s, 3H), 3.65-3.62 (m, 1H), 3.62 (s, 3H), 3.38-3.25 (m, 1H).

### Example 28: Syntheses of 2-[2-methoxyethanesulfinyl]-4-(propan-2-yl)-6-(quinoxalin-6-yl)thieno[2,3-b]pyridin-3-amine and its Enantiomers (Compounds 24 and 25)

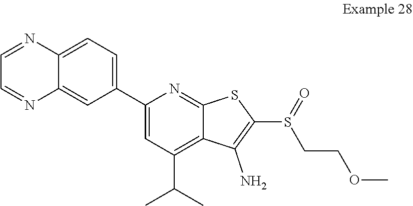 [SMILES: COCCS(=O)c1sc2nc(-c3ccc4nccnc4c3)cc(C(C)C)c2c1N]

Example 28 was prepared in a manner analogous to that used for Example 7. The target compound was isolated as a yellow solid. ¹H NMR (400 MHz, CDCl₃) δ 8.90-8.88 (m, 2H), 8.71 (d, J=1.6 Hz, 1H), 8.66-8.64 (m, 1H), 8.22-8.19 (m, 1H), 7.86 (s, 1H), 5.12 (s, 2H), 4.02-3.91 (m, 1H), 3.83-3.80 (m, 1H), 3.72-3.63 (m, 2H), 3.42 (s, 3H), 3.35-3.31 (m, 1H), 1.50 (dd, J1=6.8 Hz; J2=8.8 Hz, 6H).

The enantiomers (Compounds 24 and 25) were separated by SFC (column: DAICEL CHIRALPAK AD (250 mm*30 mm, 10 um); mobile phase: [0.1% NH3H2O EtOH]; B %: 50%-50%, 4.3 min; 60 min) to give the (+) enantiomer (107.5 mg, 84% yield, 98% purity, 100% ee) and the (−) enantiomer (58.7 mg, 46% yield, 99% purity, 98% ee) as yellow solids. Optical Rotation determination showed the Specific Rotations were +49.022° and −46.314°; LCMS: (ES+) m/z (M+H)+=427.1. ¹H NMR (400 MHz, CDCl₃) δ 8.88-8.85 (m, 2H), 8.67 (d, J=2.0 Hz, 1H), 8.63-8.60 (m, 1H), 8.19-8.15 (m, 1H), 7.84 (s, 1H), 5.11 (s, 2H), 3.89-3.80 (m, 1H), 3.73-3.70 (m, 1H), 3.67-3.62 (m, 2H), 3.42 (s, 3H), 3.32-3.29 (m, 1H), 1.50 (dd, J1=6.8 Hz; J2=21.6 Hz, 6H).

### Numbered Embodiments

Embodiment 1. A compound of formula (I):

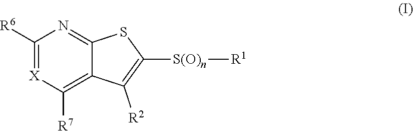

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

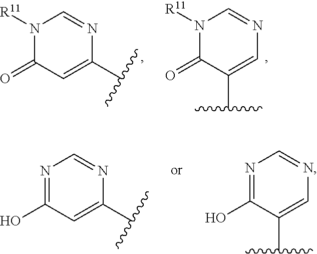

each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —OH, —O-alkeylene-OH, —O-alkeylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆alkoxy;
R⁹ is H or C₁-C₆ alkyl;
R¹⁰ is —OH, halogen, C₁-C₆ alkyl, or C₁-C₆alkoxy;
R¹¹ is H or C₁-C₆ alkyl;
X is N or CH;
m is 0, 1, or 2; and
n is 0, 1, or 2;
wherein the compound is not:

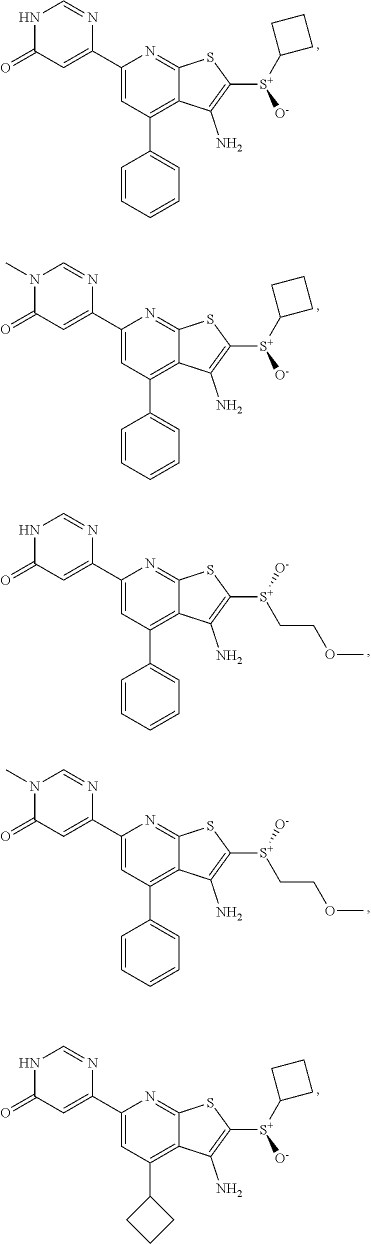 [SMILES: Nc1c([S@+]([O-])C2CCC2)sc2nc(-c3cc(=O)[nH]cn3)cc(-c3ccccc3)c12 ; Cn1cnc(-c2cc(-c3ccccc3)c3c(N)c([S@+]([O-])C4CCC4)sc3n2)cc1=O ; COCC[S@@+]([O-])c1sc2nc(-c3cc(=O)[nH]cn3)cc(-c3ccccc3)c2c1N ; COCC[S@@+]([O-])c1sc2nc(-c3cc(=O)n(C)cn3)cc(-c3ccccc3)c2c1N ; Nc1c([S@+]([O-])C2CCC2)sc2nc(-c3cc(=O)[nH]cn3)cc(C3CCC3)c12]

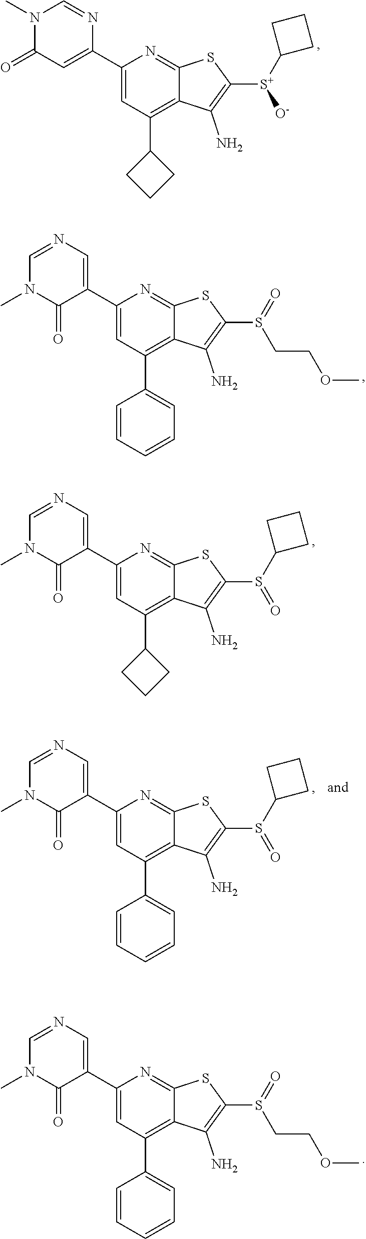

Embodiment 2. The compound of Embodiment 1, wherein R¹ is C₁-C₆ alkyl, C₃-C₆ cycloalkyl, or —(C₁-C₃ alkylene)-(C₁-C₃ alkoxy).

Embodiment 3. The compound of Embodiments 1 or 2, wherein R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

Embodiment 4. The compound of any one of Embodiments 1-3, wherein R² is NH₂.

Embodiment 5. The compound of any one of Embodiments 1-4, wherein R⁶ is

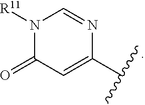

Embodiment 6. The compound of any one of Embodiments 1-5, wherein R¹¹ is H or methyl.

Embodiment 7. The compound of any one of Embodiments 1-6, wherein R⁷ is phenyl, alkyl, or cycloalkyl, each of which is optionally substituted with one or more R⁴.

Embodiment 8. The compound of any one of Embodiments 1-7, wherein R⁷ is a linear or branched, non-cyclic C₁-C₆ alkyl.

Embodiment 9. The compound of any one of Embodiments 1-8, wherein R⁷ is methyl, ethyl, n-propyl, n-butyl, s-butyl, or t-butyl.

Embodiment 10. The compound of any one of Embodiments 1-9, wherein X is CH.

Embodiment 11. The compound of any one of Embodiments 1-10, wherein n is 1.

Embodiment 12. A compound of formula (II):

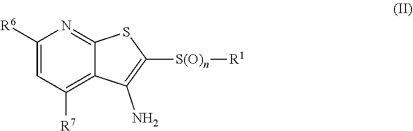

or a pharmaceutically acceptable salt, tautomer, or solvate thereof, wherein:
R¹ is C₁-C₆ alkyl, C₃-C₆ cycloalkyl, or —(C₁-C₃ alkylene)-(C₁-C₃ alkoxy);
R⁶ is

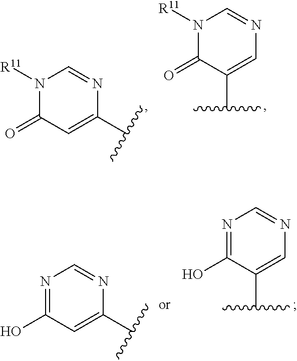

R⁷ is a linear or branched, non-cyclic C₁-C₆ alkyl;
R¹¹ is H or C₁-C₆ alkyl; and
n is 0, 1, or 2.

Embodiment 13. The compound of Embodiment 1 or 12 selected from:

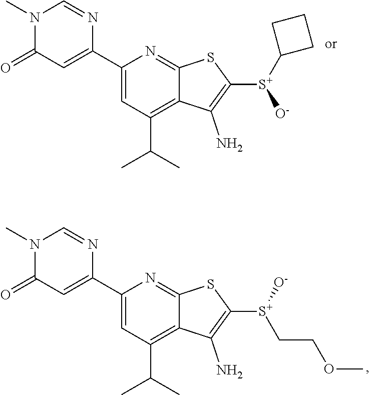

or a pharmaceutically acceptable salt, tautomer, or solvate thereof.

Embodiment 14. A compound of formula (III):

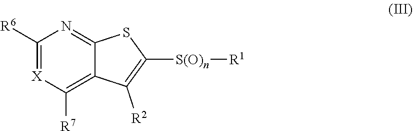

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

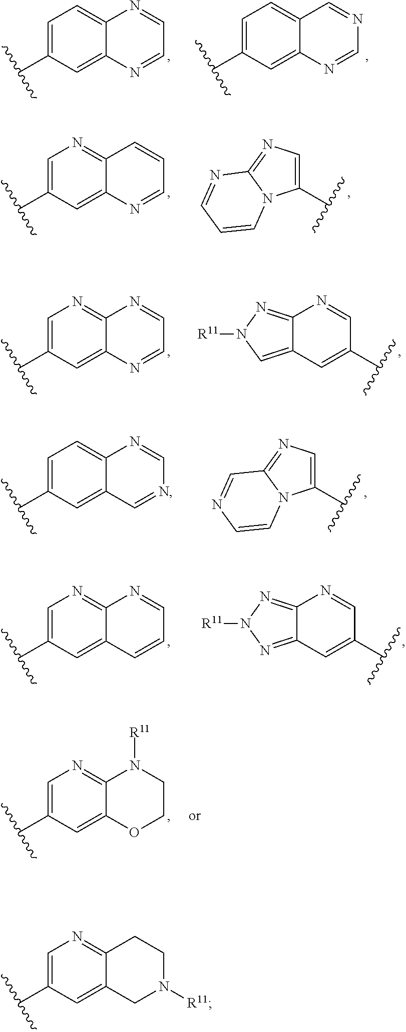

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2;
wherein the compound is not:

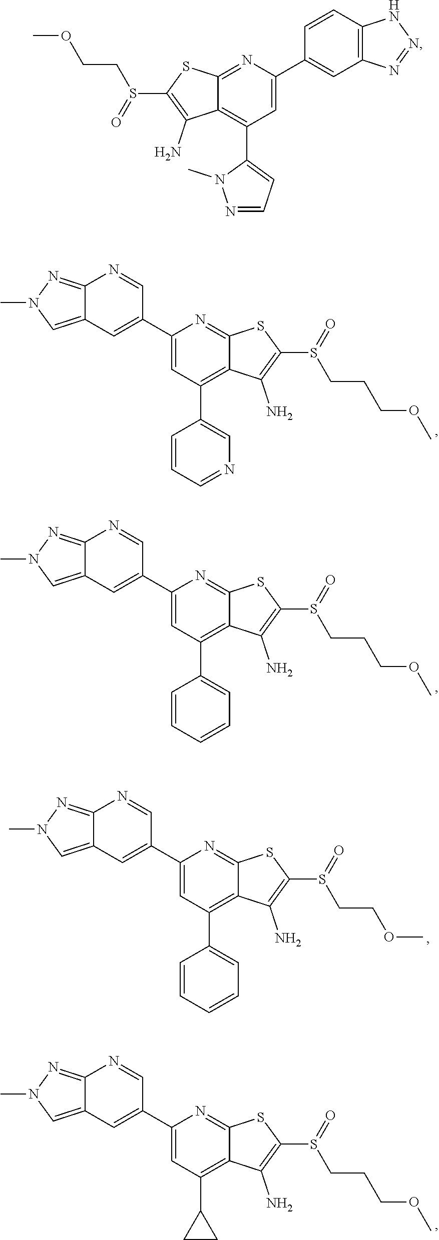

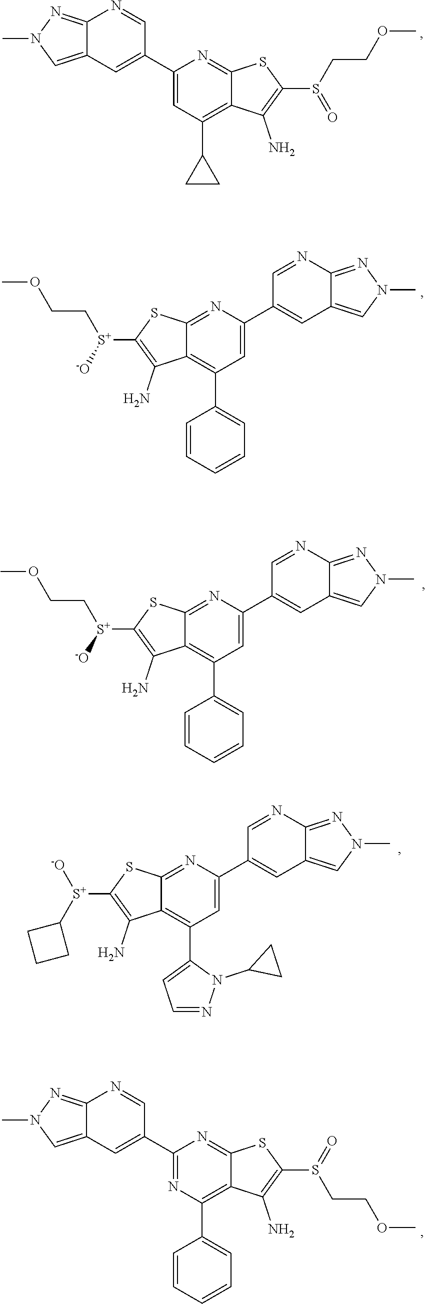

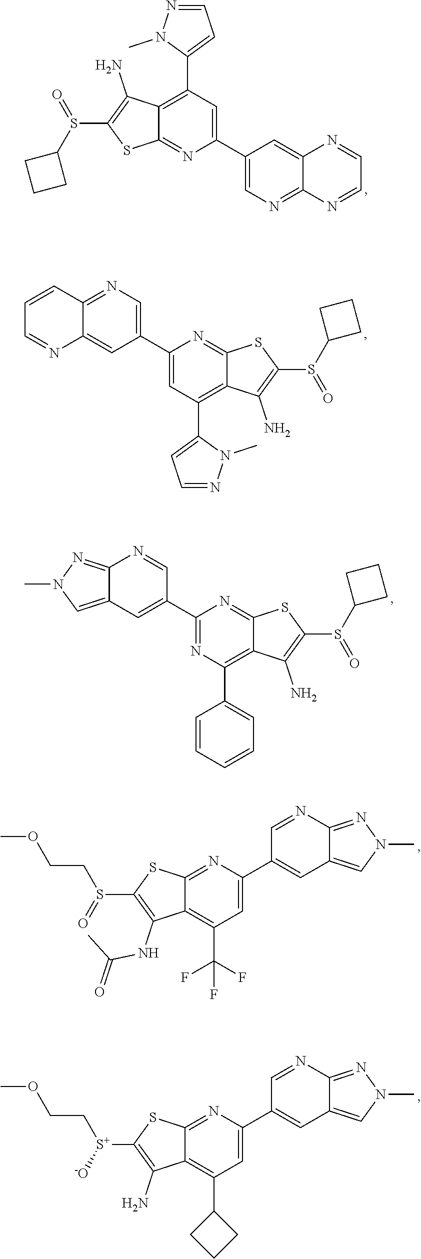

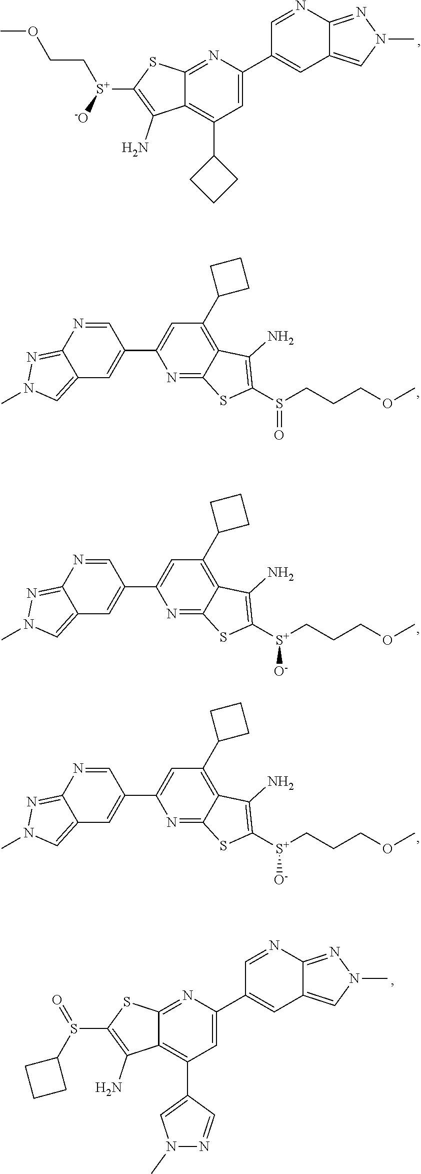

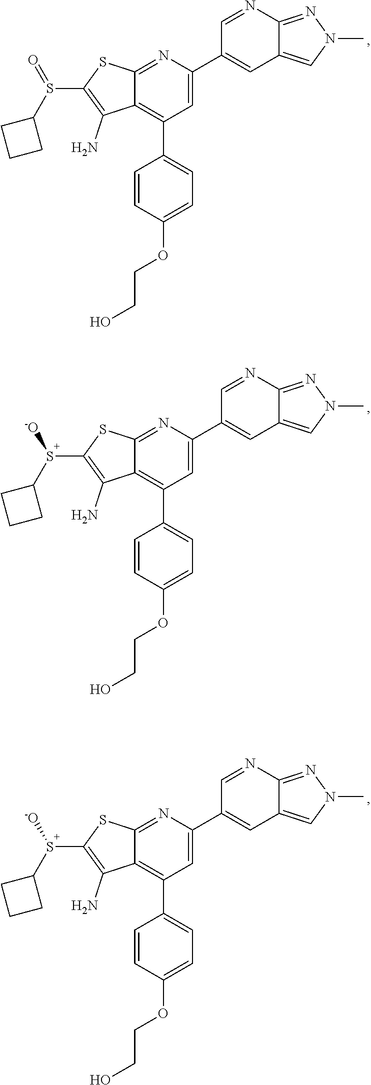

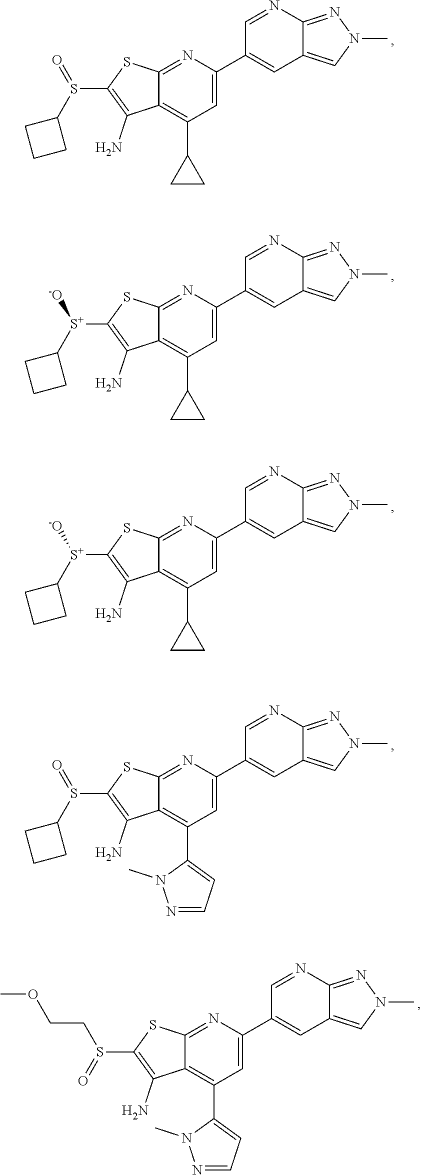

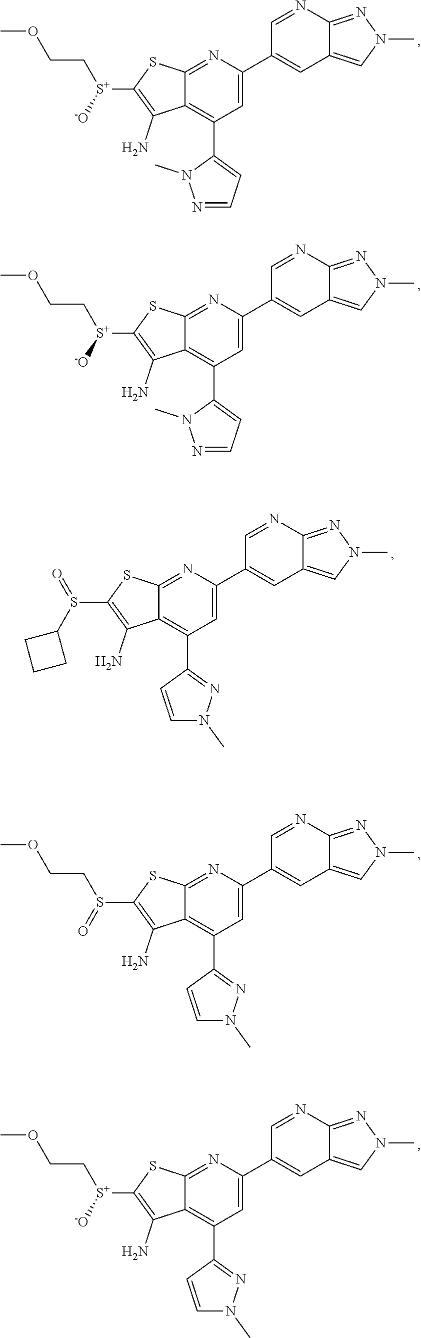

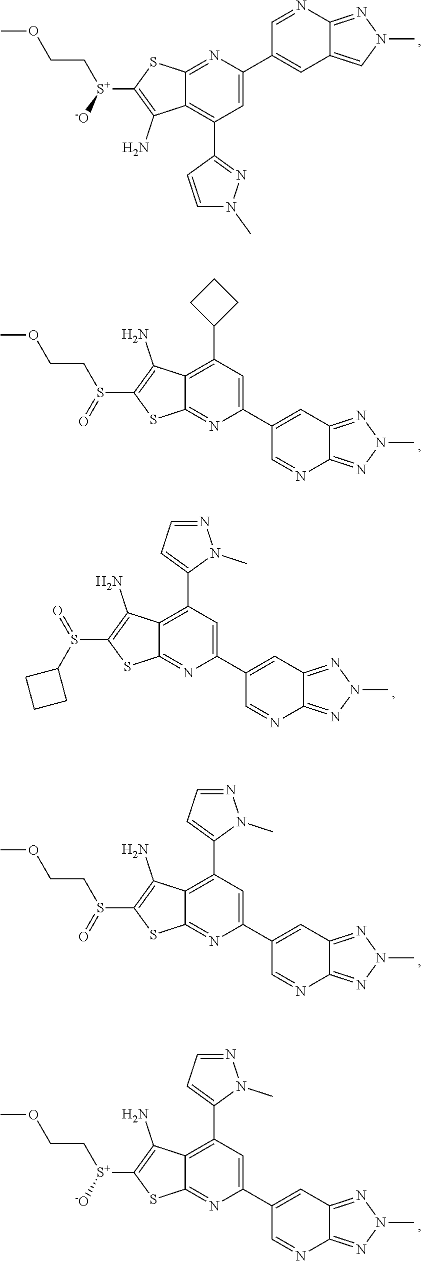

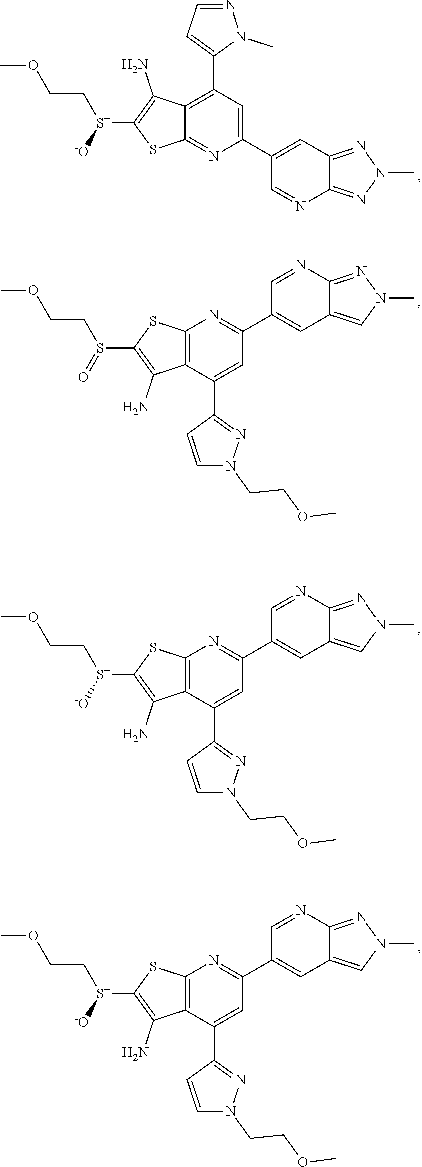

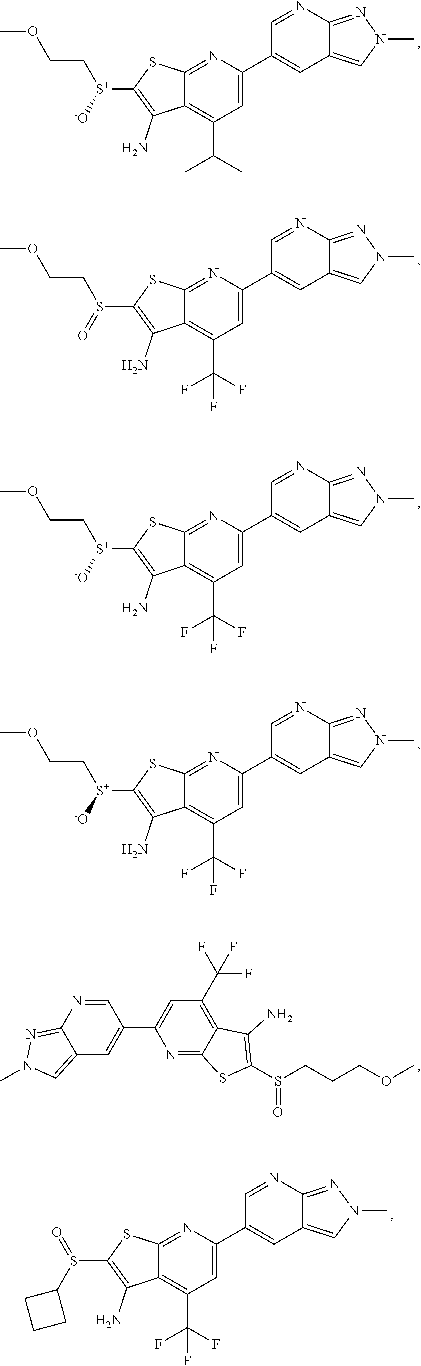

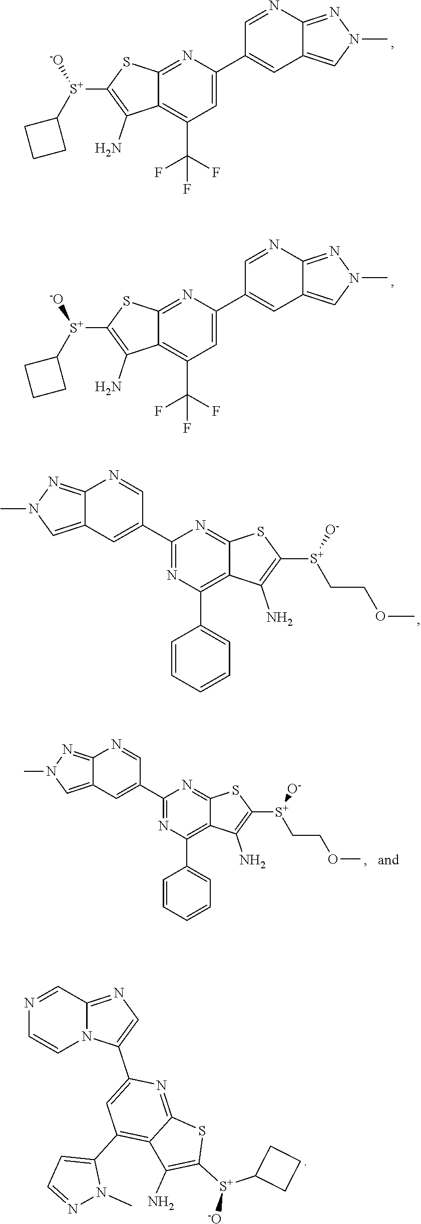

Embodiment 15. The compound of Embodiment 14, wherein R¹ is C₁-C₆ alkyl, C₃-C₆ cycloalkyl, or —(C₁-C₃ alkylene)-(C₁-C₃ alkoxy).

Embodiment 16. The compound of Embodiment 14 or 15, wherein R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

Embodiment 17. The compound of any one of Embodiments 14-16, wherein R² is NH₂ or —CN.

Embodiment 18. The compound of any one of Embodiments 14-17, wherein R⁶ is

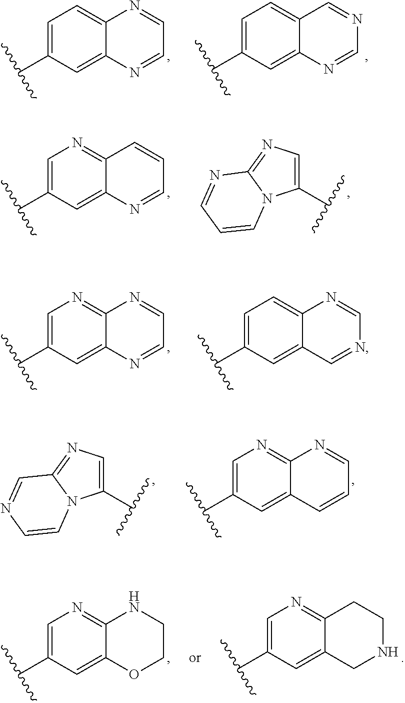

Embodiment 19. The compound of any one of Embodiments 14-18, wherein R⁷ is alkyl, cycloalkyl, aryl, heterocyclyl, or heteroaryl, each of which is optionally substituted with one or more R⁴.

Embodiment 20. The compound of any one of Embodiments 14-19, wherein n is 1.

Embodiment 21. The compound of Embodiment 14 selected from:

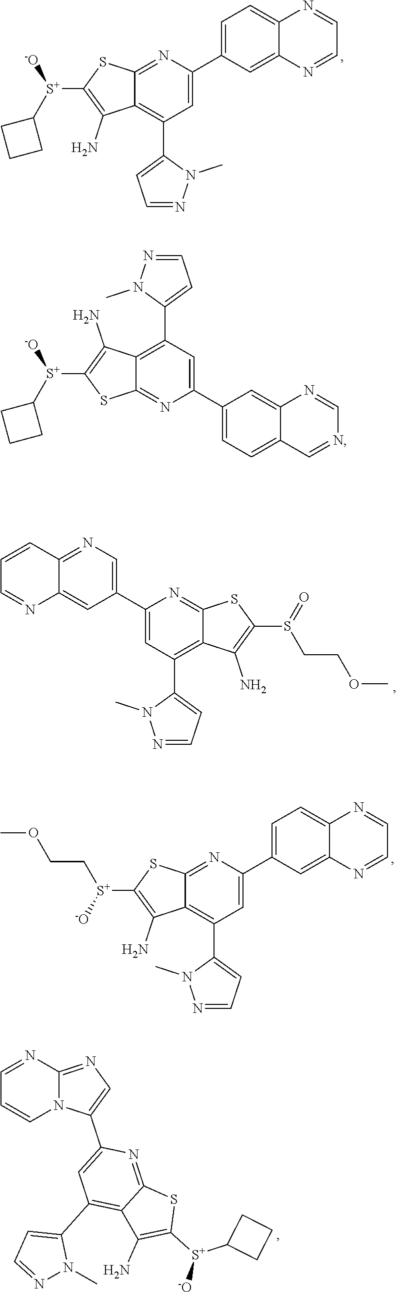

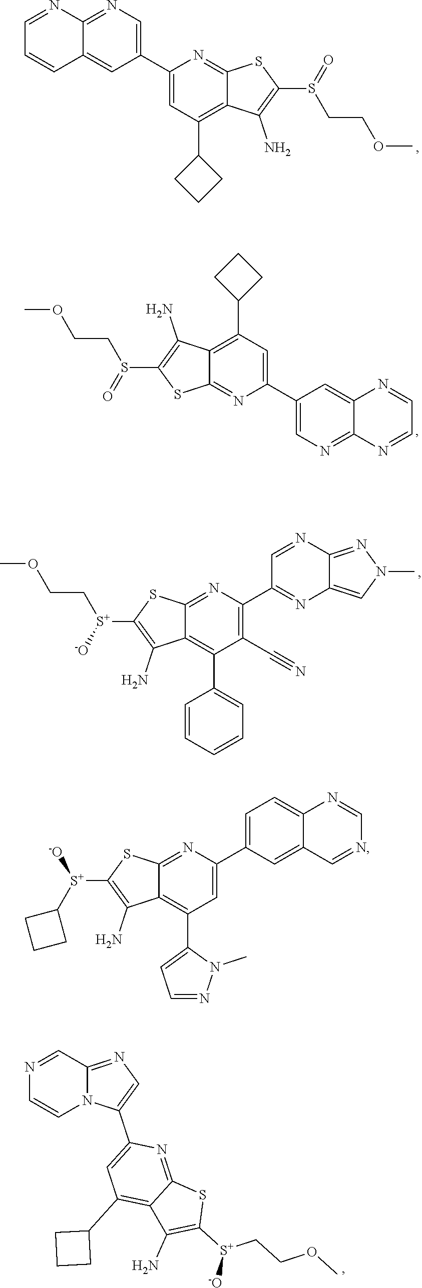

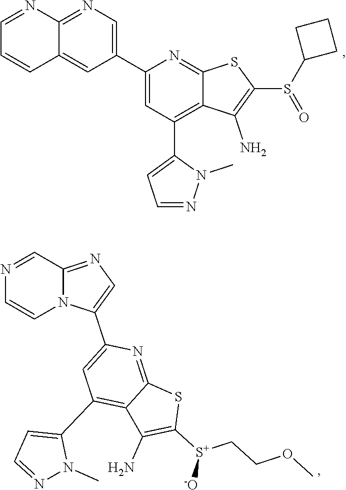

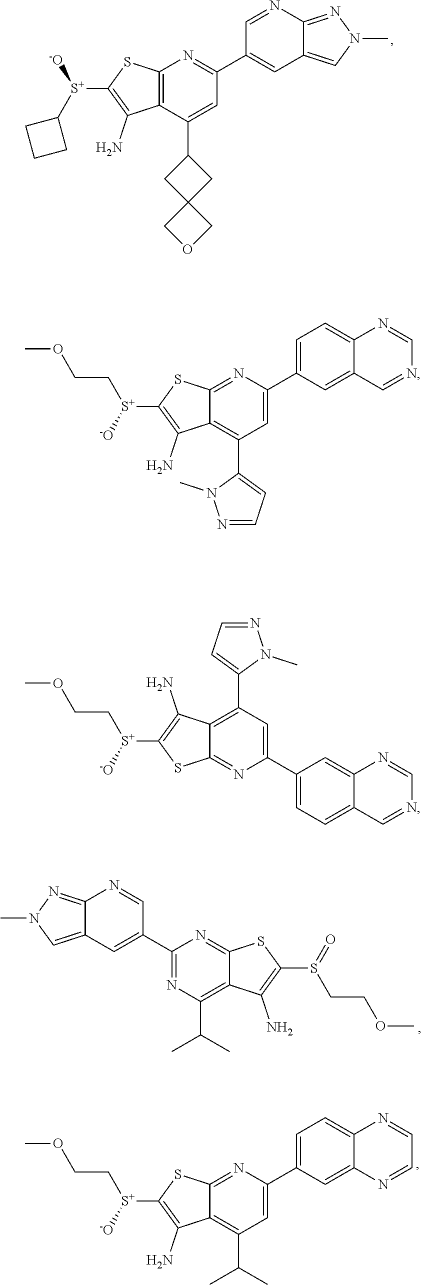

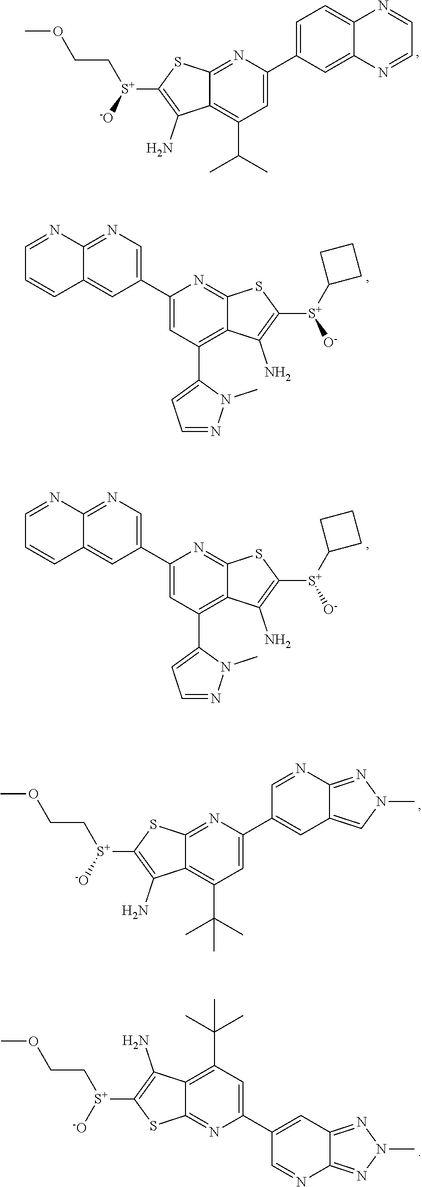

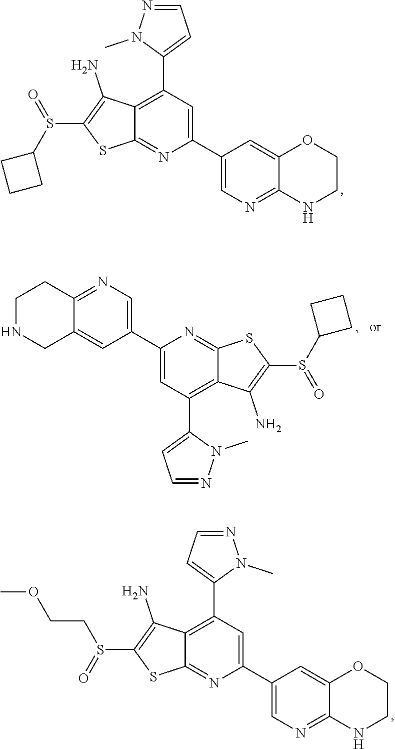 [SMILES: Cn1nccc1-c1cc(-c2cnc3c(c2)OCCN3)nc2sc(S(=O)C3CCC3)c(N)c12 ; Cn1nccc1-c1cc(-c2cnc3c(c2)CNCC3)nc2sc(S(=O)C3CCC3)c(N)c12 ; COCCS(=O)c1sc2nc(-c3cnc4c(c3)OCCN4)cc(-c3ccnn3C)c2c1N]

or a pharmaceutically acceptable salt, tautomer, or solvate thereof.

Embodiment 22. A compound of formula (IV):

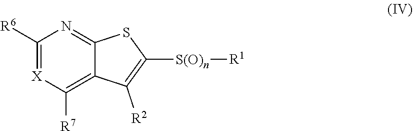

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is

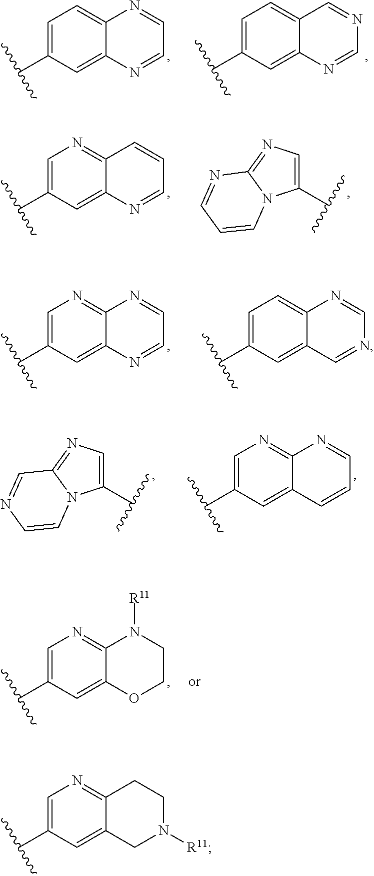

R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R¹ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2;
wherein the compound is not:

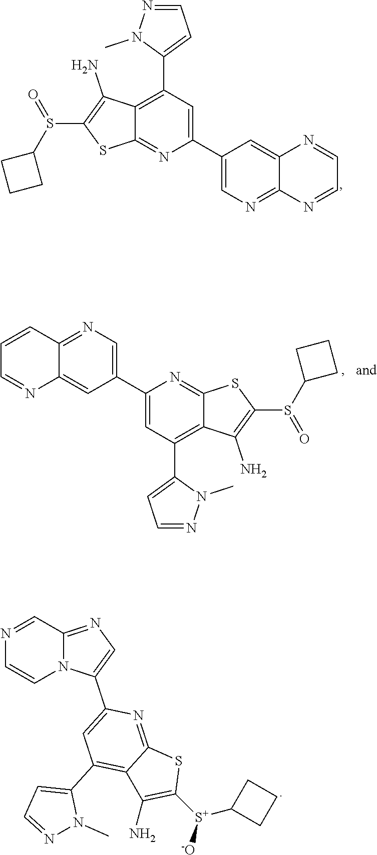

Embodiment 23. A compound selected from:

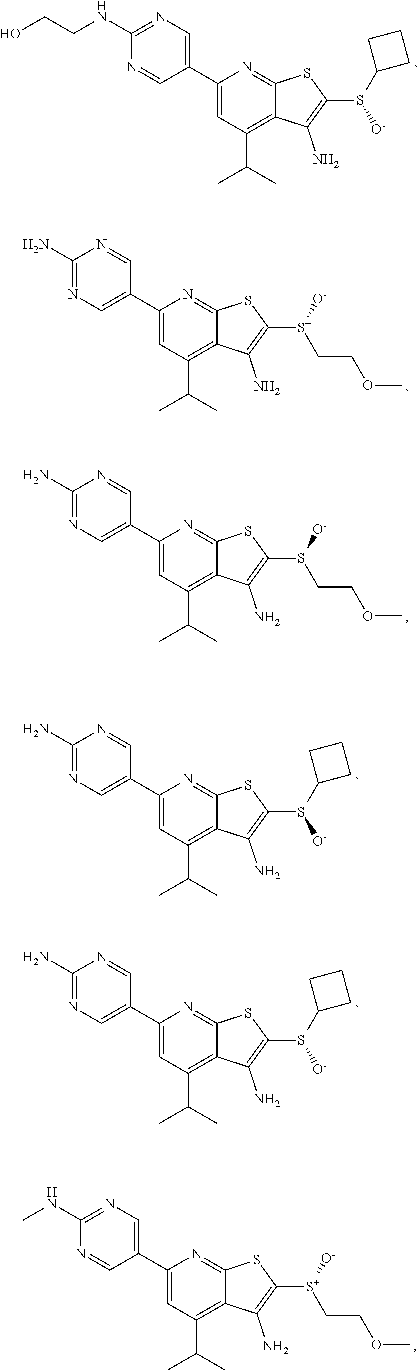

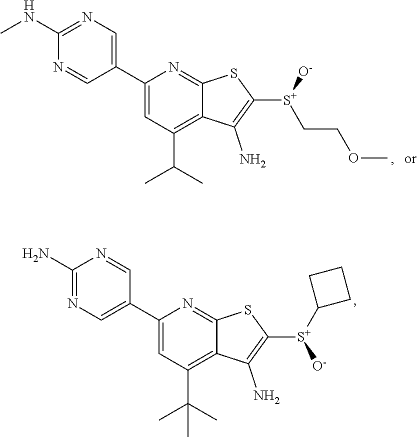

or a pharmaceutically acceptable salt, tautomer, or solvate thereof.

Embodiment 24. A pharmaceutical composition comprising a compound of any one of Embodiments 1-23 and a pharmaceutically acceptable carrier or excipient.

Embodiment 25. Use of a compound of any one of Embodiments 1 to 23 as a short chain dehydrogenase inhibitor for inhibiting the activity of a short chain dehydrogenase enzyme.

Embodiment 28. Use of a compound of any one of Embodiments 1 to 23 as a 15-PGDH inhibitor for inhibiting the activity of a 15-PGDH enzyme.

Embodiment 29. A method of treating a subject in need of cell therapy comprising administering to the subject a therapeutically effective amount of a preparation comprising human hematopoietic stem cell administered a compound of any one of Embodiments 1 to 23 and/or a therapeutic composition comprising human hematopoietic stem cells and a compound of any one of Embodiments 1 to 23.

Embodiment 30. A method of treating a subject having at least one symptom associated with an ischemic tissue or a tissue damaged by ischemia comprising administering to the subject a therapeutically effective amount of a preparation comprising human hematopoietic stem cell administered a compound of any one of Embodiments 1 to 23 and/or a therapeutic composition comprising human hematopoietic stem cells and a compound of any one of Embodiments 1 to 23.

Embodiment 31. A method of increasing neutrophils in a subject in need thereof, the method comprising administering to the subject a compound of any one of Embodiments 1 to 23.

Embodiment 32. A method increasing numbers of and/or of mobilizing peripheral blood hematopoietic stem cells in a subject in need thereof, the method comprising administering to the subject a compound of any one of Embodiments 1 to 23.

Embodiment 33. A method of increasing numbers of hematopoietic stem cells in blood or bone marrow, the method comprising: administering to blood or bone marrow of the subject a compound of any one of Embodiments 1 to 23.

Embodiment 34. A method of treating or preventing a fibrotic disease, disorder or condition in a subject in need thereof, the method comprising administering to the subject a therapeutically effective amount of a compound of any one of Embodiments 1 to 23.

Embodiment 35. A method of treating intestinal, gastrointestinal, or bowel disorders in a subject in need thereof, the method comprising administering to the subject a therapeutically effective amount of a compound of any one of Embodiments 1 to 23 alone or in combination with a corticosteroid and/or a tumor necrosis factor α (TNFα) inhibitor.

Embodiment 36. A method of treating intestinal, gastrointestinal, or bowel disorders in a subject in need thereof, the method comprising: administering to the subject therapeutically effective amounts of a compound of any one of Embodiments 1 to 23 and a corticosteroid.

Embodiment 37. A method of treating inflammation and/or reducing the activity of the immune system in a subject in need thereof, the method comprising: administering to the subject a therapeutically effective amounts of a compound of any one of Embodiments 1 to 23 and a corticosteroid.

Embodiment 38. A method for the treatment of glucocorticoid insensitivity, restoring corticosteroid sensitivity, enhancing glucocorticoid sensitivity or reversing the glucocorticoid insensitivity in a subject experiencing corticosteroid dependence or corticoid resistance or unresponsiveness or intolerance to corticosteroids, comprising: administering a pharmaceutical composition comprising a compound of any one of Embodiments 1 to 23 in combination with a corticosteroid to the subject exhibiting one or more glucocorticoid insensitivity related conditions, wherein the glucocorticoid insensitivity related conditions comprise a range of immune-inflammatory disorders/diseases treated with steroids when the therapy fails to achieve disease control or is not effective or intolerant or dependent to corticosteroids, and combinations thereof.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

What is claimed:

1. A compound of formula (I): 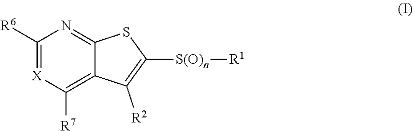
or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is 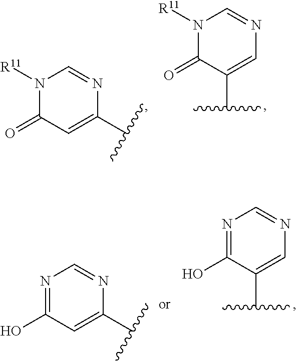
each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —OH, —O-alkeylene-OH, —O-alkeylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹⁰ is —OH, halogen, C₁-C₆ alkyl, or C₁-C₆alkoxy;
R¹¹ is H or C₁-C₆ alkyl;
X is N or CH;
m is 0, 1, or 2; and
n is 0, 1, or 2.

2. The compound of claim 1, wherein the compound is not:

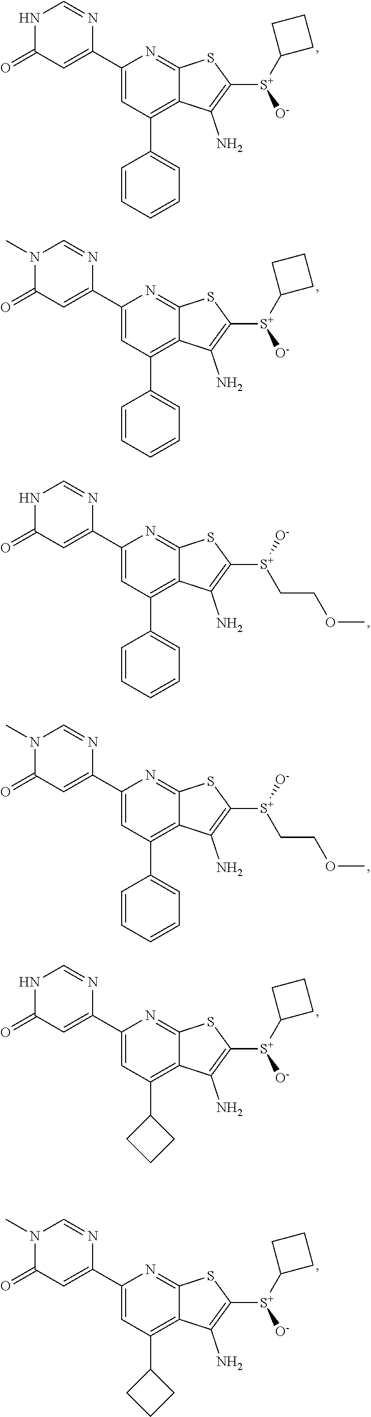 [SMILES: Nc1c([S@+]([O-])C2CCC2)sc2nc(-c3cc(=O)[nH]cn3)cc(-c3ccccc3)c12 ; Cn1cnc(-c2cc(-c3ccccc3)c3c(N)c([S@+]([O-])C4CCC4)sc3n2)cc1=O ; COCC[S@@+]([O-])c1sc2nc(-c3cc(=O)[nH]cn3)cc(-c3ccccc3)c2c1N ; COCC[S@@+]([O-])c1sc2nc(-c3cc(=O)n(C)cn3)cc(-c3ccccc3)c2c1N ; Nc1c([S@+]([O-])C2CCC2)sc2nc(-c3cc(=O)[nH]cn3)cc(C3CCC3)c12 ; Cn1cnc(-c2cc(C3CCC3)c3c(N)c([S@+]([O-])C4CCC4)sc3n2)cc1=O]

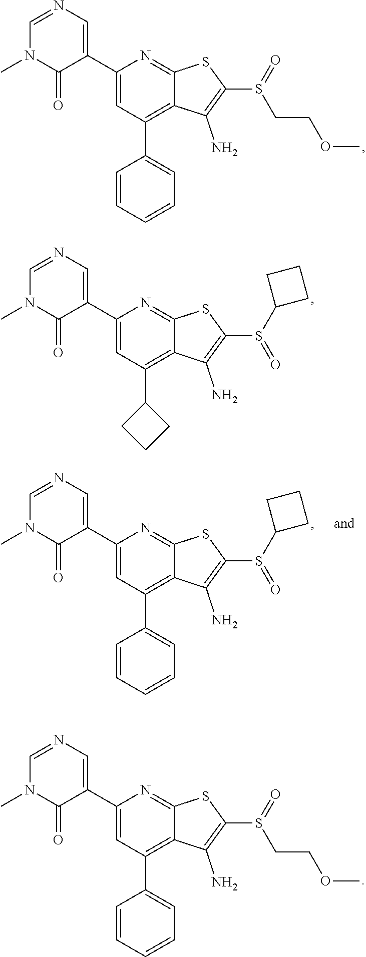

3. The compound of claim 1, wherein R¹ is C₁-C₆ alkyl, C₃-C₆ cycloalkyl, or —(C₁-C₃ alkylene)-(C₁-C₃ alkoxy).

4. The compound of claim 1 or 3, wherein R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

5. The compound of any one of claims 1, 3, and 4, wherein R² is NH₂.

6. The compound of any one of claims 1 and 3-5, wherein R⁶ is

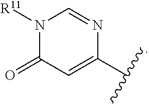

7. The compound of any one of claims 1 and 3-6, wherein R¹¹ is H or methyl.

8. The compound of any one of claims 1 and 3-7, wherein R⁷ is phenyl, alkyl, or cycloalkyl, each of which is optionally substituted with one or more R⁴.

9. The compound of any one of claims 1 and 3-8, wherein R⁷ is a linear or branched, non-cyclic C₁-C₆ alkyl.

10. The compound of any one of claims 1 and 3-9, wherein R⁷ is methyl, ethyl, n-propyl, i-propyl, n-butyl, s-butyl, or t-butyl.

11. The compound of any one of claims 1 and 3-10, wherein X is CH.

12. The compound of any one of claims 1 and 3-11 wherein n is 1.

13. A compound of formula (II): 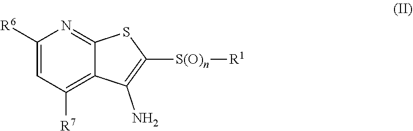
or a pharmaceutically acceptable salt, tautomer, or solvate thereof, wherein:
R¹ is C₁-C₆ alkyl, C₃-C₆ cycloalkyl, or —(C₁-C₃ alkylene)-(C₁-C₃ alkoxy);
R⁶ is 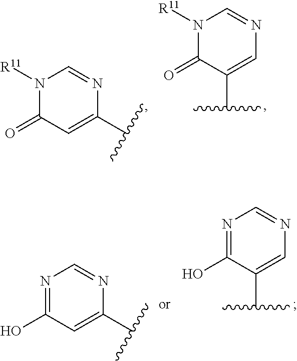
R⁷ is a linear or branched, non-cyclic C₁-C₆ alkyl;
R¹¹ is H or C₁-C₆ alkyl; and
n is 0, 1, or 2.

14. The compound of claim 1 or 13 selected from: 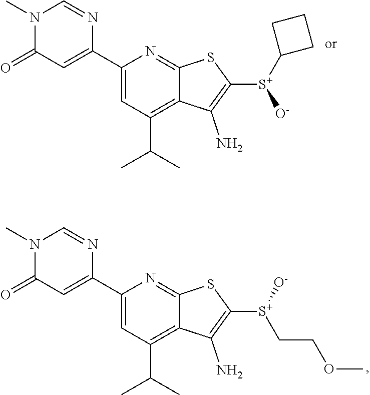
or a pharmaceutically acceptable salt, tautomer, or solvate thereof.

15. A compound of formula (III): 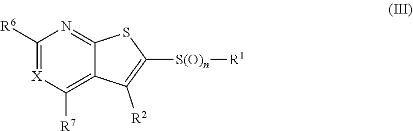
or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is 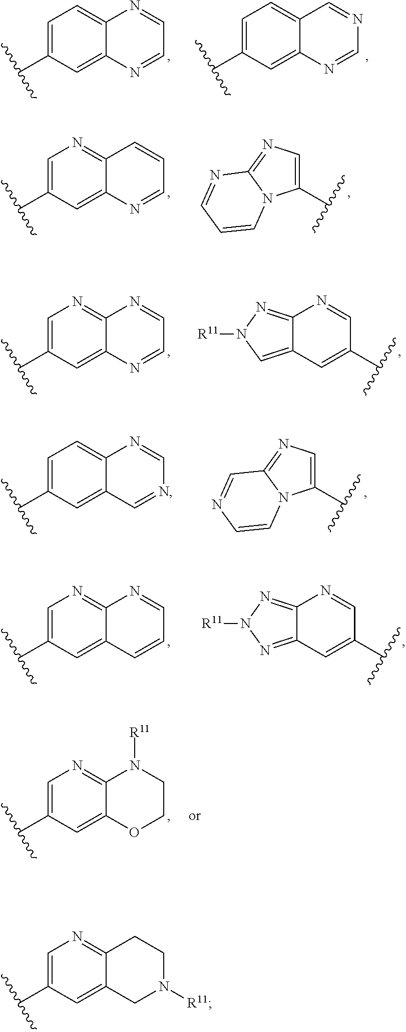
R⁷ is alkyl, haloalkyl, cycloalkyl, aryl, heterocyclyl, heteroaryl, —C(O)-alkyl,
—C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2.

16. The compound of claim 15, wherein the compound is not:

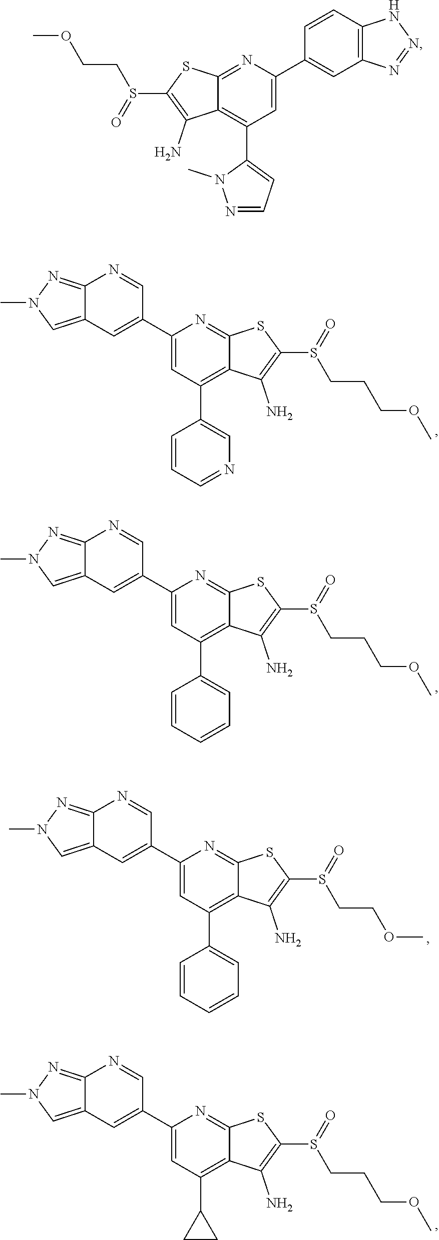

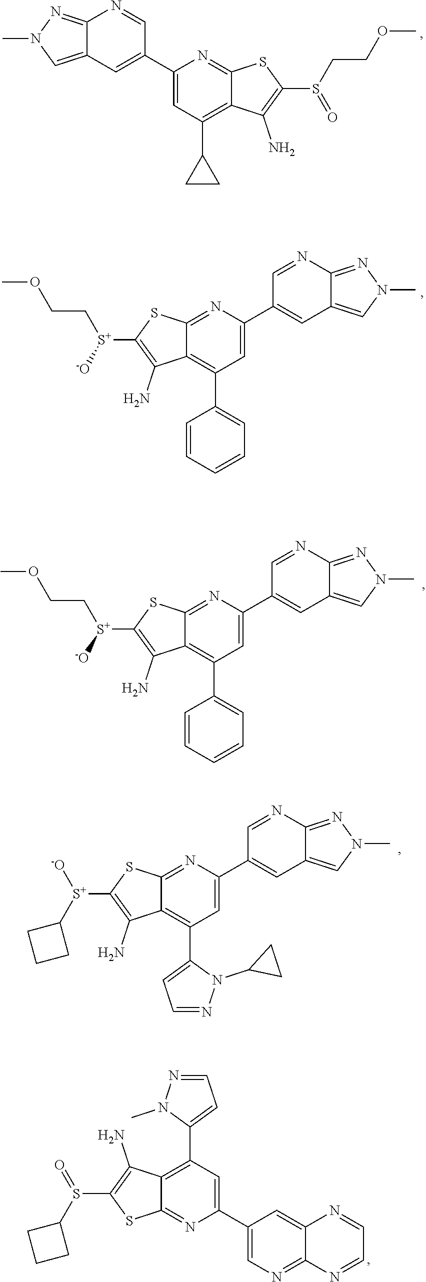

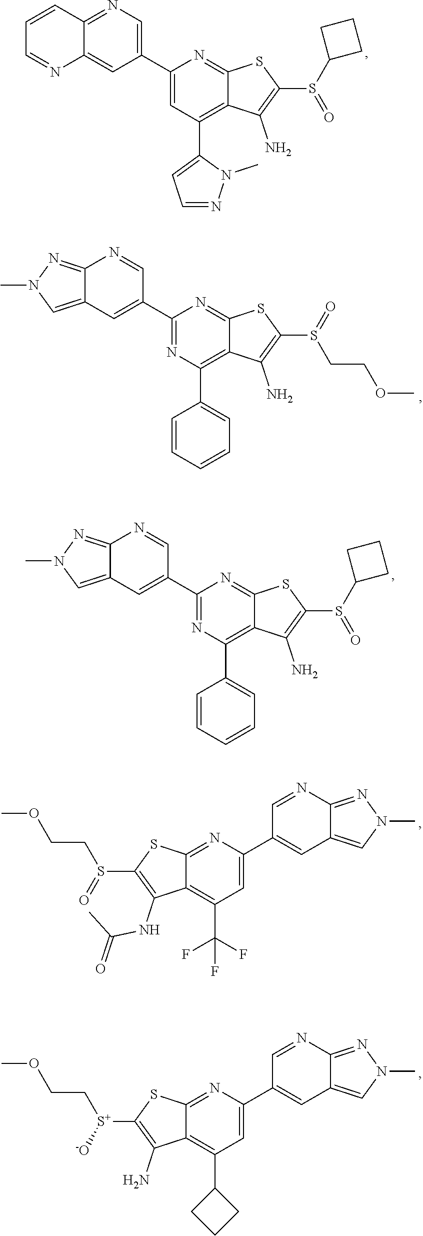

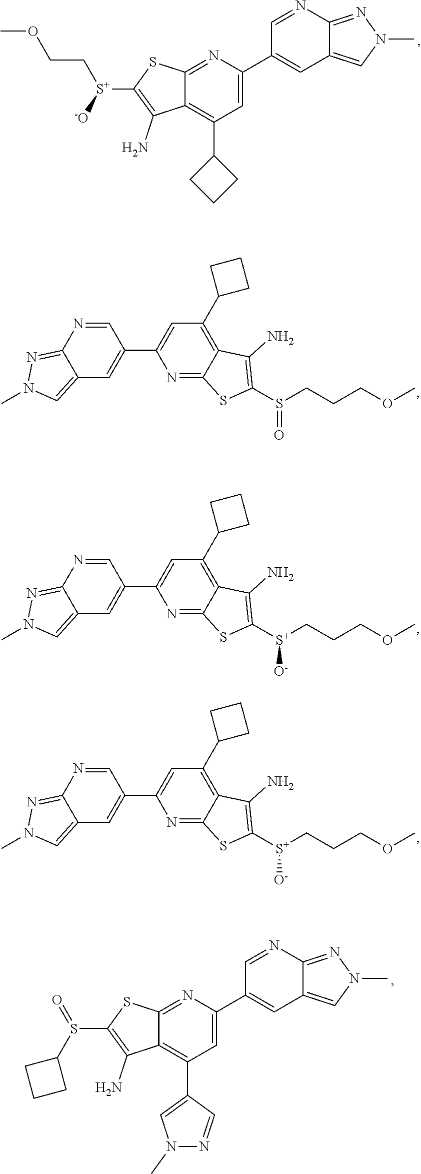

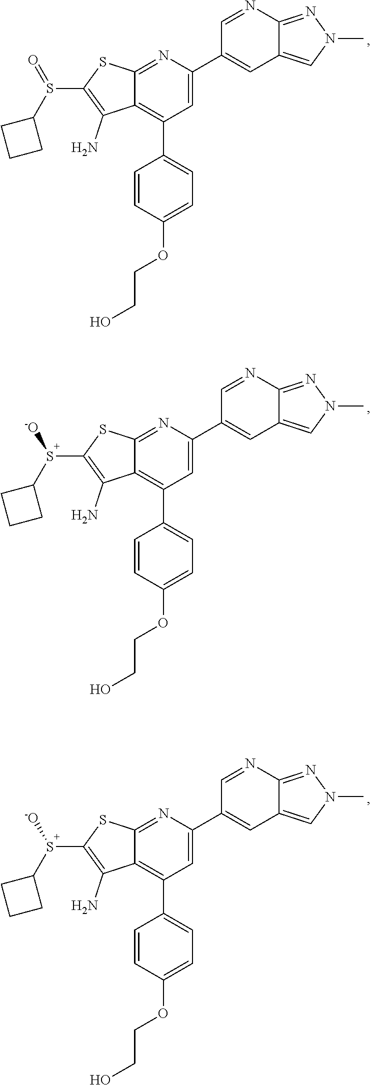

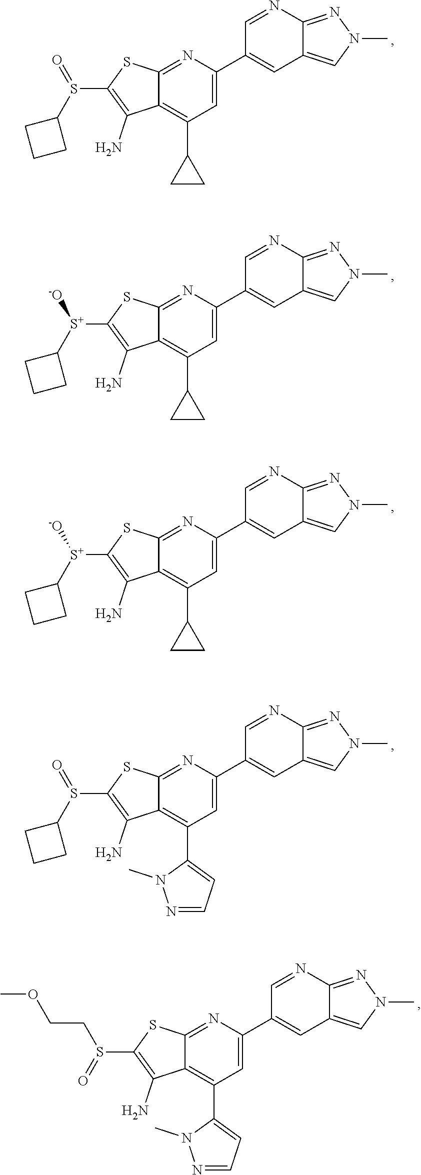

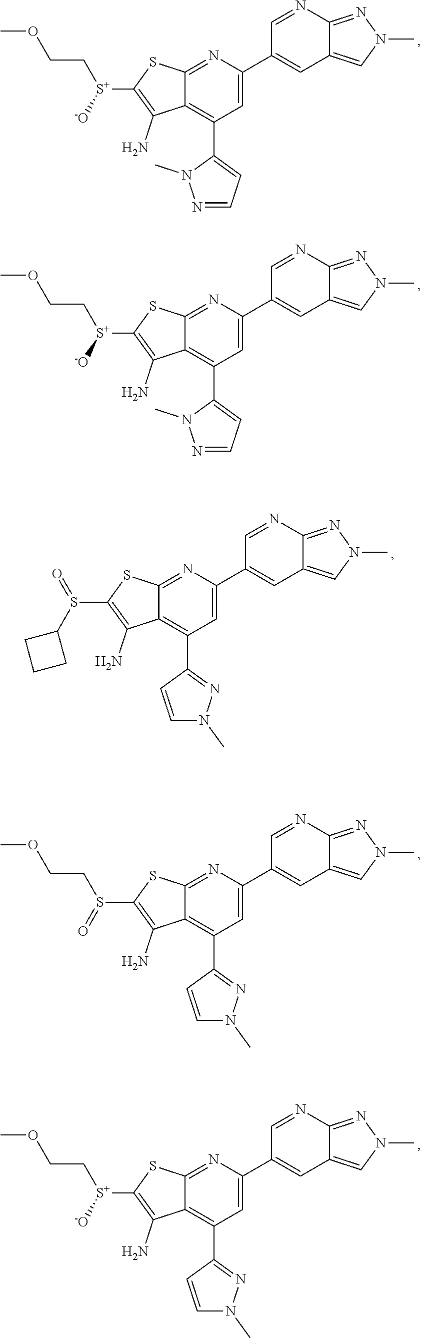

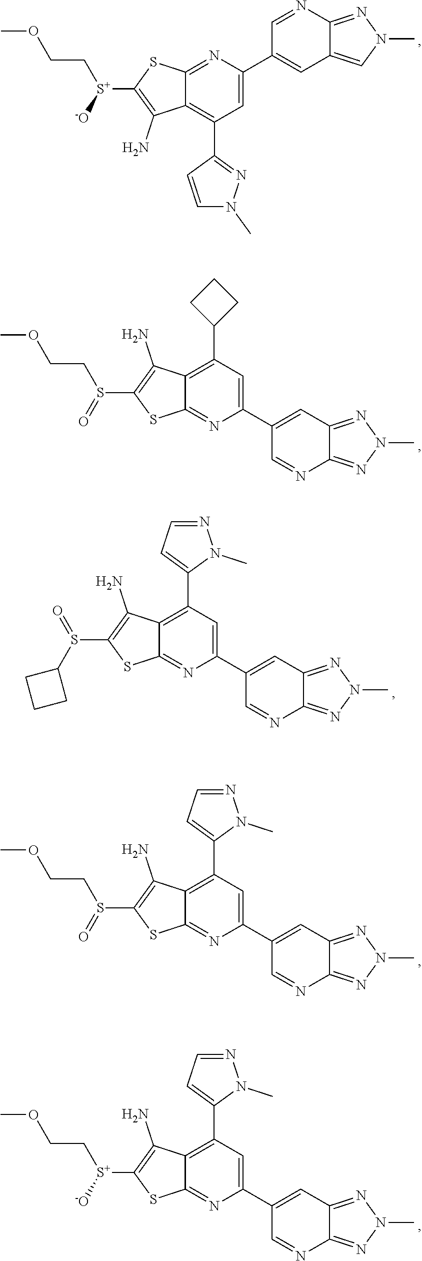

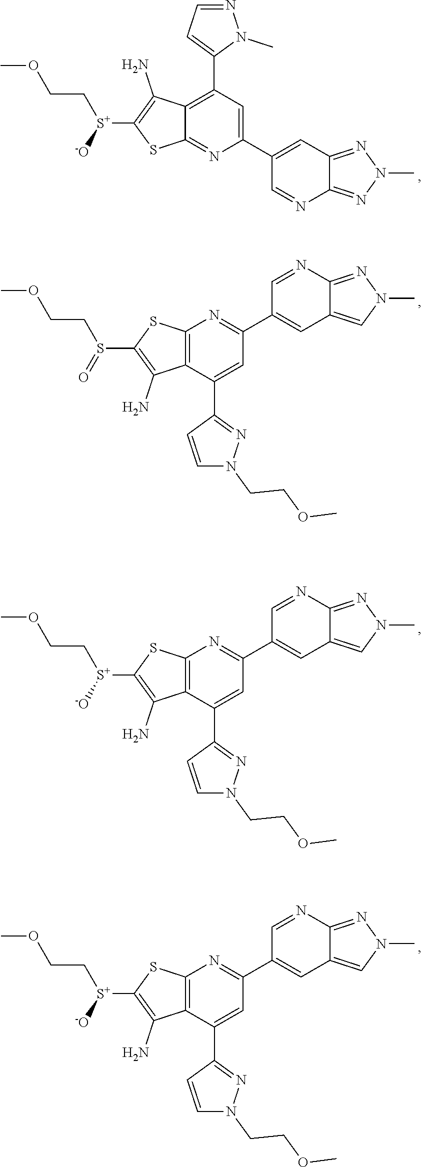

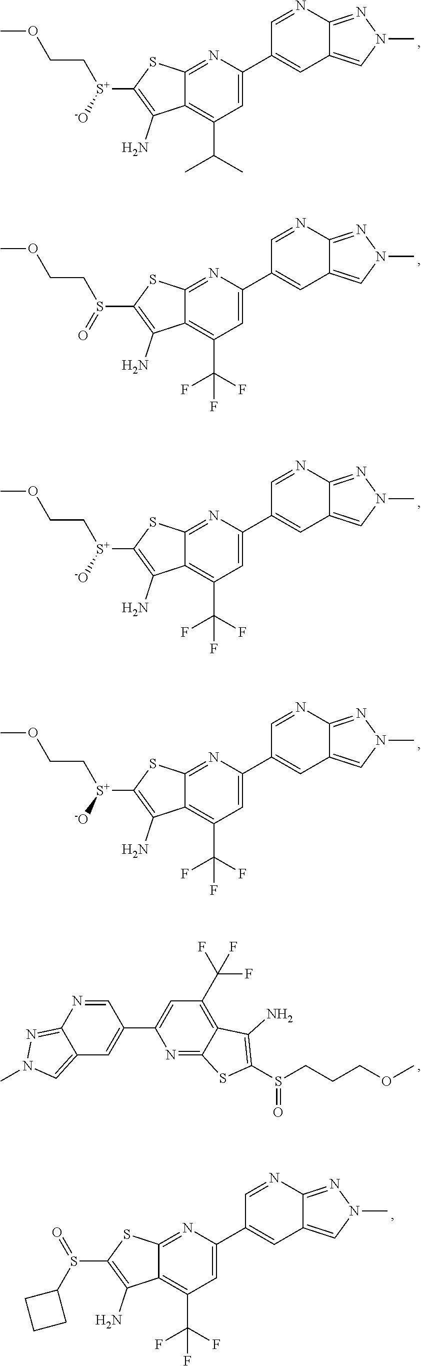

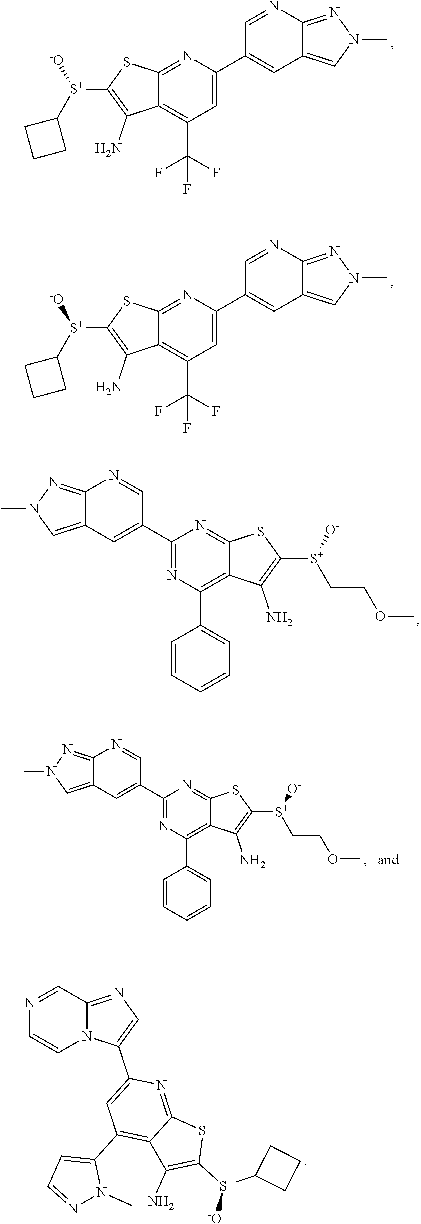

17. The compound of claim 15, wherein R¹ is C₁-C₆ alkyl, C₃-C₆ cycloalkyl, or —(C₁-C₃ alkylene)-(C₁-C₃ alkoxy).

18. The compound of claim 15 or 17, wherein R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, —(CH₂)ₚ-cyclohexyl, or —(CH₂)ₚ—OCH₃; wherein p is 1, 2, or 3.

19. The compound of any one of claims 15, 17 and 18, wherein R² is NH₂ or —CN.

20. The compound of any one of claims 15 and 17-19, wherein R⁶ is

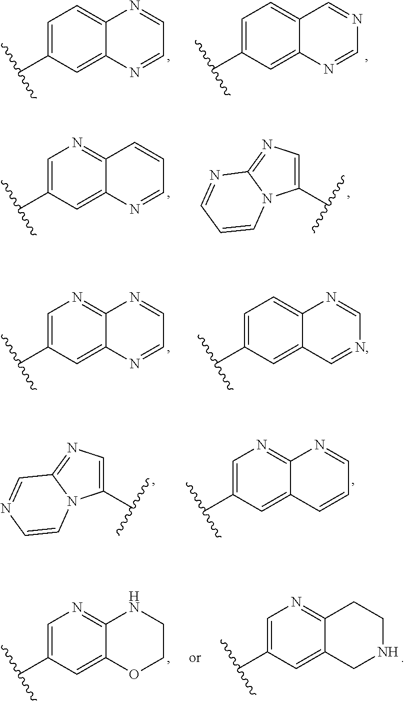

21. The compound of any one of claims 15 and 17-20, wherein R⁷ is alkyl, cycloalkyl, aryl, heterocyclyl, or heteroaryl, each of which is optionally substituted with one or more R⁴.

22. The compound of any one of claims 15 and 17-21, wherein n is 1.

23. The compound of claim 15 selected from: 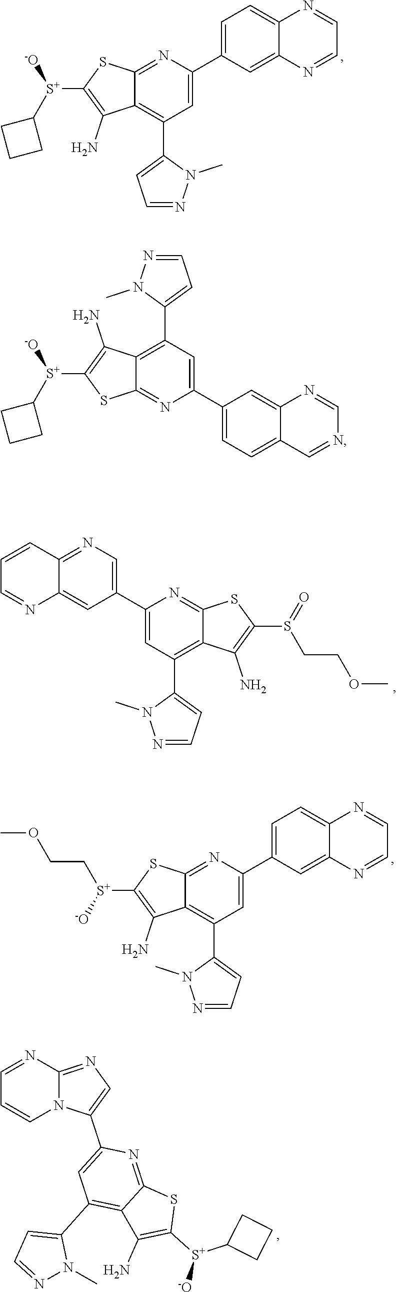 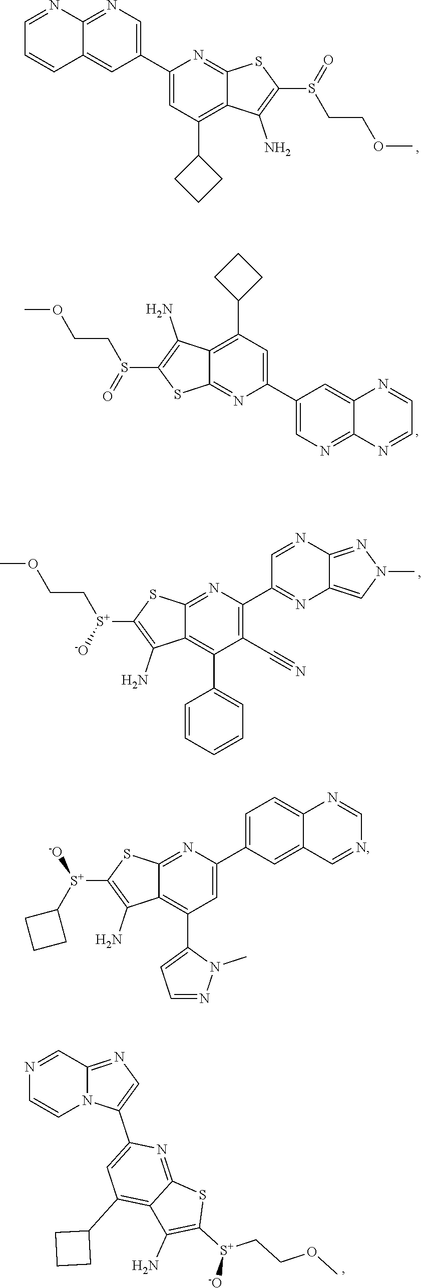 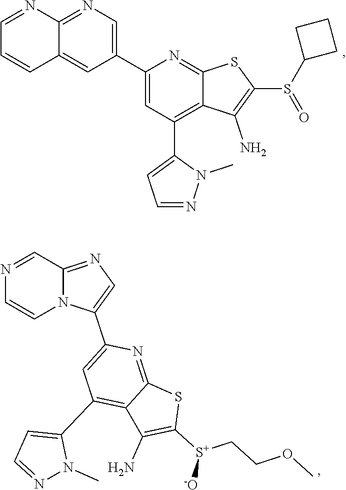 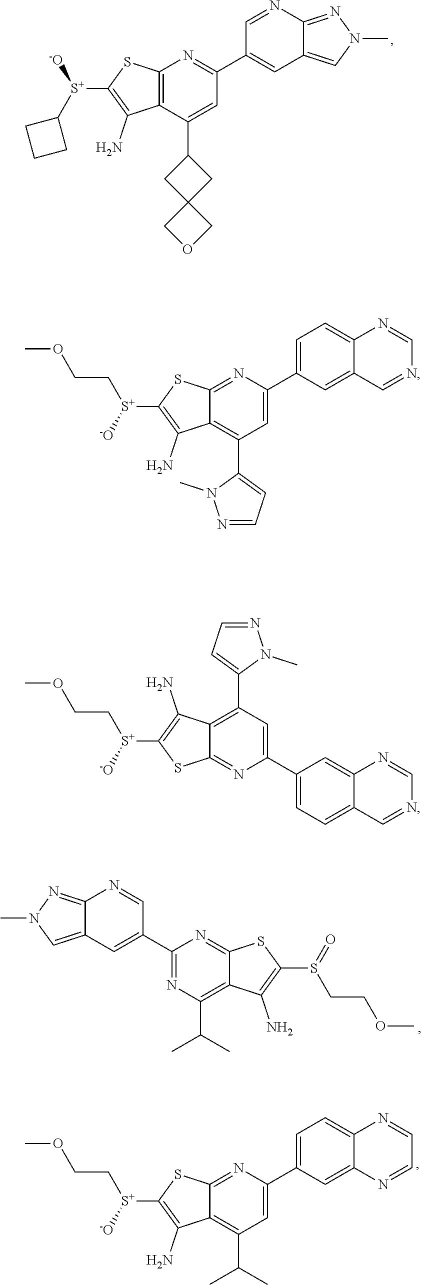 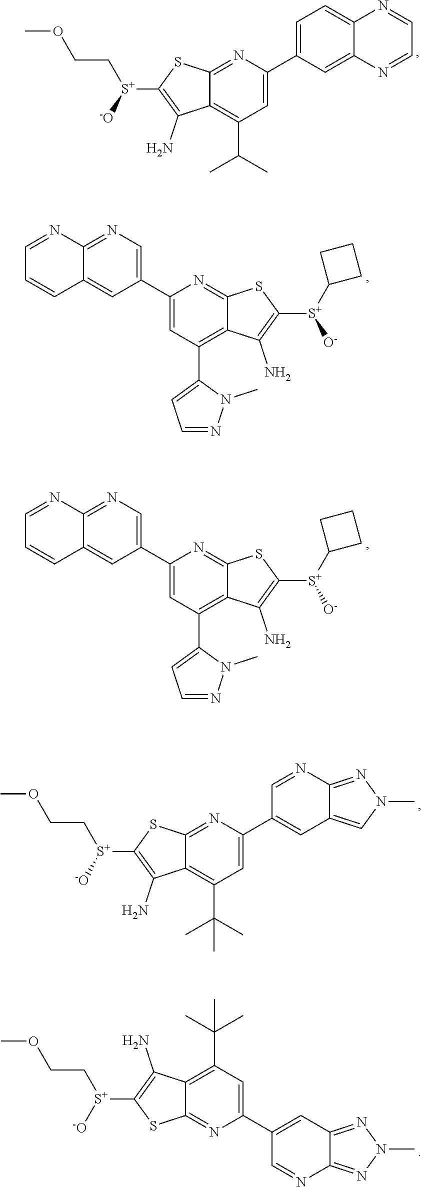 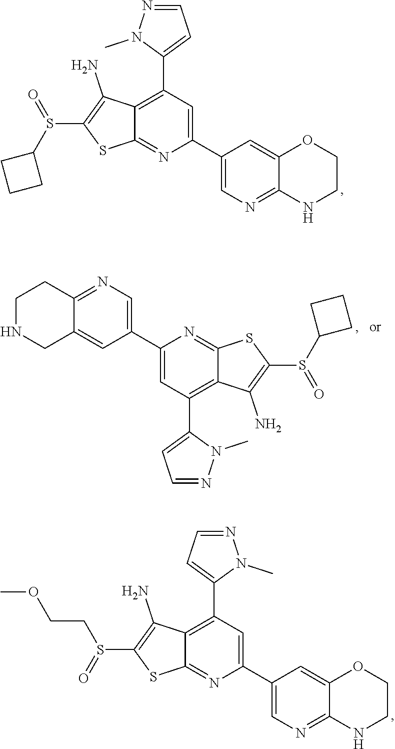 [SMILES: Cn1nccc1-c1cc(-c2cnc3c(c2)OCCN3)nc2sc(S(=O)C3CCC3)c(N)c12 ; Cn1nccc1-c1cc(-c2cnc3c(c2)CNCC3)nc2sc(S(=O)C3CCC3)c(N)c12 ; COCCS(=O)c1sc2nc(-c3cnc4c(c3)OCCN4)cc(-c3ccnn3C)c2c1N]
a pharmaceutically acceptable salt, tautomer, or solvate thereof.

24. A compound of formula (IV): 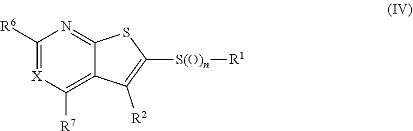
or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)(C₁-C₆ alkyl);
R⁶ is 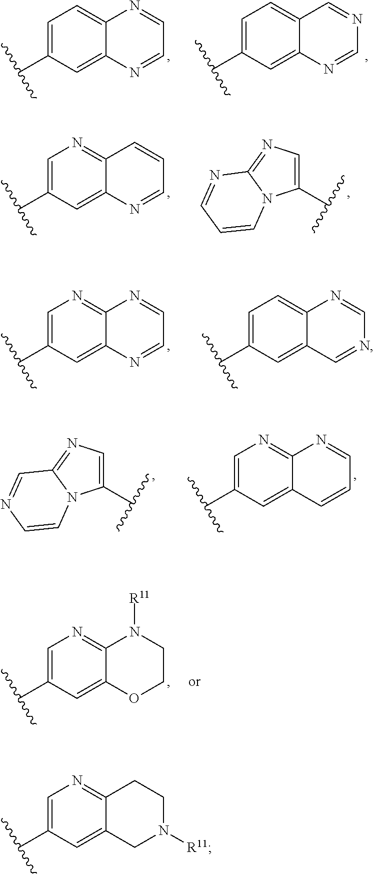
R⁷ is alkyl, haloalkyl, cycloalkyl, aryl, heterocyclyl, heteroaryl, —C(O)-alkyl,
—C(O)O-alkyl, or —C(O)NR⁵-alkyl, each of which is optionally substituted with one or more R⁴;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸, wherein when R⁴ is oxo and R⁷ is aryl or heteroaryl, oxo does not violate the valency of the aryl or the heteroaryl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
R⁸ is halogen, C₁-C₆ alkyl, or C₁-C₆ alkoxy;
R⁹ is H or C₁-C₆ alkyl;
R¹¹ is H or C₁-C₆ alkyl;
X is N or CH;
m is 0, 1, or 2; and
n is 0, 1, or 2;
wherein the compound is not:

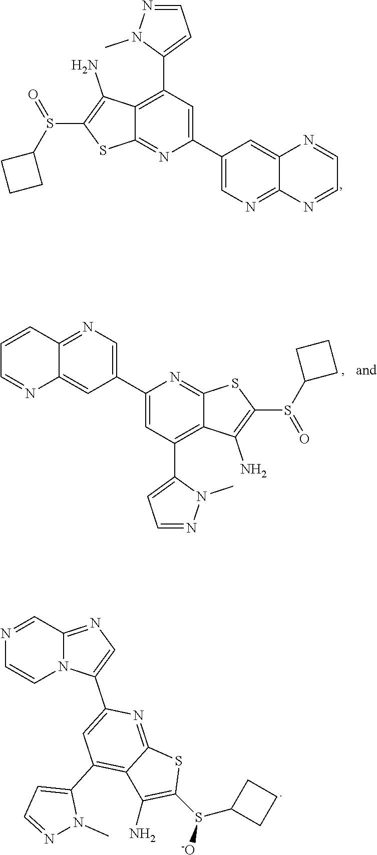

25. A compound selected from: 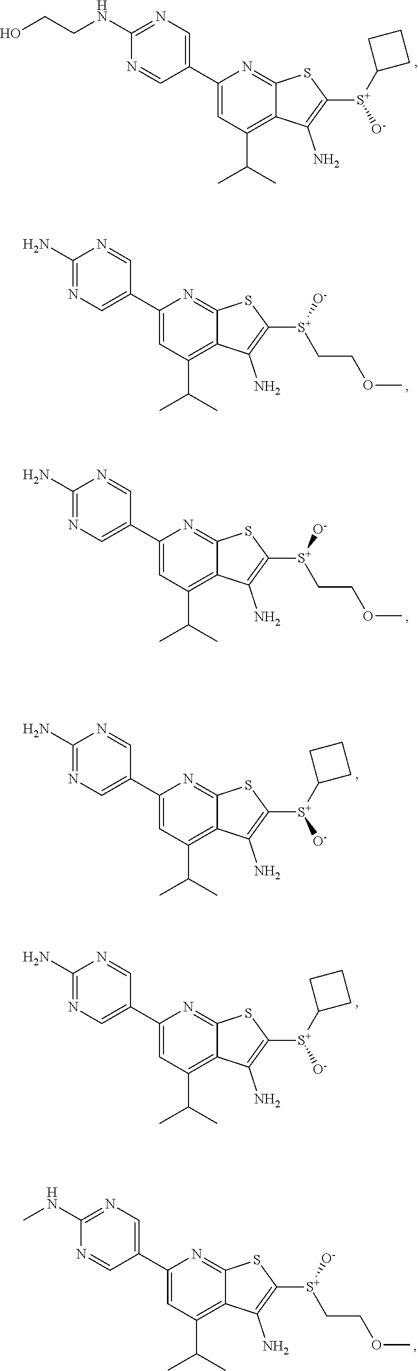 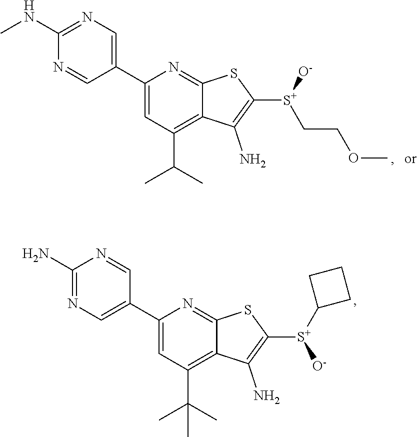
or a pharmaceutically acceptable salt, tautomer, or solvate thereof.

26. A pharmaceutical composition comprising a compound of any one of claims 1 to 25 and a pharmaceutically acceptable excipient or a carrier.

27. Use of a compound of any one of claims 1 to 25 as a short chain dehydrogenase inhibitor for inhibiting the activity of a short chain dehydrogenase enzyme.

28. Use of a compound of any one of claims 1 to 25 as a 15-PGDH inhibitor for inhibiting the activity of a 15-PGDH enzyme.

29. The use of claim 27 or 28, wherein the compound inhibits the enzymatic activity of recombinant 15-PGDH at an IC₅₀ of less than 1 or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

30. The use of claim 27 or 28, wherein the compound is administered to a tissue of a subject at an amount effective to increase prostaglandin levels in the tissue.

31. The use of claim 27 or 28, wherein the compound is provided in a topical composition.

32. The use of claim 27 or 28, wherein the compound is applied to skin of a subject to promote and/or stimulate pigmentation of the skin and/or hair growth and/or inhibiting hair loss, and/or treat skin damage or inflammation.

33. The use of claim 27 or 28, wherein the compound is administered to a subject to promote wound healing, tissue repair, and/or tissue regeneration.

34. The use of claim 27 or 28, wherein the compound is administered to a subject to treat at least one of oral ulcers, gum disease, colitis, ulcerative colitis, gastrointestinal ulcers, inflammatory bowel disease, vascular insufficiency, Raynaud's disease, Buerger's disease, diabetic neuropathy, pulmonary artery hypertension, cardiovascular disease, and renal disease.

35. The use of claim 27 or 28, wherein the compound is administered to a subject in combination with a prostanoid agonist for the purpose of enhancing the therapeutic effect of the agonist in prostaglandin responsive conditions.

36. The use of claim 27 or 28, wherein the compound is administered to tissue of the subject to increase tissue stem cells.

37. The use of claim 27 or 28, wherein the compound is administered to a tissue graft donor, bone marrow graft donor, and/or a hematopoietic stem cell donor to increase the fitness of a donor tissue graft, a donor bone marrow graft, and/or a donor hematopoietic stem cell graft.

38. The use of claim 27 or 28, wherein the compound is administered to bone marrow of a subject to increase stem cells in the subject.

39. The use of claim 27 or 28, wherein the compound is administered to bone marrow of a subject to increase the fitness of the marrow as a donor graft.

40. The use of claim 27 or 28, wherein the compound is administered to a preparation of hematopoietic stem cells of a subject to increase the fitness of the stem cell preparation as a donor graft.

41. The use of claim 27 or 28, wherein the compound is administered to a preparation of peripheral blood hematopoietic stem cells of a subject to increase the fitness of the stem cell preparation as a donor graft.

42. The use of claim 27 or 28, wherein the compound is administered to a preparation of umbilical cord blood stem cells to increase the fitness of the stem cell preparation as a donor graft.

43. The use of claim 27 or 28, wherein the compound is administered to a preparation of umbilical cord blood stem cells to decrease the number of units of umbilical cord blood required for transplantation.

44. The use of claim 27 or 28, wherein the compound is administered to a subject to mitigate tissue graft rejection.

45. The use of claim 27 or 28, wherein the compound is administered to a subject to enhance tissue and/or bone marrow graft engraftment.

46. The use of claim 27 or 28, wherein the compound is administered to a subject to enhance bone marrow graft engraftment, following treatment of the subject or the marrow of the subject with radiation therapy, chemotherapy, or immunosuppressive therapy.

47. The use of claim 27 or 28, wherein the compound is administered to a subject to enhance engraftment of a progenitor stem cell graft, hematopoietic stem cell graft, or an umbilical cord blood stem cell graft.

48. The use of claim 27 or 28, wherein the compound is administered to a subject to enhance engraftment of a hematopoietic stem cell graft, or an umbilical cord stem cell graft, following treatment of the subject or the marrow of the subject with radiation therapy, chemotherapy, or immunosuppressive therapy.

49. The use of claim 27 or 28, wherein the compound is administered to a subject in order to decrease the number of units of umbilical cord blood required for transplantation into the subject.

50. The use of claim 27 or 28, wherein the compound is administered to a recipient of a tissue graft transplant, bone marrow transplant, and/or hematopoietic stem cell transplant, or of an umbilical cord stem cell transplant, in order to decrease the administration of other treatments or growth factors.

51. The use of claim 27 or 28, wherein the compound is administered to a subject or to a tissue graft of a subject to mitigate graft rejection.

52. The use of claim 27 or 28, wherein the compound is administered to a subject or to a tissue graft of a subject to enhance graft engraftment.

53. The use of claim 27 or 28, wherein the compound is administered to a subject or to a tissue graft of a subject to enhance graft engraftment following treatment of the subject or the marrow of the subject with radiation therapy, chemotherapy, or immunosuppressive therapy.

54. The use of claim 27 or 28, wherein the compound is administered to a subject or to the bone marrow of a subject to confer resistance to toxic or lethal effects of exposure to radiation.

55. The use of claim 27 or 28, wherein the compound is administered to a subject or to the bone marrow of a subject to confer resistance to the toxic effect of Cytoxan, the toxic effect of fludarabine, the toxic effect of chemotherapy, or the toxic effect of immunosuppressive therapy.

56. The use of claim 27 or 28, wherein the compound is administered to a subject or to the bone marrow of a subject to decrease infection.

57. The use of claim 27 or 28, wherein the compound is administered to a subject to increase neutrophil counts following a hematopoetic cell transplant with bone marrow, hematopoetic stem cells, or umbilical cord blood.

58. The use of claim 27 or 28, wherein the compound is administered to a subject to increase neutrophil counts in a subject with neutropia following chemotherapy administration or radiation therapy.

59. The use of claim 27 or 28, wherein the compound is administered to a subject to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections.

60. The use of claim 27 or 28, wherein the compound is administered to a subject to increase neutrophil counts in a subject with neutropia.

61. The use of claim 27 or 28, wherein the compound is administered to a subject to increase platelet counts following a hematopoetic cell transplant with bone marrow, hematopoetic stem cells, or umbilical cord blood.

62. The use of claim 27 or 28, wherein the compound is administered to a subject to increase platelet counts in a subject with thrombocytopenia following chemotherapy administration or radiation therapy.

63. The use of claim 27 or 28, wherein the compound is administered to a subject to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections.

64. The use of claim 27 or 28, wherein the compound is administered to a subject to increase platelet counts in a subject with thrombocytopenia.

65. The use of claims of 27 or 28, wherein the compound is administered to a subject to increase red blood cell counts, or hematocrit, or hemoglobin level, following a hematopoetic cell transplant with bone marrow, hematopoetic stem cells, or umbilical cord blood.

66. The use of claim 27 or 28, wherein the compound is administered to a subject to increase red blood cell counts, or hematocrit, or hemoglobin level in a subject with anemia following chemotherapy administration or radiation therapy.

67. The use of claim 27 or 28, wherein the compound is administered to a subject to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause.

68. The use of claim 27 or 28, wherein the compound is administered to a subject to increase red blood cell counts, or hematocrit, or hemoglobin level in a subject with anemia.

69. The use of claim 27 or 28, wherein the compound is administered to a subject to increase bone marrow stem cells, following a hematopoetic cell transplant with bone marrow, hematopoetic stem cells, or umbilical cord blood.

70. The use of claim 27 or 28, wherein the compound is administered to a subject to increase bone marrow stem cells in a subject following chemotherapy administration or radiation therapy.

71. The use of claim 27 or 28, wherein the compound is administered to a subject to increase bone marrow stem cells in a subject with aplastic anemia, myelodysplasia, myelofibrosis, other disorder of bone marrow, drug induced cytopenias, immune cytopenias, cytopenias following viral infections, or cytopenias.

72. The use of claim 27 or 28, wherein the compound is administered to a subject to increase responsiveness to cytokines in the presence of cytopenias, with cytopenias including any of: neutropenia, thrombocytopenia, lymphocytopenia and anemia; and with cytokines having increased responsiveness potentiated by the 15-PGDH inhibitor including any of: G-CSF, GM-CSF, EPO, IL-3, IL-6, TPO, TPO-RA (thrombopoietin receptor agonist), and SCF.

73. The use of claim 27 or 28, wherein the compound is administered to a subject or the bone marrow of a subject to decrease pulmonary toxicity from radiation.

74. The use of claim 27 or 28, wherein the compound is administered to a subject to increase bone density, treat osteoporosis, promote healing of fractures, or promote healing after bone surgery or joint replacement.

75. The use of claim 27 or 28, wherein the compound is administered to a subject to promote healing of bone to bone implants, bone to artificial implants, dental implants, and bone grafts.

76. The use of claim 27 or 28, wherein the compound is administered to a subject or to the intestine of a subject to increase stem cells in the intestine.

77. The use of claim 27 or 28, wherein the compound is administered to a subject or to intestine of a subject to increase stem cells in the intestine and confer resistance to toxic or lethal effects of exposure to radiation or the toxic, lethal, or mucositis effects resultant from treatment with chemotherapy.

78. The use of claim 27 or 28, wherein the compound is administered to a subject or to the intestines of a subject to confer resistance to toxic or lethal effects of exposure to radiation or the toxic, lethal, or mucositis effects resultant from treatment with chemotherapy.

79. The use of claim 27 or 28, wherein the compound is administered to a subject or to intestine of a subject as a treatment for colitis, ulcerative colitis, or inflammatory bowel disease.

80. The use of claim 27 or 28, wherein the compound is administered to a subject to increase liver regeneration following liver surgery, following live liver donation, following liver transplantation, or following liver injury by toxins.

81. The use of claim 27 or 28, wherein the compound is administered to a subject to promote recovery from or resistance to liver toxins, including acetaminophen and related compounds.

82. The use of claim 27 or 28, wherein the compound is administered to a subject to treat erectile dysfunction.

83. The use of claim 27 or 28, wherein the compound is administered to inhibit at least one of the growth, proliferation, or metastasis of 15-PGDH expressing cancers.

84. A method of treating a subject in need of cell therapy comprising administering to the subject a therapeutically effective amount of a preparation comprising human hematopoietic stem cell administered a compound of any one of claims 1 to 25 and/or a therapeutic composition comprising human hematopoietic stem cells and a compound of any one of claims 1 to 25.

85. The method of claim 84, further comprising administering of any one of claims 1 to 71 to a subject who has received human hematopoietic stem cells and/or has received the preparation and/or the therapeutic composition.

86. The method of claim 84, wherein the subject has acute myelogenous leukemia (AML), acute lymphoblastic leukemia (ALL), chronic myelogenous leukemia (CML), chronic lymphocytic leukemia (CLL), juvenile myelomonocytic leukemia, Hodgkin's lymphoma, non-Hodgkin's lymphoma, multiple myeloma, severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, mucopolysaccharidoses, or Diamond Blackfan anemia.

87. A method of treating a subject having at least one symptom associated with an ischemic tissue or a tissue damaged by ischemia comprising administering to the subject a therapeutically effective amount of a preparation comprising human hematopoietic stem cell administered a compound of any one of claims 1 to 25 and/or a therapeutic composition comprising human hematopoietic stem cells and a compound of any one of claims 1 to 25.

88. The method of claim 87, wherein the ischemia is associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

89. A method of increasing neutrophils in a subject in need thereof, the method comprising administering to the subject a compound of any one of claims 1 to 25.

90. The method of claim 89, further comprising administering a hematopoietic cytokine in combination with the compound.

91. A method increasing numbers of and/or of mobilizing peripheral blood hematopoietic stem cells in a subject in need thereof, the method comprising administering to the subject a compound of any one of claims 1 to 25.

92. The method of claim 91, further comprising administering G-CSF in combination with the compound.

93. The method of claim 92, further comprising administering a hematopoietic cytokine in combination with the compound.

94. The method of claim 93, further comprising administering Plerixafor in combination with the compound.

95. The method of any of claims 91 to 94, wherein increasing numbers of and/or of mobilizing peripheral blood hematopoietic stem cells is used in hematopoietic stem cell transplantation.

96. A method of increasing numbers of hematopoietic stem cells in blood or bone marrow, the method comprising: administering to blood or bone marrow of the subject a compound of any one of claims 1 to 25.

97. The method of claim 96, further comprising administering G-CSF in combination with the compound.

98. The method of claim 96, further comprising administering a hematopoietic cytokine in combination with the compound.

99. The method of claim 96, further comprising administering Plerixafor in combination with the compound.

100. A method of treating or preventing a fibrotic disease, disorder or condition in a subject in need thereof, the method comprising administering to the subject a therapeutically effective amount of a compound of any one of claims 1 to 25.

101. The method of claim 100, wherein the fibrotic disease, disorder or condition is characterized, in whole or in part, by the excess production of fibrous material, including excess production of fibrotic material within the extracellular matrix, or the replacement of normal tissue elements by abnormal, non-functional, and/or excessive accumulation of matrix-associated components.

102. The method of claim 100, wherein the fibrotic disease, disorder, or condition is selected from the group consisting of systemic sclerosis, multifocal fibrosclerosis, nephrogenic systemic fibrosis, scleroderma, sclerodermatous graft-vs-host-disease, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis, bone marrow fibrosis, drug induced ergotism, glioblastoma in Li-Fraumeni syndrome, sporadic glioblastoma, myeloid leukemia, acute myelogenous leukemia, myelodysplastic syndrome, myeloproferative syndrome, gynecological cancer, Kaposi's sarcoma, Hansen's disease, collagenous colitis, acute fibrosis, and organ specific fibrosis.

103. The method of claim 100, wherein the fibrotic disease, disorder, or condition comprises lung fibrosis.

104. The method of claim 103, wherein the lung fibrosis is selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations there.

105. The method of claim 104, wherein the lung fibrosis is cystic fibrosis.

106. The method of claim 104, wherein the fibrotic disease, disorder or condition comprises kidney fibrosis.

107. The method of claim 104, wherein the fibrotic disease, disorder or condition comprises liver fibrosis.

108. The method of claim 107, wherein the liver fibrosis results from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

109. The method of claim 100, wherein the fibrotic disease, disorder or condition comprises heart fibrosis.

110. The method of claim 100, wherein the fibrotic disease, disorder or condition is systemic sclerosis.

111. The method of claim 100, wherein the fibrotic disease, disorder or condition is caused by post-surgical adhesion formation.

112. The method of claim 100, wherein the compound is administered at amount effective to reduce or inhibit collagen deposition, inflammatory cytokine expression, and/or inflammatory cell infiltration in a tissue or organ of the subject being treated.

113. A method of treating intestinal, gastrointestinal, or bowel disorders in a subject in need thereof, the method comprising administering to the subject a therapeutically effective amount of a compound of any one of claims 1 to 25 alone or in combination with a corticosteroid and/or a tumor necrosis factor α (TNFα) inhibitor.

114. The method of claim 113, wherein the disorder comprises at least one of oral ulcers, gum disease, gastritis, colitis, ulcerative colitis, gastric ulcers, inflammatory bowel disease, and Crohn's disease.

115. The method of claim 113, wherein the disease is inflammatory bowel disease.

116. The method of claim 113, wherein the corticosteroid induces 15-PGDH expression.

117. The method of claim 113, wherein the 15-PGDH inhibitor is effective to attenuate corticosteroid induced adverse and/or cytotoxic effects in a subject, or to increase therapeutic efficacy.

118. The method of claim 113, wherein the corticosteroid is selected from the group consisting of aclovate, alclometasone dipropionate, amcinafel, amcinafide, amcinonide, aristocort A, augmented betamethasone dipropionate, beclamethasone, beclopmethasone dipropionate, betamethasone, betamethasone benzoate, betamethasone-17-benzoate, betamethasone dipropionate, betamethasone sodium phosphate and acetate, betamethasone valerate, betamethasone-17-valerate, chloroprednisone, clobetasol propionate, clobetasone propionate, clocortelone, cordran, corticosterone, cortisol, cortisol acetate, cortisol cypionate, cortisol sodium phosphate, cortisol sodium succinate, cortisone, cortisone acetate, cortodoxone, cyclocort, deflazacort, defluprednate, descinolone, desonide, desowen, desoximetasone, desoxycorticosterone acetate, desoxycorticosterone pivalate, 11-desoxycortisol, dexamethasone, dexamethasone acetate, dexamethasone sodium phosphate, dichlorisone, diflorasone diacetate, dihydroxycortisone, diprolen, diprolene, diprosone, esters of betamethasone, florone, flucetonide, flucloronide, flucortolone, fludrocortisone, fludrocortisone acetate, flumethalone, flumethasone, flumethasone pivalate, flunisolide, fluocinolone acetonide, fluocinolone acetonide acetate, fluocinonide, fluorametholone, fluorocortisone, fluperolone, fluprednisolone, flurandrenolide, fluroandrenolone acetonide, fluticasone propionate, fuprednisolone, halcinonide, halobetasol propionate, halog, hydrocortamate, hydrocortisone, hydrocortisone acetate, hydrocortisone butyrate, hydrocortisone valerate, hydrocortisone-17-valerate, kenalog, lidex, locold, locorten, maxiflor, medrysone, meprednisone, methylprednisolone, 6.alpha.-methylprednisolone, methylprednisolone acetate, methylprednisolone sodium succinate, methylprednisone, mometasone furoate, paramethasone, paramethasone acetate, prednisone, prednisone, prednisolone, prednisolone acetate, prednisolone sodium phosphate, prednisolone sodium succinate, prednisolone tebutate, prednisone, psorcon, synalar, temovate, tetrahydrocortisol, topicort, topicort LP, triamcinolone, triamcinolone acetonide, triamcinolone diacetate, triamcinolone hexacotonide, tridesilone, valisone, and westcort.

119. A method of treating intestinal, gastrointestinal, or bowel disorders in a subject in need thereof, the method comprising:
administering to the subject therapeutically effective amounts of a compound of any one of claims 1 to 25 and a corticosteroid.

120. The method of claim 119, wherein the disorder comprises at least one of oral ulcers, gum disease, gastritis, colitis, ulcerative colitis, gastric ulcers, inflammatory bowel disease, and Crohn's disease.

121. The method of claim 119, wherein the disorder comprises inflammation of the esophagus, inflammation of the glottis, inflammation of the epiglottis, inflammation of the tonsils, inflammation of the oropharynx, eosinophilic esophagitis, gastroesophageal reflux disease (GERD), non-erosive reflux disease (NERD), erosive esophagitis, Barrett's esophagus, eosinophilic gastroenteritis, hypereosinophilic syndrome, corrosive (caustic) chemical esophagitis, radiation-induced esophagitis, chemotherapy-induced esophagitis, transient drug-induced esophagitis, persistent drug-induced esophagitis, Crohn's disease of the esophagus, and pseudomembranous esophagitis.

122. A method of treating inflammation and/or reducing the activity of the immune system in a subject in need thereof, the method comprising:
administering to the subject a therapeutically effective amounts of a compound of any one of claims 1 to 25 and a corticosteroid.

123. The method of claim 122, wherein the inflammation and/or immune system activity is associated with and/or results from atopic dermatitis, psoriasis, eczematous dermatitis, nummular dermatitis, irritant contact dermatitis, allergic contact dermatitis, seborrheic dermatitis, stasis dermatitis, and other steroid responsive dermatoses, acne vulgaris, alopecia, alopecia greata, vitiligo, eczema, xerotic eczema, keratosis pilaris, lichen planus, lichen sclerosus, lichen striatus, lichen simplex chronicus, prurigo nodularis, discoid lupus erythematosus, lymphocytic infiltrate of Jessner/Kanof, lymphacytoma cutis, pyoderma gangrenosum, pruritis ani, sarcoidosis, chondrodermatitis nodularis helices, keloids, hypertrophic scars, pretibial myxedema, other infiltrative dermatological disorders, granuloma annulare, necrobiosis lipoidica diabeticorum, sarcoidosis, other noninfectious granulomas, scleroderma, scleroderma sine scleroderma, systemic lupus erythematosus, systemic vasculitides, leukocytoclastic vasculitis, polyarteritis nodosa, Churg-Strauss syndrome, and rheumatoid vasculitis.

124. A method for the treatment of glucocorticoid insensitivity, restoring corticosteroid sensitivity, enhancing glucocorticoid sensitivity or reversing the glucocorticoid insensitivity in a subject experiencing corticosteroid dependence or corticoid resistance or unresponsiveness or intolerance to corticosteroids, comprising:
administering a pharmaceutical composition comprising a compound of any one of claims 1 to 25 in combination with a corticosteroid to the subject exhibiting one or more glucocorticoid insensitivity related conditions, wherein the glucocorticoid insensitivity related conditions comprise a range of immune-inflammatory disorders/diseases treated with steroids when the therapy fails to achieve disease control or is not effective or intolerant or dependent to corticosteroids, and combinations thereof.

125. The use of claim 27 or 28, the compound being administered ex vivo to a tissue graft donor, bone marrow graft donor, and/or a hematopoietic stem cell donor to increase the fitness of a donor tissue graft, a donor bone marrow graft, and/or a donor hematopoietic stem cell graft.
